(19) United States
(12) Patent Application Publication
(10) Pub. No.: US 2025/0223319 A1
(43) Pub. Date: Jul. 10, 2025

(54) IMPROVED CHIMERIC POLYPEPTIDES AND USES THEREOF

(71) Applicant: THE UNIVERSITY OF QUEENSLAND, St. Lucia (AU)

(72) Inventors: Keith CHAPPELL, Kenmore, Queensland (AU); Daniel WATTERSON, Taringa, Queensland (AU); Nicolas TARDIOTA, Norman Park, Queensland (AU); Mark Andrew SCHEMBRI, Middle Park, Queensland (AU)

(73) Assignee: THE UNIVERSITY OF QUEENSLAND, St. Lucia (AU)

(21) Appl. No.: 18/850,709

(22) PCT Filed: Mar. 31, 2023
(86) PCT No.: PCT/IB2023/053263

§ 371 Date: Sep. 25, 2024

(30) Foreign Application Priority Data
Mar. 31, 2022 (EP) 22166085.5

(51) Int. Cl. C07K 14/005; A61K 39/00; A61K 39/02; A61K 39/095; A61K 39/102; A61K 39/108; A61K 39/12; A61K 39/215; C07K 14/195; C07K 14/22; C07K 14/245; C07K 14/285

(52) U.S. Cl. CPC C07K 14/005; A61K 39/0258; A61K 39/0291; A61K 39/095; A61K 39/102; A61K 39/12; A61K 39/215; C07K 14/195; C07K 14/22; C07K 14/245; C07K 14/285; A61K 2039/53; C07K 2319/35; C12N 2760/18522; C12N 2760/18534; C12N 2770/20022; C12N 2770/20034

(57) ABSTRACT

The present disclosure relates generally to chimeric polypeptides that comprise a microbial polypeptide (preferably (a) a virion surfaced exposed portion of an enveloped viral fusion protein or (b) a bacterial outer membrane polypeptide) and a heterologous structure-stabilizing moiety, and to complexes comprising those chimeric polypeptides. The present disclosure also relates to the use of these chimeric polypeptides and complexes thereof in compositions and methods for eliciting an immune response to a microbial polypeptide (preferably a fusion protein of an enveloped virus or a bacterial outer membrane polypeptide), or to respective complexes thereof and/or for treating or preventing related microbial infection (preferably an enveloped virus infection or a bacterial infection). Moreover, the disclosure further relates to compositions and methods for producing an antigen-binding molecule that specifically binds to such a microbial polypeptide or a complex thereof (preferably to an enveloped viral fusion protein or a complex thereof, or to a bacterial outer membrane polypeptide or a complex thereof).

### CROSS-REFERENCE TO RELATED APPLICATIONS

The present application claims the benefit of priority from the earlier European patent application EP 22 166 085.5 which was filed on Mar. 31, 2022, together with a sequence listing. The contents of this earlier application and the accompanying sequence listing are incorporated herein by reference in their entirety.

### FIELD

The disclosure relates generally to chimeric polypeptides that comprise a microbial polypeptide (preferably (a) a virion surfaced exposed portion of an enveloped viral fusion protein or (b) a bacterial outer membrane polypeptide) and a heterologous structure-stabilizing moiety, and to complexes comprising those chimeric polypeptides. The present disclosure also relates to the use of these chimeric polypeptides and complexes thereof in compositions and methods for eliciting an immune response to a microbial polypeptide (preferably a fusion protein of an enveloped virus or a bacterial outer membrane polypeptide), or to respective complexes thereof and/or for treating or preventing related microbial infection (preferably an enveloped virus infection or a bacterial infection). Moreover, the disclosure further relates to compositions and methods for producing an antigen-binding molecule that specifically binds to such a microbial polypeptide or a complex thereof (preferably to an enveloped viral fusion protein or a complex thereof, or to a bacterial outer membrane polypeptide or a complex thereof).

### BACKGROUND

Enveloped viruses, such as respiratory syncytial virus (RSV), influenza virus and human immunodeficiency virus (HIV) require fusion of viral membrane with a host cell's membrane to enter and infect the host cell. Viral fusion proteins facilitate this process by undergoing energy favorable structural rearrangements from a metastable ‘pre-fusion’-conformation to a highly stable ‘post-fusion’-conformation. This structural change drives fusion of the virus and host cell membranes resulting in the release of viral genome into the host cell.

Viral fusion proteins are currently classified into three major classes based on their individual structural architecture and molecular features that drive the fusion process. Class I and class III fusion proteins are trimeric in both their pre- and post-fusion conformations, while class II fusion proteins are dimeric in their pre-fusion conformation which is then rearranged into a trimeric post-fusion form. It is possible, however, that new classes of viral fusion proteins may be identified in the future that share some key features in common with these currently defined classes. Class I and class III fusion proteins share substantial structural features, including an N-terminal signal sequence and a C-terminal transmembrane and cytoplasmic domain. They also share similar fusion mechanisms, with the initial pre-fusion trimer undergoing partial dissociation to allow the significant structural rearrangement required to form the post-fusion trimer.

Viral fusion proteins are excellent subunit vaccine candidates, as they are the primary targets of protective neutralizing antibody responses for many medically important enveloped viruses. However, the intrinsic metastable nature of these fusion proteins especially when recombinantly expressed as soluble proteins in isolation is a major obstacle for effective subunit vaccine design. Evidence has shown that the majority of the broadly cross-reactive and potently neutralizing antibodies elicited during a natural infection target primarily the pre-fusion form, not the post-fusion form. In addition, the pre-fusion forms of viral envelope fusion proteins have been shown to contain epitopes that are either absent from the post-fusion forms, or structurally not accessible (e.g., Magro et al., 2012. Proc. Natl. Acad. Sci. USA 109(8):3089-3094). On account of these known observations, for the development of vaccines, the stabilized pre-fusion form is generally considered more desirable antigenically. However, conventional recombinant expression of these proteins typically results in premature triggering and a conformational shift to the structurally more stable post-fusion form. Strategies have since been sought which would overcome these previous impediments arising from the intrinsic structural propensities of this particular class of proteins, mostly through stabilization in their antigenically more potent pre-fusion state.

Accumulating structural information on many relevant viral targets has paved the way for structure-based design of pre-fusion state-stabilized viral fusion protein antigens. One reported advance to that end was the development of an engineered form of the respiratory syncytial virus (RSV) fusion (F) protein (RSV F), also known as DS-Cav1 Foldon, wherein a stabilization of the pre-fusion state was achieved through structure-guided introduction of stabilizing mutations, including an artificial disulfide bond (DS) and hydrophobic cavity-filling (Cav1) mutations, and C-terminal fusion to the T4 bacteriophage fibritin trimerization domain (commonly known as “Foldon”) (McLellan et al., Science. 2013; 342(6158):592-598; Zhang et al., Vaccine. 2018; 36(52):8119-8130. DOI: 10.1016/j.vaccine.2018.10.032).

However, a major drawback of such “structural vaccinology”-based approaches is their dependency on high-resolution structural data from each individual virus target, thus limiting the process for development of vaccines to targets of yet unknown structure. In view of the continued emergence of new virus variants, as prominently evident, e.g., for SARS-CoV-2 in the presently ongoing COVID-19 pandemic, there is a particular need for vaccine design strategies that can be readily applied, i.e., as a universally applicable platform technology, without prior availability of respective 3D structural information.

Seeking to address this need, some of the present inventors recently developed an approach for stabilizing viral fusion protein antigens in their pre-fusion conformation (WO 2018/176103; WO 2022/043908). Their technology is based on the finding that a viral fusion protein can be maintained in its pre-fusion form by operably connecting a heterologous moiety that comprises a pair of complementary heptad repeat regions (HRRs) downstream of the fusion protein virion surface exposed domain. These HRRs facilitate trimerization with two further chimeric polypeptide subunits, whereby the pairs of HRRs of each of the three subunits associate into a six-helix bundle structure. The resulting trimeric structure acts as a kind of ‘molecular clamp’ that ‘locks’ the fusion ectodomain polypeptide in the pre-fusion conformation, thereby inhibiting it from rearranging into a post-fusion conformation. The first generation of this technology centered on the utilization of a clamp derived from the 6-helix bundle of the human immunodeficiency virus (HIV) glycoprotein 41 (gp41).

Whereas this technology was successfully applied to the development of a SARS-CoV-2-vaccine candidate which in a subsequent phase 1 clinical trial was proven to elicit potent neutralizing antibody responses against the enveloped virus fusion protein ectodomain, it was also found that additional antibodies were generated in all recipients against the small HIV gp41-derived clamp domain. Although titers of these antibodies were low, they were sufficient to generate false-positive results on some rapid HIV point-of-care diagnostic tests. Because of this observed HIV diagnostic interference, the further development and clinical investigation of this vaccine candidate was suspended.

Hence, there is still a pressing need for new viral fusion protein subunit vaccines which circumvent the risk of HIV diagnostic interference caused by the clamp domain while having a comparable or even improved capacity to elicit neutralizing antibody responses towards the targeted antigen, and, in particular, for new vaccine designs that can be readily applied as a universal platform technology to viral targets in the absence of structural information.

Moreover, on account of the increasing threat of antibiotic resistance and the resurgence of numerous related infections, there is also an urgent demand for novel vaccines against bacterial pathogens. In recent years, bacterial outer membrane proteins have become a major interest for vaccine development, as they are the proteins which interact with the extracellular environment. A specific kind of outer membrane proteins found in many pathologically relevant Gram-negative bacteria are the so-called trimeric autotransporter adhesins (TAAs) which mediate the first adherence to host cells in the course of infection. TAAs are therefore considered to constitute important virulence factors and have thus gained increasing interest as potential vaccine targets. Most related bacterial infections, however, are not covered by any of the current vaccines, and new developments have even decelerated in the last decades (see, e.g., review by Thibau A. et al. Immunogenicity of trimeric autotransporter adhesins and their potential as vaccine targets. Med Microbiol Immunol. 2020; 209(3):243-263. doi: 10.1007/s00430-019-00649-y). There is, hence, also a need in the art for vaccines against related bacterial targets.

The present invention addresses these needs and provides related advantages as well.

### SUMMARY OF THE INVENTION

Accordingly, the invention provides, in a first aspect, a chimeric polypeptide comprising a microbial polypeptide (preferably (a) an enveloped virus fusion ectodomain polypeptide or (b) a bacterial outer membrane polypeptide) operably connected downstream to a heterologous, structure-stabilizing moiety (SSM), wherein the structure-stabilizing moiety is a polypeptide comprising, in an N- to C-terminal order, a first heptad repeat region (FHRR) and second heptad repeat region (SHRR), wherein (i) the FHRR comprises or consists of an amino acid sequence having at least 60% sequence identity to the amino acid sequence set forth in SEQ ID NO: 80 or 81, and the SHRR comprises or consists of an amino acid sequence having at least 40% sequence identity to the amino acid sequence set forth in SEQ ID NO: 82 or 83; and/or (ii) the FHRR comprises or consists of an amino acid sequence having at least 90% sequence similarity to the amino acid sequence set forth in SEQ ID NO: 80 or 81, and the SHRR comprises or consists of an amino acid sequence having at least 70% sequence similarity to the amino acid sequence set forth in SEQ ID NO: 82 or 83.

The invention provides, in a second aspect, a chimeric polypeptide comprising a first (poly)peptide operably connected downstream to a structure-stabilizing moiety, wherein said structure-stabilizing moiety is as defined in connection with the first aspect of the invention; wherein preferably the first polypeptide is a therapeutic polypeptide.

The invention provides, in a third aspect, a nucleic acid comprising a polynucleotide sequence encoding a chimeric polypeptide as defined in embodiments disclosed herein in connection with the first or second aspect of the invention.

The invention provides, in a fourth aspect, a host cell comprising the nucleic acid as defined in accordance with the third aspect of the invention.

The invention provides, in a fifth aspect, a method of producing a chimeric polypeptide complex, wherein the method comprises: combining chimeric polypeptides as defined in accordance with the first or the second aspect of the invention under conditions suitable for the formation of a chimeric polypeptide complex, whereby a chimeric polypeptide complex is produced that comprises three chimeric polypeptide subunits and is characterized by a six-helix bundle formed by homo-trimerization of the structure-stabilizing moieties of the three chimeric polypeptides.

The invention provides, in a sixth aspect, a chimeric polypeptide complex that comprises three chimeric polypeptide subunits, wherein each subunit is a chimeric polypeptide as defined in accordance with the first or the second aspect of the invention, and wherein the complex is characterized by a six-helix bundle formed by homo-trimerization of the structure-stabilizing moieties of the three chimeric polypeptides.

The invention provides, in a seventh aspect, a composition comprising a chimeric polypeptide as defined in accordance with the first or second aspect of the invention, or a chimeric polypeptide complex as defined in accordance with the sixth aspect of the invention, and a pharmaceutically acceptable carrier, diluent or adjuvant.

The invention provides, in an eighth aspect, a method of identifying an agent that binds with: a microbial polypeptide or a complex thereof, wherein the method comprises:
(i) contacting a candidate agent with a microbial polypeptide-containing chimeric polypeptide as defined in accordance with the first aspect of the invention or a microbial polypeptide-containing chimeric polypeptide complex as defined in accordance with the sixth aspect of the invention; and
(ii) detecting binding of the candidate agent to the chimeric polypeptide or chimeric polypeptide complex; wherein preferably the candidate agent is part of a compound library (e.g., small molecule or macromolecule library).

In preferred embodiments of the latter aspect, the microbial polypeptide or the complex thereof is:
(a) a fusion protein of an enveloped virus, or a complex of the fusion protein, respectively, wherein the method comprises: (i) contacting the candidate agent with an enveloped virus fusion ectodomain polypeptide-containing chimeric polypeptide as defined in accordance with the first aspect of the invention or an enveloped virus fusion ectodomain polypeptide-containing chimeric polypeptide complex as defined in accordance with the sixth aspect of the invention, wherein the enveloped virus fusion ectodomain polypeptide corresponds to the fusion protein of the enveloped virus; and (ii) detecting binding of the candidate agent to the chimeric polypeptide or chimeric polypeptide complex;
or
(b) an outer membrane polypeptide of a bacterium or a complex of the outer membrane polypeptide, respectively, wherein the method comprises: (i) contacting the candidate agent with a bacterial outer membrane polypeptide-containing chimeric polypeptide as defined in accordance with the first aspect of the invention or a bacterial outer membrane polypeptide-containing chimeric polypeptide complex as defined in accordance with the sixth aspect of the invention, wherein the bacterial outer membrane polypeptide corresponds to the outer membrane polypeptide of the bacterium; and (ii) detecting binding of the candidate agent to the chimeric polypeptide or chimeric polypeptide complex.

In preferred embodiments of the latter embodiment, the outer membrane polypeptide of a bacterium or the complex thereof is:
(a) a trimeric autotransporter adhesin (TAA) polypeptide of a bacterium, or a complex of the TAA polypeptide, respectively, wherein the method comprises: (i) contacting the candidate agent with a TAA polypeptide-containing chimeric polypeptide as defined in accordance with the first aspect of the invention or a TAA polypeptide-containing chimeric polypeptide complex as defined in accordance with the sixth aspect of the invention, wherein the TAA polypeptide corresponds to the TAA polypeptide of the bacterium; and (ii) detecting binding of the candidate agent to the chimeric polypeptide or chimeric polypeptide complex;
or
(b) a major outer membrane protein (MOMP) polypeptide of a Chlamydia bacterium or a complex thereof, respectively, wherein the method comprises: (i) contacting the candidate agent with a Chlamydia MOMP polypeptide-containing chimeric polypeptide as defined in accordance with the first aspect of the invention or a Chlamydia MOMP polypeptide-containing chimeric polypeptide complex as defined in accordance with the sixth aspect of the invention, wherein the Chlamydia MOMP polypeptide corresponds to the MOMP polypeptide of the Chlamydia bacterium; and (ii) detecting binding of the candidate agent to the chimeric polypeptide or chimeric polypeptide complex.

The invention provides, in a ninth aspect, a method of producing an antigen-binding molecule that specifically binds to:
a microbial polypeptide, or a complex thereof, wherein the method comprises:
(1) immunizing a subject with a microbial polypeptide-containing chimeric polypeptide as defined in accordance with the first aspect of the invention, or a microbial polypeptide-containing chimeric polypeptide complex as defined in accordance with the sixth aspect of the invention, or a composition thereof as defined in accordance with the seventh aspect of the invention;
(2) identifying and/or isolating a B cell from the immunised subject, which specifically binds to the microbial polypeptide or complex thereof; and
(3) producing the antigen-binding molecule expressed by that B cell.

In preferred embodiments of the latter aspect, the microbial polypeptide or the complex thereof is:
(a) an ectodomain of a fusion protein of an enveloped virus, or complex of the fusion protein, respectively, wherein the method comprises: (1) immunizing a subject with an ectodomain polypeptide-containing chimeric polypeptide as defined in accordance with the first aspect of the invention, or an ectodomain polypeptide-containing chimeric polypeptide complex as defined in accordance with the sixth aspect of the invention, or a composition thereof as defined in accordance with the seventh aspect of the invention, wherein the ectodomain polypeptide corresponds to the fusion protein of the enveloped virus; (2) identifying and/or isolating a B cell from the immunised subject, which specifically binds to the ectodomain of the fusion protein or complex thereof; and (3) producing the antigen-binding molecule expressed by that B cell; or
(b) an outer membrane polypeptide of a bacterium, or a complex of the outer membrane polypeptide, wherein the method comprises: (1) immunizing a subject with a bacterial outer membrane polypeptide-containing chimeric polypeptide as defined in accordance with the first aspect of the invention, or a bacterial outer membrane polypeptide-containing chimeric polypeptide complex as defined in accordance with the sixth aspect of the invention, or a composition thereof as defined in accordance with the seventh aspect of the invention, wherein the bacterial outer membrane polypeptide corresponds to the outer membrane polypeptide of the bacterium; (2) identifying and/or isolating a B cell from the immunised subject, which specifically binds to the bacterial outer membrane polypeptide or complex thereof; and (3) producing the antigen-binding molecule expressed by that B cell.

In preferred embodiments of the latter embodiment, the outer membrane polypeptide or the complex thereof is:
(i) a TAA polypeptide of a bacterium or a complex thereof, respectively, wherein the method comprises: (1) immunizing a subject with a TAA polypeptide-containing chimeric polypeptide as defined in accordance with the first aspect of the invention, or a TAA polypeptide-containing chimeric polypeptide complex as defined in accordance with the sixth aspect of the invention, or a composition thereof as defined in accordance with the seventh aspect of the invention, wherein the TAA polypeptide corresponds to the TAA polypeptide of the bacterium; (2) identifying and/or isolating a B cell from the immunised subject, which specifically binds to the TAA polypeptide or complex thereof; and (3) producing the antigen-binding molecule expressed by that B cell;
or
(ii) a major outer membrane protein (MOMP) polypeptide of a Chlamydia bacterium or a complex thereof, respectively, wherein the method comprises: (1) immunizing a subject with a Chlamydia MOMP polypeptide-containing chimeric polypeptide as defined in accordance with the first aspect of the invention, or a Chlamydia MOMP polypeptide-containing chimeric polypeptide complex as defined in accordance with the sixth aspect of the invention, or a composition thereof as defined in accordance with the seventh aspect of the invention, wherein the Chlamydia MOMP polypeptide corresponds to the MOMP polypeptide of the Chlamydia bacterium; (2) identifying and/or isolating a B cell from the immunised subject, which specifically binds to the Chlamydia MOMP polypeptide or complex thereof; and (3) producing the antigen-binding molecule expressed by that B cell.

The invention provides, in a tenth aspect, an antigen-binding molecule that specifically binds to: the microbial polypeptide-containing chimeric polypeptide as defined in accordance with the first aspect of the invention and/or the microbial polypeptide of one or more subunits of a microbial polypeptide-containing chimeric polypeptide complex as defined in accordance with the sixth aspect of the invention.

In preferred embodiments of the latter aspect, the antigen-binding molecule specifically binds to:
(i) the ectodomain of an enveloped virus fusion ectodomain polypeptide-containing chimeric polypeptide as defined in accordance with the first aspect of the invention; and/or the ectodomain of one or more subunits of an enveloped virus fusion ectodomain polypeptide-containing chimeric polypeptide complex as defined in accordance with the sixth aspect of the invention;
or
(ii) the bacterial outer membrane polypeptide of a bacterial outer membrane polypeptide-containing chimeric polypeptide as defined in accordance with the first aspect of the invention; and/or the bacterial outer membrane polypeptide of a bacterial outer membrane polypeptide-containing chimeric polypeptide complex as defined in accordance with the sixth aspect of the invention.

In preferred embodiments of the latter embodiment, the antigen-binding molecule specifically binds to:
(i) the TAA polypeptide of a TAA polypeptide-containing chimeric polypeptide as defined in accordance with the first aspect of the invention; and/or the TAA polypeptide of a TAA polypeptide-containing chimeric polypeptide complex as defined in accordance with the sixth aspect of the invention; or
(ii) the Chlamydia major outer membrane protein (MOMP) polypeptide of a Chlamydia MOMP polypeptide-containing chimeric polypeptide as defined in accordance with the first aspect of the invention; and/or the Chlamydia MOMP polypeptide of a Chlamydia MOMP polypeptide-containing chimeric polypeptide complex as defined in accordance with the sixth aspect of the invention.

The invention provides, in an eleventh aspect, an antigen-binding molecule that is obtainable by the method according to the ninth aspect of the invention.

The invention provides, in a twelfth aspect, a composition comprising an antigen-binding molecule as defined in accordance with the tenth or eleventh aspect of the invention, and a pharmaceutically acceptable carrier, diluent or adjuvant.

The invention provides, in a thirteenth aspect, a composition comprising the nucleic acid as defined in accordance with the third aspect of the invention.

The invention provides, in a fourteenth aspect, a chimeric polypeptide as defined in accordance with the first or second aspect of the invention, a nucleic acid as defined in accordance with the third aspect of the invention, a host cell as defined in accordance with the fourth aspect of the invention, a chimeric polypeptide complex as defined in accordance with the sixth aspect of the invention, a composition as defined in accordance with the seventh aspect of the invention, an antigen-binding molecule as defined in accordance with the tenth or eleventh aspect of the invention, or a composition as defined in accordance with the twelfth or thirteenth aspect of the invention for use as a medicament.

The invention provides, in a fifteenth aspect, a method of eliciting an immune response to: a microbial polypeptide, or complex thereof, in a subject, wherein the method comprises administering to the subject:
(i) a microbial polypeptide-containing chimeric polypeptide as defined in accordance with the first aspect of the invention, a microbial polypeptide-containing chimeric polypeptide complex as defined in accordance with the sixth aspect of the invention, or a composition thereof as defined in accordance with the seventh aspect of the invention; or
(ii) a composition as defined in accordance with the thirteenth aspect of the invention.

In preferred embodiments of the latter aspect, the microbial polypeptide, or the complex thereof, is:
(a) a fusion protein of an enveloped virus, or complex of the fusion protein, respectively, wherein the method comprises administering to the subject (i) an enveloped virus fusion ectodomain-containing chimeric polypeptide as defined in accordance with the first aspect of the invention, an enveloped virus fusion ectodomain-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; wherein an ectodomain polypeptide subunit of the chimeric polypeptide complex corresponds to the fusion protein of the enveloped virus;
or
(b) an outer membrane polypeptide of a bacterium, or a complex of the outer membrane polypeptide, respectively, wherein the method comprises administering to the subject: (i) a bacterial outer membrane polypeptide-containing chimeric polypeptide as defined in accordance with the first aspect of the invention, a bacterial outer membrane polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; wherein a bacterial outer membrane polypeptide subunit of the chimeric polypeptide complex corresponds to, or substantially corresponds to, a bacterial outer membrane polypeptide expressed by the bacterium.

In preferred embodiments of the latter embodiment, the bacterial outer membrane polypeptide, or the complex thereof, is:
(a) TAA polypeptide of a bacterium, or a complex of the TAA polypeptide, respectively, wherein the method comprises administering to the subject: (i) a TAA polypeptide-containing chimeric polypeptide as defined in accordance with the first aspect of the invention, a TAA polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; wherein a TAA polypeptide subunit of the chimeric polypeptide complex corresponds to, or substantially corresponds to, a TAA polypeptide expressed by the bacterium; or
(b) Chlamydia major outer membrane protein (MOMP) polypeptide, or a complex of the Chlamydia MOMP polypeptide, respectively, wherein the method comprises administering to the subject: (i) a Chlamydia MOMP polypeptide-containing chimeric polypeptide as defined in accordance with the first aspect of the invention, a Chlamydia MOMP polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; wherein a Chlamydia MOMP polypeptide subunit of the chimeric polypeptide complex corresponds to, or substantially corresponds to, a Chlamydia MOMP polypeptide expressed by the bacterium.

The invention provides, in a sixteenth aspect, a method for treating or preventing a microbial infection in a subject, wherein the method comprises administering to the subject an effective amount of:
(i) a microbial polypeptide-containing chimeric polypeptide as defined in accordance with the first aspect of the invention, a microbial polypeptide-containing chimeric polypeptide complex as defined in accordance with the sixth aspect of the invention, or a composition thereof as defined in accordance with the seventh aspect of the invention;
(ii) an antigen-binding molecule as defined in accordance with the tenth or eleventh aspect of the invention, or a composition thereof as defined in accordance with the twelfth aspect of the invention; or
(iii) a composition as defined in accordance with the thirteenth aspect of the invention.

In preferred embodiments of the latter aspect, the microbial infection is: (a) an enveloped virus infection in a subject, wherein the method comprises administering to the subject an effective amount of: (i) an enveloped virus fusion ectodomain-containing chimeric polypeptide as defined in accordance with the first aspect of the invention, an enveloped virus fusion ectodomain-containing chimeric polypeptide complex as defined in accordance with the sixth aspect of the invention, or a composition thereof as defined in accordance with the seventh aspect of the invention; (ii) an antigen-binding molecule as defined in accordance with the tenth or eleventh aspect of the invention, or a composition thereof as defined in accordance with the twelfth aspect of the invention; or (iii) a composition as defined in accordance with the thirteenth aspect of the invention;
or
(b) a bacterial infection in a subject, wherein the method comprises administering to the subject an effective amount of: (i) a bacterial outer membrane polypeptide-containing chimeric polypeptide as defined in accordance with the first aspect of the invention, a bacterial outer membrane polypeptide-containing chimeric polypeptide complex as defined in accordance with the sixth aspect of the invention, or a composition thereof as defined in accordance with the seventh aspect of the invention; (ii) an antigen-binding molecule as defined in accordance with the tenth or eleventh aspect of the invention, or a composition thereof as defined in accordance with the twelfth aspect of the invention; or (iii) a composition as defined in accordance with the thirteenth aspect of the invention.

In preferred embodiments of the latter embodiment, the bacterial infection is:
(a) an infection by a TAA-expressing bacterium, wherein the method comprises administering to the subject an effective amount of: (i) a TAA polypeptide-containing chimeric polypeptide as defined in accordance with the first aspect of the invention, a TAA polypeptide-containing chimeric polypeptide complex as defined in accordance with the sixth aspect of the invention, or a composition thereof as defined in accordance with the seventh aspect of the invention; (ii) an antigen-binding molecule as defined in accordance with the tenth or eleventh aspect of the invention or a composition thereof as defined in accordance with the twelfth aspect of the invention; or (iii) a composition as defined in accordance with the thirteenth aspect of the invention; or
(b) an infection by a Chlamydia bacterium, wherein the method comprises administering to the subject an effective amount of: (i) a Chlamydia MOMP polypeptide-containing chimeric polypeptide as defined in accordance with the first aspect of the invention, a Chlamydia MOMP polypeptide-containing chimeric polypeptide complex as defined in accordance with the sixth aspect of the invention, or a composition thereof as defined in accordance with the seventh aspect of the invention; (ii) an antigen-binding molecule as defined in accordance with the tenth or eleventh aspect of the invention, or a composition thereof as defined in accordance with the twelfth aspect of the invention; or (iii) a composition as defined in accordance with the thirteenth aspect of the invention.

The invention provides, in a seventeenth aspect, a vaccine comprising:
(i) a microbial polypeptide-containing chimeric polypeptide as defined in accordance with the first aspect of the invention, a microbial polypeptide-containing chimeric polypeptide complex as defined in accordance with the sixth aspect of the invention, or a composition thereof as defined in accordance with the seventh aspect of the invention; or
(ii) a composition as defined in accordance with the thirteenth aspect of the invention;
for use in a method of eliciting an immune response to a microbial polypeptide, or a complex of the microbial polypeptide, in a subject.

In preferred embodiments of the latter aspect, the microbial polypeptide is:
(a) an enveloped virus fusion ectodomain polypeptide, and the vaccine comprises: (i) an enveloped virus fusion ectodomain polypeptide-containing chimeric polypeptide as defined in accordance with the first aspect of the invention, an enveloped virus fusion ectodomain polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; for use in a method of eliciting an immune response to a fusion protein of an enveloped virus, or a complex of the fusion protein, in a subject, and wherein an ectodomain polypeptide subunit of the chimeric polypeptide complex corresponds to the fusion protein of the enveloped virus;
or
(b) a bacterial outer membrane polypeptide, and the vaccine comprises: (i) a bacterial outer membrane polypeptide-containing chimeric polypeptide as defined in accordance with the first aspect of the invention, a bacterial outer membrane polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; for use in a method of eliciting an immune response to an outer membrane polypeptide of a bacterium, or a complex of the outer membrane polypeptide, in a subject, and wherein a bacterial outer membrane polypeptide subunit of the chimeric polypeptide complex corresponds to an outer membrane polypeptide of the bacterium.

In preferred embodiments of the latter embodiment, the bacterial outer membrane polypeptide is:
(a) a trimeric autotransporter adhesin (TAA) polypeptide, and the vaccine comprises: (i) a TAA polypeptide-containing chimeric polypeptide as defined in accordance with the first aspect of the invention, a TAA polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; for use in a method of eliciting an immune response to a TAA polypeptide of a bacterium, or a complex of the TAA polypeptide, in a subject, and wherein a TAA polypeptide subunit of the chimeric polypeptide complex corresponds to a TAA polypeptide of the bacterium;
or
(b) a Chlamydia MOMP polypeptide, and the vaccine comprises: (i) a Chlamydia MOMP polypeptide-containing chimeric polypeptide as defined in accordance with the first aspect of the invention, a Chlamydia MOMP polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; for use in a method of eliciting an immune response to a MOMP polypeptide of a Chlamydia bacterium, or a complex of the MOMP polypeptide, in a subject, and wherein a MOMP polypeptide subunit of the chimeric polypeptide complex corresponds to a MOMP polypeptide of the Chlamydia bacterium.

The invention provides, in an eighteenth aspect, a microbial polypeptide-containing chimeric polypeptide as defined in accordance with the first aspect of the invention, a microbial polypeptide-containing chimeric polypeptide complex as defined in accordance with the sixth aspect of the invention, or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing a microbial infection in a subject.

In preferred embodiments of the latter aspect, the microbial polypeptide is:
(a) an enveloped virus fusion ectodomain polypeptide, and provided is an enveloped virus fusion ectodomain polypeptide-containing chimeric polypeptide as defined in accordance with the first aspect of the invention, an enveloped virus fusion ectodomain-containing chimeric polypeptide complex as defined in accordance with the sixth aspect of the invention or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing an enveloped virus infection in a subject;
or
(b) a bacterial outer membrane polypeptide, and provided is a bacterial outer membrane polypeptide-containing chimeric polypeptide as defined in accordance with the first aspect of the invention, a bacterial outer membrane polypeptide-containing chimeric polypeptide complex as defined in accordance with the sixth aspect of the invention or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing a bacterial infection in a subject.

In preferred embodiments of the latter embodiment, the bacterial outer membrane polypeptide is:
(a) a bacterial trimeric autotransporter adhesin (TAA) polypeptide, and provided is a bacterial trimeric autotransporter adhesin (TAA) polypeptide-containing chimeric polypeptide as defined in accordance with the first aspect of the invention, a TAA polypeptide-containing chimeric polypeptide complex as defined in accordance with the sixth aspect of the invention or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing a bacterial infection by a TAA-expressing bacterium in a subject;
or
(b) a Chlamydia MOMP polypeptide, and provided is a Chlamydia MOMP polypeptide-containing chimeric polypeptide as defined in accordance with the first aspect of the invention, a Chlamydia MOMP polypeptide-containing chimeric polypeptide complex as defined in accordance with the sixth aspect of the invention or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing a Chlamydia infection in a subject.

### BRIEF DESCRIPTION OF THE DRAWINGS

FIG. 1: Nineteen putative clamp sequences derived from the trimerization domains of proteins from animal viruses known not to commonly infect humans were designed. Potential second-generation clamp sequences are aligned with the original HIV-based clamp, denoted HIV-clamp. CD and CK codes were assigned to the various second-generation clamps. The viruses that the sequences are derived from are indicated.

FIG. 2: Size exclusion HPLC (SE-HPLC) analysis of selected VL22_66K clamp antigens. AUC=area under curve. Antigens labelled CDX or CK8 are VL22_66K CDX or VL22_K66 CK8, respectively. Fsol is the non-stabilized soluble form of RSV ectodomain and is a post-fusion F control (SEQ ID NO: 27). F HIV-clamp is the control antigen stabilised by an HIV-clamp (SEQ ID NO: 26).

FIG. 3: Transmission electron microscopy (TEM) of selected VL22_66K clamp antigens. Samples were coated at 10 μg/ml on glow discharged carbon coated grids. Antigens labelled CDX are VL22_K66 CDX.

FIG. 4: Thermal stability analysis of selected VL22_66K clamps by SE-HPLC. Antigens were incubated at 4° C. or 25° C. for 72 hours. Antigens labelled CDX are VL22_66K CDX.

FIG. 5: Comparison of antigen yield recovered from transient ExpiCHO (Thermo Fisher) protein expression of the VL22_66K clamp antigens (A) and VL22_66E clamp antigens (B). ND=not determined. In each graph, the dotted line indicates the yield recovered for the F HIV-clamp. For simplicity, in the graph labels, VL22 is omitted from the VL22 clamp antigen names.

FIG. 6: Thermal stability of VL22_CD10 and VL22_CD11, and F HIV-clamp as assessed by binding of RSV F specific mAbs (101F and MPE8) via ELISA. Prior to ELISA, antigens were incubated at 4, 25, 40 and 60° C. for 72 hours.

FIG. 7: Thermal stability of VL22_CD10 and VL22_CD11, and F HIV-clamp as assessed by SE-HPLC analysis in duplicate. Prior to SE-HPLC, antigens were incubated at 4, 25, 40 and 60° C. for 72 h.

FIG. 8: Cryo-Electron Microscopy of VL22_CD11 trimer and low-resolution model produced from images.

FIG. 9: Cryo-Electron Microscopy of VL22_CD11 trimer in complex with Motavizumab Fab and high-resolution model produced

FIG. 10: Binding of sera from eight mice immunised with SARS-CoV-2 S HIV-clamp to VL22_CD10, VL22_CD11 and F HIV-clamp by ELISA (panels A-C). Endpoint titres (EPTs) are plotted as geometric means with geometric standard deviations (panel D).

FIG. 11: Analysis of binding of Bio-Rad Geenius HIV 1/2 control human plasma (panel A and B) and NIBSC International HIV reference standard (panel C and D) to SARS-CoV-2 S HIV-clamp and SARS-CoV-2 S CD11 via ELISA.

FIG. 12: Analysis of binding for sera from 8 mice immunised with VL22_CD10 (A), VL22_CD11 (B) and F HIV-clamp (C) to HIV gp41 (#ab49070, Abcam, Cambridge, United Kingdom), the identical antigen used for vaccination (self-antigen), to the clamp domain within the antigen when paired with an alternate ectodomain (SARS-CoV-2 S HIV-clamp, SARS-CoV-2 S CD10 and SARS-CoV-2 S CD11), and to the ectodomain within the antigen when paired with an alternate clamp (F HIV-clamp or SARS-CoV-2 S CD11) by ELISA.

FIG. 13: Serum neutralisation of RSV A2 assessed by Plaque Reduction Neutralization Test (PRNT) following two vaccinations of BALB/c mice (n=5 or n=3 for PBS), three weeks apart, with each dose consisting of 25 μl AddaVax (Invivogen) and 5 μg of VL22_CD10, VL22_CD11 or F HIV-clamp or PBS control. International standard refers to the WHO 1^{st} International Standard for Antiserum to Respiratory Syncytial Virus (NIBSC; #16-284).

FIG. 14: SE HPLC of partial CD panel with SARS-CoV-2 Spike antigen. Dotted lines indicate elusion time for Molecular Weight Standards. A=Thyroglobin (670 kDa), B=Gama Globulin (158 kDa), C=Ovalbumin (44 kDa).

FIG. 15: SE-FPLC of viral antigens stabilised with HIV-clamp and CD11. Comparison between SARS-CoV-2 S HIV-clamp and SARS-CoV-2 S CD11 on Superose 6 Increase 10/300GL (Cytiva) (panel A). Comparison between Nipah virus F HIV-clamp and Nipah F CD11 on Superdex 200 Increase 10/300GL (Cytiva) (panel B). Comparison between Influenza HA HIV-clamp and Influenza HA CD11 on Superdex 200 Increase 10/300GL (Cytiva) (panel c).

FIG. 16: Serum neutralisation assessed by PRNT or pseudovirus neutralisation following two vaccinations of BALB/c mice (n=8), three weeks apart, with each dose consisting of 25 μl AddaVax (Invivogen) and 5 μg of each antigen or PBS control. Neutralisation is assessed against live viruses, SARS-CoV-2 614G mutant and influenza A H1N1pdm California09 by PRNT and lenti pseudovirus for Nipah virus.

FIG. 17: SE-FPLC of SARS-CoV-2 S CD11 (GSG-linker) (SEQ ID NO: 29) and SARS-CoV-2 S CD11-SG (G-linker) (SEQ ID NO: 34) on Superose 6 Increase 10/300GL (Cytiva). Arrows indicate expected sizes for trimer and monomeric protein.

FIG. 18: A model of CD11 trimer (grey) with selected regions for potential incorporation of N-linked glycosylation sites (black).

FIG. 19: Alignment of silenced CD11 sequences. Shading indicates modified residues, arrows indicate glycosylation sites and numbering used to define each site. Naming of variants reflects site numbering (e.g., 159 has glycosylation sites 1, 5 and 9). The grey bar indicates the samples incubated at 4 and 40° C. for 1 week and subsequently analysed by SE-HPLC.

FIG. 20: Binding affinity (KD) for 101F and MPE8 attachment to VL22 antigens containing CD11, CD11 silenced variants or to non-stabilised Fsol.

FIG. 21: Protein yield for VL22 antigens containing CD11 or CD11 silenced variants. Mean yields with standard deviation of two expression runs are graphed for antigens except CD11_12456, CD11_1245T68, CD11_15T, CD11_1 and CD11_5T where results are from one expression run only.

FIG. 22: SE-HPLC of VL22 antigens containing CD11 or CD11 silenced variants. Duplicate traces are overlayed for each sample.

FIG. 23: SE-HPLC traces of VL22 antigens containing CD11 or CD11 silenced variants following 1 week incubation at 4 or 40° C.

FIG. 24: SE-HPLC traces of VL22 antigens containing CD11 or CD11 silenced variants following incubation at 4 or 40° C. for 38 days. Duplicate runs are plotted for each temperature incubation.

FIG. 25: Glycan composition of recombinant antigens. (A) Alignment of N-linked sites in each vaccine candidate and (B) N-linked site-specific occupancy. Note CD11 refers to VL22_CD11 and CD11145T8 and CD11 1245T8 refer to silenced clamp variants of VL22_CD11. N518=site 1; N523=site 2; N530=site 4; N534=site 5; N544=site 8 (e.g., 145T8 has N518, N530, N534 and N544).

FIG. 26: SE-HPLC traces for lead silenced CD11-based antigens VL22_CD11_1245T8 and VL22_CD11_145T8 and unsilenced VL22_CD11, F HIV-clamp, and controls DS-Cav1 Foldon and SC9-10 DS-Cav AY Foldon.

FIG. 27: Yields of VL22_CD11_145T8, VL22_CD11, VL22_HIV-clamp, DS-Cav1 Foldon and SC9-10 DS-Cav AY Foldon. Average yield and standard deviation from 3-5 separate transient expression runs is presented.

FIG. 28: RSV A2 PRNT analysis of terminal sera from mice receiving two IM injections of 1 μg of VL22_CD11_1245T8, VL22_CD11_145T8, VL22_CD11, F HIV-clamp, DS-Cav1 Foldon or SC9-10 DS-Cav AY Foldon mixed with Addavax adjuvant. Error bars represent geometric mean titres and standard deviation, with geometric mean titres also labelled above each data set. Significance relative to PBS vaccinated control was determined by One-way ANOVA with Dunn's Multiple Comparison Test using GraphPad Prism version 9.2.0 for Windows. Indicated significance: ****P<0.0001; ***P<0.001; **P<0.01; *P<0.05 and ns=not significant. International standard refers to the WHO 1^{st} International Standard for Antiserum to Respiratory Syncytial Virus (NIBSC; #16-284). LoD indicates the limit of detection of the assay.

FIG. 29: Binding of terminal sera from mice receiving two IM injections of 1 μg of of VL22_CD11_1245T8, VL22_CD11_145T8, VL22_CD11, F HIV-clamp, DS-Cav1 Foldon or SC9-10 DS-Cav AY Foldon mixed with Addavax adjuvant to either the identical antigen used for vaccination (self-antigen), to the RSV F ectodomain with an alternate (stabilisation domain (CD11 or HIV-clamp) or the Nipah F antigen containing the identical stabilisation domain (Foldon, HIV-clamp, CD11, CD11_145T8 or CD11_1245T8) by ELISA. Geometric mean end point titres (EPTs) with geometric standard deviations are plotted.

FIG. 30: Alignment of amino acid sequences of CD11 and site directed modifications (SEQ ID NO: 55-72) with the HIV-clamp sequence provided for reference.

FIG. 31: Expression level for CD11 site directed modifications relative to unmodified CD11 control. Three measurements via ELISA presented for each of the SARS-CoV-2 S CD11 panel, SARS-CoV-2 (Delta) S CD11 panel SARS-CoV-2 (Delta) S CD11-QS panel and 2 measurements via Nanodrop, post purification for each of the SARS-CoV-2 (Delta) S CD11 panel SARS-CoV-2 (Delta) S CD11-QS panel. Geometric mean with geometric standard deviation plotted.

FIG. 32: Percentage of antigen found to be in trimeric conformation, HMW aggregate and LMW product by SEC for the SARS-CoV-2 (Delta) S CD11 site directed modification panel (A) and SARS-CoV-2 (Delta) S CD11-QS site directed modifications panel (B).

FIG. 33: Percentage of antigen found to be in trimeric conformation, HMW aggregate and LMW product by SEC following incubation at 40° C. for 48 h for the SARS-CoV-2 (Delta) S CD11 site directed modification panel (A) and SARS-CoV-2 (Delta) S CD11-QS site directed modifications panel (B).

FIG. 34: Antigen integrity assessed via conformation specific ELISA for VL22 Clamp2 (D25 mAb) or SARS2-S Clamp2 (CR3022 mAb) at time 0 or at 6 weeks of incubation at 4° C., either each antigen alone or for the mixed products. nr=no result.

FIG. 35: Serum neutralisation of RSV A2 (A) or SARS-CoV-2 (B) assessed by PRNT following two vaccinations of BalB/C mice (n=8), three weeks apart, with each dose consisting of 25 μl AddaVax (Invivogen) and either 1 μg of VL22 Clamp2, 1 μg of SARS2-S Clamp2, or 1 μg of VL22 Clamp2+1 μg of SARS2-S Clamp2 or PBS control.

FIG. 36: Expression of CD11 stabilized bacterial autotransporters analysed by SDS-PAGE of E. coli lysate (A) or Western blot (WB) with anti-CD11 monoclonal antibody (B). Lane 1=Lysate of negative control E. coli transformed with pSU vector backbone only, Lane 2=Molecular Weight Marker (MW listed on left), Lanes 3-9=Lysate E. coli transformed with pSU expression vector containing CD11 stabilized antigens EhaG, HiA, NadA, NhhA, YadA, EibD, and UpaG.

FIG. 37: CD11 stabilized bacterial autotransporters, EhaG (A), UpaG (B) and HiA (C), purified by affinity chromatography and analysed by SDS-PAGE.

FIG. 38: Purified CD11 stabilized bacterial autotransporters, EhaG (A), UpaG (B) and HiA (C), analysed by size exclusion chromatography on Superose 6 Increase 10/300GL (Cytiva). Also shown linear regression of Log Marker MW and elution volume and table comparing expected and observed MW for each autotransporter.

FIG. 39: Purified CD11 stabilized bacterial autotransporter, UpaG, analysed by negative stain Transmission Electron Microscopy (TEM).

FIG. 40: Serum neutralisation of RSV A2 assessed by PRNT following two vaccinations of BaIB/C mice (n=8), three weeks apart, with each dose consisting of 25 μl AddaVax (Invivogen) and 1 μg of VL22 Clamp2, 1 μg of SARS2-S Clamp2, 1 μg of VL22 Clamp2+1 μg of SARS2-S Clamp2 1 (CD11), or PBS control.

FIG. 41: Illustrative for generation of a membrane tethered clamp stabilized antigen to be delivered by LNP encapsulated mRNA, DNA or alternated vaccine vector. A) Standard soluble RSV F Clamp2 design—following administration, the encoded antigen will be secreted from the cell into the extracellular medium. B) Design for a membrane tethered RSV F Clamp2 in which there is a hydrophobic sequence inserted within the linker region between the FHRR and SHRR regions of Clamp2—following administration, the encoded antigen will be secreted but remain tethered to the cell surface. C) Design for a membrane embedded RSV F clamp2 in which the Clamp2 sequence is inserted following the native transmembrane domain so that the clamp is present on the cytoplasmic face of the membrane—following administration, the encoded antigen will be secreted but remain tethered to the cell surface.

FIG. 42: Overview of candidate membrane tethering (poly)peptides, including origin, sequence and structural information.

FIG. 43: Qualitative assessment of soluble vs membrane bound Clamp2 stabilized protein. A) Soluble antigen as measured in cell supernatant by cature ELISA. B) Cell surface bound antigen as measured by Flow Cytometry.

FIG. 44: Westernblot of CHO cell extract following transfection with membrane tethered clamp constructs and controls. Each cell extract was run on SDS-PAGE with or without prior treatment with PNGase to remove N-linked glycans. Gel 1 (from left to right)—molecular weight marker, purified VL22-Clamp2 (SEQ ID NO: 75), cell extract mFwt (SEQ ID NO: 234), cell extract VL22-CT5s (SEQ ID NO: 78), cell extract mVL22-TM-CD11 (SEQ ID NO: 233), mDSCavAY (SEQ ID NO: 235), 2^{nd} cell extract mFwt (SEQ ID NO: 234). Gel 2 (from left to right)—molecular weight marker, purified VL22-Clamp2 (SEQ ID NO: 75), cell extract VL22_CT5s_alpha (SEQ ID NO: 225), cell extract VL22_CT5s_gamma (SEQ ID NO: 227), cell extract VL22_CT5s_eta (SEQ ID NO: 232), cell extract VL22_CT5s_epsilon (SEQ ID NO: 229), cell extract non-transfected.

FIG. 45: Quantification of expression level for membrane tethered clamp constructs. Detergent extract from cellular membranes quantified by capture ELISA, A) Motavizumab, B) D25. ELISA curves assessed by non-linear regression and 50% effective concentration (EC₅₀) shown.

### DETAILED DESCRIPTION OF THE INVENTION

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

Further, the terms “about” and “approximate”, as used herein, when referring to a measurable value such as an amount, dose, time, temperature, activity, level, number, frequency, percentage, dimension, size, amount, weight, position, length and the like, is meant to encompass variations of, e.g., ±15%, ±10%, ±5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polypeptide, these terms encompass variations of, e.g., ± up to 20 amino acid residues, up to 15 amino acid residues, ± up to 10 amino acid residues, up to 5 amino acid residues, ± up to 4 amino acid residues, ± up to 3 amino acid residues, ± up to 2 amino acid residues, or even ±1 amino acid residue. Moreover, whenever the term “about” or “approximate” is used, the present invention also specifically relates to the corresponding exact value (without variation).

The term “adjuvant” as used herein refers to a compound that, when used in combination with a specific immunogen (e.g., a (poly)peptide, chimeric polypeptide, chimeric polypeptide complex, polynucleotide and nucleic acid construct of the present disclosure) in a composition, will augment the resultant immune response, including intensification and/or broadening the specificity of either or both antibody and cellular immune responses. In the context of the present disclosure, an adjuvant will preferably enhance the specific immunogenic effect of the active agents of the present disclosure. The term “adjuvant” is typically understood not to comprise agents which confer immunity by themselves. An adjuvant assists the immune system unspecifically to enhance the antigen-specific immune response by, e.g., promoting presentation of an antigen to the immune system or induction of an innate immune response. Furthermore, an adjuvant may preferably, e.g., modulate the antigen-specific immune response by, e.g., shifting the dominating Th2-based antigen specific response to a more Th1-based antigen specific response or vice versa. Accordingly, an adjuvant may favorably modulate cytokine expression/secretion, antigen presentation, type of immune response etc.

The term “agent” as used herein interchangeably with “compound”, refers to any compound or substance such as, but not limited to, a small molecule, nucleic acid, polypeptide, peptide, drug, ion, etc. An “agent” can be any chemical entity, including without limitation synthetic and naturally-occurring proteinaceous and non-proteinaceous entities. In some embodiments, an agent is selected from nucleic acids, nucleic acid analogues, proteins, antibodies, peptides, aptamers, oligomers of nucleic acids, amino acids, carbohydrates, oligonucleotides, ribozymes, DNAzymes, glycoproteins, glycolipids, siRNAs, lipoproteins, and modifications and combinations thereof. In some embodiments, the nucleic acid is DNA or RNA; nucleic acid analogues, for example, can be selected from PNA, pcPNA and LNA. A nucleic acid may be single or double stranded, and can be selected from nucleic acids encoding a protein of interest, oligonucleotides, etc. Such nucleic acids include, for example, nucleic acids encoding proteins that act as transcriptional repressors, antisense molecules, ribozymes, small inhibitory nucleic acid sequences, for example but not limited to RNAi, shRNA, siRNA, microRNA, antisense oligonucleotides etc. A protein can be any protein of interest, for example, mutated proteins; therapeutic proteins; truncated proteins, wherein the protein is normally absent or expressed at lower levels in the cell. Proteins and peptides can be selected from mutated proteins, genetically engineered proteins, synthetic peptides, recombinant proteins, chimeric proteins, antibodies, humanized proteins, humanized antibodies, chimeric antibodies, modified proteins and fragments thereof. A carbohydrate may be, e.g., a monosaccharide, a disaccharide, an oligosaccharide, or a polysaccharide.

As used herein, the term “antigen” and its grammatically equivalent expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

By “antigen-binding molecule” is meant a molecule that has binding affinity for a target antigen. It will be understood that this term extends to immunoglobulins, immunoglobulin fragments and non-immunoglobulin derived protein or other non-protein frameworks that exhibit antigen-binding activity. Representative antigen-binding molecules that are useful in the practice of the present disclosure include polyclonal and monoclonal antibodies as well as their fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and ρ, respectively. The subunit structures and three-dimensional configurations of different classes of immunoglobulins are well known. Antigen-binding molecules also encompass dimeric antibodies, as well as multivalent forms of antibodies. In some embodiments, the antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., 1984, Proc. Natl. Acad. Sci. USA 81:6851-6855). Also contemplated, are humanized antibodies, which are generally produced by transferring complementarity determining regions (CDRs) from heavy and light variable chains of a non-human (e.g., rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions. General techniques of cloning non-human, particularly murine, immunoglobulin variable domains are described, for example, by Orlandi et al. (1989, Proc. Natl. Acad. Sci. USA 86: 3833). Techniques for producing humanized monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies,” in Protein Engineering: Principles and Practice Cleland et al. (eds.), pages 399-434 (John Wiley & Sons, Inc. 1996), and by Queen et al., U.S. Pat. No. 5,693,762 (1997). Humanized antibodies include “primatized” antibodies in which the antigen-binding region of the antibody is derived from an antibody produced by immunizing macaque monkeys with the antigen of interest. Also contemplated as antigen-binding molecules are humanized antibodies. Further examples of an “antigen-binding molecule” include any of the above-described agents, which can be obtained, e.g., by using the method according to the eighth aspect of the invention.

The term “anti-parallel”, as used herein, refers to a proteinaceous polymer in which regions or segments of the polymer are in a parallel orientation but have opposite polarities.

As used herein, the term “binds specifically” refers to a binding reaction which is determinative of the presence of a chimeric polypeptide or complex of the present disclosure in the presence of a heterogeneous population of molecules including macromolecules such as proteins and other biologics. In specific embodiments, the term “binds specifically” when referring to an antigen-binding molecule is used interchangeably with the term “specifically immuno-interactive” and the like to refer to a binding reaction which is determinative of the presence of a chimeric polypeptide or complex of the present disclosure in the presence of a heterogeneous population of proteins and other biologics. Under designated assay conditions, a molecule binds specifically to a chimeric polypeptide or complex of the disclosure and does not bind in a significant amount to other molecules (e.g., proteins or antigens) present in the sample. In antigen-binding molecule embodiments, a variety of immunoassay formats may be used to select antigen-binding molecules that are specifically immuno-interactive with a chimeric polypeptide or complex of the disclosure. For example, solid-phase ELISA immunoassays are routinely used to select monoclonal antibodies that are specifically immuno-interactive with a protein. See Harlow and Lane (1988) Antibodies, A Laboratory Manual, Cold Spring Harbor Publications, New York, for a description of immunoassay formats and conditions that can be used to determine specific immunoreactivity.

The term “chimeric”, when used in reference to a molecule, means that the molecule contains portions that are derived from, obtained or isolated from, or based upon two or more different origins or sources. Thus, a polypeptide is chimeric when it comprises two or more amino acid sequences of different origin and includes (1) polypeptide sequences that are not found together in nature (i.e., at least one of the amino acid sequences is heterologous with respect to at least one of its other amino acid sequences), or (2) amino acid sequences that are not naturally adjoined.

By “coding sequence” is meant any nucleic acid sequence that contributes to the code for the polypeptide product of a gene or for the final mRNA product of a gene (e.g., the mRNA product of a gene following splicing).

By contrast, the term “non-coding sequence” refers to any nucleic acid sequence that does not contribute to the code for the polypeptide product of a gene or for the final mRNA product of a gene.

The terms “coiled coil” or “coiled coil structure” are used interchangeably herein to refer to a structural motif in proteins, in which two or more α-helices (most often 2-7 α-helices) are coiled together like the strands of a rope (dimers and trimers are the most common types). Many coiled coil type proteins are involved in important biological functions such as the regulation of gene expression, e.g., transcription factors. Coiled coils often, but not always, contain a repeated pattern, hpphppp or hppphpp, of hydrophobic (h) and polar (p) amino-acid residues, referred to as a heptad repeat (see herein below). This repeating pattern in a (poly)peptide sequence naturally folds into an α-helical secondary structure resulting in the presentation of the hydrophobic residues along one face of the helix and the hydrophilic residues along the opposite face forming an amphipathic structure. The most favorable way for two or three such helices to arrange themselves in a water-filled environment is to wrap or sequester the hydrophobic faces of the helix against each other leaving the hydrophilic amino acids solvent exposed. It is thus the burial of hydrophobic surfaces, which provides the thermodynamic driving force for oligomerization of the α-helices and the stability of the structure. The packing in a coiled-coil interface is exceptionally tight. The α-helices may be parallel or anti-parallel, and usually adopt a left-handed super-coil. Although disfavored, a few right-handed coiled coils have also been observed in nature and in designed proteins. The terms “coiled coil” or “coiled coil structure” will be clear to the person skilled in the art based on the common general knowledge. Particular reference in this regard is made to review papers concerning coiled-coil structures, such as for example, Cohen and Parry (1990. Proteins 7:1-15); Kohn and Hodges (1998. Trends Biotechnol 16:379-389); Schneider et al. (1998. Fold Des 3:R29-R40); Harbury et al. (1998. Science 282:1462-1467); Mason and Arndt (2004. Chem-BioChem 5:170-176); Lupas and Gruber (2005. Adv Protein Chem 70:37-78); Woolfson (2005. Adv Protein Chem 70:79-112); Parry et al. (2008. J Struct Biol 163:258-269); and Mcfarlane et al. (2009. Eur J Pharmacol 625:101-107).

As used herein the term “complementary” and grammatically equivalent expressions thereof refer to the characteristic of two or more structural elements (e.g., peptide, polypeptide, nucleic acid, small molecule, or portions thereof etc.) of being able to hybridize, oligomerize (e.g., dimerize), interact or otherwise form a complex with each other. For example, “complementary regions of a polypeptide” are capable of coming together to form a complex, which is characterized in specific embodiments by an anti-parallel, two-helix bundle. As used herein, the term “complex” refers to an assemblage or aggregate of molecules (e.g., peptides, polypeptides, etc.) in direct and/or indirect contact with one another. In specific embodiments, “contact”, or more particularly, “direct contact” means that two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favored (e.g., compared to a non-aggregated, or non-complexed, state of its component molecules). As used herein the term “complex”, unless described otherwise, refers to the assemblage of two or more molecules (e.g., peptides, polypeptides or a combination thereof). In specific embodiments, the term “complex” refers to the assemblage of three polypeptides.

Throughout this specification, unless the context requires otherwise, the words “comprise,” “comprises” and “comprising”, as well as “contain”, “contains” and “containing”, will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. Throughout the present specification, the term “comprising” (or “containing” or the like) also includes the narrower meanings of “consisting essentially of” and “consisting of”. Accordingly, whenever the term “comprising” (or “containing” or the like) is used, the invention also specifically relates to the corresponding subject-matter defined by the term “consisting essentially of” as well as the corresponding subject-matter defined by the term “consisting of” (in place of “comprising” or “containing”).

As used herein, the terms “conjugated”, “linked”, “fused” or “fusion” and their grammatical equivalents, in the context of joining together of two or more elements or components or domains by whatever means including chemical conjugation or recombinant means (e.g., by genetic fusion) are used interchangeably. Methods of chemical conjugation (e.g., using heterobifunctional crosslinking agents) are known in the art. More specifically, as used herein, a “(poly)peptide”-“structure-stabilizing moiety” fusion or conjugate refers to the genetic or chemical conjugation of the (poly)peptide, which is suitably in a metastable, pre-fusion conformation, to a structure-stabilizing moiety.

In specific embodiments, the structure-stabilizing moiety is fused indirectly to a polypeptide, e.g., via a hinge, particularly a flexible linker, comprising, for example, one or more glycine (Gly) and/or one or more serine (Ser) residues. In other embodiments, the structure-stabilizing moiety is fused directly to a polypeptide disclosed herein.

A “conservative amino acid substitution” is one in which the amino acid residue is replaced with an amino acid residue having a similar side chain. Families of amino acid residues having similar side chains have been defined in the art, which can be generally sub-classified as shown in Table 1:

TABLE 1
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Hydrophilic | Asparagine, Histidine, Glutamine, Cysteine, |
| (polar)/neutral | Serine, Threonine |
| Hydrophilic | Asparagine, Glutamine |
| (polar)/large | |
| Hydrophobic | Tyrosine, Valine, Isoleucine, Leucine, |
| (non-polar) | Methionine, Phenylalanine, Tryptophan |
| Aromatic | Tryptophan, Tyrosine, Phenylalanine |
| Residues that | Glycine and Proline |
| influence | |
| chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity. Conservative substitutions are shown in Table 2 under the heading of exemplary and preferred substitutions. Amino acid substitutions falling within the scope of the disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
Exemplary and Preferred Amino Acid Substitutions

| Original | Exemplary | Preferred |
|---|---|---|
| Residue | Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present disclosure will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the disclosure, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present disclosure, conventional compositions and methods for preparing and using constructs and host cells are well known to one skilled in the art, see for example, Molecular Cloning: A Laboratory Manual, 3^{rd} edition Volumes 1, 2, and 3. J. F. Sambrook, D. W. Russell, and N. Irwin, Cold Spring Harbor Laboratory Press, 2000.

By “corresponds to” or “corresponding to” is meant a nucleic acid sequence or an amino acid sequence that displays substantial sequence similarity or identity to a reference nucleic acid sequence or amino acid sequence, respectively. In general, the nucleic acid sequence or amino acid sequence will display, with increasing preference, at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even up to 100% sequence similarity or identity to at least a portion of the reference nucleic acid sequence or amino acid sequence or to the entire reference nucleic acid sequence or amino acid sequence.

The term “domain”, as used herein, refers to a part of a molecule or structure that shares common physicochemical features, such as, but not limited to, hydrophobic, polar, globular and helical domains or properties such as ligand-binding, membrane fusion, signal transduction, cell penetration and the like. Often, a domain has a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain. Domains may be co-extensive with regions or portions thereof; domains may also include distinct, non-contiguous regions of a molecule. Examples of protein domains include, but are not limited to, a cellular or extracellular localization domain (e.g., signal peptide; SP), an immunoglobulin (Ig) domain, a membrane fusion (e.g., fusion peptide; FP) domain, an ectodomain, a membrane proximal external region (MPER) domain, a transmembrane (TM) domain, and a cytoplasmic (C) domain.

By “effective amount”, in the context of treating, inhibiting the development of, or preventing a condition is meant the administration of an amount of an agent or composition to an individual in need of such treatment, inhibition or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of the individual to be treated, the formulation of the composition, the assessment of the medical situation, and other relevant factors. It is expected that the amount will fall in a relatively broad range that can be determined through routine trials.

The term “endogenous” refers to a polypeptide or part thereof that is present and/or naturally expressed within a host organism or cell thereof. For example, an “endogenous” ectodomain polypeptide or part thereof refers to an ectodomain polypeptide of an enveloped fusion protein or a part of that ectodomain that is naturally expressed in enveloped virus.

The term “endogenous production” refers to expression from a nucleic acid in an organism and the associated production and/or secretion of an expression product of the nucleic acid in the organism. In specific embodiments, the organism is multicellular (e.g., a vertebrate animal, preferably a mammal, more preferably a primate such as a human) and the nucleic acid is expressed within cells or tissues of the multicellular organism.

The terms “epitope” and “antigenic determinant” are used interchangeably herein to refer to an antigen, typically a protein determinant, that is capable of specific binding to an antibody (such epitopes are often referred to as “B cell epitopes”) or of being presented by a Major Histocompatibility Complex (MHC) protein (e.g., Class I or Class II) to a T-cell receptor (such epitopes are often referred to as “T cell epitopes”). Where a B cell epitope is a peptide or polypeptide, it typically comprises three or more amino acids, generally at least 5 and more usually at least 8 to 10 amino acids. The amino acids may be adjacent amino acid residues in the primary structure of the polypeptide (often referred to as contiguous peptide sequences) or may become spatially juxtaposed in the folded protein (often referred to as non-contiguous peptide sequences). T cell epitopes may bind to MHC Class I or MHC Class II molecules. Typically, MHC Class I-binding T cell epitopes are 8 to 11 amino acids long. Class II molecules bind peptides that may be 10 to 30 residues long or longer, the optimal length being 12 to 16 residues. The ability of a putative T cell epitope to bind to an MHC molecule can be predicted and confirmed experimentally (Dimitrov et al., 2010. Bioinformatics 26(16):2066-8).

The term “flexible linker” as used herein refers to a proteinaceous molecule containing at least one amino acid residue, usually at least two amino acids residues joined by peptide bond(s), which molecule permits two polypeptides linked thereby to move more freely relative to one another, as compared to their movement without the flexible linker. In certain embodiments, the flexible linker provides increased rotational freedom for two polypeptides linked thereby than the two linked polypeptides would have in the absence of the flexible linker. Such freedom of relative movement or rotational freedom allows polypeptides joined by the flexible linker to perform their individual functions or elicit their activities with less structural hindrance. A flexible linker may be characterized by the absence of secondary structures such as helices or β-sheets or a maximal secondary structure content of 10%, 20% 30% or 40%. Non-limiting examples of flexible linkers include the amino acid sequences GS, GSG, GGSGG (SEQ ID NO: 84), GGSG (SEQ ID NO: 149), GSGS (SEQ ID NO: 150), AS, GGGS (SEQ ID NO: 151), G₄S (SEQ ID NO: 152), (G₄S)₂ (SEQ ID NO: 153), (G₄S)₃ (SEQ ID NO: 154), (G₄S)₄ (SEQ ID NO: 155), G₄SG (SEQ ID NO: 156), GSGG (SEQ ID NO: 157) and GSGGS (SEQ ID NO: 158). In alternative preferred embodiments, the linker comprises or consists of the amino acid sequence G, GG, GGG, GGGG (SEQ ID NO: 159) or GGGGG (SEQ ID NO: 160). Additional flexible linker sequences are well known in the art. In various embodiments, the flexible linker contains or consists of at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 amino acid residues. In some of the same and other embodiments, the flexible linker contains or consists of up to 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 amino acid residues. In some of the same and other embodiments, the flexible linker contains or consists of between about 1 to about 30 amino acid residues, between about 1 to about 25 amino acid residues, between about 1 to about 20 amino acid residues, between about 1 to about 15 amino acid residues, between about 1 to about 12 amino acid residues, between about 1 to about 10 amino acid residues, between about 1 to about 8 amino acid residues, between about 1 to about 6 amino acid residues, between about 1 to about 5 amino acid residues, between about 1 to about 4 amino acid residues, or between about 1 to about 3 amino acid residues. In some of the same and other embodiments, the flexible linker contains or consists of between about 2 to about 30 amino acid residues, between about 2 to about 25 amino acid residues, between about 2 to about 20 amino acid residues, between about 2 to about 15 amino acid residues, between about 2 to about 12 amino acid residues, between about 2 to about 10 amino acid residues, between about 2 to about 8 amino acid residues, between about 2 to about 6 amino acid residues, between about 2 to about 5 amino acid residues, or between about 2 to about 4 amino acid residues. In some of the same and other embodiments, the flexible linker contains or consists of between about 3 to about 30 amino acid residues, between about 3 to about 25 amino acid residues, between about 3 to about 20 amino acid residues, between about 3 to about 15 amino acid residues, between about 3 to about 12 amino acid residues, between about 3 to about 10 amino acid residues, between about 3 to about 8 amino acid residues, between about 3 to about 6 amino acid residues, or between about 3 to about 5 amino acid residues. In certain embodiments, the flexible linker contains or consists of 1, 2, 3, 4, 5, 6, 7, 8, 9 or 10 amino acid residues. In particular, a flexible linker may be composed of amino acid residues (e.g., having any of the above-mentioned exemplary numbers of amino acid residues), wherein preferably at least about 70% (more preferably at least about 80%, even more preferably at least about 90%, even more preferably at least about 95%, still more preferably 100%) of said amino acid residues are selected from glycine, serine and alanine; more preferably, at least about 70% (more preferably at least about 80%, even more preferably at least about 90%, even more preferably at least about 95%, still more preferably 100%) of said amino acid residues are selected from glycine and serine. In some embodiments, the said amino acid residues are all glycine.

The term “helix bundle” refers to a plurality of peptide helices that fold such that the helices are substantially parallel or anti-parallel to one another. A two-helix bundle has two helices folded such that they are substantially parallel or anti-parallel to one another. Likewise, a six-helix bundle has six helices folded such that they are substantially parallel or anti-parallel to one another. By “substantially parallel or anti-parallel” is meant that the helices are folded such that the side chains of the helices are able to interact with one another. For example, the hydrophobic side chains of the helices are able to interact with one another to form a hydrophobic core.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The term “host” refers to any organism, or cell thereof, whether eukaryotic or prokaryotic into which a construct of the disclosure can be introduced. In particular embodiments, the term “host” refers to eukaryotes, including unicellular eukaryotes such as yeast and fungi as well as multicellular eukaryotes such as animals non-limiting examples of which include invertebrate animals (e.g., insects, cnidarians, echinoderms, nematodes, etc.); eukaryotic parasites (e.g., malarial parasites, such as Plasmodium falciparum, helminths, etc.); vertebrate animals (e.g., fish, amphibian, reptile, bird, mammal); and mammals (e.g., rodents, primates such as humans and non-human primates). Thus, the term “host cell” suitably encompasses cells of such eukaryotes as well as cell lines derived from such eukaryotes.

Reference herein to “immuno-interactive” includes reference to any interaction, reaction, or other form of association between molecules and in particular where one of the molecules is, or mimics, a component of the immune system.

As use herein, the term “immunogenic composition” or “immunogenic formulation” refers to a preparation which, when administered to a vertebrate, especially an animal such as a mammal, will induce an immune response.

By the term “linker”, or “flexible linker”, it is meant a molecule or group of molecules (such as a monomer or polymer) that connects two molecules and often serves to place the two molecules in a desirable configuration.

As used herein, the term “meta-stable”, as used in the context of a protein (e.g., an enveloped virus ectodomain polypeptide), refers to a labile but constrained conformational state that rapidly converts to a more stable conformational state upon a change in conditions. For example, an enveloped virus fusion protein in a pre-fusion form is in a labile, meta-stable conformation, and converts to the more stable post-fusion conformation upon, e.g., fusion to a host cell.

As used herein, the term “moiety” refers to a portion of a molecule, which may be a functional group, a set of functional groups, and/or a specific group of atoms within a molecule, that is responsible for a characteristic chemical, biological, and/or medicinal property of the molecule.

The term “neutralizing antigen-binding molecule” refers to an antigen-binding molecule that binds to or interacts with a target molecule or ligand and prevents binding or association of the target antigen to a binding partner such as a receptor or substrate, thereby interrupting the biological response that otherwise would result from the interaction of the molecules. In the case of the instant disclosure a neutralizing antigen-binding molecule suitably associates with a metastable or pre-fusion form of an enveloped virus fusion protein and preferably interferes or reduces binding and/or fusion of the spike protein to a cell membrane.

The term “oligomer” refers to a molecule that consists of more than one but a limited number of monomer units in contrast to a polymer that, at least in principle, consists of an unlimited number of monomers. Oligomers include, but are not limited to, dimers, trimers, tetramers, pentamers, hexamers, heptamers, octamers, nonamers, decamers and the like. An oligomer can be a macromolecular complex formed by non-covalent bonding of macromolecules like proteins. In this sense, a homo-oligomer would be formed by identical molecules and by contrast, a hetero-oligomer would be made of at least two different molecules. In specific embodiments, an oligomer of the disclosure is a trimeric polypeptide complex consisting of three polypeptide subunits. In these embodiments, the trimeric polypeptide may be a “homotrimeric polypeptide complex” consisting of three identical polypeptide subunits, or a “heterotrimeric polypeptide complex” consisting of three polypeptide subunits in which at least one subunit polypeptide is non-identical. A “polypeptide subunit” is a single amino acid chain or monomer that in combination with two other polypeptide subunits forms a trimeric polypeptide complex.

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the regulatory sequence relative to the nucleotide sequence of interest to permit expression of that sequence under conditions compatible with the regulatory sequence. The regulatory sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence. Likewise, “operably connecting” an enveloped virus fusion ectodomain polypeptide to a heterologous, structure-stabilizing moiety (SSM) encompasses positioning and/or orientation of the structure-stabilizing moiety (SSM) such that it can, under suitable conditions (e.g., in aqueous solution and/or physiological conditions), associate with the structure-stabilizing moieties (SSMs) of two further chimeric polypeptides to form a trimer, wherein preferably, in the trimer, the FHRRs and SHRRs of the three SSMs are associated in the form of a six-helix bundle.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the disclosure include, but are not restricted to, any member of the subphylum Chordata including primates (e.g., humans, monkeys and apes, and includes species of monkeys such from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice, rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, or other poultry, companion birds such as canaries, budgerigars etc.), marine mammals (e.g., dolphins, whales), reptiles (snakes, frogs, lizards etc.), and fish. A preferred subject is a human, particularly a human in need of eliciting an immune response to a fusion protein of an enveloped virus, or complex thereof. However, it will be understood that the aforementioned terms do not imply that symptoms are present.

By “pharmaceutically acceptable carrier” is meant a solid or liquid filler, diluent or encapsulating substance that can be safely used in topical or systemic administration to an animal, preferably a mammal, including humans. Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The term “polynucleotide” or “nucleic acid”, as used herein, encompasses any molecule containing two or more nucleotides, particularly a polymer of nucleotides, such as, e.g., a deoxyribonucleic acid (DNA) or a ribonucleic acid (RNA). The nucleotides may be, e.g., deoxyribonucleotides, ribonucleotides or nucleotide analogs, and they may optionally be substituted or modified. The nucleotides can be linked by phosphodiester bonds/linkages or, e.g., by phosphorothioate linkages, methylphosphonate linkages or boranophosphate linkages. The term “polynucleotide” or “nucleic acid” particularly relates to DNA or RNA, such as, e.g., mRNA, cRNA, or cDNA. The term typically refers to polymeric forms of nucleotides of, e.g., at least 10 bases in length, either ribonucleotides or deoxynucleotides or a modified form of either type of nucleotide. A “polynucleotide” or “nucleic acid” can be single stranded or double stranded.

The terms “peptide”, “polypeptide”, “(poly)peptide” and “protein” are used herein interchangeably and refer to a polymer of two or more amino acids linked via amide bonds (i.e., peptide bonds) that are formed between an amino group of one amino acid and a carboxyl group of another amino acid. The amino acids comprised in the peptide, polypeptide, (poly)peptide, or protein, which are also referred to as amino acid residues, may be selected from the 20 standard proteinogenic α-amino acids (i.e., Ala, Arg, Asn, Asp, Cys, Glu, Gln, Gly, His, lie, Leu, Lys, Met, Phe, Pro, Ser, Thr, Trp, Tyr, and Val) but also from non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., γ-alanine), γ-amino acids (e.g., γ-aminobutyric acid, isoglutamine, or statine) and δ-amino acids. Preferably, the amino acid residues comprised in the peptide, polypeptide or protein are selected from α-amino acids, more preferably from the 20 standard proteinogenic α-amino acids (which can be present as the L-isomer or the D-isomer, and are preferably all present as the L-isomer). The peptide, polypeptide or protein may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts P G & Greene T W, Greene's protective groups in organic synthesis, John Wiley & Sons, 2006. Such modifications may also include the covalent attachment of one or more polyethylene glycol (PEG) chains (forming a PEGylated peptide, polypeptide or protein), the covalent attachment of albumin, the glycosylation and/or the acylation with one or more fatty acids (e.g., one or more C₈₋₃₀ alkanoic or alkenoic acids; forming a fatty acid acylated peptide, polypeptide or protein). Moreover, such modified peptides, polypeptide or proteins may also include peptidomimetics, provided that they contain at least two amino acids that are linked via an amide bond (formed between an amino group of one amino acid and a carboxyl group of another amino acid). The amino acid residues comprised in the peptide, polypeptide or protein may, e.g., be present as a linear molecular chain (forming a linear peptide, polypeptide or protein) or may form one or more rings (corresponding to a cyclic peptide, polypeptide or protein). The peptide, polypeptide or protein may also form oligomers consisting of two or more identical or different molecules. Accordingly, peptides, polypeptides and proteins may form dimers, trimers and higher oligomers, wherein the peptide, polypeptide or protein molecules forming such dimers, trimers etc. may be identical or non-identical. The corresponding higher order structures are, consequently, termed homo- or heterodimers, homo- or heterotrimers, and homo- or heterooligomers (etc.). Such dimers, trimers and oligomers are likewise embraced by the terms “peptide”, “polypeptide”, “(poly)peptide” and “protein”.

The term “amino acid” refers, in particular, to any one of the 20 standard proteinogenic α-amino acids (i.e., Ala, Arg, Asn, Asp, Cys, Glu, Gln, Gly, His, lie, Leu, Lys, Met, Phe, Pro, Ser, Thr, Trp, Tyr, and Val) but also to non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and/or δ-amino acids as well as any other compound comprising at least one carboxylic acid group and at least one amino group. Unless defined otherwise, an “amino acid” preferably refers to an α-amino acid, more preferably to any one of the 20 standard proteinogenic α-amino acids (which can be present as the L-isomer or the D-isomer, and are preferably present as the L-isomer).

As used herein, the term “post-fusion conformation” of a fusion protein of an enveloped virus refers to the structure of an enveloped virus fusion protein, which is in a terminal conformation (i.e., formed at the end of the fusion process) and is the most energetically favorable state. In the post-fusion conformation, the fusion peptides or loops of the fusion protein are brought into close proximity with the fusion protein transmembrane domain. The specific structural elements that facilitate formation of the hairpin structure vary according to the class of enveloped fusion protein. For example, the post-fusion conformation of a Class I fusion protein is characterized by interaction between the endogenous FHRR region and the endogenous SHRR region of individual Class I fusion proteins to form a hairpin structure characterized by a six-helix bundle, comprising three endogenous SHRR and three endogenous FHRR regions. Alternatively, the post-fusion conformation of a Class III fusion protein is characterized by interaction between the internal fusion loops and the C-terminal transmembrane region which facilitates the formation of a hairpin structure. Post-fusion conformations of individual viral fusion proteins have been determined by electron microscopy and/or x-ray crystallography, such structures are readily identifiable when viewed in negatively stained electron micrographs and/or by a lack of pre-fusion epitopes.

As used herein, the term “pre-fusion conformation” of a fusion protein of an enveloped virus refers to the structure of an enveloped virus fusion protein, which is in a meta-stable confirmation (i.e., in a semi-stable conformation that is not the most energetically favorable terminal conformation) and upon appropriate triggering is able to undergo conformational rearrangement to the terminal post-fusion conformation. Typically, pre-fusion conformations of viral fusion proteins contain a hydrophobic sequence, referred to as the fusion peptide or fusion loop, that is located internally within the pre-fusion conformation and cannot interact with either the viral or host cell membranes. Upon triggering this hydrophobic sequence is inserted into the host cell membrane and the fusion protein collapses into the post-fusion hairpin like conformation. The pre-fusion conformation of viral fusion proteins varies according to the class of enveloped fusion protein. Each class is characterized by non-interacting structural elements that subsequently associate in the energetically favorable post-fusion conformation. For example, the pre-fusion conformation of a Class I fusion protein is dependent on the endogenous FHRR region not interacting with the endogenous SHRR region of individual fusion proteins of the trimer, thereby not permitting formation of a hairpin structure characterized by a six-helix bundle. Alternatively, the pre-fusion conformation of a Class III fusion protein is dependent a central a-helical coiled coil not interacting with fusion loop(s) at the C-terminal region of individual fusion proteins of the trimer, thereby not permitting formation of a hairpin structure. Pre-fusion conformations of individual viral fusion proteins have been determined by electron microscopy and/or X-ray crystallography, such structures are readily identifiable when viewed in negatively stained electron micrographs and/or by pre-fusion epitopes that are not present on post-fusion conformations.

“Regulatory elements”, “regulatory sequences”, “control elements”, “control sequences” and the like are used interchangeably herein to refer to nucleotide sequences located upstream (5′ non-coding sequences), within, or downstream (3′ non-coding sequences) of a coding sequence, and which influence the transcription, RNA processing or stability, or translation of the associated coding sequence, either directly or indirectly. Regulatory elements include enhancers, promoters, translation leader sequences, introns, Rep recognition element, intergenic regions and polyadenylation signal sequences. They include natural and synthetic sequences as well as sequences which may be a combination of synthetic and natural sequences.

The term “replicon” refers to any genetic element, e.g., a plasmid, a chromosome, a virus, a cosmid, etc., that behaves as an autonomous unit of polynucleotide replication within a cell, i.e., capable of replication under its own control.

“Self-assembly” refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

The term “sequence identity”, as used herein, refers to the sequence match between two (poly)peptides or nucleic acids. The (poly)peptide or nucleic acid sequences to be compared are aligned to give maximum identity, for example, using bioinformatics tools for pair wise alignment such as EMBOSS Needle (https://www.ebi.ac.uk/Tools/psa/emboss_needle/; see also Madeira F, et al. The EMBL-EBI search and sequence analysis tools APIs in 2019. Nucleic Acids Research. 2019 July; 47(W1):W636-W641. DOI: 10.1093/nar/gkz268). When the same position in the sequences to be compared is occupied by the same nucleobase or amino acid residue, then the respective molecules are identical at that very position. Accordingly, the “percent identity” is a function of the number of matching positions divided by the number of positions compared and multiplied by 100%. For example, if 6 out of 10 sequence positions are identical, then the identity is 60%. The “identity” or “percent (%) identity” between two amino acid sequences can, e.g., be determined by using the Needleman-Wunsch algorithm (Needleman, S. B. and Wunsch, CD. A general method applicable to the search for similarities in the amino acid sequence of two proteins. J Mol Biol. 1970; 48(3):443-53. DOI: 10.1016/0022-2836(70)90057-4.) which has been incorporated into EMBOSS Needle, using a BLOSUM62 matrix, a “gap open penalty” of 10, a “gap extend penalty” of 0.5, a false “end gap penalty”, an “end gap open penalty” of 10 and an “end gap extend penalty” of 0.5. The percent (%) identity is typically determined over the entire length of the query sequence on which the analysis is performed. Two molecules having the same primary amino acid or nucleic acid sequence are identical irrespective of any chemical and/or biological modification. For example, two antibodies having the same primary amino acid sequence, but different glycosylation patterns are identical by this definition. In case of nucleic acids, for example, two molecules having the same sequence but different linkage components such as thiophosphate instead of phosphate are identical by this definition.

“Similar” (poly)peptide sequences are those which, when aligned, share similar amino acid residues and most often, but not mandatorily, identical amino acid residues at the same positions of the sequences to be compared. Similar amino acid residues are grouped by chemical characteristics of their side chains into families. Said families are described below for “conservative amino acid substitutions”. The “similarity” or “percent (%) similarity” between sequences is the number of positions that contain identical or similar residues at the same sequence positions of the sequences to be compared divided by the total number of positions compared and multiplied by 100%. For example, if 6 out of 10 sequence positions have identical amino acid residues and 2 out of 10 positions contain similar residues, then the sequences have 80% similarity. The % similarity between two sequences can, e.g., be determined using EMBOSS Needle (https://www.ebi.ac.uk/Tools/psa/emboss_needle/), using a BLOSUM62 matrix, a “gap open penalty” of 10, a “gap extend penalty” of 0.5, a false “end gap penalty”, an “end gap open penalty” of 10 and an “end gap extend penalty” of 0.5. The percent (%) similarity is typically determined over the entire length of the query sequence on which the analysis is performed.

As used herein, the term “single-chain” refers to a molecule comprising amino acid monomers linearly linked by peptide bonds.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “wild-type”, “native” and “naturally occurring” are used interchangeably herein to refer to a gene or gene product that has the characteristics of that gene or gene product when isolated from a naturally occurring source. A wild type, native or naturally occurring gene or gene product (e.g., a polypeptide) is that which is most frequently observed in a population and is thus arbitrarily designed the “normal” or “wild-type” form of the gene or gene product.

Each embodiment described herein is to be applied mutatis mutandis to each and every embodiment unless specifically stated otherwise.

The term “enveloped virus fusion ectodomain polypeptide”, as used herein, refers to a polypeptide that contains a virion surface exposed portion of a mature enveloped virus fusion protein, with or without the signal peptide, but which lacks the transmembrane domain and cytoplasmic tail of the naturally occurring enveloped virus fusion protein.

### 2. Chimeric Polypeptides

The present invention is predicated on a further advancement of the strategy for artificially stabilizing or “clamping” an enveloped virus fusion protein ectodomain polypeptide in a pre-fusion conformation. Generally, this “molecular clamping” strategy employs fusion or linkage of a structure-stabilizing moiety (SSM) to an ectodomain polypeptide to form a chimeric polypeptide.

The structure-stabilizing moiety (SSM) is typically a single-chain polypeptide, which comprises complementary heptad repeats that lack complementarity to the ectodomain polypeptide and that therefore preferentially associate with each other rather than with structural elements of the ectodomain polypeptide. Association of the complementary heptad repeats to one another under conditions suitable for their association (e.g., in aqueous solution) results in formation of an anti-parallel, two-helix bundle that inhibits rearrangement of the ectodomain polypeptide to a post-fusion conformation. The two-helix bundle of the structure-stabilizing moiety can trimerize to form a highly stable six-helix bundle, thus permitting self-assembly of the chimeric polypeptide to form an artificial enveloped virus fusion protein complex. The complex so assembled can mimic the pre-fusion conformation of a native enveloped virus fusion protein complex and comprises three chimeric polypeptides, characterized by a six-helix bundle formed by the coiled coil structures of the respective structure-stabilizing moieties of the chimeric polypeptides.

Whereas the originally established clamp technology, due to the utilization of a structure-stabilizing moiety (SSM) derived from the human immunodeficiency virus (HIV) glycoprotein 41 (gp41), was found to be disadvantageously associated with the induction of antibodies that led to HIV diagnostic interference, the present inventors set out to develop alternative clamp constructs which overcome these impediments while providing at least comparable or even improved capacities to stabilize the targeted viral fusion protein antigens in their “pre-fusion”-conformation and thus to induce protective neutralizing antibody responses upon vaccination. To that end, the inventors designed a panel of nineteen putative clamp sequences derived from the trimerization domains of fusion proteins from selected viruses known to not commonly infect humans. The nineteen candidate clamps were fused to the RSV fusion protein ectodomain. Subsequent assessment by thorough biophysical characterization led to the identification of lead molecules derived from the fusion proteins of two genetically related caprine lentiviruses, i.e., two constructs (CD9, CD11; see Example 1) derived from the Visna virus (also known as Visna-Maedi virus, Maedi-Visna virus (MVV) and Ovine lentivirus) and one further construct (CD10; see Example 1) derived from Caprine Arthritis-Encephaltis-Virus (CAEV).

The results presented herein demonstrate that the novel vaccine leads generated are viable alternatives to the previous HIV-clamp-based molecules. Furthermore, the data indicate that these leads are superior in terms of stability and at least equivalent in terms of their capacity to elicit neutralizing antibody responses compared to the earlier developed HIV-clamp-based vaccine candidate.

In accordance with the newly identified lead structures, the chimeric polypeptide as defined by the herein disclosed invention is characterized by comprising a microbial polypeptide, preferably an enveloped virus fusion ectodomain polypeptide, that is operably connected downstream to a heterologous, structure-stabilizing moiety (SSM) with specific sequence peculiarities as detailed below.

In a similar vein, the herein disclosed “molecular clamping” strategy can also be suitably employed for holding other (poly)peptides in a trimeric state. For example, according to a preferred embodiment of the first aspect of the invention, a bacterial outer membrane polypeptide (preferably a bacterial trimeric autotransporter adhesin (TAA) polypeptide) is fused with the structure-stabilizing moiety (SSM). The two-helix bundle of the structure-stabilizing moiety can then also trimerize to form a highly stable six-helix bundle, thus also permitting self-assembly of a trimer of the bacterial outer membrane polypeptide (e.g., a trimer of TAA polypeptides).

### 2.1 Structure-Stabilizing Moieties

The “structure-stabilizing moiety (SSM)”, as employed in connection with the present invention, is a polypeptide comprising, in an N- to C-terminal order, a first heptad repeat region (FHRR) and second heptad repeat region (SHRR), wherein the FHRR and SHRR may optionally be interconnected by a linker region (preferably in the following N- to C-terminal order: FHRR-linker-SHRR) as further defined herein below (e.g., in section 2.1.2).

### 2.1.1 Heptad Repeats

Alpha-helical coiled coils have been characterized at the level of their amino acid sequences, in that, each helix is constituted of a series of heptad repeats. A heptad repeat (heptad unit, heptad) is a 7-residue sequence motif which can be encoded as hpphppp, and wherein each ‘h’ represents a hydrophobic residue and each ‘p’ is a polar (i.e., hydrophilic) residue. Occasionally, p-residues are observed at h-positions, and vice versa. A heptad repeat is also often encoded by the patterns a-b-c-d-e-f-g (abcdefg) or d-e-f-g-a-b-c (defgabc), in which case the indices ‘a’ to ‘g’ refer to the conventional heptad positions at which typical amino acid types are observed. By convention, indices ‘a’ and ‘d’ denote the positions of the core residues (central, buried residues) in a coiled coil. The typical amino acid types that are observed at core a- and d-positions are hydrophobic amino acid residue types; at all other positions (non-core positions), predominantly polar (hydrophilic) residue types are observed. Thus, conventional heptad patterns ‘hpphppp’ match with the pattern notation ‘abcdefg’ (‘hppphpp’ patterns match with the pattern notation ‘defgabc’, this notation being used for coiled coils starting with a hydrophobic residue at a d-position).

The heptad repeat regions (HRRs) as referred to in accordance with the present invention include at least 2, and suitably 3 or more (preferably consecutive, i.e. uninterrupted) heptad repeats in individual α-helices of the coiled coil structure. Each series of consecutive heptad repeats in a helix is denoted a ‘heptad repeat sequence’ (HRS). The start and end of a heptad repeat sequence is preferably determined on the basis of the experimentally determined three-dimensional (3-D) structure, if available. If a 3-D structure is not available, the start and end of a heptad repeat sequence is preferably determined on the basis of an optimal overlay of a (hpphppp)n or (hppphpp)n pattern with the actual amino acid sequence, where ‘h’ and ‘p’ denote hydrophobic and polar (hydrophilic) residues, respectively, and where ‘n’ is a number equal to or greater than 2. The start and end of each heptad repeat sequence is taken to be the first and last hydrophobic residue at an a- or d-position, respectively. Conventional H-residues are preferably selected from the group consisting of valine, isoleucine, leucine, methionine, phenylalanine, tyrosine, tryptophan, histidine, glutamine, threonine, serine and alanine, more preferably from the group consisting of valine, isoleucine, leucine and methionine, and most preferably isoleucine. Conventional p-residues are preferably selected from the group consisting of glycine, alanine, cysteine, serine, threonine, histidine, asparagine, aspartic acid, glutamine, glutamic acid, lysine and arginine. In case this method does not permit unambiguous assignment of amino acid residues to a heptad repeat sequence, a more specialized analysis method can be applied, such as the COILS method of Lupas et al. (1991. Science 252: 1162-1164; http://www.russell.embl-heidelberg.de/cgi-bin/coils-svr.pl).

In the chimeric polypeptide according to the present invention, the “structure-stabilizing moiety (SSM)” comprises, in an N- to C-terminal order, a first heptad repeat region (FHRR) and second heptad repeat region (SHRR), wherein (i) the FHRR comprises or consists of an amino acid sequence having at least 60% (or, with increasing preference, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the amino acid sequence set forth in SEQ ID NO: 80 or 81, and the SHRR comprises or consists of an amino acid sequence having at least 40% (or, with increasing preference, at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the amino acid sequence set forth in SEQ ID NO: 82 or 83; and/or (ii) the FHRR comprises or consists of an amino acid sequence having at least 90% (or, with increasing preference, at least 91%, 92%, 93%, 94%, 95%, 96%, 97%, 98%, or 99%, most preferably 100%) sequence similarity to the amino acid sequence set forth in SEQ ID NO: 80 or 81, and the SHRR comprises or consists of an amino acid sequence having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence similarity to the amino acid sequence set forth in SEQ ID NO: 82 or 83.

In accordance with the present invention, (i) the FHRR comprises or consists of an amino acid sequence having at least 60% (or, with increasing preference, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the amino acid sequence set forth in SEQ ID NO: 80, and the SHRR comprises or consists of an amino acid sequence having at least 40% (or, with increasing preference, at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the amino acid sequence set forth in SEQ ID NO: 82; and/or (ii) the FHRR comprises or consists of an amino acid sequence having at least 90% (or, with increasing preference, at least 91%, 92%, 93%, 94%, 95%, 96%, 97%, 98%, or 99%, most preferably 100%) sequence similarity to the amino acid sequence set forth in SEQ ID NO: 80, and the SHRR comprises or consists of an amino acid sequence having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence similarity to the amino acid sequence set forth in SEQ ID NO: 82.

In some embodiments, (i) the FHRR comprises or consists of an amino acid sequence having at least 60% (or, with increasing preference, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the amino acid sequence set forth in SEQ ID NO: 80, and the SHRR comprises or consists of an amino acid sequence having at least 40% (or, with increasing preference, at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the amino acid sequence set forth in SEQ ID NO: 82; and/or (ii) the FHRR comprises or consists of an amino acid sequence having at least 90% (or, with increasing preference, at least 91%, 92%, 93%, 94%, 95%, 96%, 97%, 98%, or 99%, most preferably 100%) sequence similarity to the amino acid sequence set forth in SEQ ID NO: 80, and the SHRR comprises or consists of an amino acid sequence having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence similarity to the amino acid sequence set forth in SEQ ID NO: 82.

In some embodiments, the structure-stabilizing moiety is capable of homo-trimerization with the structure-stabilizing moieties of two further chimeric polypeptides; wherein preferably, by the homo-trimerization, a six-helix bundle is formed, wherein the six-helix bundle is composed of an inner trimer of three parallel oriented, substantially α-helical FHRRs against which three substantially α-helical SHRRs are packed in an anti-parallel orientation relative to the FHRRs.

In some embodiments, the FHRR and SHRR each comprise an independently selected, n-times repeated 7-residue motif characterized by a pattern of amino acids, represented as (a-b-c-d-e-f-g-)ₙ or (d-e-f-g-a-b-c-)ₙ, wherein the pattern elements ‘a’ to ‘g’ denote positions at which the amino acids are located and n is a number equal to or greater than 2, and at least 50% of the positions ‘a’ and ‘d’ are occupied by hydrophobic amino acids and at least 50% of the positions ‘b’, ‘c’, ‘e’, ‘f’ and ‘g’ are occupied by polar (hydrophilic) amino acids.

With respect to which amino acids in the context of the present disclosure are categorized as falling within the groups of hydrophilic (polar) or hydrophobic (non-polar) amino acids, respectively, reference is made to Table 1, supra.

In preferred embodiments of the latter embodiments, the FHRR comprises an independently selected, 4-times repeated 7-residue motif, and the SHRR comprises an independently selected, 5-times repeated 7-residue motif, wherein the 7-residue motif is characterized by a pattern of amino acids, represented as (a-b-c-d-e-f-g-) or (d-e-f-g-a-b-c-), wherein the pattern elements ‘a’ to ‘g’ denote positions at which the amino acids are located, and at least 50% of the positions ‘a’ and ‘d’ are occupied by hydrophobic amino acids and at least 50% of the positions ‘b’, ‘c’, ‘e’, ‘f’ and ‘g’ are occupied by hydrophilic amino acids.

In some embodiments, the structure-stabilizing moiety has a glutamine at the position corresponding to position 17 of SEQ ID NO: 80.

As apparent from the herein disclosed experimental data, presence of a corresponding mutation (Gln17) provides a slight further increase of soluble protein yields (see Example 4, Table 4; CD11 vs. CD9), indicative of a stabilizing effect in terms of protein folding and trimer association. Additional data consistently indicated that this mutation also advantageously enhances thermal stability (see Example 5, FIG. 4). In view of these advantageous effects provided by glutamine at position 17, it is expected that comparable positive effects are also provided by substitution to asparagine, or similarly to glutamate or aspartate, which are also contemplated herein as alternative preferred embodiments. However, as constructs having a serine or threonine at position 17 also provided a suitable trimer stabilization, it is also contemplated as further alternative embodiments that the structure-stabilizing moiety has a serine or threonine at the position corresponding to position 17 of SEQ ID NO: 80.

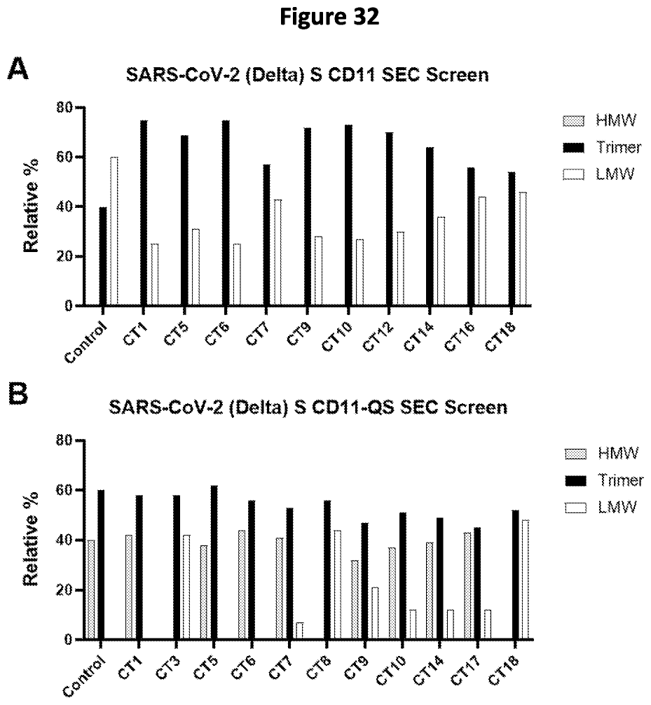

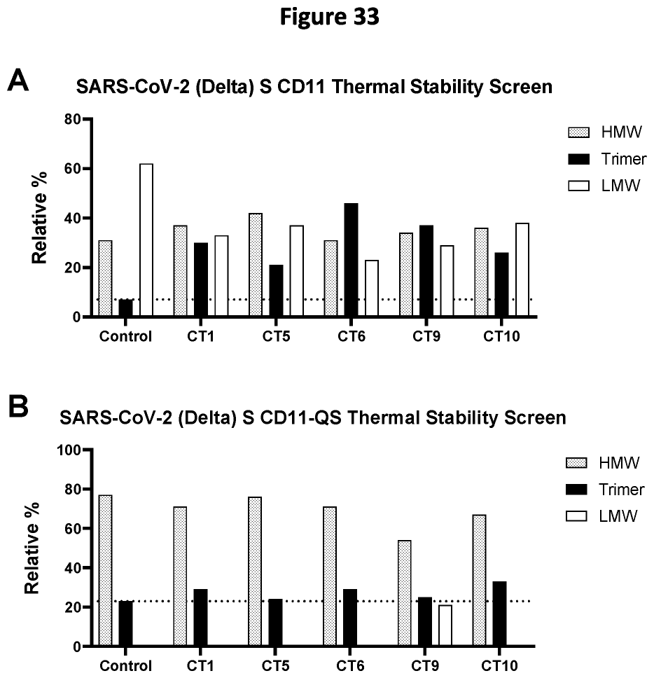

In alternative preferred embodiments, the structure-stabilizing moiety has a leucine at the position corresponding to position 17 of SEQ ID NO: 80. As is evident from the data presented in Example 16, presence of a corresponding mutation (Leu17; see construct denoted “CT9”) led to a substantial improvement in terms of presence of trimer in solution, as revealed by size exclusion chromatography (SEC) analysis (FIG. 32), and also in terms thermal stability of the trimer (FIG. 33).

In particular embodiments, the structure-stabilizing moiety comprises at least one immune-silencing moiety that reduces or inhibits elicitation of an immune response to the structure-stabilizing moiety. These embodiments are advantageous as they can permit the generation of a selective and/or enhanced immune response to the microbial polypeptides, e.g., (i) the enveloped virus fusion ectodomain polypeptide or a complex thereof; or (ii), in the instance where the chimeric polypeptide comprises bacterial outer membrane polypeptide (e.g., a TAA polypeptide), to the bacterial outer membrane polypeptide (e.g., the TAA polypeptide) or a complex thereof.

The immune-silencing moiety can be a glycosylation site that is specifically recognized and glycosylated by one or more glycosylation enzymes, in particular glycosyltransferase(s). Glycosylations can be N-linked or O-linked. N-linked refers to the attachment of the carbohydrate moiety to the side chain of an asparagine residue. The tripeptide sequences N—X—S and N—X—T (expressed in three-letter code as Asn-Xaa-Ser and Asn-Xaa-Thr, respectively), where X (Xaa) is any amino acid except P (Pro), are the recognition sequences for enzymatic attachment of the carbohydrate moiety to the asparagine (Asn) side chain, and these sequences are commonly referred to as ‘glycosylation sites’ or ‘sequons’. O-linked glycosylation refers to the attachment of one of the monosaccharide units N-acetylgalactosamine (GalNAc) or galactose (Gal) to a hydroxyamino acid, most commonly serine (Ser) or threonine (Thr), although 5-hydroxyproline or 5-hydroxylysine may also be used, which monosaccharide units may be further elongated by additional monosaccharide units, such as Gal, GalNAc or N-acetylglucosamine (GlcNAc). The immune-silencing moiety may be inserted into the structure stabilizing moiety, including one or both of the heptad repeat regions.

In particularly preferred embodiments of the latter embodiments, the at least one immune silencing moiety is an N-linked glycosylation site.

In even more preferred embodiments, the N-linked glycosylation site is post-translationally modified and a glycan (e.g., a polysaccharide, oligosaccharide or monosaccharide) is attached. The glycan attached may correspond to those known to be commonly utilised in the particular mammal (preferably, Homo sapiens) envisaged for being administered with the chimeric polypeptide or complex thereof, or with any of the compositions provided herein, for the sake of diminishing or circumventing the immune response to the corresponding protein sequence. The skilled person will be able to select suitable host cells for recombinant expression of the chimeric polypeptide(s) and complexes thereof, wherein said host cells bear a glycosylation machinery that yields such N-glycans that are endogenous to the targeted mammalian subject species and which thus are likely to provide an immune-silencing effect.

In some embodiments, the structure-stabilizing moiety comprises at least one glycosylation site; wherein preferably the at least one glycosylation site is an N-linked glycosylation site, selected from the group consisting of: (1) -Asn-Xaa-Ser-; and (2) -Asn-Xaa-Thr-; wherein Xaa is an amino acid other than Pro; wherein preferably the glycosylation site is glycosylated with an occupancy level of at least 50%.

Generally, the “occupancy level” of a glycosylation site in a given protein molecule refers to the percentage (%) level of this glycosylation site actually being glycosylated within a given population of that protein molecule. The skilled person is aware that this level is dependent, inter alia, upon the intrinsic sequence-wise and consequently structural properties of the protein, but also from the organism (expression host) and the expression route (cytoplasmic vs. secretory) that is utilized for the recombinant expression of that protein. Means and methods for determining and quantifying the “% occupancy level” of a given glycosylation site are known in the art and can be readily applied by the skilled person. One exemplary method for determining the “% occupancy level” for an N-glycosylation site is also described herein; see appended Example 14.

In some embodiments, the structure-stabilizing moiety comprises one or more N-linked glycosylation sites at the amino acid positions corresponding to: (i) positions 5-7 of SEQ ID NO: 80; (ii) positions 1-3 of SEQ ID NO: 82; (iii) positions 6-8 of SEQ ID NO: 82; (iv) positions 13-15 of SEQ ID NO: 82; (v) positions 17-19 of SEQ ID NO: 82; and/or (vi) positions 27-29 of SEQ ID NO: 82; wherein preferably each N-linked glycosylation site is independently -Asn-Xaa-Thr-, wherein Xaa is an amino acid other than Pro.

In some embodiments, the structure-stabilizing moiety comprises N-linked glycosylation sites at the amino acid positions corresponding to: (i) (i-a) positions 5-7 of SEQ ID NO: 80; (i-b) positions 1-3 of SEQ ID NO: 82; and (i-c) positions 17-19 of SEQ ID NO: 82; or (ii) (ii-a) positions 5-7 of SEQ ID NO: 80; (ii-b) positions 1-3 of SEQ ID NO: 82; (ii-c) positions 17-19 of SEQ ID NO: 82; and (ii-d) positions 27-29 of SEQ ID NO: 82; or (iii) (iii-a) positions 5-7 of SEQ ID NO: 80; (iii-b) positions 1-3 of SEQ ID NO: 82; (iii-c) positions 13-15 of SEQ ID NO: 82; (iii-d) positions 17-19 of SEQ ID NO: 82; and (iii-e) positions 27-29 of SEQ ID NO: 82; or (iv) (iv-a) positions 5-7 of SEQ ID NO: 80; (iv-b) positions 1-3 of SEQ ID NO: 82; (iv-c) positions 6-8 of SEQ ID NO: 82; (iv-d) positions 13-15 of SEQ ID NO: 82; (iv-e) positions 17-19 of SEQ ID NO: 82; and (iv-f) positions 27-29 of SEQ ID NO: 82; wherein preferably each N-linked glycosylation site is independently -Asn-Xaa-Thr-, wherein Xaa is an amino acid other than Pro.

In some embodiments, the structure stabilizing moiety has (i) an arginine at the amino acid position corresponding to glutamine 22 of SEQ ID NO: 80; (ii) a histidine at the amino acid position corresponding to asparagine 1 of SEQ ID NO: 82; (iii) a threonine at the amino acid position corresponding to histidine 2 of SEQ ID NO: 82; (iv) a serine at the amino acid position corresponding to alanine 25 of SEQ ID NO: 82; (v) a glutamine at the amino acid position corresponding to alanine 26 of SEQ ID NO: 82; (vi) a glutamine at the amino acid position corresponding to leucine 27 of SEQ ID NO: 82; (vii) a leucine at the amino acid position corresponding to glutamine 17 of SEQ ID NO: 80; (viii) a deletion of the amino acid residue at the position corresponding to arginine 37 of SEQ ID NO: 80; and/or (ix) a deletion of the amino acid residues at the positions corresponding to glutamine 1 and serine 2 of SEQ ID NO: 80.

In preferred embodiments of the letter embodiments, the structure stabilizing moiety has (i) an arginine at the amino acid position corresponding to glutamine 22 of SEQ ID NO: 80; (ii) a histidine at the amino acid position corresponding to asparagine 1 of SEQ ID NO: 82 and a threonine at the amino acid position corresponding to histidine 2 of SEQ ID NO: 82; (iii) a serine at the amino acid position corresponding to alanine 25 of SEQ ID NO: 82, a glutamine at the amino acid position corresponding to alanine 26 of SEQ ID NO: 82, and a glutamine at the amino acid position corresponding to leucine 27 of SEQ ID NO: 82; (iv) a leucine at the amino acid position corresponding to glutamine 17 of SEQ ID NO: 80; (v) a deletion of the amino acid residue at the position corresponding to arginine 37 of SEQ ID NO: 80; and/or (vi) a deletion of the amino acid residues at the positions corresponding to glutamine 1 and serine 2 of SEQ ID NO: 80. As is apparent from Example 16, specifically the size exclusion chromatography (SEC) data presented in FIGS. 32 and 33, each of the above-indicated site-specific mutations (i) to (v) (referred to in Example 16 as CT1, CT5, CT6, CT9 and CT10, respectively) provides a further enhancement of trimer stabilization relative to unmodified CD11.

In preferred embodiments, the structure stabilizing moiety has (i) an arginine at the amino acid position corresponding to glutamine 22 of SEQ ID NO: 80, and (ii) a deletion of the amino acid residues at the positions corresponding to glutamine 1 and serine 2 of SEQ ID NO: 80.

In a preferred embodiment, the structure stabilizing moiety has (i) a leucine at the amino acid position corresponding to position 20 of SEQ ID NO: 82; and/or (ii) a glutamine at the amino acid position corresponding to position 11 of SEQ ID NO: 80. As is apparent from the mutagenesis studies in Example 16, maintaining these amino acids as in the wild-type sequence of CD11 is beneficial in terms of stabilization of the trimer.

In some embodiments, the structure-stabilizing moiety comprises one or more unnatural amino acids.

A “unnatural” or “non-natural amino acid”, as used herein, refers to an amino acid that is not one of the 20 common naturally occurring amino acids or the rare naturally occurring amino acids e.g., selenocysteine (Sec) or pyrrolysine (Pyl). Other terms that may be used synonymously with the term non-natural amino acid is non-naturally encoded amino acid, unnatural amino acid, non-naturally-occurring amino acid, and variously hyphenated and non-hyphenated versions thereof. The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated into a growing (poly)peptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term non-natural amino acid includes, but is not limited to, amino acids which do not occur naturally and may be obtained synthetically or may be obtained by modification of non-natural amino acids. Non-natural amino acids can be incorporated into the chimeric polypeptide (e.g., into one or both of the therein comprised heptad repeat regions) or any other (poly)peptide contemplated herein by using an expanded genetic code. The non-natural amino acids are biosynthetically incorporated into a desired location using tyrosyl-tRNA/aminoacyl-tRNA synthetase orthogonal pair and a nonsense codon at the desired site. The non-natural amino acids are supplied to cells expressing a construct from which the chimeric polypeptide is expressible, from an external source and this strategy can incorporate side chains with a wide range of physical attributes including, but not limited to, chemical crosslinking group (e.g., azide or haloalkane), a trackable maker (e.g., fluorescent or radioactive) and photosensitive groups to enable temporally controlled modifications. To these unnatural amino acids various moieties can be covalently linked by chemical addition to the structure-stabilizing moiety to provide advantageous properties.

In preferred embodiments of the latter embodiments, the one or more unnatural amino acids permit coupling of (i) polyethylene glycol (PEG); (ii) an immune-stimulating moiety; or (iii) a lipid.

Further embodiments may include any possible combination of the above examples, or additional unnatural chemical addition, covalently linked to the structure-stabilizing moiety.

Optionally, one or more additional cysteine residues may be inserted into the FHRR and/or SHRR to form disulfide bonds and further stabilize the anti-parallel, α-helical coiled coil structure of the structure stabilizing moiety.

### 2.1.2 Linkers

The structure-stabilizing moiety (SSM) of the present invention can suitably comprise a linker that spaces the first and second heptad repeat regions (also referred to herein as FHRR and SHRR, respectively).

The linker generally includes any amino acid residue that cannot be unambiguously assigned to a heptad repeat sequence. Linkers are frequently used in the field of protein engineering to interconnect different functional units, e.g., in the creation of single-chain variable fragment (scFv) constructs derived from antibody variable light (VL) and variable heavy (VH) chains. They are generally conformationally flexible in solution and are suitably and predominantly composed of polar amino acid residue types. Typical (frequently used) amino acids in flexible linkers are serine and glycine. Less preferably, flexible linkers may also include alanine, threonine and proline. Thus, an intervening linker of the structure-stabilizing moiety is preferably flexible in conformation to ensure relaxed (unhindered) association of FHRR and SHRR as two-helix bundle that suitably adopts an α-helical coiled coil structure. Suitable linkers for use in the polypeptides envisaged herein will be clear to the skilled person, and may generally be any linker used in the art to link amino acid sequences, as long as the linkers are structurally flexible, in the sense that they permit, and suitably do not impair, assembly of the characteristic two-helix bundle structure of the structure-stabilizing moiety.

The skilled person will be able to determine the optimal linkers, optionally after performing a limited number of routine experiments. The intervening linker is suitably an amino acid sequence generally consisting of at least 1 amino acid residue and usually consisting of at least 2 amino acid residues, with a non-critical upper limit chosen for reasons of convenience being about 100 amino acid residues. In particular embodiments, the linker consists of about 1 to about 50 amino acid residues, or about 50 to about 100 amino acid residues, usually about 1 to about 40 amino acid residues, typically about 1 to about 30 amino acid residues. In non-limiting examples, the linker has about the same number of amino acids as the number of amino acids connecting complementary FHRR and SHHR regions of a Class I enveloped virus fusion protein. In particular non-limiting embodiments, at least 50% of the amino acid residues of a linker sequence are selected from the group proline, glycine, and serine. In further non-limiting embodiments, at least 60%, such as at least 70%, such as for example 80% and more particularly 90% of the amino acid residues of a linker sequence are selected from the group proline, glycine, and serine. In other particular embodiments, the linker sequences essentially consist of polar amino acid residues; in such particular embodiments, at least 50%, such as at least 60%, such as for example 70% or 80% and more particularly 90% or up to 100% of the amino acid residues of a linker sequence are selected from the group consisting of glycine, serine, threonine, alanine, proline, histidine, asparagine, aspartic acid, glutamine, glutamic acid, lysine and arginine. In specific embodiments, linker sequences may include [GGSG]ₙGG (SEQ ID NOs: 161-170), [GGGGS]ₙ (SEQ ID NOs: 152-155; 171-176), [GGGGG]ₙ (SEQ ID NOs: 160; 177-185), [GGGKGGGG]ₙ (SEQ ID NOs: 186-195), [GGGNGGGG]ₙ (SEQ ID NOs: 196-205), [GGGCGGGG]ₙ (SEQ ID NOs: 206-215), wherein n is an integer from 1 to 10, suitably 1 to 5, more suitably 1 to 3.

In preferred embodiments, the FHRR and the SHRR comprised in the structure-stabilizing moiety (SSM) are connected by a linker. Preferably, the linker comprises or consists of a peptide with an amino acid sequence identical to SEQ ID NO: 84 or 85, more preferably an amino acid sequence defined by SEQ ID NO: 84.

In addition to the spacing of the heptad repeat regions (i.e., the FHRR and SHRR) of the structure-stabilizing moiety (SSM), and preferably introducing structural flexibility to facilitate anti-parallel association of those regions, the linker may comprise one or more ancillary functionalities.

For example, the linker may comprise a purification moiety that facilitates purification of the chimeric polypeptide and/or at least one immune-modulating moiety that modulates an immune response to the chimeric polypeptide.

Purification moieties typically comprise a stretch of amino acids that enables recovery of the chimeric polypeptide through affinity binding. Numerous purification moieties or ‘tags’ are known in the art, illustrative examples of which include biotin carboxyl carrier protein-tag (BCCP-tag), Myc-tag (c-myc-tag), Calmodulin-tag, FLAG-tag, HA-tag, His-tag (Hexahistidine-tag, His6, 6H, 6×His), Maltose binding protein-tag (MBP-tag), Nus-tag, Chitin-binding protein-tag (CBP-tag) Glutathione-S-transferase-tag (GST-tag), Green fluorescent protein-tag (GFP-tag), Polyglutamate-tag, Amyloid beta-tag, Thioredoxin-tag, S-tag, Softag 1, Softag 3, Strep-tag, Streptavidin-binding peptide-tag (SBP-tag), biotin-tag, streptavidin-tag and V5-tag.

Immune-modulating moieties can be introduced into the linker to modulate the immune response elicited by the chimeric polypeptide or complex thereof. Non-limiting examples of such moieties include immune-silencing or suppressing moieties as described for example above, antigenic moieties, including antigenic moieties derived from pathogenic organisms, or other disease associated antigenic moieties such as cancer or tumor-associated antigens. Exemplary pathogenic organisms include, but are not limited to, viruses, bacteria, fungi parasites, algae and protozoa and amoebae. In specific embodiments, the antigenic moieties are derived from antigens of pathogenic viruses. Illustrative viruses responsible for diseases include, but are not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus and other herpesviruses such as papillomavirus, Ebola virus, influenza virus, Japanese encephalitis (e.g., GenBank Accession No. E07883), dengue (e.g., GenBank Accession No. M24444), hantavirus, Sendai virus, respiratory syncytial virus, orthomyxoviruses, vesicular stomatitis virus, Visna virus, cytomegalovirus, human immunodeficiency virus (HIV) (e.g., GenBank Accession No. U18552). Any suitable antigen derived from such viruses are useful in the practice of the present invention. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g., hepatitis A, B, and C. Illustrative examples of influenza viral antigens include, but are not limited to, antigens such as hemagglutinin and neuraminidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS 1, NS 1, NS 1-NS2A, 80% E, and other Japanese encephalitis viral antigen components.

Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, See Fundamental Virology, Second Edition, eds. Fields, B. N. and Knipe, D. M., 1991, Raven Press, New York, for additional examples of viral antigens. In particular embodiments, the viral antigen is an antigen of an enveloped virus to which the ectodomain polypeptide corresponds. In other embodiments, the viral antigen is an antigen of a different enveloped virus to which the ectodomain polypeptide corresponds.

In some embodiments, one or more cancer- or tumor-associated antigens are inserted into the linker. Such antigens include, but are not limited to, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV 18, TUAN, alpha-feto protein (AFP), OFP, CA-125, CA-50, CA-19-9, renal tumor-associated antigen G250, EGP-40 (also known as EpCAM), S100 (malignant melanoma-associated antigen), p53, prostate tumor-associated antigens (e.g., PSA and PSMA), p21ras, Her2/neu, EGFR, EpCAM, VEGFR, FGFR, MUC-I, CA 125, CEA, MAGE, CD20, CD19, CD40, CD33, A3, antigen specific to A33 antibodies, BrE3 antigen, CD1, CD1a, CD5, CD8, CD14, CD15, CD16, CD21, CD22, CD23, CD30, CD33, CD37, CD38, CD40, CD45, CD46, CD52, CD54, CD74, CD79a, CD126, CD138, CD154, B7, la, Ii, HMI.24, HLA-DR (e.g., HLA-DR10), NCA95, NCA90, HCG and sub-units, CEA (CEACAMS), CEACAM-6, CSAp, EGP-I, EGP-2, Ba 733, KC4 antigen, KS-I antigen, KS1-4, Le-Y, MUC2, MUC3, MUC4, PIGF, ED-B fibronectin, NCA 66a-d, PAM-4 antigen, PSA, PSMA, RS5, SIOO, TAG-72, TO, TAG TRAIL-RI, TRAIL-R2, p53, tenascin, insulin growth factor-1 (IGF-I), Tn antigen etc.

The antigenic moiety or moieties included in the linker may correspond to full-length antigens or part antigens. When part antigens are employed, the part antigens may comprise one or more epitopes of an antigen of interest, including B cell epitopes and/or T cell epitopes (e.g., cytotoxic T lymphocyte (CTL) epitopes and/or T helper (Th) epitopes). Epitopes of numerous antigens are known in the literature or can be determined using routine techniques known to persons of skill in the art. In other embodiments the linker may include another cell targeting moiety which can provide delivery to a specific cell type within the immunized individual. Cell populations of interest include, but are not limited to, B-cells, Microfold cells and antigen-presenting cells (APC). In the later example the targeting moiety facilitates enhanced recognition of the chimeric polypeptide or complex thereof to an APC such as a dendritic cell or macrophage. Such targeting sequences can enhance APC presentation of epitopes of an associated ectodomain polypeptide, which can in turn augment the resultant immune response, including intensification or broadening the specificity of either or both of antibody and cellular immune responses to the ectodomain polypeptide. Non-limiting examples of APC-targeting moieties include ligands that bind to APC surface receptors such as, but not limited to, mannose-specific lectin (mannose receptor), IgG Fc receptors, DC-SIGN, BDCA3 (CD141), 33D1, SIGLEC-H, DCIR, CD11c, heat shock protein receptors and scavenger receptors. In particular embodiments, the APC-targeting moiety is a dendritic cell targeting moiety, which comprises, consists or consists essentially of the sequence FYPSYHSTPQRP (Uriel et al., J. Immunol. 2004 172: 7425-7431) or NWYLPWLGTNDW (Sioud et al., FASEB J 2013 27(8): 3272-83).

### 2.1.3 Particularly Preferred Structure-Stabilizing Moieties (SSMs)

Two structure-stabilizing moieties were selected by the inventors as lead constructs, and designated “Clamp2” and “Clamp2s”:
Clamp2 (alternatively designated herein “CD11-QS”) is defined by SEQ ID NO: 265:

[SEQ ID NO: 265]
LANATAAQQEVLEAQYAMVQHIAKGIRILEARVARGGSGGNHTWQQWEE
EIEQHEGNLSLLLREAALQVHIAQRDARRI

Clamp2s (alternatively designated herein as “CD11_145T8-QS_CT5”, which corresponds to a silenced variant of Clamp2) is defined by SEQ ID NO: 266:

[SEQ ID NO: 266]
LANATAAQQEVLEAQYAMVQHIAKGIRILEARVARGGSGGNHTTWQQWE
EEIENHTGNLTLLLREAANQTHIAQRDARRI

### 2.1.4 Membrane Tethering

In may be advantageous, in certain embodiments, to provide a chimeric polypeptide, or a complex thereof, which is tethered to the cell surface, such as the cell surface of a cell expressing the chimeric polypeptide.

For example, in the chimeric polypeptides according to the first aspect of the invention, which are intended for being employed as a vaccine, membrane tethering to the cell surface (e.g., of a host cell, which upon vaccination with an mRNA, viral vector or other nucleic acid encoding the chimeric polypeptide expresses the chimeric polypeptide) will allow displaying the chimeric polypeptide (or at least the antigen portion thereof, i.e., the microbial polypeptide, preferably an enveloped virus fusion ectodomain polypeptide or a bacterial outer membrane polypeptide (e.g., a TAA polypeptide) onto the cell surface.

Without wishing to be bound by any theory, it is thought that a corresponding setup can be advantageous in terms of providing an enhanced stimulation of the immune response and thus for eliciting a more potent, persistent broadly neutralizing antibody response. This is because the cells and/or cell-derived organelles expressing and consequently displaying high concentration and/or high density of chimeric polypeptide may help to recruit and activate naïve and memory B cells and thereby enhance the stimulation of a potent B-cell mediated neutralizing antibody response. Moreover, an interaction with B cells may additionally be amplified through avidity effects resulting from the display of multiple chimeric polypeptides, or multiple complexes thereof, on the cell surface. An efficient formation of neutralizing antibodies by B cells also requires helper functions by T cells (in particular, CD4 T cells). Whereas an induction of a B cell response requires a direct interaction with the antigen, CD4T cells are stimulated by peptides derived from the same antigen in complex with MHCII molecules. It is hence presumed that tethering of the chimeric polypeptide, or its complexes, to the surface of cells and/or cell-derived organelles can indirectly lead to higher epitope presentation through MHCII which might also help to recruit CD4 T cells and thereby additionally enhance the immune response and formation of potent neutralizing antibodies.

Two general kinds of embodiments of membrane-tethered constructs particularly contemplated herein include:
(i) embodiments, wherein the SSM additionally comprises a membrane-tethering polypeptide; and
(ii) embodiments, wherein the chimeric polypeptide additionally comprises a transmembrane (TM) domain upstream of (i.e., N-terminal to) the SSM.

Representative embodiments relating to (i) and (ii) are set out in sections 2.1.4.1 and 2.1.3.2, respectively, following hereafter.

### 2.1.4.1 Membrane Tethering (Poly)Peptide

In particularly preferred embodiments, the chimeric polypeptide of the invention additionally comprises a “membrane tethering (poly)peptide”. As used herein, the term “membrane tethering (poly)peptide”, “membrane anchoring (poly)peptide”, “membrane tether” or “membrane anchor”, “membrane localisation (poly)peptide”, “membrane targeting (poly)peptide”, or further equivalents thereof refers to a (poly)peptide sequence that can act as an anchor, tethering the chimeric polypeptide of the invention to the extracellular surface of the cell membrane (e.g., a lipid bilayer of a cell membrane). In its broadest sense, the term “membrane tethering (poly)peptide” or its herein referred equivalents encompasses such (poly)peptides which capacity to at least partially insert into a membrane (such as the lipid bilayer of a cell or of a (nano)lipid particle) is due to the intrinsic nature and physicochemical properties of the specific amino acid residues comprised therein (e.g., amino acids having sidechains characterized by having high intrinsic hydrophobicity, such as, in particular, tryptophan, phenylalanine, tyrosine, isoleucine, leucine, and valine). However, the term also embraces such (poly)peptides, where this capacity is conferred through the presence of certain chemical or posttranslational modifications of one or more of the amino acid residues comprised. For example, one or more amino acids may carry covalently attached lipids/fatty acids. Such an amino acid may, for example, but without intention to be limiting, be myristoylated, palmitoylated or prenylated.

In general, a membrane-tethering (poly)peptide may be suitably included at any position within the chimeric polypeptide as long as its presence does not, or at least substantially not, negatively interfere with the folding of the remaining portions of the chimeric polypeptide and/or sterically impart its capacity to trimerize. Moreover, the skilled person will understand that, in such instances where the chimeric polypeptide includes a microbial polypeptide (preferably an enveloped virus fusion ectodomain polypeptide or a bacterial outer membrane polypeptide (e.g., a TAA polypeptide)), a membrane-tethering (poly)peptide should only be included into the chimeric polypeptide at a position where its presence does not, or at least substantially not, sterically interfere with the surface accessibility of the microbial polypeptide (preferably the enveloped virus fusion ectodomain polypeptide or the bacterial outer membrane polypeptide (e.g., the TAA polypeptide)) in order to not negatively interfere with the immunogenic capacity of these antigenic portions.

As shown in the herein disclosed experimental evidence (see, e.g., Example 21), the present inventors found that constructs having a membrane tethering (poly)peptide inserted as a linker between the FHRR and the SHRR of the SSM were particularly effective for tethering the chimeric polypeptide or complexes thereof to the surface of cells expressing the chimeric polypeptide. Corresponding constructs are presumed to result in a structural arrangement as illustrated in FIG. 41B, wherein the SSM is oriented proximal to the cell membrane and the portion of the chimeric polypeptide which is upstream of the SSM (such as the enveloped virus fusion ectodomain polypeptide or the bacterial outer membrane polypeptide (e.g., a TAA polypeptide)) is displayed distal to the cell surface protruding away from it.

Thus, in preferred embodiments of those embodiments where the FHRR and the SHRR comprised in the SSM are connected by a linker, the linker comprises or consists of a membrane tethering (poly)peptide.

For being particularly suitable for constructs according to the latter embodiment, a membrane tethering (poly)peptide should be of a length and structural conformation that is compatible with the structural arrangement of the FHRR and the SSHR in the SSM. More specifically, the membrane tethering (poly)peptide should be of sufficient length to allow its stable insertion into the cell membrane, while its N- and C-termini should be spatially oriented/separated in a way that matches the C- and N-termini, respectively, of the FHRR and the SHRR of the SSM. It may, in addition, be beneficial in terms of providing a further stabilization and/or rigidification, if the membrane tethering (poly)peptide, or at least a substantial portion thereof, is itself capable to trimerize.

In preferred embodiments, the membrane-tethering polypeptide:
(i) comprises or consists of between, with increasing preference, 10 and 50, 15 and 40, 16 and 30, 18 and 28, 20 and 26, 22 and 24, most preferably 23 amino acid residues; and/or
(ii) comprises at least, with increasing preference, 1, 2, 3, 4, 5, 6, 7, 8, 9, 10, 11, 12, 13, 14, 15 or more amino acid residues selected from tryptophan, phenylalanine, tyrosine, isoleucine, leucine, and/or valine.

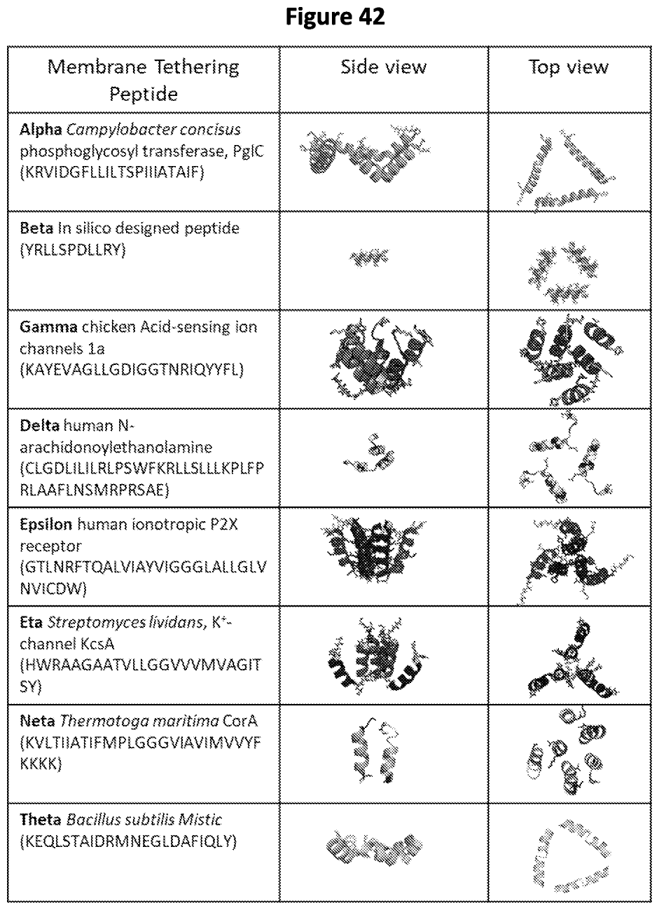

In the herein disclosed examples, a panel of eight different membrane tethering (poly)peptides (as defined by SEQ ID NO: 236-243, respectively) has been developed of which each was subsequently found to be effective for tethering the chimeric polypeptide to the exterior surface of the cell membrane (see Example 21 and FIGS. 41B and 42). Thus, in particularly preferred embodiments, the membrane tethering (poly)peptide comprises or consists of an amino acid sequence having at least 60% (or, with increasing preference, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to one of the amino acid sequences set forth in SEQ ID NOs: 236-243.

In the specific constructs as defined by SEQ ID NOs: 225-232 and employed in Example 21, a membrane tethering (poly)peptide was included into the linker region which interconnects the FHRR and the SHRR of the SSM. Accordingly, in these constructs, the included membrane tethering (poly)peptides as defined by SEQ ID NO: 236-243, respectively, were N- and C-terminally flanked by two (GG) and three (SGG) amino acids, respectively, which originated from the insertion into the original linker sequence GGSGG (as defined by SEQ ID NO: 84). Without wishing to be bound by any theory, it is presumed that the inclusion of one or more amino acids, such as glycine or serine (or other small amino acid residues), as flanking portions will be helpful to separate the membrane tethering (poly)peptide from the remaining portions of the chimeric polypeptide so that the former can insert into the cell membrane whereas the latter will be surface displayed, thus allowing an ideal antigen presentation.

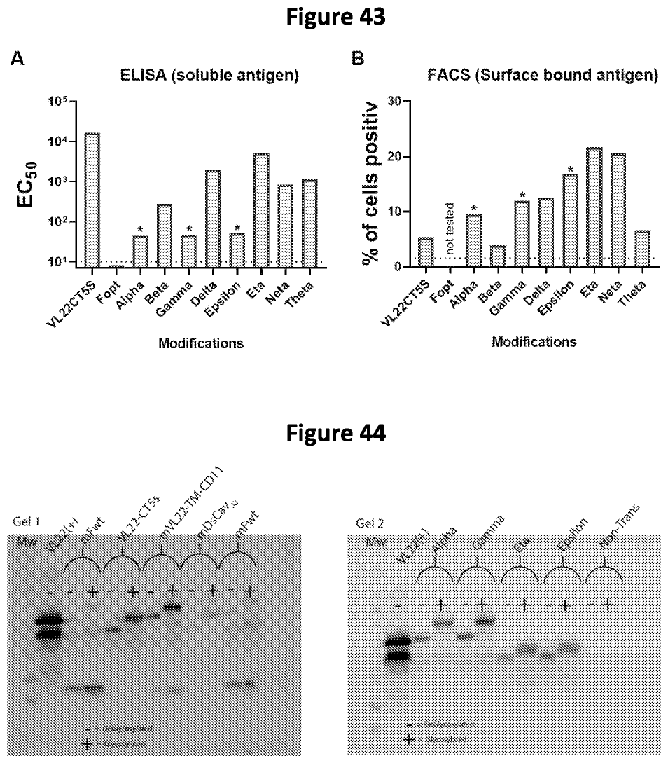

Thus, in preferred embodiments, the membrane tethering (poly)peptide comprises or consists of an amino acid sequence having at least 60% (or, with increasing preference, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to any one of the amino acid sequences set forth in SEQ ID NOs: 244-251.

Whereas each of the eight membrane tethering (poly)peptides tested proved to be effective for tethering the respective chimeric polypeptide to the cell membrane, the three constructs bearing the membrane tethering (poly)peptides referred to herein as “alpha”, “gamma” or “epsilon” (as defined by SEQ ID NOs: 236, 238 and 240, respectively) resulted in the highest levels of surface-bound chimeric polypeptides (as assessed by flow cytometry using an antigen specific monoclonal antibody; see FIG. 43B).

Thus, in preferred embodiments, the membrane tethering (poly)peptide comprises or consists of an amino acid sequence having at least 60% (or, with increasing preference, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to:
(a) any one of the amino acid sequences set forth in SEQ ID NOs: 236, 238 and 240; or
(b) any one of the amino acid sequences set forth in SEQ ID NOs: 244, 246 and 248.

A further analysis of the exemplified panel of constructs revealed that the inclusion of the membrane tethering (poly)peptide designated herein as “gamma” (as defined by SEQ ID NO: 238) was also most effective in terms of stabilizing the viral fusion protein (RSV F) in the pre-fusion conformation. Thus, in even more preferred embodiments, the membrane tethering (poly)peptide comprises or consists of an amino acid sequence having at least 60% (or, with increasing preference, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to SEQ ID NO: 238 or SEQ ID NOs: 246. In alternative, yet less preferred embodiments, however, a membrane tethering (poly)peptide may be comprised in the C-terminal portion of the chimeric polypeptide, i.e., C-terminal to the SHRR of the SSM. For example, in an exemplary embodiment of the latter embodiment, the chimeric polypeptide may comprise, C-terminal to the SHIRR of the SSM, a (poly)peptide which comprises a glycosylphosphatidylinositol (GPI)-anchor.

### 2.1.4.2 Membrane Tethering by Inclusion of a Transmembrane Region Upstream of the SSM

In alternative embodiments, membrane tethering of the chimeric polypeptide of the invention may be established through inclusion of a transmembrane (poly)peptide upstream of (i.e., N-terminal to) the SSM. Corresponding constructs are presumed to give rise to a structural arrangement as illustrated in FIG. 41C, wherein the SSM is oriented at the interior (i.e., the cytoplasmic side) of the cell membrane and the portion of the chimeric polypeptide which is upstream of (i.e., N-terminal to) the SSM, such as the enveloped virus fusion protein ectodomain polypeptide or the TAA polypeptide, is displayed extracellularly, i.e., on the cell surface and protruding away from it.

The term “transmembrane (poly)peptide” or “transmembrane domain”, as used herein, refers in its broadest sense to any (poly)peptide which can span (i.e., traverse) the lipid bilayer of a cell membrane and thus function to link the extracellular and intracellular portions of a polypeptide chain. This may be a single alpha helix, a transmembrane beta barrel, a beta-helix or any other structure. Typically, the transmembrane domain denotes a single transmembrane alpha helix of a transmembrane protein, also known as an integral protein.

For example, in related embodiments, where the chimeric polypeptide comprises a microbial polypeptide (e.g., an enveloped virus fusion ectodomain polypeptide or a bacterial surface polypeptide (e.g., a bacterial outer membrane polypeptide)), the chimeric polypeptide may additionally comprise a transmembrane (poly)peptide C-terminal of the microbial polypeptide (e.g., the enveloped virus fusion ectodomain polypeptide or the bacterial surface polypeptide) and N-terminal of the SSM. In particularly preferred embodiments of the latter embodiments, the transmembrane (poly)peptide is comprised in the chimeric polypeptide immediately C-terminal of the microbial polypeptide (e.g., C-terminal of the enveloped virus fusion ectodomain polypeptide or of the bacterial surface polypeptide) and N-terminal of the SSM. In even more preferred embodiments, if applicable, the transmembrane (poly)peptide corresponds, or substantially corresponds, to the transmembrane (poly)peptide which is naturally comprised in the microbial polypeptide (e.g., the enveloped virus fusion polypeptide).

In preferred embodiments of the chimeric polypeptide of the first or second aspect of the invention, the chimeric polypeptide further comprises a hinge region which operably connects the microbial polypeptide (preferably the enveloped virus fusion ectodomain polypeptide or the bacterial outer membrane polypeptide) with the structure-stabilizing moiety (SSM). Thus, in particularly preferred embodiments of the latter embodiments, where an inclusion of a transmembrane (poly)peptide into the chimeric polypeptide is envisaged, the hinge region may comprise, further comprise, or consist of a transmembrane (poly)peptide.

Moreover, in such instances where the chimeric polypeptide comprises an enveloped virus fusion ectodomain polypeptide, the transmembrane (poly)peptide preferably corresponds to the transmembrane (poly)peptide which is naturally comprised in the enveloped virus fusion polypeptide. In any of the latter embodiments, the transmembrane (poly)peptide may be operably connected with the SSM through a peptide consisting of 3 to 5 amino acid residues selected independently from serine and glycine.

As demonstrated in the herein disclosed examples, such a construct (referred to in Example 21 as “mVL22-TM-CD11” and as defined by SEQ ID NO: 233) has been generated as a ‘proof-of-concept’ and was shown to express at high yields and to be capable of stabilizing the enveloped virus fusion protein ectodomain in the pre-fusion conformation. Thus, in particularly preferred embodiments of the chimeric polypeptide according to the first aspect of the invention, where the ectodomain polypeptide corresponds to, or is a variant of, an ectodomain of a fusion protein from a respiratory syncytial virus (RSV), the chimeric polypeptide further comprises a hinge region which operably connects the ectodomain polypeptide with the SSM, and the hinge region comprises a transmembrane (poly)peptide corresponding to the amino acid residues 486 to 512 of SEQ ID NO: 233. In even more preferred embodiments, the hinge region comprises, or consists of, a transmembrane (poly)peptide corresponding to the amino acid residues 486 to 514 of SEQ ID NO: 233.

### 2.2 Microbial Polypeptide

In accordance with the most general embodiment of the first aspect of the invention, the chimeric polypeptide comprises a microbial polypeptide operably connected downstream to a heterologous, structure-stabilizing moiety (SSM).

The term “microbial polypeptide”, as used herein, refers broadly to a polypeptide which corresponds to, or substantially corresponds to a polypeptide of (or derived from) a microorganism, wherein the microorganism is preferably one selected from the group consisting of bacteria, archaea, protists, fungi, and viruses.

In particularly preferred embodiments, the microorganism is a pathogenic microorganism (i.e., the microorganism is a pathogen of a mammal, preferably a human pathogen); preferably one selected from the group consisting of bacteria and viruses.

In view of the capacity of the herein disclosed structure-stabilizing moiety (SSM) to trimerize and, thus, when being employed as a fusion partner for other polypeptides to also present these in a trimeric conformation, it is thought that the herein disclosed technology will be particularly suitable for effecting a stabilization of such polypeptides which also naturally exist as a trimer. Thus, in preferred embodiments, the microbial polypeptide (or any more specific form thereof, as defined hereafter) is a polypeptide which is capable to trimerize.

Whereas preferably the chimeric polypeptide of the invention is a single polypeptide chain, wherein the structure stabilizing moiety (SSM) is C-terminal of (downstream to) the microbial polypeptide, the present disclosure also contemplates as an alternative arrangement that the structure-stabilizing moiety (SSM) is N-terminal of (upstream to) the microbial polypeptide. In both instances, the structure-stabilizing moiety (SSM) and the microbial polypeptide may be connected by a hinge as defined herein.

In other or even more preferred embodiments, the microbial polypeptide is a viral surface polypeptide or a bacterial surface polypeptide.

In even more preferred embodiments, (i) the viral surface polypeptide is an enveloped virus fusion ectodomain polypeptide; or (ii) the bacterial surface polypeptide is a bacterial outer membrane polypeptide.

Where reference is made to a “variant” of a microbial polypeptide (including any of the specific microbial polypeptides described herein), this preferably refers to a polypeptide having at least 70% or, with increasing preference, at least 80%, 85%, 90%, 95%, or 98% sequence identity to the corresponding microbial polypeptide.

### 2.2.1 Enveloped Virus Fusion Proteins and Ectodomain Polypeptides

In some embodiments, the enveloped virus fusion ectodomain polypeptide corresponds to, or is a variant of: (i) a Class I enveloped virus fusion protein ectodomain; wherein preferably said ectodomain is from a virus selected from orthomyxoviruses, paramyxoviruses, orthopneumoviruses, metapneumoviruses, retroviruses, coronaviruses, filoviruses and arenaviruses; or (ii) a Class III enveloped virus fusion protein ectodomain; or wherein preferably said ectodomain is from a virus selected from rhabdoviruses and herpesviruses.

In preferred embodiments of the latter embodiments, the enveloped virus fusion ectodomain polypeptide corresponds to, or is a variant of, an ectodomain of a fusion protein from: (i) a respiratory syncytial virus (RSV); (ii) a metapneumovirus; (iii) a coronavirus, preferably a betacoronavirus, more preferably a severe acute respiratory syndrome coronavirus 2 (SARS-CoV-2) or a Middle East respiratory syndrome-related coronavirus (MERS-CoV); (iv) a henipavirus, preferably a Hendra virus (HeV) or Nipah virus (NiV); (v) an influenza virus, preferably influenza A or influenza B; (vi) a parainfluenza virus (PIV), preferably a human parainfluenza virus (HPIV); (vii) an arena virus, preferably Lassa Fever virus; or (viii) a retrovirus, preferably human T-lymphotropic virus-1(HTLV-1).

In some embodiments, the chimeric polypeptide further comprises a hinge region which operably connects the microbial polypeptide (preferably the bacterial or viral surface polypeptide, more preferably the enveloped virus fusion ectodomain polypeptide or the bacterial outer membrane polypeptide (e.g., the TAA polypeptide)) with the structure stabilizing moiety; wherein preferably the hinge region comprises or consists of (i) a (poly)peptide consisting of 3 to 5 amino acid residues selected independently from serine and glycine; (ii) serine and glycine residues; (iii) GGSG (SEQ ID NO: 149); (iv) GSG; or (v) G.

Whereas most preferably the chimeric polypeptide of the invention is a single polypeptide chain, wherein the structure stabilizing moiety (SSM) is C-terminal of (downstream to) the enveloped virus fusion ectodomain polypeptide, the present disclosure also contemplates as an alternative arrangement that the structure-stabilizing moiety is N-terminal of (upstream to) the enveloped virus fusion protein ectodomain. In both instances, the structure-stabilizing moiety and the enveloped virus fusion protein ectodomain may be connected by a hinge as defined herein.

Non-limiting examples of an enveloped virus fusion ectodomain polypeptide are presented in the following:

### 2.2.1.1 Inf A HA

Non-limiting examples of Inf A HA ectodomain polypeptides include:

Ectodomain 1-529:
[SEQ ID NO: 86]
MKTIIAFSCILCLIFAQKLPGSDNSMATLCLGHHAVPNGTLVKTITDDQI
EVTNATELVQSSSTGRICNSPHQILDGKNCTLIDALLGDPHCDDFQNKEW
DLFVERSTAYSNCYPYYVPDYATLRSLVASSGNLEFTQESFNWTGVAQDG
SSYACRRGSVNSFFSRLNWLYNLNYKYPEQNVTMPNNDKFDKLYIWGVHH
PGTDKDQTNLYVQASGRVIVSTKRSQQTVIPNIGSRPWVRGVSSIISIYW
TIVKPGDILLINSTGNLIAPRGYFKIQSGKSSIMRSDAHIDECNSECITP
NGSIPNDKPFQNVNKITYGACPRYVKQNTLKLATGMRNVPEKQTRGIFGA
IAGFIENGWEGMVDGWYGFRHQNSEGTGQAADLKSTQAAINQITGKLNRV
IKKTNEKFHQIEKEFSEVEGRIQDLEKYVEDTKIDLWSYNAELLVALENQ
HTIDLTDSEMSKLFERTRRQLRENAEDMGNGCFKIYHKCDNACIGSIRNG
TYDHDIYRNEALNNRFQIKGVQLKSGYKD.
(GenPept gbAEC23340.1)

This sequence comprises the following domains/moieties:
SP=1-16
Ectodomain=17-529
Furin cleavage sites=345-346
FP=346-355
MPER=470-529
Head region=51-328, 403-444,
Stem region=17-58, 327-401, 442-509.

```
Ectodomain minus SP 18-529:
  [SEQ ID NO: 87]
KLPGSDNSMATLCLGHHAVPNGTLVKTITDDQIEVTNATELVQSSSTGRICNSPHQILDGKNCTLIDALLGDPHCDDFQNKEWD
LFVERSTAYSNCYPYYVPDYATLRSLVASSGNLEFTQESFNWTGVAQDGSSYACRRGSVNSFFSRLNWLYNLNYKYPEQNVTMP
NNDKFDKLYIWGVHHPGTDKDQTNLYVQASGRVIVSTKRSQQTVIPNIGSRPWVRGVSSIISIYWTIVKPGDILLINSTGNLIAPR
GYFKIQSGKSSIMRSDAHIDECNSECITPNGSIPNDKPFQNVNKITYGACPRYVKQNTLKLATGMRNVPEKQTRGIFGAIAGFIEN
GWEGMVDGWYGFRHQNSEGTGQAADLKSTQAAINQITGKLNRVIKKTNEKFHQIEKEFSEVEGRIQDLEKYVEDTKIDLWSYN
AELLVALENQHTIDLTDSEMSKLFERTRRQLRENAEDMGNGCFKIYHKCDNACIGSIRNGTYDHDIYRNEALNNRFQIKGVQLKS
GYKD.
(GenPept gbAEC23340.1)
Ectodomain minus SP, minus MPER18-469:
  [SEQ ID NO: 88]
KLPGSDNSMATLCLGHHAVPNGTLVKTITDDQIEVTNATELVQSSSTGRICNSPHQILDGKNCTLIDALLGDPHCDDFQNKEWD
LFVERSTAYSNCYPYYVPDYATLRSLVASSGNLEFTQESFNWTGVAQDGSSYACRRGSVNSFFSRLNWLYNLNYKYPEQNVTMP
NNDKFDKLYIWGVHHPGTDKDQTNLYVQASGRVIVSTKRSQQTVIPNIGSRPWVRGVSSIISIYWTIVKPGDILLINSTGNLIAPR
GYFKIQSGKSSIMRSDAHIDECNSECITPNGSIPNDKPFQNVNKITYGACPRYVKQNTLKLATGMRNVPEKQTRGIFGAIAGFIEN
GWEGMVDGWYGFRHQNSEGTGQAADLKSTQAAINQITGKLNRVIKKTNEKFHQIEKEFSEVEGRIQDLEKYVEDTKIDLWSYN
AELLVALENQHTIDLTDSEMSKLFERTRR.
(GenPept gbAEC23340.1)
Ectodomain 18-341, 346-529 plus altered furin cleavage sites:
  [SEQ ID NO: 89]
KLPGSDNSMATLCLGHHAVPNGTLVKTITDDQIEVTNATELVQSSSTGRICNSPHQILDGKNCTLIDALLGDPHCDDFQNKEWD
LFVERSTAYSNCYPYYVPDYATLRSLVASSGNLEFTQESFNWTGVAQDGSSYACRRGSVNSFFSRLNWLYNLNYKYPEQNVTMP
NNDKFDKLYIWGVHHPGTDKDQTNLYVQASGRVIVSTKRSQQTVIPNIGSRPWVRGVSSIISIYWTIVKPGDILLINSTGNLIAPR
GYFKIQSGKSSIMRSDAHIDECNSECITPNGSIPNDKPFQNVNKITYGACPRYVKQNTLKLATGMRNVPERRRKKRGIFGAIAGFI
ENGWEGMVDGWYGFRHQNSEGTGQAADLKSTQAAINQITGKLNRVIKKTNEKFHQIEKEFSEVEGRIQDLEKYVEDTKIDLWS
YNAELLVALENQHTIDLTDSEMSKLFERTRRQLRENAEDMGNGCFKIYHKCDNACIGSIRNGTYDHDIYRNEALNNRFQIKGVQ
LKSGYKD.
(GenPept gbAEC23340.1)
Stem domain 1-58, 327-401, 442-509 plus linker regions:
  [SEQ ID NO: 90]
MKTIIALSYILCLVFAQKLPGNDNSTATLCLGHHAVPNGTIVKTITNDQIEVTNATELGFGQNTLKLATGMRNVPEKQTRGIFGAI
AGFIENGWEGMLDGWYGFRHQNSEGRGQAADLKSTQAAIDQINGMLNRLIGSGGSGELLVALLNQHTIDLTDSEMNKLFEKT
KKQLRENAEDMGNGCFKIYHKCDNACIGSIRNGTYDHDVYRDEALNNRFQIKGVELKSGYKD.
(GenPept gbAEC23340.1)
Head domain 1-18, 51-328, 403-444 plus linker regions:
  [SEQ ID NO: 91]
MKTIIALSYILCLVFAQKEVTNATELVQNSSTGGICDSPHQILDGENCTLIDALLGDPQCDGFQNKKWDLFVERSKAYSNCYPYDV
PDYASLRSLVASSGTLEFNNESFNWTGVTQNGTSSACKRGSNNSFFSRLNWLTHSKFKYPALNVTMPNNEEFDKLYIWGVHHPGTD
NDQIFLYAQASGRITVSTKRSQQTVIPNIGSRPRVRNIPSRISIYWTIVKPGDILLINSTGNLIAPRGYFKIRSGKSSIMRSDA
PIGKCNSECITPNGSIPNDKPFQNVNRITYGACPRYVKQNGSGGSGKTNEKFHQIEKEFSEVEGRIQDLEKYVEDTKIDLWSYNAEL
L.
(GenPept gbAEC23340.1)
```

### 2.2.1.2 Inf BHA

Non-limiting examples of Inf B HA ectodomain polypeptides include:

Ectodomain 1-547:
[SEQ ID NO: 92]
MKAIIVLLMVVTSNADRICTGITSSNSPHVVKTATQGEVNVTGVIPLTT
TPTKSHFANLKGTQTRGKLCPNCFNCTDLDVALGRPKCMGNTPSAKVSI
LHEVKPATSGCFPIMHDRTKIRQLPNLLRGYENIRLSTSNVINTETAPG
GPYKVGTSGSCPNVANGNGFFNTMAWVIPKDNNKTAINPVTVEVPYICS
EGEDQITVWGFHSDDKTQMERLYGDSNPQKFTSSANGVTTHYVSQIGGF
PNQTEDEGLKQSGRIVVDYMVQKPGKTGTIVYQRGILLPQKVWCASGRS
KVIKGSLPLIGEADCLHEKYGGLNKSKPYYTGEHAKAIGNCPIWVKTPL
KLANGTKYRPPAKLLKERGFFGAIAGFLEGGWEGMIAGWHGYTSHGAHG
VAVAADLKSTQEAINKITKNLNYLSELEVKNLQRLSGAMNELHDEILEL
DEKVDDLRADTISSQIELAVLLSNEGIINSEDEHLLALERKLKKMLGPS
AVEIGNGCFETKHKCNQTCLDRIAAGTFNAGDFSLPTFDSLNITAASLN
DDGLDNHT.
(GenPept gbAFH57854.1)

This sequence comprises the following domains/moieties:
SP=1-16
Ectodomain=17-547
Furin cleavage sites=361-362
FP=362-382
MPER=488-547
Head region=48-344, 418-456
Stem region=17-47, 345-417, 457-547

```
Ectodomain minus SP 17-547:
  [SEQ ID NO: 93]
RICTGITSSNSPHVVKTATQGEVNVTGVIPLTTTPTKSHFANLKGTQTRGKLCPNCFNCTDLDVALGRPKCMGNTPSAKVSILHE
VKPATSGCFPIMHDRTKIRQLPNLLRGYENIRLSTSNVINTETAPGGPYKVGTSGSCPNVANGNGFFNTMAWVIPKDNNKTAIN
PVTVEVPYICSEGEDQITVWGFHSDDKTQMERLYGDSNPQKFTSSANGVTTHYVSQIGGFPNQTEDEGLKQSGRIVVDYMVQ
KPGKTGTIVYQRGILLPQKVWCASGRSKVIKGSLPLIGEADCLHEKYGGLNKSKPYYTGEHAKAIGNCPIWVKTPLKLANGTKYRP
PAKLLKERGFFGAIAGFLEGGWEGMIAGWHGYTSHGAHGVAVAADLKSTQEAINKITKNLNYLSELEVKNLQRLSGAMNELHD
EILELDEKVDDLRADTISSQIELAVLLSNEGIINSEDEHLLALERKLKKMLGPSAVEIGNGCFETKHKCNQTCLDRIAAGTFNAGD
FSLPTFDSLNITAASLNDDGLDNHT.
(GenPept gbAFH57854.1)
Ectodomain minus SP, minus MPER 17-487:
  [SEQ ID NO: 94]
RICTGITSSNSPHVVKTATQGEVNVTGVIPLTTTPTKSHFANLKGTQTRGKLCPNCFNCTDLDVALGRPKCMGNTPSAKVSILHE
VKPATSGCFPIMHDRTKIRQLPNLLRGYENIRLSTSNVINTETAPGGPYKVGTSGSCPNVANGNGFFNTMAWVIPKDNNKTAIN
PVTVEVPYICSEGEDQITVWGFHSDDKTQMERLYGDSNPQKFTSSANGVTTHYVSQIGGFPNQTEDEGLKQSGRIVVDYMVQ
KPGKTGTIVYQRGILLPQKVWCASGRSKVIKGSLPLIGEADCLHEKYGGLNKSKPYYTGEHAKAIGNCPIWVKTPLKLANGTKYRP
PAKLLKERGFFGAIAGFLEGGWEGMIAGWHGYTSHGAHGVAVAADLKSTQEAINKITKNLNYLSELEVKNLQRLSGAMNELHD
EILELDEKVDDLRADTISSQIELAVLLSNEGIINSEDEHLLALERKLKKML.
(GenPept gbAFH57854.1)
Ectodomain minus SP plus altered furin cleavage sites 17-355, 362-547:
  [SEQ ID NO: 95]
RICTGITSSNSPHVVKTATQGEVNVTGVIPLTTTPTKSHFANLKGTQTRGKLCPNCFNCTDLDVALGRPKCMGNTPSAKVSILHE
VKPATSGCFPIMHDRTKIRQLPNLLRGYENIRLSTSNVINTETAPGGPYKVGTSGSCPNVANGNGFFNTMAWVIPKDNNKTAIN
PVTVEVPYICSEGEDQITVWGFHSDDKTQMERLYGDSNPQKFTSSANGVTTHYVSQIGGFPNQTEDEGLKQSGRIVVDYMVQ
KPGKTGTIVYQRGILLPQKVWCASGRSKVIKGSLPLIGEADCLHEKYGGLNKSKPYYTGEHAKAIGNCPIWVKTPLKLANGTKYRP
PARRRKKRAGFFGAIAGFLEGGWEGMIAGWHGYTSHGAHGVAVAADLKSTQEAINKITKNLNYLSELEVKNLQRLSGAMNEL
HDEILELDEKVDDLRADTISSQIELAVLLSNEGIINSEDEHLLALERKLKKMLGPSAVEIGNGCFETKHKCNQTCLDRIAAGTFNA
GDFSLPTFDSLNITAASLNDDGLDNHT.
(GenPept gbAFH57854.1)
Stem domain 1-47, 345-417, 457-547 plus linker regions:
  [SEQ ID NO: 96]
MKAIIVLLMVVTSNADRICTGITSSNSPHVVKTATQGEVNVTGVIPLGSGLANGTKYRPPAKLLKERGFFGAIAGFLEGGWEGMI
AGWHGYTSHGAHGVAVAADLKSTQEAINKITKNLNYLSGSGGSGIELAVLLSNEGIINSEDEHLLALERKLKKMLGPSAVEIGNG
CFETKHKCNQTCLDRIAAGTFNAGDFSLPTFDSLNITAASLNDDGLDNHT.
(GenPept gbAFH57854.1)
Head domain 1-17, 48-344, 418-456 plus linker regions:
  [SEQ ID NO: 97]
MKAIIVLLMVVTSNADRTTTPTKSHFANLKGTQTRGKLCPNCFNCTDLDVALGRPKCMGNTPSAKVSILHEVKPATSGCFPIMH
DRTKIRQLPNLLRGYENIRLSTSNVINTETAPGGPYKVGTSGSCPNVANGNGFFNTMAWVIPKDNNKTAINPVTVEVPYICSEGE
DQITVWGFHSDDKTQMERLYGDSNPQKFTSSANGVTTHYVSQIGGFPNQTEDEGLKQSGRIVVDYMVQKPGKTGTIVYQRGI
LLPQKVWCASGRSKVIKGSLPLIGEADCLHEKYGGLNKSKPYYTGEHAKAIGNCPIWVKTPLKGSGGSGELEVKNLQRLSGAMN
ELHDEILELDEKVDDLRADTISSQ.
(GenPept gbAFH57854.1)
```

### 2.2.1.3 RSV F

Non-limiting examples of RSV F ectodomain polypeptides include:

Ectodomain 1-524:
[SEQ ID NO: 98]
MELLILKANAITTILTAVTFCFASGQNITEEFYQSTCSAVSKGYLSALR
TGWYTSVITIELSNIKKNKCNGTDAKVKLIKQELDKYKNAVTELQLLMQ
STQATNNRARRELPRFMNYTLNNAKKTNVTLSKKRKRRFLGFLLGVGSA
IASGVAVSKVLHLEGEVNKIKSALLSTNKAVVSLSNGVSVLTSKVLDLK
NYIDKQLLPIVNKQSCSISNIETVIEFQQKNNRLLEITREFSVNAGVTT
PVSTYMLTNSELLSLINDMPITNDQKKLMSNNVQIVRQQSYSIMSIIKE
EVLAYVVQLPLYGVIDTPCWKLHTSPLCTTNTKEGSNICLTRTDRGWYC
DNAGSVSFFPQAETCKVQSNRVFCDTMNSLTLPSEVNLCNVDIFNPKYD
CEIMTSKTDVSSSVITSLGAIVSCYGKTKCTASNKNRGIIKTFSNGCDY
VSNKGVDTVSVGNTLYYVNKQEGKSLYVKGEPIINFYDPLVFPSDEFDA
SISQVNEKINQSLAFIRKSDELLHNVNAGKSTTN.
(GenPept gbAHL84194.1)

This sequence comprises the following domains/moieties:
SP=1-23
Ectodomain=24-524
Furin cleavage sites=109-110, 136-137
FP=137-163
D25 interaction domain=61-97, 193-240

```
Ectodomain of RSV F (1-520):
[SEQ ID NO: 99]
MELLILKANAITTILTAVTFCFASGQNITEEFYQSTCSAVSKGYLSALRTGWYTSVITIELSNIKKNK
CNGTDAKVKLIKQELDKYKNAVTELQLLMQSTQATNNRARRELPRFMNYTLNNAKKTNVTLSKKRKRR
FLGFLLGVGSAIASGVAVSKVLHLEGEVNKIKSALLSTNKAVVSLSNGVSVLTSKVLDLKNYIDKQLL
PIVNKQSCSISNIETVIEFQQKNNRLLEITREFSVNAGVTTPVSTYMLTNSELLSLINDMPITNDQKK
LMSNNVQIVRQQSYSIMSIIKEEVLAYVVQLPLYGVIDTPCWKLHTSPLCTTNTKEGSNICLTRTDRG
WYCDNAGSVSFFPQAETCKVQSNRVFCDTMNSLTLPSEVNLCNVDIFNPKYDCEIMTSKTDVSSSVIT
SLGAIVSCYGKTKCTASNKNRGIIKTFSNGCDYVSNKGVDTVSVGNTLYYVNKQEGKSLYVKGEPIIN
FYDPLVFPSDEFDASISQVNEKINQSLAFIRKSDELLHNVNAGK.
Ectodomain minus SP 24-524:
  [SEQ ID NO: 100]
SGQNITEEFYQSTCSAVSKGYLSALRTGWYTSVITIELSNIKKNKCNGTDAKVKLIKQELDKYKNAVT
ELQLLMQSTQATNNRARRELPRFMNYTLNNAKKTNVTLSKKRKRRFLGFLLGVGSAIASGVAVSKVLH
LEGEVNKIKSALLSTNKAVVSLSNGVSVLTSKVLDLKNYIDKQLLPIVNKQSCSISNIETVIEFQQKN
NRLLEITREFSVNAGVTTPVSTYMLTNSELLSLINDMPITNDQKKLMSNNVQIVRQQSYSIMSIIKEE
VLAYVVQLPLYGVIDTPCWKLHTSPLCTTNTKEGSNICLTRTDRGWYCDNAGSVSFFPQAETCKVQSN
RVFCDTMNSLTLPSEVNLCNVDIFNPKYDCEIMTSKTDVSSSVITSLGAIVSCYGKTKCTASNKNRGI
IKTFSNGCDYVSNKGVDTVSVGNTLYYVNKQEGKSLYVKGEPIINFYDPLVFPSDEFDASISQVNEKI
NQSLAFIRKSDELLHNVNAGKSTTN.
(GenPept gbAHL84194.1)
Ectodomain minus SP plus altered furin cleavage sites 24-524:
  [SEQ ID NO: 101]
SGQNITEEFYQSTCSAVSKGYLSALRTGWYTSVITIELSNIKKNKCNGTDAKVKLIKQELDKYKNAVT
ELQLLMQSTQATNNNANNELPRFMNYTLNNAKKTNVTLSNNNNNNFLGFLLGVGSAIASGVAVSKVLH
LEGEVNKIKSALLSTNKAVVSLSNGVSVLTSKVLDLKNYIDKQLLPIVNKQSCSISNIETVIEFQQKN
NRLLEITREFSVNAGVTTPVSTYMLTNSELLSLINDMPITNDQKKLMSNNVQIVRQQSYSIMSIIKEE
VLAYVVQLPLYGVIDTPCWKLHTSPLCTTNTKEGSNICLTRTDRGWYCDNAGSVSFFPQAETCKVQSN
RVFCDTMNSLTLPSEVNLCNVDIFNPKYDCEIMTSKTDVSSSVITSLGAIVSCYGKTKCTASNKNRGI
IKTFSNGCDYVSNKGVDTVSVGNTLYYVNKQEGKSLYVKGEPIINFYDPLVFPSDEFDASISQVNEKI
NQSLAFIRKSDELLHNVNAGKSTTN.
(GenPept gbAHL84194.1)
D25 interaction domain 61-97, 193-240 plus linker regions:
[SEQ ID NO: 102]
LSNIKKNKCNGTDAKVKLIKQELDKYKNAVTELQLLMGGLDLKNYIDKQLLPIVNKQSCSISNIETVI
EFQQKNNRLLEITREFSVN.
(GenPept gbAHL84194.1)
```

2.2.1.4 hMPV F

An illustrative hMPV F precursor has the following amino acid sequence:

[SEQ ID NO: 103]
MSWKVVIIFSLLITPQHGLKESYLEESCSTITEGYLSVLRTGWYTNVFTL
EVGDVENLTCADGPSLIKTELDLTKSALRELRTVSADQLAREEQIENPRQ
SRFVLGAIALGVATAAAVTAGVAIAKTIRLESEVTAIKNALKKTNEAVST
LGNGVRVLATAVRELKDFVSKNLTRAINKNKCDIADLKMAVSFSQFNRRF
LNVVRQFSDNAGITPAISLDLMTDAELARAVSNMPTSAGQIKLMLENRAM
VRRKGFGILIGVYGSSVIYMVQLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPVK
FPEDQFNVALDQVFESIENSQALVDQSNRILSSAEKGNTGFIIVIILIAV
LGSTMILVSVFIIIKKTKKPTGAPPELSGVTNNGFIPHN.
(GenPept gbAAN52913.1)

This sequence comprises the following domains/moieties:
SP=1-19
Ectodomain=1-490
Furin cleavage sites=102-103
FP=103-125
FHRR=126-169
SHIRR=456-490
TM=491-514
C=515-539

Non-limiting examples of hMPV F ectodomain polypeptides include:

```
Ectodomain 1-490:
  [SEQ ID NO: 104]
MSWKVVIIFSLLITPQHGLKESYLEESCSTITEGYLSVLRTGWYTNVFTLEVGDVENLTCADGPSLI
KTELDLTKSALRELRTVSADQLAREEQIENPRQSRFVLGAIALGVATAAAVTAGVAIAKTIRLESEV
TAIKNALKKTNEAVSTLGNGVRVLATAVRELKDFVSKNLTRAINKNKCDIADLKMAVSFSQFNRRFL
NVVRQFSDNAGITPAISLDLMTDAELARAVSNMPTSAGQIKLMLENRAMVRRKGFGILIGVYGSSVI
YMVQLPIFGVIDTPCWIVKAAPSCSEKKGNYACLLREDQGWYCQNAGSTVYYPNEKDCETRGDHVFC
DTAAGINVAEQSKECNINISTTNYPCKVSTGRHPISMVALSPLGALVACYKGVSCSIGSNRVGIIKQ
LNKGCSYITNQDADTVTIDNTVYQLSKVEGEQHVIKGRPVSSSFDPVKFPEDQFNVALDQVFESIEN
SQALVDQSNRILSSAEKGNTG.
(GenPept gbAAN52913.1)
Ectodomain minus SP 20-490:
  [SEQ ID NO: 105]
KESYLEESCSTITEGYLSVLRTGWYTNVFTLEVGDVENLTCADGPSLIKTELDLTKSALRELRTVSA
DQLAREEQIENPRQSRFVLGAIALGVATAAAVTAGVAIAKTIRLESEVTAIKNALKKTNEAVSTLGN
GVRVLATAVRELKDFVSKNLTRAINKNKCDIADLKMAVSFSQFNRRFLNVVRQFSDNAGITPAISLD
LMTDAELARAVSNMPTSAGQIKLMLENRAMVRRKGFGILIGVYGSSVIYMVQLPIFGVIDTPCWIVK
AAPSCSEKKGNYACLLREDQGWYCQNAGSTVYYPNEKDCETRGDHVFCDTAAGINVAEQSKECNINI
STTNYPCKVSTGRHPISMVALSPLGALVACYKGVSCSIGSNRVGIIKQLNKGCSYITNQDADTVTID
NTVYQLSKVEGEQHVIKGRPVSSSFDPVKFPEDQFNVALDQVFESIENSQALVDQSNRILSSAEKGN
TG.
(GenPept gbAAN52913.1)
Ectodomain minus SP plus altered furin cleavage sites 20-490:
  [SEQ ID NO: 106]
KESYLEESCSTITEGYLSVLRTGWYTNVFTLEVGDVENLTCADGPSLIKTELDLTKSALRELRTVSA
DQLAREEQIENPNQSNFVLGAIALGVATAAAVTAGVAIAKTIRLESEVTAIKNALKKTNEAVSTLGN
GVRVLATAVRELKDFVSKNLTRAINKNKCDIADLKMAVSFSQFNRRFLNVVRQFSDNAGITPAISLD
LMTDAELARAVSNMPTSAGQIKLMLENRAMVRRKGFGILIGVYGSSVIYMVOLPIFGVIDTPCWIVK
AAPSCSEKKGNYACLLREDQGWYCQNAGSTVYYPNEKDCETRGDHVFCDTAAGINVAEQSKECNINI
STTNYPCKVSTGRHPISMVALSPLGALVACYKGVSCSIGSNRVGIIKQLNKGCSYITNQDADTVTID
NTVYQLSKVEGEQHVIKGRPVSSSFDPVKFPEDQFNVALDQVFESIENSQALVDQSNRILSSAEKGN
TG.
(GenPept gbAAN52913.1)
```

### 2.2.1.5 PIV F

Non-limiting examples of PIV F ectodomain polypeptides include:

Ectodomain 1-493:
[SEQ ID NO: 107]
MPTSILLIITTMIMASFCQIDITKLQHVGVLVNSPKGMKISQNFETRYL
ILSLIPKIEDSNSCGDQQIKQYKRLLDRLIIPLYDGLRLQKDVIVSNQE
SNENTDPRTKRFFGGVIGTIALGVATSAQITAAVALVEAKQARSDIEKL
KEAIRDTNKAVQSVQSSIGNLIVAIKSVQDYVNKEIVPSIARLGCEAAG
LQLGIALTQHYSELTNIFGDNIGSLQEKGIKLQGIASLYRTNITEIFTT
STVDKYDIYDLLFTESIKVRVIDVDLNDYSITLQVRLPLLTRLLNTQIY
KVDSISYNIQNREWYIPLPSHIMTKGAFLGGADVKECIEAFSSYICPSD
PGFVLNHEMESCLSGNISQCPRTVVTSDIVPRYAFVNGGVVANCITTTC
TCNGIGNRINQPPDQGVKIITHKECNTIGINGMLFNTNKEGTLAFYTPN
DITLNNSVALDPIDISIELNKAKSDLEESKEWIRRSNQKLDSIGNWHQS
STT.
(GenPept gbAAB21447.1)

This sequence comprises the following domains/moieties:
SP=1-19
Ectodomain=1-493
Furin cleavage sites=109-110
FP=110-135

```
Ectodomain minus SP 20-493:
  [SEQ ID NO: 108]
IDITKLQHVGVLVNSPKGMKISQNFETRYLILSLIPKIEDSNSCGDQQIKQYKRLLDRLIIPLYDG
LRLQKDVIVSNQESNENTDPRTKRFFGGVIGTIALGVATSAQITAAVALVEAKQARSDIEKLKEAI
RDTNKAVQSVQSSIGNLIVAIKSVQDYVNKEIVPSIARLGCEAAGLQLGIALTQHYSELTNIFGDN
IGSLQEKGIKLQGIASLYRTNITEIFTTSTVDKYDIYDLLFTESIKVRVIDVDLNDYSITLQVRLP
LLTRLLNTQIYKVDSISYNIQNREWYIPLPSHIMTKGAFLGGADVKECIEAFSSYICPSDPGFVLN
HEMESCLSGNISQCPRTVVTSDIVPRYAFVNGGVVANCITTTCTCNGIGNRINQPPDQGVKIITHK
ECNTIGINGMLFNTNKEGTLAFYTPNDITLNNSVALDPIDISIELNKAKSDLEESKEWIRRSNQKL
DSIGNWHQSSTT.
(GenPept gbAAB21447.1)
Ectodomain minus SP plus altered furin cleavage sites 20-493:
  [SEQ ID NO: 109]
IDITKLQHVGVLVNSPKGMKISQNFETRYLILSLIPKIEDSNSCGDQQIKQYKRLLDRLIIPLYDG
LRLQKDVIVSNQESNENTDPNTKNFFGGVIGTIALGVATSAQITAAVALVEAKQARSDIEKLKEAI
RDTNKAVQSVQSSIGNLIVAIKSVQDYVNKEIVPSIARLGCEAAGLQLGIALTQHYSELTNIFGDN
IGSLQEKGIKLQGIASLYRTNITEIFTTSTVDKYDIYDLLFTESIKVRVIDVDLNDYSITLQVRLP
LLTRLLNTQIYKVDSISYNIQNREWYIPLPSHIMTKGAFLGGADVKECIEAFSSYICPSDPGFVLN
HEMESCLSGNISQCPRTVVTSDIVPRYAFVNGGVVANCITTTCTCNGIGNRINQPPDQGVKIITHK
ECNTIGINGMLFNTNKEGTLAFYTPNDITLNNSVALDPIDISIELNKAKSDLEESKEWIRRSNQKL
DSIGNWHQSSTT.
(GenPept gbAAB21447.1)
```

### 2.2.1.6 MeV F

Non-limiting examples of MeV F ectodomain polypeptides include:

Ectodomain 1-493:
[SEQ ID NO: 110]
MGLKVNVSAIFMAVLLTLQTPTGQIHWGNLSKIGVVGIGSASYKVMTRSS
HQSLVIKLMPNITLLNNCTRVEIAEYRRLLRTVLEPIRDALNAMTQNIRP
VQSVASSRRHKRFAGVVLAGAALGVATAAQITAGIALHQSMLNSQAIDNL
RASLETTNQAIEAIRQAGQEMILAVQGVQDYINNELIPSMNQLSCDLIGQ
KLGLKLLRYYTEILSLFGPSLRDPISAEISIQALSYALGGDINKVLEKLG
YSGGDLLGILESRGIKARITHVDTESYLIVLSIAYPTLSEIKGVIVHRLE
GVSYNIGSQEWYTTVPKYVATQGYLISNFDESSCTFMPEGTVCSQNALYP
MSPLLQECLRGSTKSCARTLVSGSFGNRFILSQGNLIANCASILCKCYTT
GTIINQDPDKILTYIAADHCPVVEVNGVTIQVGSRRYPDAVYLHRIDLGP
PILLERLDVGTNLGNAIAKLEDAKELLESSDQILRSMKGLSST.
(GenPept dbjBAB60865.1)

This sequence comprises the following domains/moieties:
SP=1-24
Ectodomain=1-493
Furin cleavage sites=112-113
FP=113-137

```
Ectodomain minus SP 25-493:
  [SEQ ID NO: 111]
IHWGNLSKIGVVGIGSASYKVMTRSSHQSLVIKLMPNITLLNNCTRVEIA
EYRRLLRTVLEPIRDALNAMTQNIRPVQSVASSRRHKRFAGVVLAGAALG
VATAAQITAGIALHQSMLNSQAIDNLRASLETTNQAIEAIRQAGQEMILA
VQGVQDYINNELIPSMNQLSCDLIGQKLGLKLLRYYTEILSLFGPSLRDP
ISAEISIQALSYALGGDINKVLEKLGYSGGDLLGILESRGIKARITHVDT
ESYLIVLSIAYPTLSEIKGVIVHRLEGVSYNIGSQEWYTTVPKYVATQGY
LISNFDESSCTFMPEGTVCSQNALYPMSPLLQECLRGSTKSCARTLVSGS
FGNRFILSQGNLIANCASILCKCYTTGTIINQDPDKILTYIAADHCPVVE
VNGVTIQVGSRRYPDAVYLHRIDLGPPILLERLDVGTNLGNAIAKLEDAK
ELLESSDQILRSMKGLSST
(GenPept dbjBAB60865.1)
Ectodomain minus SP plus altered furin cleavage
sites:
  [SEQ ID NO: 112]
IHWGNLSKIGVVGIGSASYKVMTRSSHQSLVIKLMPNITLLNNCTRVEIA
EYRRLLRTVLEPIRDALNAMTQNIRPVQSVASSNNHKNFAGVVLAGAALG
VATAAQITAGIALHQSMLNSQAIDNLRASLETTNQAIEAIRQAGQEMILA
VQGVQDYINNELIPSMNQLSCDLIGQKLGLKLLRYYTEILSLFGPSLRDP
ISAEISIQALSYALGGDINKVLEKLGYSGGDLLGILESRGIKARITHVDT
ESYLIVLSIAYPTLSEIKGVIVHRLEGVSYNIGSQEWYTTVPKYVATQGY
LISNFDESSCTFMPEGTVCSQNALYPMSPLLQECLRGSTKSCARTLVSGS
FGNRFILSQGNLIANCASILCKCYTTGTIINQDPDKILTYIAADHCPVVE
VNGVTIQVGSRRYPDAVYLHRIDLGPPILLERLDVGTNLGNAIAKLEDAK
ELLESSDQILRSMKGLSST.
(GenPept dbjBAB60865.1)
```

### 2.2.1.7 HeV F

Non-limiting examples of HeV F ectodomain polypeptides include:

Ectodomain 1-487:
[SEQ ID NO: 113]
MATQEVRLKCLLCGIIVLVLSLEGLGILHYEKLSKIGLVKGITRKYKIKS
NPLTKDIVIKMIPNVSNVSKCTGTVMENYKSRLTGILSPIKGAIELYNNN
THDLVGDVKLAGVVMAGIAIGIATAAQITAGVALYEAMKNADNINKLKSS
IESTNEAVVKLQETAEKTVYVLTALQDYINTNLVPTIDQISCKQTELALD
LALSKYLSDLLFVFGPNLQDPVSNSMTIQAISQAFGGNYETLLRTLGYAT
EDFDDLLESDSIAGQIVYVDLSSYYIIVRVYFPILTEIQQAYVQELLPVS
FNNDNSEWISIVPNFVLIRNTLISNIEVKYCLITKKSVICNQDYATPMTA
SVRECLTGSTDKCPRELVVSSHVPRFALSGGVLFANCISVTCQCQTTGRA
ISQSGEQTLLMIDNTTCTTVVLGNIIISLGKYLGSINYNSESIAVGPPVY
TDKVDISSQISSMNQSLQQSKDYIKEAQKILDTVNPS.
(GenPept NP_047111.2)

This sequence comprises the following domains/moieties:
SP=1-20
Ectodomain=1-487
Furin cleavage sites=109-110
FP=110-135
FHRR=136-169
SHRR=456-587
TM=488-518
C=519-546

```
Ectodomain minus SP 21-487:
  [SEQ ID NO: 114]
SLEGLGILHYEKLSKIGLVKGITRKYKIKSNPLTKDIVIKMIPNVSNVSK
CTGTVMENYKSRLTGILSPIKGAIELYNNNTHDLVGDVKLAGVVMAGIAI
GIATAAQITAGVALYEAMKNADNINKLKSSIESTNEAVVKLQETAEKTVY
VLTALQDYINTNLVPTIDQISCKQTELALDLALSKYLSDLLFVFGPNLQD
PVSNSMTIQAISQAFGGNYETLLRTLGYATEDFDDLLESDSIAGQIVYVD
LSSYYIIVRVYFPILTEIQQAYVQELLPVSFNNDNSEWISIVPNFVLIRN
TLISNIEVKYCLITKKSVICNQDYATPMTASVRECLTGSTDKCPRELVVS
SHVPRFALSGGVLFANCISVTCQCQTTGRAISQSGEQTLLMIDNTTCTTV
VLGNIIISLGKYLGSINYNSESIAVGPPVYTDKVDISSQISSMNQSLQQS
KDYIKEAQKILDTVNPS.
(GenPept NP_047111.2)
Ectodomain minus SP plus altered furin cleavage
sites 21-487:
  [SEQ ID NO: 115]
SLEGLGILHYEKLSKIGLVKGITRKYKIKSNPLTKDIVIKMIPNVSNVSK
CTGTVMENYKSRLTGILSPIKGAIELYNNNTHDLVGDVNLAGVVMAGIAI
GIATAAQITAGVALYEAMKNADNINKLKSSIESTNEAVVKLQETAEKTVY
VLTALQDYINTNLVPTIDQISCKQTELALDLALSKYLSDLLFVFGPNLQD
PVSNSMTIQAISQAFGGNYETLLRTLGYATEDFDDLLESDSIAGQIVYVD
LSSYYIIVRVYFPILTEIQQAYVQELLPVSFNNDNSEWISIVPNFVLIRN
TLISNIEVKYCLITKKSVICNQDYATPMTASVRECLTGSTDKCPRELVVS
SHVPRFALSGGVLFANCISVTCQCQTTGRAISQSGEQTLLMIDNTTCTTV
VLGNIIISLGKYLGSINYNSESIAVGPPVYTDKVDISSQISSMNQSLQQS
KDYIKEAQKILDTVNPS.
(GenPept NP_047111.2)
```

### 2.2.1.8 NiV F

Non-limiting examples of NiV F ectodomain polypeptides include:

Ectodomain 1-487:
[SEQ ID NO: 116]
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPS.
(GenPept NP 112026)

This sequence comprises the following domains/moieties:
SP=1-20
Ectodomain=1-487
Furin cleavage sites=109-110
FP=110-135

```
Ectodomain minus SP:
  [SEQ ID NO: 117]
SECSVGILHYEKLSKIGLVKGVTRKYKIKSNPLTKDIVIKMIPNVSNMSQ
CTGSVMENYKTRLNGILTPIKGALEIYKNNTHDLVGDVRLAGVIMAGVAI
GIATAAQITAGVALYEAMKNADNINKLKSSIESTNEAVVKLQETAEKTVY
VLTALQDYINTNLVPTIDKISCKQTELSLDLALSKYLSDLLFVFGPNLQD
PVSNSMTIQAISQAFGGNYETLLRTLGYATEDFDDLLESDSITGQIIYVD
LSSYYIIVRVYFPILTEIQQAYIQELLPVSFNNDNSEWISIVPNFILVRN
TLISNIEIGFCLITKRSVICNQDYATPMTNNMRECLTGSTEKCPRELVVS
SHVPRFALSNGVLFANCISVTCQCQTTGRAISQSGEQTLLMIDNTTCPTA
VLGNVIISLGKYLGSVNYNSEGIAIGPPVFTDKVDISSQISSMNQSLQQS
KDYIKEAQRLLDTVNPS.
(GenPept NP 112026)
Ectodomain minus SP plus altered furin cleavage sites:
  [SEQ ID NO: 118]
SECSVGILHYEKLSKIGLVKGVTRKYKIKSNPLTKDIVIKMIPNVSNMSQ
CTGSVMENYKTRLNGILTPIKGALEIYKNNTHDLVGDVNLAGVIMAGVAI
GIATAAQITAGVALYEAMKNADNINKLKSSIESTNEAVVKLQETAEKTVY
VLTALQDYINTNLVPTIDKISCKQTELSLDLALSKYLSDLLFVFGPNLQD
PVSNSMTIQAISQAFGGNYETLLRTLGYATEDFDDLLESDSITGQIIYVD
LSSYYIIVRVYFPILTEIQQAYIQELLPVSFNNDNSEWISIVPNFILVRN
TLISNIEIGFCLITKRSVICNQDYATPMTNNMRECLTGSTEKCPRELVVS
SHVPRFALSNGVLFANCISVTCQCQTTGRAISQSGEQTLLMIDNTTCPTA
VLGNVIISLGKYLGSVNYNSEGIAIGPPVFTDKVDISSQISSMNQSLQQS
KDYIKEAQRLLDTVNPS.
(GenPept NP 112026)
```

### 2.2.1.9 HIV GP160

Non-limiting examples of HIV GP160 ectodomain polypeptides include:

Ectodomain 1-688:
[SEQ ID NO: 119]
MRVKGTRKNYWWRWGTMLLGMLMICSAAEQLWVTVYYGVPVWKEATTTLF
CASDAKAVNTEVHNVWATHACVPTDPNPQEVVLENVTENFNMWKNDMVEQ
MQEDIISLWDQSLKPCVKLTPLCVTLNCTNWDGRNGTMNTTSTRNTTTAN
ISRWEMEGEIKNCSFNVTTSIRNKMHKEYALFYKLDVMPIDNGSSYTLIN
CNTSVITQACPKVSFEPIPIHYCTPAGFALLKCNDKKFNGTGPCKNVSTV
QCTHGIRPVVSTQLLLNGSLAEEEIVIRSENLTDNAKTIIVQLNETVVIN
CTRPGNNTRKSIHIGPGRAFYATGDIIGDIRQAHCNLSEASWNKTLKQIA
TKLREQFVNKTIIFNQSSGGDPEIVMHSFNCGGEFFYCDTTQLFNSAWFS
NNTGLNYNNGSNTGGNITLPCRIKQIVNRWQEVGKAMYAPPIRGNITCSS
NITGLLLTRDGGNNVTNESEIFRPGGGNMKDNWRSELYKYKVVKIEPLGV
APTRAKRRVVQREKRAVGTIGAMFLGFLGAAGSTMGAASLTLTVQARQLL
SGIVQQQNNLLRAIEAQQHLLQLTVWGIKQLQARVLAVERYLKDQQLLGI
WGCSGRLICTTAVPWNASWSNKSLDDIWNNMTWMQWEKEIDNYTGLIYRL
IEESQTQQEKNEQDLLQLDTWASLWNWFSISNWLWYIK.
(GenPept dbjBAF31430.1)

This sequence comprises the following domains/moieties:
SP=1-28
Ectodomain=1-688
Furin cleavage sites=508-509
FP=509-538
MPER=668-688
GP120=1-508

```
Ectodomain minus SP:
  [SEQ ID NO: 120]
EQLWVTVYYGVPVWKEATTTLFCASDAKAVNTEVHNVWATHACVPTDPNPQEVVLENVTENFNMWKND
MVEQMQEDIISLWDQSLKPCVKLTPLCVTLNCTNWDGRNGTMNTTSTRNTTTANISRWEMEGEIKNCS
FNVTTSIRNKMHKEYALFYKLDVMPIDNGSSYTLINCNTSVITQACPKVSFEPIPIHYCTPAGFALLK
CNDKKFNGTGPCKNVSTVQCTHGIRPVVSTQLLLNGSLAEEEIVIRSENLTDNAKTIIVQLNETVVIN
CTRPGNNTRKSIHIGPGRAFYATGDIIGDIRQAHCNLSEASWNKTLKQIATKLREQFVNKTIIFNQSS
GGDPEIVMHSFNCGGEFFYCDTTQLFNSAWFSNNTGLNYNNGSNTGGNITLPCRIKQIVNRWQEVGKA
MYAPPIRGNITCSSNITGLLLTRDGGNNVTNESEIFRPGGGNMKDNWRSELYKYKVVKIEPLGVAPTR
AKRRVVQREKRAVGTIGAMFLGFLGAAGSTMGAASLTLTVQARQLLSGIVQQQNNLLRAIEAQQHLLQ
LTVWGIKQLQARVLAVERYLKDQQLLGIWGCSGRLICTTAVPWNASWSNKSLDDIWNNMTWMQWEKEI
DNYTGLIYRLIEESQTQQEKNEQDLLQLDTWASLWNWFSISNWLWYIK.
(GenPept dbjBAF31430.1)
Ectodomain minus SP, minus MPER:
  [SEQ ID NO: 121]
EQLWVTVYYGVPVWKEATTTLFCASDAKAVNTEVHNVWATHACVPTDPNPQEVVLENVTENFNMWKND
MVEQMQEDIISLWDQSLKPCVKLTPLCVTLNCTNWDGRNGTMNTTSTRNTTTANISRWEMEGEIKNCS
FNVTTSIRNKMHKEYALFYKLDVMPIDNGSSYTLINCNTSVITQACPKVSFEPIPIHYCTPAGFALLK
CNDKKFNGTGPCKNVSTVQCTHGIRPVVSTQLLLNGSLAEEEIVIRSENLTDNAKTIIVQLNETVVIN
CTRPGNNTRKSIHIGPGRAFYATGDIIGDIRQAHCNLSEASWNKTLKQIATKLREQFVNKTIIFNQSS
GGDPEIVMHSFNCGGEFFYCDTTQLFNSAWFSNNTGLNYNNGSNTGGNITLPCRIKQIVNRWQEVGKA
MYAPPIRGNITCSSNITGLLLTRDGGNNVTNESEIFRPGGGNMKDNWRSELYKYKVVKIEPLGVAPTR
AKRRVVQREKRAVGTIGAMFLGFLGAAGSTMGAASLTLTVQARQLLSGIVQQQNNLLRAIEAQQHLLQ
LTVWGIKQLQARVLAVERYLKDQQLLGIWGCSGRLICTTAVPWNASWSNKSLDDIWNNMTWMQWEKEI
DNYTGLIYRLIEESQTQQEKNEQDLLQ.
(GenPept dbjBAF31430.1)
Ectodomain minus SP plus altered furin cleavage sites:
  [SEQ ID NO: 122]
EQLWVTVYYGVPVWKEATTTLFCASDAKAVNTEVHNVWATHACVPTDPNPQEVVLENVTENFNMWKND
MVEQMQEDIISLWDQSLKPCVKLTPLCVTLNCTNWDGRNGTMNTTSTRNTTTANISRWEMEGEIKNCS
FNVTTSIRNKMHKEYALFYKLDVMPIDNGSSYTLINCNTSVITQACPKVSFEPIPIHYCTPAGFALLK
CNDKKFNGTGPCKNVSTVQCTHGIRPVVSTQLLINGSLAEEEIVIRSENLTDNAKTIIVQLNETVVIN
CTRPGNNTRKSIHIGPGRAFYATGDIIGDIRQAHCNLSEASWNKTLKQIATKLREQFVNKTIIFNQSS
GGDPEIVMHSFNCGGEFFYCDTTQLFNSAWFSNNTGLNYNNGSNTGGNITLPCRIKQIVNRWQEVGKA
MYAPPIRGNITCSSNITGLLLTRDGGNNVTNESEIFRPGGGNMKDNWRSELYKYKVVKIEPLGVAPTN
ANNNVVQREKRAVGTIGAMFLGFLGAAGSTMGAASLTLTVQARQLLSGIVQQQNNLLRAIEAQQHLLQ
LTVWGIKQLQARVLAVERYLKDQQLLGIWGCSGRLICTTAVPWNASWSNKSLDDIWNNMTWMQWEKEI
DNYTGLIYRLIEESQTQQEKNEQDLLQLDTWASLWNWFSISNWLWYIK.
(GenPept dbjBAF31430.1)
GP41 ectodomain 509-688:
  [SEQ ID NO: 123]
VVQREKRAVGTIGAMFLGFLGAAGSTMGAASLTLTVQARQLLSGIVQQQNNLLRAIEAQQHLLQLTVW
GIKQLQARVLAVERYLKDQQLLGIWGCSGRLICTTAVPWNASWSNKSLDDIWNNMTWMQWEKEIDNYT
GLIYRLIEESQTQQEKNEQDLLQLDTWASLWNWFSISNWLWYIK.
(GenPept dbjBAF31430.1)
```

### 2.2.1.10 EBOV GP

Non-limiting examples of EBOV GP ectodomain polypeptides include:

Ectodomain 1-650:
[SEQ ID NO: 124]
MGVTGILQLPRDRFKRTSFFLWVIILFQRTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEELSFTVVSNGAKNISGQSPARTSSDPGTNTTTEDHKIMASENSSAM
VQVHSQGREAAVSHLTTLATISTSPQSLTTKPGPDNSTHNTPVYKLDISE
ATQVEQHHRRTDNDSTASDTPSATTAAGPPKAENTNTSKSTDFLDPATTT
SPQNHSETAGNNNTHHQDTGEESASSGKLGLITNTIAGVAGLITGGRRTR
REAIVNAQPKCNPNLHYWTTQDEGAAIGLAWIPYFGPAAEGIYIEGLMHN
QDGLICGLRQLANETTQALQLFLRATTELRTFSILNRKAIDFLLQRWGGT
CHILGPDCCIEPHDWTKNITDKIDQIIHDFVDKTLPDQGDNDNWWTGWRQ
(GenPept NP_066246.1)

This sequence comprises the following domains/moieties:
SP=1-27
Ectodomain=1-650
Furin cleavage sites=501-502
Cathepsin cleavage sites=191-192, 201-202
FP=511-556
MPER=636-650
Mucin-like domain=312-461

```
Ectodomain minus SP:
  [SEQ ID NO: 125]
QRTFSIPLGVIHNSTLQVSDVDKLVCRDKLSSTNQLRSVGLNLEGNGVATDVPSATKRWGFRSGVPP
KVVNYEAGEWAENCYNLEIKKPDGSECLPAAPDGIRGFPRCRYVHKVSGTGPCAGDFAFHKEGAFFL
YDRLASTVIYRGTTFAEGVVAFLILPQAKKDFFSSHPLREPVNATEDPSSGYYSTTIRYQATGFGTN
ETEYLFEVDNLTYVQLESRFTPQFLLQLNETIYTSGKRSNTTGKLIWKVNPEIDTTIGEWAFWETKK
NLTRKIRSEELSFTVVSNGAKNISGQSPARTSSDPGTNTTTEDHKIMASENSSAMVQVHSQGREAAV
SHLTTLATISTSPQSLTTKPGPDNSTHNTPVYKLDISEATQVEQHHRRTDNDSTASDTPSATTAAGP
PKAENTNTSKSTDFLDPATTTSPQNHSETAGNNNTHHQDTGEESASSGKLGLITNTIAGVAGLITGG
RRTRREAIVNAQPKCNPNLHYWTTQDEGAAIGLAWIPYFGPAAEGIYIEGLMHNQDGLICGLRQLAN
ETTQALQLFLRATTELRTFSILNRKAIDFLLQRWGGTCHILGPDCCIEPHDWTKNITDKIDQIIHDF
VDKTLPDQGDNDNWWTGWRQ.
(GenPept NP_066246.1)
Ectodomain minus SP, minus MPER:
  [SEQ ID NO: 126]
QRTFSIPLGVIHNSTLQVSDVDKLVCRDKLSSTNQLRSVGLNLEGNGVATDVPSATKRWGFRSGVPP
KVVNYEAGEWAENCYNLEIKKPDGSECLPAAPDGIRGFPRCRYVHKVSGTGPCAGDFAFHKEGAFFL
YDRLASTVIYRGTTFAEGVVAFLILPQAKKDFFSSHPLREPVNATEDPSSGYYSTTIRYQATGFGTN
ETEYLFEVDNLTYVQLESRFTPQFLLQLNETIYTSGKRSNTTGKLIWKVNPEIDTTIGEWAFWETKK
NLTRKIRSEELSFTVVSNGAKNISGQSPARTSSDPGTNTTTEDHKIMASENSSAMVQVHSQGREAAV
SHLTTLATISTSPQSLTTKPGPDNSTHNTPVYKLDISEATQVEQHHRRTDNDSTASDTPSATTAAGP
PKAENTNTSKSTDFLDPATTTSPQNHSETAGNNNTHHQDTGEESASSGKLGLITNTIAGVAGLITGG
RRTRREAIVNAQPKCNPNLHYWTTQDEGAAIGLAWIPYFGPAAEGIYIEGLMHNQDGLICGLRQLAN
ETTQALQLFLRATTELRTFSILNRKAIDFLLQRWGGTCHILGPDCCIEPHDWTKNITDKIDQIIHDF
VDKTL.
(GenPept NP_066246.1)
Ectodomain minus SP plus altered furin cleavage sites:
  [SEQ ID NO: 127]
QRTFSIPLGVIHNSTLQVSDVDKLVCRDKLSSTNQLRSVGLNLEGNGVATDVPSATKRWGFRSGVPP
KVVNYEAGEWAENCYNLEIKKPDGSECLPAAPDGIRGFPRCRYVHKVSGTGPCAGDFAFHKEGAFFL
YDRLASTVIYRGTTFAEGVVAFLILPQAKKDFFSSHPLREPVNATEDPSSGYYSTTIRYQATGFGTN
ETEYLFEVDNLTYVQLESRFTPQFLLQLNETIYTSGKRSNTTGKLIWKVNPEIDTTIGEWAFWETKK
NLTRKIRSEELSFTVVSNGAKNISGQSPARTSSDPGTNTTTEDHKIMASENSSAMVQVHSQGREAAV
SHLTTLATISTSPQSLTTKPGPDNSTHNTPVYKLDISEATQVEQHHRRTDNDSTASDTPSATTAAGP
PKAENTNTSKSTDFLDPATTTSPQNHSETAGNNNTHHQDTGEESASSGKLGLITNTIAGVAGLITGG
NNTNNEAIVNAQPKCNPNLHYWTTQDEGAAIGLAWIPYFGPAAEGIYIEGLMHNQDGLICGLRQLAN
ETTQALQLFLRATTELRTFSILNRKAIDFLLQRWGGTCHILGPDCCIEPHDWTKNITDKIDQIIHDF
VDKTL.
(GenPept NP_066246.1)
Ectodomain minus SP, minus mucin-like domain:
  [SEQ ID NO: 128]
QRTFSIPLGVIHNSTLQVSDVDKLVCRDKLSSTNQLRSVGLNLEGNGVATDVPSATKRWGFRSGVPP
KVVNYEAGEWAENCYNLEIKKPDGSECLPAAPDGIRGFPRCRYVHKVSGTGPCAGDFAFHKEGAFFL
YDRLASTVIYRGTTFAEGVVAFLILPQAKKDFFSSHPLREPVNATEDPSSGYYSTTIRYQATGFGTN
ETEYLFEVDNLTYVQLESRFTPQFLLQLNETIYTSGKRSNTTGKLIWKVNPEIDTTIGEWAFWETKK
NLTRKIRSEELSFTVVGGNNTHHQDTGEESASSGKLGLITNTIAGVAGLITGGRRTRREAIVNAQPK
CNPNLHYWTTQDEGAAIGLAWIPYFGPAAEGIYIEGLMHNQDGLICGLRQLANETTQALQLFLRATT
ELRTFSILNRKAIDFLLQRWGGTCHILGPDCCIEPHDWTKNITDKIDQIIHDFVDKTLPDQGDNDNW
WTGWRQ.
(GenPept NP_066246.1)
Ectodomain minus mucin-like domain:
  [SEQ ID NO: 129]
MGVTGILQLPRDRFKRTSFFLWVIILFQRTFSIPLGVIHNSTLQVSDVDKLVCRDKLSSTNQLRSVG
LNLEGNGVATDVPSATKRWGFRSGVPPKVVNYEAGEWAENCYNLEIKKPDGSECLPAAPDGIRGFPR
CRYVHKVSGTGPCAGDFAFHKEGAFFLYDRLASTVIYRGTTFAEGVVAFLILPQAKKDFFSSHPLRE
PVNATEDPSSGYYSTTIRYQATGFGTNETEYLFEVDNLTYVQLESRFTPQFLLQLNETIYTSGKRSN
TTGKLIWKVNPEIDTTIGEWAFWETKKNLTRKIRSEESASSGKLGLITNTIAGVAGLITGGRRTRRE
AIVNAQPKCNPNLHYWTTQDEGAAIGLAWIPYFGPAAEGIYIEGLMHNQDGLICGLRQLANETTQAL
QLFLRATTELRTFSILNRKAIDFLLQRWGGTCHILGPDCCIEPHDWTKNITDKIDQIIHDFVDKTL
```

### 2.2.1.11 MARV GP

Non-limiting examples of MARV GP ectodomain polypeptides include:

Ectodomain 1-650:
[SEQ ID NO: 130]
MKTTCFLISLILIQGTKNLPILEIASNNQPQNVDSVCSGTLQKTEDVHLM
GFTLSGQKVADSPLEASKRWAFRTGVPPKNVEYTEGEEAKTCYNISVTDP
SGKSLLLDPPTNIRDYPKCKTIHHIQGQNPHAQGIALHLWGAFFLYDRIA
STTMYRGKVFTEGNIAAMIVNKTVHKMIFSRQGQGYRHMNLTSTNKYWTS
SNGTQTNDTGCFGALQEYNSTKNQTCAPSKIPPPLPTARPEIKLTSTPTD
ATKLNTTDPSSDDEDLATSGSGSGEREPHTTSDAVTKQGLSSTMPPTPSP
QPSTPQQGGNNTNHSQDAVTELDKNNTTAQPSMPPHNTTTISTNNTSKHN
FSTLSAPLQNTTNDNTQSTITENEQTSAPSITTLPPTGNPTTAKSTSSKK
GPATTAPNTTNEHFTSPPPTPSSTAQHLVYFRRKRSILWREGDMFPFLDG
LINAPIDFDPVPNTKTIFDESSSSGASAEEDQHASPNISLTLSYFPNINE
NTAYSGENENDCDAELRIWSVQEDDLAAGLSWIPFFGPGIEGLYTAVLIK
NQNNLVCRLRRLANQTAKSLELLLRVTTEERTFSLINRHAIDFLLTRWGG
TCKVLGPDCCIGIEDLSKNISEQIDQIKKDEQKEGTGWGLGGKWWTSDW
G.
(GenPept YP_001531156.1)

This sequence comprises the following domains/moieties:
SP=1-19
Ectodomain=1-650
Furin cleavage sites=434-435
FP=526-549
MPER=628-650
Mucin-like domain=244-425

Ectodomain minus SP 20-650:
[SEQ ID NO: 131]
PILEIASNNQPQNVDSVCSGTLQKTEDVHLMGFTLSGQKVADSPL
EASKRWAFRTGVPPKNVEYTEGEEAKTCYNISVTDPSGKSLLLDP
PTNIRDYPKCKTIHHIQGQNPHAQGIALHLWGAFFLYDRIASTTM
YRGKVFTEGNIAAMIVNKTVHKMIFSRQGQGYRHMNLTSTNKYWT
SSNGTQTNDTGCFGALQEYNSTKNQTCAPSKIPPPLPTARPEIKL
TSTPTDATKLNTTDPSSDDEDLATSGSGSGEREPHTTSDAVTKQG
LSSTMPPTPSPQPSTPQQGGNNTNHSQDAVTELDKNNTTAQPSMP
PHNTTTISTNNTSKHNFSTLSAPLQNTTNDNTQSTITENEQTSAP
SITTLPPTGNPTTAKSTSSKKGPATTAPNTTNEHFTSPPPTPSST
AQHLVYFRRKRSILWREGDMFPFLDGLINAPIDFDPVPNTKTIFD
ESSSSGASAEEDQHASPNISLTLSYFPNINENTAYSGENENDCDA
ELRIWSVQEDDLAAGLSWIPFFGPGIEGLYTAVLIKNQNNLVCRL
RRLANQTAKSLELLLRVTTEERTFSLINRHAIDFLLTRWGGTCKV
LGPDCCIGIEDLSKNISEQIDQIKKDEQKEGTGWGLGGKWWTSDW
G
(GenPept YP_001531156.1).
Ectodomain minus SP, minus MPER 20-627:
[SEQ ID NO: 132]
PILEIASNNQPQNVDSVCSGTLQKTEDVHLMGFTLSGQKVADSPL
EASKRWAFRTGVPPKNVEYTEGEEAKTCYNISVTDPSGKSLLLDP
PTNIRDYPKCKTIHHIQGQNPHAQGIALHLWGAFFLYDRIASTTM
YRGKVFTEGNIAAMIVNKTVHKMIFSRQGQGYRHMNLTSTNKYWT
SSNGTQTNDTGCFGALQEYNSTKNQTCAPSKIPPPLPTARPEIKL
TSTPTDATKLNTTDPSSDDEDLATSGSGSGEREPHTTSDAVTKQG
LSSTMPPTPSPQPSTPQQGGNNTNHSQDAVTELDKNNTTAQPSMP
PHNTTTISTNNTSKHNFSTLSAPLQNTTNDNTQSTITENEQTSAP
SITTLPPTGNPTTAKSTSSKKGPATTAPNTTNEHFTSPPPTPSST
AQHLVYFRRKRSILWREGDMFPFLDGLINAPIDFDPVPNTKTIFD
ESSSSGASAEEDQHASPNISLTLSYFPNINENTAYSGENENDCDA
ELRIWSVQEDDLAAGLSWIPFFGPGIEGLYTAVLIKNQNNLVCRL
RRLANQTAKSLELLLRVTTEERTFSLINRHAIDFLLTRWGGTCKV
LGPDCCIGIEDLSKNISEQIDQI
(GenPept YP_001531156.1).
Ectodomain minus SP plus altered furin
cleavage sites:
[SEQ ID NO: 133]
PILEIASNNQPQNVDSVCSGTLQKTEDVHLMGFTLSGQKVADSPL
EASKRWAFRTGVPPKNVEYTEGEEAKTCYNISVTDPSGKSLLLDP
PTNIRDYPKCKTIHHIQGQNPHAQGIALHLWGAFFLYDRIASTTM
YRGKVFTEGNIAAMIVNKTVHKMIFSRQGQGYRHMNLTSTNKYWT
SSNGTQTNDTGCFGALQEYNSTKNQTCAPSKIPPPLPTARPEIKL
TSTPTDATKLNTTDPSSDDEDLATSGSGSGEREPHTTSDAVTKQG
LSSTMPPTPSPQPSTPQQGGNNTNHSQDAVTELDKNNTTAQPSMP
PHNTTTISTNNTSKHNFSTLSAPLQNTTNDNTQSTITENEQTSAP
SITTLPPTGNPTTAKSTSSKKGPATTAPNTTNEHFTSPPPTPSST
AQHLVYFNNNNSILWREGDMFPFLDGLINAPIDFDPVPNTKTIFD
ESSSSGASAEEDQHASPNISLTLSYFPNINENTAYSGENENDCDA
ELRIWSVQEDDLAAGLSWIPFFGPGIEGLYTAVLIKNQNNLVCRL
RRLANQTAKSLELLLRVTTEERTFSLINRHAIDFLLTRWGGTCKV
LGPDCCIGIEDLSKNISEQIDQIKKDEQKEGTGWGLGGKWWTSDW
G
(GenPept YP_001531156.1).
Ectodomain minus SP, minus mucin-like domain:
[SEQ ID NO: 134]
PILEIASNNQPQNVDSVCSGTLQKTEDVHLMGFTLSGQKVADSPL
EASKRWAFRTGVPPKNVEYTEGEEAKTCYNISVTDPSGKSLLLDP
PTNIRDYPKCKTIHHIQGQNPHAQGIALHLWGAFFLYDRIASTTM
YRGKVFTEGNIAAMIVNKTVHKMIFSRQGQGYRHMNLTSTNKYWT
SSNGTQTNDTGCFGALQEYNSTKNQTCAPSKIPPPLPTARPEIKL
GGAQHLVYFRRKRSILWREGDMFPFLDGLINAPIDFDPVPNTKTI
FDESSSSGASAEEDQHASPNISLTLSYFPNINENTAYSGENENDC
DAELRIWSVQEDDLAAGLSWIPFFGPGIEGLYTAVLIKNQNNLVC
RLRRLANQTAKSLELLLRVTTEERTFSLINRHAIDFLLTRWGGTC
KVLGPDCCIGIEDLSKNISEQIDQIKKDEQKEGTGWGLGGKWWTS
DWG
(GenPept YP_001531156.1).

### 2.2.1.12 SARS-CoVS

Non-limiting examples of SARS-CoV S ectodomain polypeptides include:

Ectodomain 1-1199:
[SEQ ID NO: 135]
MFIFLLFLTLTSGSDLDRCTTFDDVQAPNYTQHTSSMRGVYYPDE
IFRSDTLYLTQDLFLPFYSNVTGFHTINHTFGNPVIPFKDGIYFA
ATEKSNVVRGWVFGSTMNNKSQSVIIINNSTNVVIRACNFELCDN
PFFAVSKPMGTQTHTMIFDNAFNCTFEYISDAFSLDVSEKSGNFK
HLREFVFKNKDGFLYVYKGYQPIDVVRDLPSGFNTLKPIFKLPLG
INITNFRAILTAFSPAQDIWGTSAAAYFVGYLKPTTFMLKYDENG
TITDAVDCSQNPLAELKCSVKSFEIDKGIYQTSNFRVVPSGDVVR
FPNITNLCPFGEVFNATKFPSVYAWERKKISNCVADYSVLYNSTF
FSTFKCYGVSATKLNDLCFSNVYADSFVVKGDDVRQIAPGQTGVI
ADYNYKLPDDFMGCVLAWNTRNIDATSTGNYNYKYRYLRHGKLRP
FERDISNVPFSPDGKPCTPPALNCYWPLNDYGFYTTTGIGYQPYR
VVVLSFELLNAPATVCGPKLSTDLIKNQCVNFNFNGLTGTGVLTP
SSKRFQPFQQFGRDVSDFTDSVRDPKTSEILDISPCAFGGVSVIT
PGTNASSEVAVLYQDVNCTNVSAAIHADQLTPAWRIYSTGNNVFQ
TQAGCLIGAEHVDTSYECDIPIGAGICASYHTVSLLRSTSQKSIV
AYTMSLGADSSIAYSNNTIAIPTNFSISITTEVMPVSMAKTSVDC
NMYICGDSTECANLLLQYGSFCTQLNRALSGIAAEQDRNTREVFA
QVKQMYKTPTLKYFGGFNFSQILPDPLKPTKRSFIEDLLFNKVTL
ADAGFMKQYGECLGDINARDLICAQKFNGLTVLPPLLTDDMIAAY
TAALVSGTATAGWTFGAGAALQIPFAMQMAYRFNGIGVTQNVLYE
NQKQIANQFNKAISQIQESLTTTSTALGKLQDVVNQNAQALNTLV
KQLSSNFGAISSVLNDILSRLDKVEAEVQIDRLITGRLQSLQTYV
TQQLIRAAEIRASANLAATKMSECVLGQSKRVDFCGKGYHLMSFP
QAAPHGVVFLHVTYVPSQERNFTTAPAICHEGKAYFPREGVFVFN
GTSWFITQRNFFSPQIITTDNTFVSGNCDVVIGIINNTVYDPLQP
ELDSFKGELDKYFKNHTSPDVDLGDISGINASVVNIQKEIDRLNE
VAKNLNESLIDLQELGKYEQYIKWPWYVW
(GenPept gbAAR86788.1).

This sequence comprises the following domains/moieties:
SP=1-13
Ectodomain=1-1199
human airway trypsin-like protease cleavage sites=667-668
FP=770-788
MPER=1188-1199

Ectodomain minus SP:
[SEQ ID NO: 136]
SDLDRCTTFDDVQAPNYTQHTSSMRGVYYPDEIFRSDTLYLTQDL
FLPFYSNVTGFHTINHTFGNPVIPFKDGIYFAATEKSNVVRGWVF
GSTMNNKSQSVIIINNSTNVVIRACNFELCDNPFFAVSKPMGTQT
HTMIFDNAFNCTFEYISDAFSLDVSEKSGNFKHLREFVFKNKDGF
LYVYKGYQPIDVVRDLPSGFNTLKPIFKLPLGINITNFRAILTAF
SPAQDIWGTSAAAYFVGYLKPTTFMLKYDENGTITDAVDCSQNPL
AELKCSVKSFEIDKGIYQTSNFRVVPSGDVVRFPNITNLCPFGEV
FNATKFPSVYAWERKKISNCVADYSVLYNSTFFSTFKCYGVSATK
LNDLCFSNVYADSFVVKGDDVRQIAPGQTGVIADYNYKLPDDFMG
CVLAWNTRNIDATSTGNYNYKYRYLRHGKLRPFERDISNVPFSPD
GKPCTPPALNCYWPLNDYGFYTTTGIGYQPYRVVVLSFELLNAPA
TVCGPKLSTDLIKNQCVNFNFNGLTGTGVLTPSSKRFQPFQQFGR
DVSDFTDSVRDPKTSEILDISPCAFGGVSVITPGTNASSEVAVLY
QDVNCTNVSAAIHADQLTPAWRIYSTGNNVFQTQAGCLIGAEHVD
TSYECDIPIGAGICASYHTVSLLRSTSQKSIVAYTMSLGADSSIA
YSNNTIAIPTNFSISITTEVMPVSMAKTSVDCNMYICGDSTECAN
LLLQYGSFCTQLNRALSGIAAEQDRNTREVFAQVKQMYKTPTLKY
FGGFNFSQILPDPLKPTKRSFIEDLLFNKVTLADAGFMKQYGECL
GDINARDLICAQKFNGLTVLPPLLTDDMIAAYTAALVSGTATAGW
TFGAGAALQIPFAMQMAYRFNGIGVTQNVLYENQKQIANQFNKAI
SQIQESLTTTSTALGKLQDVVNQNAQALNTLVKQLSSNFGAISSV
LNDILSRLDKVEAEVQIDRLITGRLQSLQTYVTQQLIRAAEIRAS
ANLAATKMSECVLGQSKRVDFCGKGYHLMSFPQAAPHGVVFLHVT
YVPSQERNFTTAPAICHEGKAYFPREGVFVFNGTSWFITQRNFFS
PQIITTDNTFVSGNCDVVIGIINNTVYDPLQPELDSFKGELDKYF
KNHTSPDVDLGDISGINASVVNIQKEIDRLNEVAKNLNESLIDLQ
ELGKYEQYIKWPWYVW
(GenPept gbAAR86788.1).
Ectodomain minus SP, minus MPER:
[SEQ ID NO: 137]
SDLDRCTTFDDVQAPNYTQHTSSMRGVYYPDEIFRSDTLYLTQDL
FLPFYSNVTGFHTINHTFGNPVIPFKDGIYFAATEKSNVVRGWVF
GSTMNNKSQSVIIINNSTNVVIRACNFELCDNPFFAVSKPMGTQT
HTMIFDNAFNCTFEYISDAFSLDVSEKSGNFKHLREFVFKNKDGF
LYVYKGYQPIDVVRDLPSGFNTLKPIFKLPLGINITNFRAILTAF
SPAQDIWGTSAAAYFVGYLKPTTFMLKYDENGTITDAVDCSQNPL
AELKCSVKSFEIDKGIYQTSNFRVVPSGDVVRFPNITNLCPFGEV
FNATKFPSVYAWERKKISNCVADYSVLYNSTFFSTFKCYGVSATK
LNDLCFSNVYADSFVVKGDDVRQIAPGQTGVIADYNYKLPDDFMG
CVLAWNTRNIDATSTGNYNYKYRYLRHGKLRPFERDISNVPFSPD
GKPCTPPALNCYWPLNDYGFYTTTGIGYQPYRVVVLSFELLNAPA
TVCGPKLSTDLIKNQCVNFNFNGLTGTGVLTPSSKRFQPFQQFGR
DVSDFTDSVRDPKTSEILDISPCAFGGVSVITPGTNASSEVAVLY
QDVNCTNVSAAIHADQLTPAWRIYSTGNNVFQTQAGCLIGAEHVD
TSYECDIPIGAGICASYHTVSLLRSTSQKSIVAYTMSLGADSSIA
YSNNTIAIPTNFSISITTEVMPVSMAKTSVDCNMYICGDSTECAN
LLLQYGSFCTQLNRALSGIAAEQDRNTREVFAQVKQMYKTPTLKY
FGGFNFSQILPDPLKPTKRSFIEDLLFNKVTLADAGFMKQYGECL
GDINARDLICAQKFNGLTVLPPLLTDDMIAAYTAALVSGTATAGW
TFGAGAALQIPFAMQMAYRFNGIGVTQNVLYENQKQIANQFNKAI
SQIQESLTTTSTALGKLQDVVNQNAQALNTLVKQLSSNFGAISSV
LNDILSRLDKVEAEVQIDRLITGRLQSLQTYVTQQLIRAAEIRAS
ANLAATKMSECVLGQSKRVDFCGKGYHLMSFPQAAPHGVVFLHVT
YVPSQERNFTTAPAICHEGKAYFPREGVFVFNGTSWFITQRNFFS
PQIITTDNTFVSGNCDVVIGIINNTVYDPLQPELDSFKGELDKYF
KNHTSPDVDLGDISGINASVVNIQKEIDRLNEVAKNLNESLIDLQ
ELGK
(GenPept gbAAR86788.1).

### 2.2.1.13 MERS-CoVS

Non-limiting examples of MERS-CoV S ectodomain polypeptides include:

Ectodomain 1-1301:
[SEQ ID NO: 138]
MIHSVFLLMFLLTPTESYVDVGPDSVKSACIEVDIQQTFFDKTWP
RPIDVSKADGIIYPQGRTYSNITITYQGLFPYQGDHGDMYVYSAG
HATGTTPQKLFVANYSQDVKQFANGFVVRIGAAANSTGTVIISPS
TSATIRKIYPAFMLGSSVGNFSDGKMGRFFNHTLVLLPDGCGTLL
RAFYCILEPRSGNHCPAGNSYTSFATYHTPATDCSDGNYNRNASL
NSFKEYFNLRNCTFMYTYNITEDEILEWFGITQTAQGVHLFSSRY
VDLYGGNMFQFATLPVYDTIKYYSIIPHSIRSIQSDRKAWAAFYV
YKLQPLTFLLDFSVDGYIRRAIDCGFNDLSQLHCSYESFDVESGV
YSVSSFEAKPSGSVVEQAEGVECDFSPLLSGTPPQVYNFKRLVFT
NCNYNLTKLLSLFSVNDFTCSQISPAAIASNCYSSLILDYFSYPL
SMKSDLGVSSAGPISQFNYKQSFSNPTCLILATVPHNLTTITKPL
KYSYINKCSRLLSDDRTEVPQLVNANQYSPCVSIVPSTVWEDGDY
YRKQLSPLEGGGWLVASGSTVAMTEQLQMGFGITVQYGTDTNSVC
PKLEFANDTKIASQLGNCVEYSLYGVSGRGVFQNCTAVGVRQQRF
VYDAYQNLVGYYSDDGNYYCLRACVSVPVSVIYDKETKTHATLFG
SVACEHISSTMSQYSRSTRSMLKRRDSTYGPLQTPVGCVLGLVNS
SLFVEDCKLPLGQSLCALPDTPSTLTPRSVRSVPGEMRLASIAFN
HPIQVDQFNSSYFKLSIPTNFSFGVTQEYIQTTIQKVTVDCKQYI
CNGFQKCEQLLREYGQFCSKINQALHGANLRQDDSVRNLFASVKS
SQSSPIIPGFGGDFNLTLLEPVSISTGSRSARSAIEDLLFDKVTI
ADPGYMQGYDDCMQQGPASARDLICAQYVAGYKVLPPLMDVNMEA
AYTSSLLGSIAGVGWTAGLSSFAAIPFAQSIFYRLNGVGITQQVL
SENQKLIANKFNQALGAMQTGFTTTNEAFRKVQDAVNNNAQALSK
LASELSNTFGAISASIGDIIQRLDVLEQDAQIDRLINGRLTTLNA
FVAQQLVRSESAALSAQLAKDKVNECVKAQSKRSGFCGQGTHIVS
FVVNAPNGLYFMHVGYYPSNHIEVVSAYGLCDAANPTNCIAPVNG
YFIKTNNTRIVDEWSYTGSSFYSPEPITSLNTKYVAPQVTYQNIS
TNLPPPLLGNSTGIDFQDELDEFFKNVSTSIPNFGSLTQINTTLL
DLTYEMLSLQQVVKALNESYIDLKELGNYTYYNKWPWYIWL
(GenPept gbAHX00711.1).

This sequence comprises the following domains/moieties:
SP=1-21
Ectodomain=1-1301
Furin cleavage sites=751-752, 887-888
FP=888-891, 951-980
MPER=1292-1301

Ectodomain minus SP 22-1301:
[SEQ ID NO: 139]
GPDSVKSACIEVDIQQTFFDKTWPRPIDVSKADGIIYPQGRTYSN
ITITYQGLFPYQGDHGDMYVYSAGHATGTTPQKLFVANYSQDVKQ
FANGFVVRIGAAANSTGTVIISPSTSATIRKIYPAFMLGSSVGNF
SDGKMGRFFNHTLVLLPDGCGTLLRAFYCILEPRSGNHCPAGNSY
TSFATYHTPATDCSDGNYNRNASLNSFKEYFNLRNCTFMYTYNIT
EDEILEWFGITQTAQGVHLFSSRYVDLYGGNMFQFATLPVYDTIK
YYSIIPHSIRSIQSDRKAWAAFYVYKLQPLTFLLDFSVDGYIRRA
IDCGFNDLSQLHCSYESFDVESGVYSVSSFEAKPSGSVVEQAEGV
ECDFSPLLSGTPPQVYNFKRLVFTNCNYNLTKLLSLFSVNDFTCS
QISPAAIASNCYSSLILDYFSYPLSMKSDLGVSSAGPISQFNYKQ
SFSNPTCLILATVPHNLTTITKPLKYSYINKCSRLLSDDRTEVPQ
LVNANQYSPCVSIVPSTVWEDGDYYRKQLSPLEGGGWLVASGSTV
AMTEQLQMGFGITVQYGTDTNSVCPKLEFANDTKIASQLGNCVEY
SLYGVSGRGVFQNCTAVGVRQQRFVYDAYQNLVGYYSDDGNYYCL
RACVSVPVSVIYDKETKTHATLFGSVACEHISSTMSQYSRSTRSM
LKRRDSTYGPLQTPVGCVLGLVNSSLFVEDCKLPLGQSLCALPDT
PSTLTPRSVRSVPGEMRLASIAFNHPIQVDQFNSSYFKLSIPTNF
SFGVTQEYIQTTIQKVTVDCKQYICNGFQKCEQLLREYGQFCSKI
NQALHGANLRQDDSVRNLFASVKSSQSSPIIPGFGGDFNLTLLEP
VSISTGSRSARSAIEDLLFDKVTIADPGYMQGYDDCMQQGPASAR
DLICAQYVAGYKVLPPLMDVNMEAAYTSSLLGSIAGVGWTAGLSS
FAAIPFAQSIFYRLNGVGITQQVLSENQKLIANKFNQALGAMQTG
FTTTNEAFRKVQDAVNNNAQALSKLASELSNTFGAISASIGDIIQ
RLDVLEQDAQIDRLINGRLTTLNAFVAQQLVRSESAALSAQLAKD
KVNECVKAQSKRSGFCGQGTHIVSFVVNAPNGLYFMHVGYYPSNH
IEVVSAYGLCDAANPTNCIAPVNGYFIKTNNTRIVDEWSYTGSSF
YSPEPITSLNTKYVAPQVTYQNISTNLPPPLLGNSTGIDFQDELD
EFFKNVSTSIPNFGSLTQINTTLLDLTYEMLSLQQVVKALNESYI
DLKELGNYTYYNKWPWYIWL
(GenPept gbAHX00711.1).
Ectodomain minus SP, minus MPER 22-1291:
[SEQ ID NO: 140]
GPDSVKSACIEVDIQQTFFDKTWPRPIDVSKADGIIYPQGRTYSN
ITITYQGLFPYQGDHGDMYVYSAGHATGTTPQKLFVANYSQDVKQ
FANGFVVRIGAAANSTGTVIISPSTSATIRKIYPAFMLGSSVGNF
SDGKMGRFFNHTLVLLPDGCGTLLRAFYCILEPRSGNHCPAGNSY
TSFATYHTPATDCSDGNYNRNASLNSFKEYFNLRNCTFMYTYNIT
EDEILEWFGITQTAQGVHLFSSRYVDLYGGNMFQFATLPVYDTIK
YYSIIPHSIRSIQSDRKAWAAFYVYKLQPLTFLLDFSVDGYIRRA
IDCGFNDLSQLHCSYESFDVESGVYSVSSFEAKPSGSVVEQAEGV
ECDFSPLLSGTPPQVYNFKRLVFTNCNYNLTKLLSLFSVNDFTCS
QISPAAIASNCYSSLILDYFSYPLSMKSDLGVSSAGPISQFNYKQ
SFSNPTCLILATVPHNLTTITKPLKYSYINKCSRLLSDDRTEVPQ
LVNANQYSPCVSIVPSTVWEDGDYYRKQLSPLEGGGWLVASGSTV
AMTEQLQMGFGITVQYGTDTNSVCPKLEFANDTKIASQLGNCVEY
SLYGVSGRGVFQNCTAVGVRQQRFVYDAYQNLVGYYSDDGNYYCL
RACVSVPVSVIYDKETKTHATLFGSVACEHISSTMSQYSRSTRSM
LKRRDSTYGPLQTPVGCVLGLVNSSLFVEDCKLPLGQSLCALPDT
PSTLTPRSVRSVPGEMRLASIAFNHPIQVDQFNSSYFKLSIPTNF
SFGVTQEYIQTTIQKVTVDCKQYICNGFQKCEQLLREYGQFCSKI
NQALHGANLRQDDSVRNLFASVKSSQSSPIIPGFGGDFNLTLLEP
VSISTGSRSARSAIEDLLFDKVTIADPGYMQGYDDCMQQGPASAR
DLICAQYVAGYKVLPPLMDVNMEAAYTSSLLGSIAGVGWTAGLSS
FAAIPFAQSIFYRLNGVGITQQVLSENQKLIANKFNQALGAMQTG
FTTTNEAFRKVQDAVNNNAQALSKLASELSNTFGAISASIGDIIQ
RLDVLEQDAQIDRLINGRLTTLNAFVAQQLVRSESAALSAQLAKD
KVNECVKAQSKRSGFCGQGTHIVSFVVNAPNGLYFMHVGYYPSNH
IEVVSAYGLCDAANPTNCIAPVNGYFIKTNNTRIVDEWSYTGSSF
YSPEPITSLNTKYVAPQVTYQNISTNLPPPLLGNSTGIDFQDELD
EFFKNVSTSIPNFGSLTQINTTLLDLTYEMLSLQQWVKALNESYI
DLKELGNYTY
(GenPept gbAHX00711.1).
Ectodomain minus SP plus altered furin
cleavage sites:
[SEQ ID NO: 141]
GPDSVKSACIEVDIQQTFFDKTWPRPIDVSKADGIIYPQGRTYSN
ITITYQGLFPYQGDHGDMYVYSAGHATGTTPQKLFVANYSQDVKQ
FANGFVVRIGAAANSTGTVIISPSTSATIRKIYPAFMLGSSVGNF
SDGKMGRFFNHTLVLLPDGCGTLLRAFYCILEPRSGNHCPAGNSY
TSFATYHTPATDCSDGNYNRNASLNSFKEYFNLRNCTFMYTYNIT
EDEILEWFGITQTAQGVHLFSSRYVDLYGGNMFQFATLPVYDTIK
YYSIIPHSIRSIQSDRKAWAAFYVYKLQPLTFLLDFSVDGYIRRA
IDCGFNDLSQLHCSYESFDVESGVYSVSSFEAKPSGSVVEQAEGV
ECDFSPLLSGTPPQVYNFKRLVFTNCNYNLTKLLSLFSVNDFTCS
QISPAAIASNCYSSLILDYFSYPLSMKSDLGVSSAGPISQFNYKQ
SFSNPTCLILATVPHNLTTITKPLKYSYINKCSRLLSDDRTEVPQ
LVNANQYSPCVSIVPSTVWEDGDYYRKQLSPLEGGGWLVASGSTV
AMTEQLQMGFGITVQYGTDTNSVCPKLEFANDTKIASQLGNCVEY
SLYGVSGRGVFQNCTAVGVRQQRFVYDAYQNLVGYYSDDGNYYCL
RACVSVPVSVIYDKETKTHATLFGSVACEHISSTMSQYSRSTRSM
LKRRDSTYGPLQTPVGCVLGLVNSSLFVEDCKLPLGQSLCALPDT
PSTLTPNSVNSVPGEMRLASIAFNHPIQVDQFNSSYFKLSIPTNF
SFGVTQEYIQTTIQKVTVDCKQYICNGFQKCEQLLREYGQFCSKI
NQALHGANLRQDDSVRNLFASVKSSQSSPIIPGFGGDFNLTLLEP
VSISTGSNSANSAIEDLLFDKVTIADPGYMQGYDDCMQQGPASAR
DLICAQYVAGYKVLPPLMDVNMEAAYTSSLLGSIAGVGWTAGLSS
FAAIPFAQSIFYRLNGVGITQQVLSENQKLIANKFNQALGAMQTG
FTTTNEAFRKVQDAVNNNAQALSKLASELSNTFGAISASIGDIIQ
RLDVLEQDAQIDRLINGRLTTLNAFVAQQLVRSESAALSAQLAKD
KVNECVKAQSKRSGFCGQGTHIVSFVVNAPNGLYFMHVGYYPSNH
IEVVSAYGLCDAANPTNCIAPVNGYFIKTNNTRIVDEWSYTGSSF
YSPEPITSLNTKYVAPQVTYQNISTNLPPPLLGNSTGIDFQDELD
EFFKNVSTSIPNFGSLTQINTTLLDLTYEMLSLQQVVKALNESYI
DLKELGNYTYYNKWPWYIWL
(GenPept gbAHX00711.1).

### 2.2.1.14 VSV G

Non-limiting examples of VSV G ectodomain polypeptides include:

Ectodomain 1-462:
[SEQ ID NO: 142]
MKCLLYLAFLFIGVNCKFTIVFPHNQKGNWKNVPSNYHYCPSSSD
LNWHNDLIGTAIQVKMPKSHKAIQADGWMCHASKWVTTCDFRWYG
PKYITQSIRSFTPSVEQCKESIEQTKQGTWLNPGFPPQSCGYATV
TDAEAVIVQVTPHHVLVDEYTGEWVDSQFINGKCSNYICPTVHNS
TTWHSDYKVKGLCDSNLISMDITFFSEDGELSSLGKEGTGFRSNY
FAYETGGKACKMQYCKHWGVRLPSGVWFEMADKDLFAAARFPECP
EGSSISAPSQTSVDVSLIQDVERILDYSLCQETWSKIRAGLPISP
VDLSYLAPKNPGTGPAFTIINGTLKYFETRYIRVDIAAPILSRMV
GMISGTTTERELWDDWAPYEDVEIGPNGVLRTSSGYKFPLYMIGH
GMLDSDLHLSSKAQVFEHPHIQDAASQLPDDESLFFGDTGLSKNP
IELVEGWFSSWK
(GenPept gbADX53329.1).

This sequence comprises the following domains/moieties:
SP=1-17
Ectodomain=1-462
MPER=421-462

Ectodomain minus SP:
[SEQ ID NO: 143]
FTIVFPHNQKGNWKNVPSNYHYCPSSSDLNWHNDLIGTAIQVKMP
KSHKAIQADGWMCHASKWVTTCDFRWYGPKYITQSIRSFTPSVEQ
CKESIEQTKQGTWLNPGFPPQSCGYATVTDAEAVIVQVTPHHVLV
DEYTGEWVDSQFINGKCSNYICPTVHNSTTWHSDYKVKGLCDSNL
ISMDITFFSEDGELSSLGKEGTGFRSNYFAYETGGKACKMQYCKH
WGVRLPSGVWFEMADKDLFAAARFPECPEGSSISAPSQTSVDVSL
IQDVERILDYSLCQETWSKIRAGLPISPVDLSYLAPKNPGTGPAF
TIINGTLKYFETRYIRVDIAAPILSRMVGMISGTTTERELWDDWA
PYEDVEIGPNGVLRTSSGYKFPLYMIGHGMLDSDLHLSSKAQVFE
HPHIQDAASQLPDDESLFFGDTGLSKNPIELVEGWFSSWK
(gbADX53329.1).
Ectodomain minus SP, minus MPER:
[SEQ ID NO: 144]
FTIVFPHNQKGNWKNVPSNYHYCPSSSDLNWHNDLIGTAIQVKMP
KSHKAIQADGWMCHASKWVTTCDFRWYGPKYITQSIRSFTPSVEQ
CKESIEQTKQGTWLNPGFPPQSCGYATVTDAEAVIVQVTPHHVLV
DEYTGEWVDSQFINGKCSNYICPTVHNSTTWHSDYKVKGLCDSNL
ISMDITFFSEDGELSSLGKEGTGFRSNYFAYETGGKACKMQYCKH
WGVRLPSGVWFEMADKDLFAAARFPECPEGSSISAPSQTSVDVSL
IQDVERILDYSLCQETWSKIRAGLPISPVDLSYLAPKNPGTGPAF
TIINGTLKYFETRYIRVDIAAPILSRMVGMISGTTTERELWDDWA
PYEDVEIGPNGVLRTSSGYKFPLYMIGHGMLDSDLHLSSKAQVF
(GenPept gbADX53329.1).

### 2.2.1.15 RABV GP

Non-limiting examples of RABV GP ectodomain polypeptides include:

Ectodomain 1-458:
[SEQ ID NO: 145]
MIPQTLLFVPLLVFSLCFGKFPIYTIPDKLGPWSPIDIHHLSCPN
NLVVEDEGCTNLSGFSYMELKVGYISAIKVNGFTCTGVVTEAETY
TNFVGYVTTTFKRKHFRPTPDACRAAYNWKMAGDPRYEESLHNPY
PDYHWLRTVKTTKESLVIISPSVSDLDPYDKSLHSRVFPSGKCSG
ITVSSTYCPTNHDYTIWMPENPRLGTSCDIFTNSRGKRASKGSKT
CGFVDERGLYKSLKGACKLKLCGVLGLRLMDGTWAAIQTSDEAKW
CPPDQLVNIHDFRSDEIEHLVVEELVKKREECLDALESIMTTKSV
SFRRLSHLRKLVPGFGKAYTIFNKTLMEADAHYKSVRTWNEIIPS
KGCLRVGGRCHPHVNGVFFNGIILGPDGHVLIPEMQSSLLQQHME
LLESSVIPLMHPLADPSTVFKDGDEAEDFVEVHLPDVHKQVSGVD
LGLPSWGK
(GenPept gbAFM52658.1).

This sequence comprises the following domains/moieties:
SP=1-20
Ectodomain=1-458

Ectodomain minus SP:
[SEQ ID NO: 146]
FPIYTIPDKLGPWSPIDIHHLSCPNNLVVEDEGCTNLSGFSYMEL
KVGYISAIKVNGFTCTGVVTEAETYTNFVGYVTTTFKRKHFRPTP
DACRAAYNWKMAGDPRYEESLHNPYPDYHWLRTVKTTKESLVIIS
PSVSDLDPYDKSLHSRVFPSGKCSGITVSSTYCPTNHDYTIWMPE
NPRLGTSCDIFTNSRGKRASKGSKTCGFVDERGLYKSLKGACKLK
LCGVLGLRLMDGTWAAIQTSDEAKWCPPDQLVNIHDFRSDEIEHL
VVEELVKKREECLDALESIMTTKSVSFRRLSHLRKLVPGFGKAYT
IFNKTLMEADAHYKSVRTWNEIIPSKGCLRVGGRCHPHVNGVFFN
GIILGPDGHVLIPEMQSSLLQQHMELLESSVIPLMHPLADPSTVF
KDGDEAEDFVEVHLPDVHKQVSGVDLGLPSWGK
(GenPept gbAFM52658.1).

### 2.2.1.16 HSV1 Gb

Non-limiting examples of HSV1 Gb ectodomain polypeptides include:

Ectodomain 1-774:
[SEQ ID NO: 147]
MRQGAPARGCRWFVVWALLGLTLGVLVASAAPSSPGTPGVAAATQ
AANGGPATPAPPALGAAPTGDPKPKKNKKPKNPTPPRPAGDNATV
AAGHATLREHLRDIKAESTDANFYVCPPPTGATVVQFEQPRRCPT
RPEGQNYTEGIAVVFKENIAPYKFKATMYYKDVTVSQVWFGHRYS
QFMGIFEDRAPVPFEEVIDKINAKGVCRSTAKYVRNNLETTAFHR
DDHETDMELKPANAATRTSRGWHTTDLKYNPSRVEAFHRYGTTVN
CIVEEVDARSVYPYDEFVLATGDFVYMSPFYGYREGSHTEHTSYA
ADRFKQVDGFYARDLTTKARATAPTTRNLLTTPKFTVAWDWVPKR
PSVCTMTKWQEVDEMLRSEYGGSFRFSSDAISTTFTTNLTEYPLS
RVDLGDCIGKDARDAMDRIFARRYNATHIKVGQPQYYLANGGFLI
AYQPLLSNTLAELYVREHLREQSRKPPNPTPPPPGASANASVERI
KTTSSIEFARLQFTYNHIQRHVNDMLGRVAIAWCELQNHELTLWN
EARKLNPNAIASATVGRRVSARMLGDVMAVSTCVPVAADNVIVQN
SMRISSRPGACYSRPLVSFRYEDQGPLVEGQLGENNELRLTRDAI
EPCTVGHRRYFTFGGGYVYFEEYAYSHQLSRADITTVSTFIDLNI
TMLEDHEFVPLEVYTRHEIKDSGLLDYTEVQRRNQLHDLRFADID
TVIHADANAAMFAGLGAFFEGMGDLGRAVGKVVMGIVGGVVSAVS
GVSSFMSNP
(GenPept gbAAF04615.1).

This sequence comprises the following domains/moieties:
SP=1-24
Ectodomain=1-774

Ectodomain minus SP 25-774:
[SEQ ID NO: 148]
VLVASAAPSSPGTPGVAAATQAANGGPATPAPPALGAAPTGDPKP
KKNKKPKNPTPPRPAGDNATVAAGHATLREHLRDIKAESTDANFY
VCPPPTGATVVQFEQPRRCPTRPEGQNYTEGIAVVFKENIAPYKF
KATMYYKDVTVSQVWFGHRYSQFMGIFEDRAPVPFEEVIDKINAK
GVCRSTAKYVRNNLETTAFHRDDHETDMELKPANAATRTSRGWHT
TDLKYNPSRVEAFHRYGTTVNCIVEEVDARSVYPYDEFVLATGDF
VYMSPFYGYREGSHTEHTSYAADRFKQVDGFYARDLTTKARATAP
TTRNLLTTPKFTVAWDWVPKRPSVCTMTKWQEVDEMLRSEYGGSF
RFSSDAISTTFTTNLTEYPLSRVDLGDCIGKDARDAMDRIFARRY
NATHIKVGQPQYYLANGGFLIAYQPLLSNTLAELYVREHLREQSR
KPPNPTPPPPGASANASVERIKTTSSIEFARLQFTYNHIQRHVND
MLGRVAIAWCELQNHELTLWNEARKLNPNAIASATVGRRVSARML
GDVMAVSTCVPVAADNVIVQNSMRISSRPGACYSRPLVSFRYEDQ
GPLVEGQLGENNELRLTRDAIEPCTVGHRRYFTFGGGYVYFEEYA
YSHQLSRADITTVSTFIDLNITMLEDHEFVPLEVYTRHEIKDSGL
LDYTEVQRRNQLHDLRFADIDTVIHADANAAMFAGLGAFFEGMGD
LGRAVGKVVMGIVGGVVSAVSGVSSFMSNP
(GenPept gbAAF04615.1).

### 2.2.2 Bacterial Outer Membrane Polypeptides

In preferred embodiments, the bacterial outer membrane polypeptide is:
(i) a Chlamydia major outer membrane protein (MOMP) polypeptide; or
(ii) a trimeric autotransporter adhesin (TAA) polypeptide.

### 2.2.2.1 Chlamydia Major Outer Membrane Protein (MOMP) Polypeptide

Chlamydial bacteria are obligate intracellular pathogens of eukaryotic cells and are known as the most common cause of bacterial sexually transmitted infections worldwide. While infections may resolve with antibiotic treatment, this is often neglected due to frequent asymptomatic infections, leading to disease progression and severe sequelae. Development of a vaccine against Chlamydia is hence considered crucial. The “Chlamydia major outer membrane protein” (commonly also referred to as “MOMP” or “Chlamydia MOMP”) has become one of the prime target molecules for vaccine development (see, e.g., review by Madico G et al., Structural and Immunological Characterization of Novel Recombinant MOMP-Based Chlamydial Antigens. Vaccines (Basel). 2017; 6(1):2. doi: 10.3390/vaccines6010002).

Chlamydia MOMP is a surface-exposed trimeric porin with a putative 16-stranded barrel transmembrane core region, 8 surface-exposed loops and 8 short periplasmic loops per monomer. Thus, in view of MOMP being naturally in a trimeric conformation, the herein disclosed technology will likely also provide a benefit in terms of stabilizing a trimeric state of polypeptides derived or corresponding to MOMP. Corresponding trimers formed upon trimerization of the herein disclosed molecular clamp may allow to present the MOMP derived antigen in a conformation which resembles the natural trimeric state of MOMP (or fragments/portions derived therefrom). Thus, when employing respective chimeric polypeptides as a vaccine, an antigen presentation resembling the natural trimeric conformation may result in the generation of a more potent broadly neutralizing antibody response as compared to antigens not presented in a trimeric state.

Molecular characterization and a topology modeling of MOMP have identified four serovar-specific domains of sequence variability (variable domains, VD) in loops 2, 3, 5 and 6, along with constant domains (CDs). Whereas the VDs have been shown to contain B- and T-cell epitopes which can elicit humoral responses (monoclonal and polyclonal), the CDs can induce T-cell responses (see, e.g., Madico G et a/Vaccines (Basel). 2017; 6(1):2).

The term “Chlamydia major outer membrane protein (MOMP) polypeptide” or “Chlamydia major outer membrane porin (MOMP) polypeptide”, as interchangeably used herein, is intended to refer to both, the full-length polypeptide that corresponds to a monomeric subunit of MOMP, as well as to fragments or portions of the latter polypeptide.

There are three known species of the family Chlamydia which infect humans: C. trachomatis, C. pneumoniae, and C. psittaci. Genomic sequences for each of these are publicly available.

Thus, in preferred embodiments, the Chlamydia major outer membrane protein (MOMP) polypeptide corresponds to, or is a variant of, a Chlamydia MOMP polypeptide from a species selected from C. trachomatis, C. pneumoniae, and C. psittaci.

In particularly preferred embodiments, the Chlamydia MOMP polypeptide comprises or consists of an amino acid sequence having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, more preferably 100%) sequence identity to one of the amino acid sequences defined by SEQ ID NO: 216 or 217.

Chlamydia trachomatis major outer membrane
protein (MOMP) (as comprised in SEQ ID NO: 263)
SEQ ID NO: 216
MKKLLKSVLVFAALSSASSLQALPVGNPAEPSLMIDGILWEGFGGDPCDP
CTTWCDAISMRVGYYGDFVFDRVLKTDVNKEFQMGAAPTTSDVAGLQNDP
TINVARPNPAYGKHMQDAEMFTNAAYMALNIWDRFDVFCTLGATTGYLKG
NSASFNLVGLFGTKTQSSSFNTAKLIPNTALNEAVVELYINTTFAWSVGA
RAALWECGCATLGASFQYAQSKPKVEELNVLCNASEFTINKPKGYVGAEF
PLNITAGTEAATGTKDASIDYHEWQASLALSYRLNMFTPYIGVKWSRVSF
DADTIRIAQPKLAEAILDVTTLNRTTAGKGSVVSAGTDNELADTMQIVSL
QLNKMKSRKSCGIAVGTTIVDADKYAVTVEARLIDERAAHVNAQFRF

Italicized text corresponds to the leader sequence

Chlamydia pneumoniae major outer membrane
protein (MOMP); Uniprot entry: P27455
SEQ ID NO: 217
MKKLLKSALLSAAFAGSVGSLQALPVGNPSDPSLLIDGTIWEGAA
GDPCDPCATWCDAISLRAGFYGDYVFDRILKVDAPKTFSMGAKPT
GSAAANYTTAVDRPNPAYNKHLHDAEWFTNAGFIALNIWDRFDVF
CTLGASNGYIRGNSTAFNLVGLFGVKGTTVNANELPNVSLSNGVV
ELYTDTSFSWSVGARGALWECGCATLGAEFQYAQSKPKVEELNVI
CNVSQFSVNKPKGYKGVAFPLPTDAGVATATGTKSATINYHEWQV
GASLSYRLNSLVPYIGVQWSRATFDADNIRIAQPKLPTAVLNLTA
WNPSLLGNATALSTTDSFSDFMQIVSCQINKFKSRKACGVTVGAT
LVDADKWSLTAEARLINERAAHVSGQFRF

Italicized text corresponds to the leader sequence

In even more preferred embodiments, the chimeric polypeptide comprises or consists of an amino acid sequence having at least 70% (or, with increasing preference, at least 71%, 72%, 73%, 740, 750, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, more preferably 100%) sequence identity to the amino acid sequence defined by SEQ ID NO: 263.

Whereas preferably the chimeric polypeptide of the invention is a single polypeptide chain, wherein the structure stabilizing moiety (SSM) is C-terminal of (i.e., downstream to) the bacterial surface polypeptide, the present disclosure also contemplates as an alternative arrangement that the structure-stabilizing moiety (SSM) is N-terminal of (i.e., upstream to) the bacterial surface polypeptide. In both instances, the structure-stabilizing moiety (SSM) and the bacterial surface polypeptide may be connected by a hinge as defined herein.

### Chlamydia PGP3 Polypeptide

Another surface polypeptide of Chlamydia and prominent antigen which is expressly contemplated herein for being included into the chimeric polypeptide as a bacterial surface polypeptide is the so-called “plasmid gene protein 3 (PGP3)”.

Thus, in preferred embodiments, the bacterial surface polypeptide is a Chlamydia PGP3 polypeptide, more preferably a Chlamydia PGP3 polypeptide from Chlamydia trachomatis.

In particular preferred embodiments of the latter embodiment, the Chlamydia trachomatis PGP3 polypeptide comprises or consists of an amino acid sequence having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, more preferably 100%) sequence identity to one of the amino acid sequences defined by SEQ ID NO: 259.

Chlamydia trachomatis PGP3 (sequence corresponds
to amino acid residues 3-264 of full-length
protein)
SEQ ID NO: 259
NSGFYLYNTENCVFADNIKVGQMTEPLKDQQIILGTTSTPVAAKM
TASDGISLTVSNNSSTNASITIGLDAEKAYQLILEKLGNQILDGI
ADTIVDSTVQDILDKITTDPSLGLLKAFNNFPITNKIQCNGLFTP
SNIETLLGGTEIGKFTVTPKSSGSMFLVSADIIASRMEGSVVLAL
VREGDSKPCAISYGYSSGVPNLCSLRTSITNTGLTPTTYSLRVGG
LESGVVWVNALSNGNDILGITNTSNVSFLEVIPQTNA

Based upon an assessment of the structural topology of the Chlamydia PGP3 polypeptide, it is thought by the inventors that an arrangement wherein the SSM is N-terminal to (i.e., upstream of) a Chlamydia PGP3 polypeptide will be particularly suitable in terms of yielding a stably folded fusion protein and in terms of providing an effective display of the Chlamydia PGP3 polypeptide as antigen.

Thus, in alternative, yet particularly preferred aspects of the invention, a chimeric polypeptide is provided which comprises a heterologous, structure-stabilizing moiety (SSM) operably connected downstream to a Chlamydia PGP3 polypeptide, wherein the structure-stabilizing moiety is a polypeptide comprising, in an N- to C-terminal order, a first heptad repeat region (FHRR) and a second heptad repeat region (SHRR), wherein (i) the FHRR comprises or consists of an amino acid sequence having at least 60% sequence identity to the amino acid sequence set forth in SEQ ID NO: 80 or 81, and the SHIRR comprises or consists of an amino acid sequence having at least 40% sequence identity to the amino acid sequence set forth in SEQ ID NO: 82 or 83; and/or (ii) the FHRR comprises or consists of an amino acid sequence having at least 90% sequence similarity to the amino acid sequence set forth in SEQ ID NO: 80 or 81, and the SHIRR comprises or consists of an amino acid sequence having at least 70% sequence similarity to the amino acid sequence set forth in SEQ ID NO: 82 or 83.

In particularly preferred embodiments of the latter aspect, the chimeric polypeptide comprises or consists of an amino acid sequence having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, more preferably 100%) sequence identity to the amino acid sequence defined by SEQ ID NO: 264.

The skilled person will readily understand that the embodiments as disclosed herein in connection with the chimeric polypeptide according to the first aspect of the invention, and the embodiments of the further aspects relating to the chimeric polypeptide according to the first aspect of the invention, apply in a far as applicable, mutatis mutandis to the chimeric polypeptide as defined in accordance with the above-mentioned alternative aspect.

### 2.2.2.2 Trimeric Autotransporter Adhesin (TAA) Polypeptides

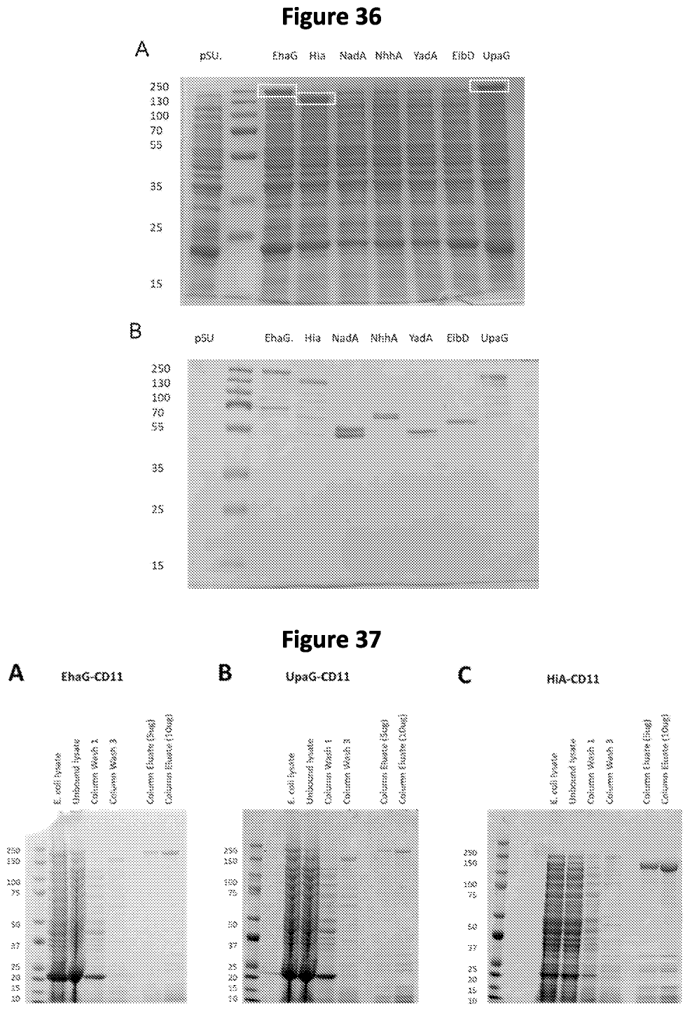

As demonstrated by the herein disclosed evidence (see, e.g., Example 19 and corresponding FIGS. 36 to 39), the applicability of the structure-stabilizing moiety of the invention is by no means confined to the stabilization of enveloped virus fusion protein ectodomain-derived antigens but can also suitably be employed for the stabilization of other (poly)peptides, in particular of those which also naturally occur in a trimeric state and which natural conformation may hence also be stabilized by the herein disclosed SSM.

Of particular interest are further (poly)peptides which also constitute target antigens for vaccines including those of bacterial origin, such as bacterial outer membrane proteins (OMPs). Among the latter are the so-called “trimeric autotransporter adhesins (TAAs)” which have recently been endorsed as promising new vaccine targets (see, e.g., review by Thibau A. et al. Immunogenicity of trimeric autotransporter adhesins and their potential as vaccine targets. Med Microbiol Immunol. 2020 June; 209(3):243-263. doi: 10.1007/s00430-019-00649-y).

The term “trimeric autotransporter adhesins (TAAs)”, commonly also known as “bacterial autotransporters”, “trimeric autotransporters”, “non-fimbrial adhesins (NFAs)”, or “oligomeric coiled-coil adhesins (Ocas)”, refers to a group of outer membrane proteins of Gram-negative bacteria which play important roles in bacterial infection and host colonization. TAAs are generally built of three identical polypeptide chains (fibers) which are assembled into long filamentous trimeric proteins. Each monomeric subunit typically consists of an N-terminal extracellular portion, commonly referred to as the “passenger domain”, and a C-terminal membrane anchor, commonly referred to as “translocator domain”, “translocation domain” or “p-domain”. The N-terminal “passenger domain” is responsible for the specific effector functions, such as adhesion to specific molecular components on host cells. The passenger domain typically comprises one or more head and neck domains, as well as one or more coiled-coil stalk domains. Dependent on the arrangement of the head domain(s), TAAs can be classified as either “lollipop” or “beads-on-a-string”-like structures. The C-terminal “translocator domain” typically consists of three subunits, each composed of one long, amphipathic helix, followed by a four-stranded β-meander which are assembled into a 12-stranded β-barrel (each of the monomeric subunits contributing four strands) that is embedded in the bacterial outer membrane. The translocator domain is believed to be responsible for insertion and translocation of the passenger domain to the outside of the cell. Whereas in some TAAs, such as YadA of Yersinia enterocolitica, the passenger domain consists of only one head region and one stalk region, more complex TAAs exist, which comprise multiple head and stalk regions in various arrangements. Moreover, although all TAAs have a translocator domain, not all of them contain both, a stalk and a head region. TAAs are synthesized as precursors that contain three functional domains: an N-terminal signal sequence, the passenger domain and the C-terminal translocator domain. The signal sequence is usually about 20-50 amino acid residues in length (hence often designated as “extended signal peptide”) and targets these proteins to the Sec transport machinery of the cytoplasmic membrane. Translocation across this membrane then proceeds via the Sec pathway utilizing ATP as an energy source and culminates with the loss of the signal peptide. From within the periplasm, the translocator domain then inserts into the outer membrane as a β-barrel structure and forms a pore through which the passenger domain translocates onto the bacterial cell surface. At this point, the passenger domain can remain associated with the outer membrane, via its covalent attachment to the translocator domain or in a noncovalent manner following cleavage from the translocator domain. Alternatively, the passenger domain can be released free into the extracellular milieu following cleavage from the β-domain (see, e.g., Linke D et al., Trimeric autotransporter adhesins: variable structure, common function. Trends Microbiol. 2006; 14(6):264-70; Bassler J et al.; A domain dictionary of trimeric autotransporter adhesins. Int J Med Microbiol. 2015; 305(2):265-75; Kiessling A R et al., Recent advances in the understanding of trimeric autotransporter adhesins. Med Microbiol Immunol. 2020; 209(3):233-242).

As shown in Example 19 and corresponding FIGS. 36 to 39, the present technology could also successfully be employed to generate chimeric polypeptides and complexes thereof which, instead of an enveloped virus fusion ectodomain polypeptide, comprised a TAA polypeptide fused to the SSM of the invention. In total, a panel of seven constructs with TAAs derived from major bacterial pathogens could be solubly and stably expressed and purified from E. coli, and a subsequent analysis (by TEM) indicated overall shapes consistent with their native long filamentous conformation. These results hence suggest that the present molecular clamping technology can also suitably stabilize TAAs in a close to native conformation, thus rendering plausible that respective constructs will similarly prove effective as vaccines, and in particular for eliciting a potent immune response towards these important class of virulence factors.

Although TAAs naturally fold into a trimer, it is understood by the skilled artisan that whenever a “TAA polypeptide” is referred to herein, the term refers to a single polypeptide chain that corresponds to, or is a variant or fragment of, a monomeric subunit of a TAA polypeptide.

Thus, in preferred embodiments, the TAA polypeptide corresponds to, or is a variant of:
(i) a TAA polypeptide from a bacterium of a genus selected from Neisseria, Escherichia, Haemophilus, Yersinia, Salmonella, Bartonella, Vibrio, Acinetobacter and Moraxella; and/or
(ii) a TAA polypeptide selected from Neisseria meningitidis adhesin A (NadA), Neisseria meningitidis hia/hsf homologue (NhhA), Escherichia coli autotransporter G (EhaG), Escherichia coli IgG-binding protein D (EibD), Uropathogenic Escherichia coli autotransporter G (UpaG), Haemophilus influenzae adhesin (HiA) and Yersinia enterocolitica adhesin (YadA).

In preferred embodiments, the bacterium of the genus Neisseria is one of the species N. meningitidis.

In other preferred embodiments, the bacterium of the genus Escherichia is selected from the group consisting of: Enterotoxigenic Escherichia coli (ETEC), Enteropathogenic Escherichia coli (EPEC), Enteroaggregative Escherichia coli (EAEC), Enteroinvasive Escherichia coli (EIEC), Enterohemorrhagic Escherichia coli (EHEC), Adherent-Invasive Escherichia coli (AIEC), and Uropathogenic Escherichia coli (UPEC).

In other preferred embodiments, the bacterium of the genus Haemophilus is selected from the group consisting of: H. influenzae, H. ducreyi, H. aegyptius, H. parainfluenzae, H. parasuis, and H. paragallinarum.

In other preferred embodiments, the bacterium of the genus Yersinia is selected from the group consisting of: Y. enterocolitica and Y. pseudotuberculosis.

In other preferred embodiments, the bacterium of the genus Salmonella is selected from the group consisting of: S. typhi, S. enterica, and S. enteritidis.

In other preferred embodiments, the bacterium of the genus Bartonella is selected from the group consisting of: B. bacilliformis, B. quintana, B. clarridgeiae, B. elizabethae, B. grahamii, B. henselae, B. koehlerae, B. naantaliensis, B. vinsonii, B. washoensis, and B. rochalimae.

In other preferred embodiments, the bacterium of the genus Vibrio is selected from the group consisting of: V. vulnificus, V. parahaemolyticus, V. cholerae and V. campbellii.

In other preferred embodiments, the bacterium of the genus Acinetobacter is selected from the group consisting of: A. calcoaceticus, A. baumannii, A. haemolyticus, A. junii, A. johnsonii, A. Iwoffii, A. radioresistens, A. schindleri, A. ursingii, A. baylyi, A. bouvetii, A. gerneri, A. grimontii, A. tandoii, A. tjernbergiae, A. towneri, and A. parvus.

In other preferred embodiments, the bacterium of the genus Moraxella is selected from the group consisting of: M. catarrhalis, M. lacunata, and M. bovis.

In the herein disclosed Example 19, the chimeric polypeptides were designed to either comprise the full-length TAA polypeptide (without signal peptide) or C-terminally truncated version thereof (which lacked the translocator domain partially or even entirely).

In other or even more preferred embodiments, the TAA polypeptide comprises or consists of:
(i) a passenger domain; and/or
(ii) a translocator domain.

However, it is also thought that constructs only comprising one (i.e., a single) head, neck, or stalk domain, or only the translocator domain can also be generated at similar yield. In view of the head domain often being responsible for the adhesion function, it might be that respective constructs only comprising a head domain as TAA polypeptide fused to the SSM will be particularly effective for eliciting a potent neutralizing antibody response toward that effector protein and related bacteria expressing said protein or similar variants thereof.

In even more preferred embodiments, the TAA polypeptide comprises or consists of at least one head domain, neck domain and/or stalk domain; or any antigenic fragment(s) thereof.

In preferred embodiments, the TAA polypeptide comprises or consists of an amino acid sequence having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, more preferably 100%) sequence identity to one of the amino acid sequences set forth in SEQ ID NOs: 252 to 258.

In even more preferred embodiments, the chimeric polypeptide comprises or consists of an amino acid sequence having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, more preferably 100%) sequence identity to one of the amino acid sequences set forth in SEQ ID NOs: 218-224.

In addition, the present technology may also suitably be employed for effecting a trimerization of other (poly)peptides, such as therapeutic (poly)peptides, which do naturally not adapt a trimeric state, but which presentation as a trimer may provide a benefit in terms of their activity and/or interaction with other molecules/binding partners, e.g., when applied as a medicament. Exemplary embodiments are described in section 2.4, below.

### 2.3 Representative Chimeric Polypeptide Constructs

Non-limiting examples of chimeric polypeptides of the present invention are set out below:

RSV Fusion protein 66K-T¹⁰³-GS-G¹⁴⁵-E⁵¹¹-GSG-VISNA-
based SSM
[SEQ ID NO: 22]
MELLILKANAITTILTAVTFCFASGQNITEEFYQSTCSAVSKGYLSALRT
GWYTSVITIELSNIKKNKCNGTDAKVKLIKQELDKYKNAVTELQLLMQST
PATGSGSAIASGVAVSKVLHLEGEVNKIKSALLSTNKAVVSLSNGVSVLT
SKVLDLKNYIDKQLLPIVNKQSCSISNIETVIEFQQKNNRLLEITREFSV
NAGVTTPVSTYMLTNSELLSLINDMPITNDQKKLMSNNVQIVRQQSYSIM
SIIKEEVLAYVVQLPLYGVIDTPCWKLHTSPLCTTNTKEGSNICLTRTDR
GWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDEFD
ASISQVNEKINQSLAFIRKSDEGSGQSLANATAAQQEVLEAQYAMVQHIA
KGIRILEARVARGGSGGNHTWQQWEEEIEQHEGNLSLLLREAALQVHIAQ
RDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
Italicized text corresponds to the signal peptide of RSV Fusion protein;
Bold text is a flexible linker; and
Underlined text corresponds to a VISNA-based SSM (CD11).

RSV Fusion protein 66E-T¹⁰³-GS-G¹⁴⁵-E⁵¹¹-GSG-VISNA-
based SSM
[SEQ ID NO: 24]
MELLILKANAITTILTAVTFCFASGQNITEEFYQSTCSAVSKGYLSALRT
GWYTSVITIELSNIKENKCNGTDAKVKLIKQELDKYKNAVTELQLLMQST
PATGSGSAIASGVAVSKVLHLEGEVNKIKSALLSTNKAVVSLSNGVSVLT
SKVLDLKNYIDKQLLPIVNKQSCSISNIETVIEFQQKNNRLLEITREFSV
NAGVTTPVSTYMLTNSELLSLINDMPITNDQKKLMSNNVQIVRQQSYSIM
SIIKEEVLAYVVQLPLYGVIDTPCWKLHTSPLCTTNTKEGSNICLTRTDR
GWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDEFD
ASISQVNEKINQSLAFIRKSDEGSGQSLANATAAQQEVLEAQYAMVQHIA
KGIRILEARVARGGSGGNHTWQQWEEEIEQHEGNLSLLLREAALQVHIAQ
RDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of RSV Fusion protein;
Bold text is a flexible linker; and
Underlined text corresponds to a VISNA-based SSM.

SARS-COV-2 Spike protein-N⁶⁷⁹-GSG-S⁶⁹¹-G¹²⁰⁴-VISNA-
based SSM CD11
[SEQ ID NO: 29]
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNGSGSIIAYTMSLGAENSVAYS
NNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPD
PSKPSKRSFIEDLLFNKVTLADAGFIKQYGDCLGDIAARDLICAQKFNGL
TVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQA
LNTLVKQLSSNFGAISSVLNDILSRLDKVEAEVQIDRLITGRLQSLQTYV
TQQLIRAAEIRASANLAATKMSECVLGQSKRVDFCGKGYHLMSFPQSAPH
GVVFLHVTYVPAQEKNFTTAPAICHDGKAHFPREGVFVSNGTHWFVTQRN
FYEPQIITTDNTFVSGNCDVVIGIVNNTVYDPLQPELDSFKEELDKYFKN
HTSPDVDLGDISGINASVVNIQKEIDRLNEVAKNLNESLIDLQELGSGQS
LANATAAQQEVLEAQYAMVQHIAKGIRILEARVARGGSGGNHTWQQWEEE
IEQHEGNLSLLLREAALQVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of SARS-CoV-2 spike protein;
Bold text is a flexible linker; and
Underlined text corresponds to a VISNA-based SSM CD11.

Nipah virus Fusion-VISNA-based SSM
[SEQ ID NO: 31]
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTGSGQSLANATAAQQEVL
EAQYAMVQHIAKGIRILEARVARGGSGGNHTWQQWEEEIEQHEGNLSLLL
REAALQVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of Nipah virus Fusion protein; and
Bold text is a flexible linker; and
Underlined text corresponds to a VISNA-based SSM CD11.

Influenza Hemagglutinin protein R⁵²⁶-VISNA-based
SSM
[SEQ ID NO: 33]
MKAILVVLLYTFATANADTLCIGYHANNSTDTVDTVLEKNVTVTHSVNLL
EDKHNGKLCKLRGVAPLHLGKCNIAGWILGNPECESLSTASSWSYIVETP
SSDNGTCYPGDFIDYEELREQLSSVSSFERFEIFPKTSSWPNHDSNKGVT
AACPHAGAKSFYKNLIWLVKKGNSYPKLSKSYINDKGKEVLVLWGIHHPS
TSADQQSLYQNADTYVFVGSSRYSKKFKPEIAIRPKVRDQEGRMNYYWTL
VEPGDKITFEATGNLVVPRYAFAMERNAGSGIIISDTPVHDCNTTCQTPK
GAINTSLPFQNIHPITIGKCPKYVKSTKLRLATGLRNIPSIQSRGLFGAI
AGFIEGGWTGMVDGWYGYHHQNEQGSGYAADLKSTONAIDEITNKVNSVI
EKMNTQFTAVGKEFNHLEKRIENLNKKVDDGFLDIWTYNAELLVLLENER
TLDYHDSNVKNLYEKVRSQLKNNAKEIGNGCFEFYHKCDNTCMESVKNGT
YDYPKYSEEAKLNREEIDGVKLESTRGSGQSLANATAAQQEVLEAQYAMV
QHIAKGIRILEARVARGGSGGNHTWQQWEEEIEQHEGNLSLLLREAALQV
HIAQRDARRI,

or an amino acid sequence corresponding thereto (e.g., an amino acid sequence having at least 75%, 76%, 77%, 78%, 79%, 80%, 81%, 8200, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of Influenza Hemagglutinin protein; and
Bold text is a flexible linker; and
Underlined text corresponds to a VISNA-based SSM CD11.

SARS-COV-2 Spike protein-N⁶⁷⁹-GSG-S⁶⁹¹-G¹²⁰⁴-VISNA-
based SSM CD11 (-SG)
[SEQ ID NO: 34]
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNGSGSIIAYTMSLGAENSVAYS
NNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPD
PSKPSKRSFIEDLLFNKVTLADAGFIKQYGDCLGDIAARDLICAQKFNGL
TVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQA
LNTLVKQLSSNFGAISSVLNDILSRLDKVEAEVQIDRLITGRLQSLQTYV
TQQLIRAAEIRASANLAATKMSECVLGQSKRVDFCGKGYHLMSFPQSAPH
GVVFLHVTYVPAQEKNFTTAPAICHDGKAHFPREGVFVSNGTHWFVTQRN
FYEPQIITTDNTFVSGNCDVVIGIVNNTVYDPLQPELDSFKEELDKYFKN
HTSPDVDLGDISGINASVVNIQKEIDRLNEVAKNLNESLIDLQELGQSLA
NATAAQQEVLEAQYAMVQHIAKGIRILEARVARGGSGGNHTWQQWEEEIE
QHEGNLSLLLREAALQVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of SARS-CoV-2 spike protein;
Bold text is a flexible linker; and
Underlined text corresponds to a VISNA-based SSM CD11.

RSV Fusion protein 66E-T¹⁰³-GS-G¹⁴⁵-E⁵¹¹-GSG-VISNA-
based SSM with additional N-linked
glycosylation sequences (CD11_1245T8)
[SEQ ID NO: 51]
MELLILKANAITTILTAVTFCFASGQNITEEFYQSTCSAVSKGYLSALRT
GWYTSVITIELSNIKENKCNGTDAKVKLIKQELDKYKNAVTELQLLMQST
PATGSGSAIASGVAVSKVLHLEGEVNKIKSALLSTNKAVVSLSNGVSVLT
SKVLDLKNYIDKQLLPIVNKQSCSISNIETVIEFQQKNNRLLEITREFSV
NAGVTTPVSTYMLTNSELLSLINDMPITNDQKKLMSNNVQIVRQQSYSIM
SIIKEEVLAYVVQLPLYGVIDTPCWKLHTSPLCTTNTKEGSNICLTRTDR
GWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDEFD
ASISQVNEKINQSLAFIRKSDEGSGQSLANATAAQQEVLEAQYAMVQHIA
KGIRILEARVARGGSGGNHTWQNWTEEIENHTGNLTLLLREAANQTHIAQ
RDARRI,

or an amino acid sequence corresponding thereto (e.g., an amino acid sequence having at least 75%, 76%, 77%, 78%, 79%, 80%, 81%, 82%, 83%, 84%, 85%, 87%, 88%, 89%, 90%, 91%, 92%, 93%, 94%, 95%, 96%, 97%, 98% or 99% sequence similarity or identity)
wherein:
Italicized text corresponds to the signal peptide of RSV Fusion protein;
Bold text is a flexible linker; and
Underlined text corresponds to a VISNA-based SSM.

RSV Fusion protein 66E-T¹⁰³-GS-G¹⁴⁵-E⁵¹¹-GSG-VISNA-
based SSM with additional N-linked
glycosylation sequences (CD11_145T8)
[SEQ ID NO: 52]
MELLILKANAITTILTAVTFCFASGQNITEEFYQSTCSAVSKGYLSALRT
GWYTSVITIELSNIKENKCNGTDAKVKLIKQELDKYKNAVTELQLLMQST
PATGSGSAIASGVAVSKVLHLEGEVNKIKSALLSTNKAVVSLSNGVSVLT
SKVLDLKNYIDKQLLPIVNKQSCSISNIETVIEFQQKNNRLLEITREFSV
NAGVTTPVSTYMLTNSELLSLINDMPITNDQKKLMSNNVQIVRQQSYSIM
SIIKEEVLAYVVQLPLYGVIDTPCWKLHTSPLCTTNTKEGSNICLTRTDR
GWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDEFD
ASISQVNEKINQSLAFIRKSDEGSGQSLANATAAQQEVLEAQYAMVQHIA
KGIRILEARVARGGSGGNHTWQQWEEEIENHTGNLTLLLREAANQTHIAQ
RDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of RSV Fusion protein;
Bold text is a flexible linker; and
Underlined text corresponds to a VISNA-based SSM.

SARS-COV-2 (Delta) Spike protein-N⁶⁷⁹-GSG-S⁶⁹¹-G¹²⁰⁴-
VISNA-based SSM CD11
[SEQ ID NO: 73]
MFVFLVLLPLVSSQCVNLRTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESGVYSSANNCTFEYVSQPFLMDLEGKQGNFKNLREFVFKNIDGYFK
IYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYA
WNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYR
YRLFRKSNLKPFERDISTEIYQAGSKPCNGVEGFNCYFPLQSYGFQPTNG
VGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIG
AEHVNNSYECDIPIGAGICASYQTQTNGSGSIIAYTMSLGAENSVAYSNN
SIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIG
VTQNVLYENQKLIANQFNSAIGKIQDSLSSTASALGKLQNVVNQNAQALN
TLVKQLSSNFGAISSVLNDILSRLDKVEAEVQIDRLITGRLQSLQTYVTQ
QLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHT
SPDVDLGDISGINASVVNIQKEIDRLNEVAKNLNESLIDLQELGSGQSLA
NATAAQQEVLEAQYAMVQHIAKGIRILEARVARGGSGGNHTWQQWEEEIE
QHEGNLSLLLREAALQVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of SARS-CoV-2 spike protein;
Bold text is a flexible linker; and
Underlined text corresponds to a VISNA-based SSM CD11.

SARS-COV-2 (Delta) Spike protein-N⁶⁷⁹-GSG-S⁶⁹¹-G¹²⁰⁴-
VISNA-based SSM (CD11) and QS removed from CD11
FHRR
[SEQ ID NO: 74]
MFVFLVLLPLVSSQCVNLRTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESGVYSSANNCTFEYVSQPFLMDLEGKQGNFKNLREFVFKNIDGYFK
IYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCT
LKSFTVEKGIYQTSNFRVQPTESIVRFPNITNLCPFGEVENATRFASVYA
WNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYR
YRLFRKSNLKPFERDISTEIYQAGSKPCNGVEGFNCYFPLQSYGFQPTNG
VGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIG
AEHVNNSYECDIPIGAGICASYQTQTNGSGSIIAYTMSLGAENSVAYSNN
SIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIG
VTQNVLYENQKLIANQFNSAIGKIQDSLSSTASALGKLQNVVNQNAQALN
TLVKQLSSNFGAISSVLNDILSRLDKVEAEVQIDRLITGRLQSLQTYVTQ
QLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHT
SPDVDLGDISGINASVVNIQKEIDRLNEVAKNLNESLIDLQELGSGLANA
TAAQQEVLEAQYAMVQHIAKGIRILEARVARGGSGGNHTWQQWEEEIEQH
EGNLSLLLREAALQVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of Influenza Hemagglutinin protein; and
Underlined text corresponds to a VISNA-based SSM CD11.

RSV Fusion protein 66E-T103-GS-G145-E511-GSG-VISNA-
based SSM (CD11) and QS removed from CD11 FHRR
[SEQ ID NO: 75]
MELLILKANAITTILTAVTFCFASGQNITEEFYQSTCSAVSKGYLSALRT
GWYTSVITIELSNIKENKCNGTDAKVKLIKQELDKYKNAVTELQLLMQST
PATGSGSAIASGVAVSKVLHLEGEVNKIKSALLSTNKAVVSLSNGVSVLT
SKVLDLKNYIDKQLLPIVNKQSCSISNIETVIEFQQKNNRLLEITREFSV
NAGVTTPVSTYMLTNSELLSLINDMPITNDQKKLMSNNVQIVRQQSYSIM
SIIKEEVLAYVVQLPLYGVIDTPCWKLHTSPLCTTNTKEGSNICLTRTDR
GWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDEFD
ASISQVNEKINQSLAFIRKSDEGSGLANATAAQQEVLEAQYAMVQHIAKG
IRILEARVARGGSGGNHTWQQWEEEIEQHEGNLSLLLREAALQVHIAQRD
ARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of RSV Fusion protein;
Bold text is a flexible linker; and
Underlined text corresponds to a VISNA-based SSM.

RSV Fusion protein 66E-T¹⁰³-GS-G¹⁴⁵-E⁵¹¹-GSG-VISNA-
based SSM with additional N-linked glycosylation
sequences and QS removed from CD11 FHRR
(CD11_145T8-QS)
[SEQ ID NO: 76]
MELLILKANAITTILTAVTFCFASGQNITEEFYQSTCSAVSKGYLSALRT
GWYTSVITIELSNIKENKCNGTDAKVKLIKQELDKYKNAVTELQLLMQST
PATGSGSAIASGVAVSKVLHLEGEVNKIKSALLSTNKAVVSLSNGVSVLT
SKVLDLKNYIDKQLLPIVNKQSCSISNIETVIEFQQKNNRLLEITREFSV
NAGVTTPVSTYMLTNSELLSLINDMPITNDQKKLMSNNVQIVRQQSYSIM
SIIKEEVLAYVVQLPLYGVIDTPCWKLHTSPLCTTNTKEGSNICLTRTDR
GWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGC
DYVSNKGVDTVSVGNTLYYVNKQEGKSLYVKGEPIINFYDPLVFPSDEFD
ASISQVNEKINQSLAFIRKSDEGSGQSLANATAAQQEVLEAQYAMVQHIA
KGIRILEARVARGGSGGNHTWQQWEEEIENHTGNLTLLLREAANQTHIAQ
RDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of RSV Fusion protein;
Bold text is a flexible linker; and
Underlined text corresponds to a VISNA-based SSM.

RSV Fusion protein 66E-T¹⁰³-GS-G¹⁴⁵-E⁵¹¹-GSG-
VISNA-based SSM with additional N-linked
glycosylation sequences and QS removed
from CD11 FHRR and CT1 mutation included
(CD11_145T8-QS)
[SEQ ID NO: 77]
MELLILKANAITTILTAVTFCFASGQNITEEFYQSTCSAVSKGYL
SALRTGWYTSVITIELSNIKENKCNGTDAKVKLIKQELDKYKNAV
TELQLLMQSTPATGSGSAIASGVAVSKVLHLEGEVNKIKSALLST
NKAVVSLSNGVSVLTSKVLDLKNYIDKQLLPIVNKQSCSISNIET
VIEFQQKNNRLLEITREFSVNAGVTTPVSTYMLTNSELLSLINDM
PITNDQKKLMSNNVQIVRQQSYSIMSIIKEEVLAYVVQLPLYGVI
DTPCWKLHTSPLCTTNTKEGSNICLTRTDRGWYCDNAGSVSFFPQ
AETCKVQSNRVFCDTMNSLTLPSEVNLCNVDIFNPKYDCKIMTSK
TDVSSSVITSLGAIVSCYGKTKCTASNKNRGIIKTFSNGCDYVSN
KGVDTVSVGNTLYYVNKQEGKSLYVKGEPIINFYDPLVFPSDEFD
ASISQVNEKINQSLAFIRKSDEGSGQSLANATAAQQEVLEAQYAM
VRHIAKGIRILEARVARGGSGGNHTWQQWEEEIENHTGNLTLLLR
EAANQTHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of RSV Fusion protein;
Bold text is a flexible linker; and
Underlined text corresponds to a VISNA-based SSM.

RSV Fusion protein 66E-T¹⁰³-GS-G¹⁴⁵-E⁵¹¹_GSG-
VISNA-based SSM with additional N-linked
glycosylation sequences and QS removed
from CD11 FHRR and CTS mutation included
(CD11_145T8-QS)
[SEQ ID NO: 78]
MELLILKANAITTILTAVTFCFASGQNITEEFYQSTCSAVSKGYL
SALRTGWYTSVITIELSNIKENKCNGTDAKVKLIKQELDKYKNAV
TELQLLMQSTPATGSGSAIASGVAVSKVLHLEGEVNKIKSALLST
NKAVVSLSNGVSVLTSKVLDLKNYIDKQLLPIVNKQSCSISNIET
VIEFQQKNNRLLEITREFSVNAGVTTPVSTYMLTNSELLSLINDM
PITNDQKKLMSNNVQIVRQQSYSIMSIIKEEVLAYVVQLPLYGVI
DTPCWKLHTSPLCTTNTKEGSNICLTRTDRGWYCDNAGSVSFFPQ
AETCKVQSNRVFCDTMNSLTLPSEVNLCNVDIFNPKYDCKIMTSK
TDVSSSVITSLGAIVSCYGKTKCTASNKNRGIIKTFSNGCDYVSN
KGVDTVSVGNTLYYVNKQEGKSLYVKGEPIINFYDPLVFPSDEFD
ASISQVNEKINQSLAFIRKSDEGSGLANATAAQQEVLEAQYAMVQ
HIAKGIRILEARVARGGSGGNHTTWQQWEEEIENHTGNLTLLLRE
AANQTHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of RSV Fusion protein;
Bold text is a flexible linker; and
Underlined text corresponds to a VISNA-based SSM.

RSV Fusion protein 668-T¹⁰³-GS-G¹⁴⁵-F⁵¹¹-GSG-
VISNA-based SSM with additional N-linked
glycosylation sequences and QS removed from
CD11 FHRR and CT9 mutation included
(CD11_145T8-QS)
[SEQ ID NO: 79]
MELLILKANAITTILTAVTFCFASGQNITEEFYQSTCSAVSKGYL
SALRTGWYTSVITIELSNIKENKCNGTDAKVKLIKQELDKYKNAV
TELQLLMQSTPATGSGSAIASGVAVSKVLHLEGEVNKIKSALLST
NKAVVSLSNGVSVLTSKVLDLKNYIDKQLLPIVNKQSCSISNIET
VIEFQQKNNRLLEITREFSVNAGVTTPVSTYMLTNSELLSLINDM
PITNDQKKLMSNNVQIVRQQSYSIMSIIKEEVLAYVVQLPLYGVI
DTPCWKLHTSPLCTTNTKEGSNICLTRTDRGWYCDNAGSVSFFPQ
AETCKVQSNRVFCDTMNSLTLPSEVNLCNVDIFNPKYDCKIMTSK
TDVSSSVITSLGAIVSCYGKTKCTASNKNRGIIKTFSNGCDYVSN
KGVDTVSVGNTLYYVNKQEGKSLYVKGEPIINFYDPLVFPSDEFD
ASISQVNEKINQSLAFIRKSDEGSGQSLANATAAQQEVLEAQYAM
VQHIAKGIRILEARVARGGSGGNHTWQQWEEEIENHTGNLTLLLR
EAANQTHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of RSV Fusion protein;
Bold text is a flexible linker; and
Underlined text corresponds to a VISNA-based SSM.

E. coli EhaG-VISNA-based SSM CD11
[SEQ ID NO: 218]
MNAGNDNGQGVDYGSGSAGDGWVAIGKGAKANTFMNTSGSSTAVG
YDAIAEGQYSSAIGSKTHAIGGASMAFGVSAISEGDRSIALGASS
YSLGQYSMALGRYSKALGKLSIAMGDSSKAEGANAIALGNATKAT
EIMSIALGDTANASKAYSMALGASSVASEENAIAIGAETEAAENA
TAIGNNAKAKGTNSMAMGFGSLADKVNTIALGNGSQALADNAIAI
GQGNKADGVDAIALGNGSQSRGLNTIALGTASNATGDKSLALGSN
SSANGINSVALGADSIADLDNTVSVGNSSLKRKIVNVKNGAIKSD
SYDAINGSQLYAISDSVAKRLGGGAAVDVDDGTVTAPTYNLKNGS
KNNVGAALAVLDENTLQWDQTKGKYSAAHGTSSPTASVITDVADG
TISASSKDAVNGSQLKATNDDVEANTANIATNTSNIATNTANIAT
NTTNITNLTDSVGDLQADALLWNETKKAFSAAHGQDTTSKITNVK
DADLTADSTDAVNGSQLKTTNDAVATNTTNIANNTSNIATNTTNI
SNLTETVTNLGEDALKWDKDNGVFTAAHGTETTSKITNVKDGDLT
TGSTDAVNGSQLKTTNDAVATNTTNIATNTTNISNLTETVTNLGE
DALKWDKDNGVFTAAHGNNTASKITNILDGTVTATSSDAINGSQL
YDLSSNIATYFGGNASVNTDGVFTGPTYKIGETNYYNVGDALAAI
NSSFSTSLGDALLWDATAGKFSAKHGTNGDASVITDVADGEISDS
SSDAVNGSQLHGVSSYVVDALGGGAEVNADGTITAPTYTIANADY
DNVGDALNAIDTTLDDALLWDADAGENGAFSAAHGKDKTASVITN
VANGAISAASSDAINGSQLYTTNKYIADALGGDAEVNADGTITAP
TYTIANAEYNNVGDALDALDDNALLWDETANGGAGAYNASHDGKA
SIITNVANGSISEDSTDAVNGSQLNATNMMIEQNTQIINQLAGNT
DATYIQENGAGINYVRTNDDGLAFNDASAQGVGATAIGYNSVAKG
DSSVAIGQGSYSDVDTGIALGSSSVSSRVIAKGSRDTSITENGVV
IGYDTTDGELLGALSIGDDGKYRQIINVADGSEAHDAVTVRQLQN
AIGAVATTPTKYFHANSTEEDSLAVGTDSLAMGAKTIVNGDKGIG
IGYGAYVDANALNGIAIGSNAQVIHVNSIAIGNGSTTTRGAQTNY
TAYNMDAPQNSVGEFSVGSADGQRQITNVAAGSADTDAVNVGQLK
VTDAQVSQNTQSITNLDNRVTNLDSRVTNIENGIGDIVTTGSTKY
FKTNTDGVDASAQGKDSVAIGSGSIAAADNSVALGTGSVATEENT
ISVGSSTNQRRITNVAAGKNATDAVNVAQLKSSEAGGVRYDTKAD
GSIDYSNITLGGGNGGTTRISNVSAGVNNNDVVNYAQLKQSVQET
KQYTDQRMVEMDNKLSKTESKLSGGIASAMAMTGLPQAYTPGGSG
QSLANATAAQQEVLEAQYAMVQHIAKGIRILEARVARGGSGGNHT
WQQWEEEIEQHEGNLSLLLREAALQVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
   Bold text is a flexible linker; and
   Underlined text corresponds to a VISNA-based SSM.

E. coli EibD-VISNA-based SSM CD11
[SEQ ID NO: 219]
MQNGTYSVLQDDSQKSGPVKYGSTYEVVKTVDNGNFRYEVKEKKN
DKRTLFKFDSEGNVTVKGKGITHTLHDPALKDFARTAEGKKNEQN
GNTPPHKLTDSAVRGVYNKVYGLEKTEITGFSVEDGENGKVSLGS
DAKASGEFSVAVGNGARATEKASTAVGSWAAADGKQSTALGVGTY
AYANASTALGSVAFVDNTATYGTAAGNRAKVDKDATEGTALGAKA
TVTNKNSVALGANSVTTRDNEVYIGYKTGTESDKTYGTRVLGGLS
DGTRNSDAATVGQLNRKVGGVYDDVKARITVESEKQKKYTDQKTS
EVNEKVEARTTVGVDSDGKLTRAEGATKTIAVNDGLVALSGRTDR
IDYAVGAIDGRVTRNTQSIEKNSKAIAANTRTLQQHSARLDSQQR
QINENHKEMKRAAAQSAALTGLFQPYSVGGSGQSLANATAAQQEV
LEAQYAMVQHIAKGIRILEARVARGGSGGNHTWQQWEEEIEQHEG
NLSLLLREAALQVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
   Bold text is a flexible linker; and
   Underlined text corresponds to a VISNA-based SSM.

E. coli UpaG-VISNA-based SSM CD11
[SEQ ID NO: 220]
MDNYTGQPTDYGDGSAGDGWVAIGKGAKANTFMNTSGASTALGYD
AIAEGEYSSAIGSKTLATGGASMAFGVSAKAMGDRSVALGASSVA
NGDRSMAFGRYAKTNGFTSLAIGDSSLADGEKTIALGNTAKAYEI
MSIALGDNANASKEYAMALGASSKAGGADSLAFGRKSTANSTGSL
AIGADSSSSNDNAIAIGNKTQALGVNSMALGNASQASGESSIALG
NTSEASEQNAIALGQGSIASKVNSIALGSNSLSSGENAIALGEGS
AAGGSNSLAFGSQSRANGNDSVAIGVGAAAATDNSVAIGAGSTTD
ASNTVSVGNSATKRKIVNMAAGAISNTSTDAINGSQLYTISDSVA
KRLGGGATVGSDGTVTAVSYALRSGTYNNVGDALSGIDNNTLQWN
KTAGAFSANHGANATNKITNVAKGTVSATSTDVVNGSQLYDLQQD
ALLWNGTAFSAAHGTEATSKITNVTAGNLTAGSTDAVNGSQLKTT
NDNVTTNTTNIATNTTNITNLTDAVNGLGDDSLLWNKAAGAFSAA
HGTEATSKITNVTAGNLTAGSTDAVNGSQLKTTNDNVTTNTTNIA
TNTTNITNLTDAVNGLGDDSLLWNKTAGAFSAAHGTDATSKITNV
TAGNLTAGSTDAVNGSQLKTTNDNVTTNTTNIATNTTNITNLTDA
VNGLGDDSLLWNKTAGAFSAAHGTDATSKITNVKAGDLTAGSTDA
VNGSQLKTTNDNVSTNTTNITNLTDAVNGLGDDSLLWNKTAGAFS
AAHGTDATSKITNVKAGDLTAGSTDAVNGSQLKTTNDNVSTNTTN
ITNLTDSVGDLKDDSLLWNKAAGAFSAAHGTEATSKITNLLAGKI
SSNSTDAINGSQLYGVADSFTSYLGGGADISDTGVLSGPTYTIGG
TDYTNVGDALAAINTSFSTSLGDALLWDATAGKFSAKHGINNAPS
VITDVANGAVSSTSSDAINGSQLYGVSDYIADALGGNAVVNTDGS
ITTPTYAIAGGSYNNVGDALEAIDTTLDDALLWDTTANGGNGAFS
AAHGKDKTASVITNVANGAVSATSNDAINGSQLYSTNKYIADALG
GDAEVNADGTITAPTYTIANTDYNNVGEALDALDNNALLWDEDAG
AYNASHDGNASKITNVAAGDLSTTSTDAVNGSQLNATNILVTQNS
QMINQLAGNTSETYIEENGAGINYVRTNDSGLAFNDASASGIGAT
AVGYNAVASHASSVAIGQDSISEVDTGIALGSSSVSSRVIVKGTR
NTSVSEEGVVIGYDTTDGELLGALSIGDDGKYRQIINVADGSEAH
DAVTVRQLQNAIGAVATTPTKYYHANSTAEDSLAVGEDSLAMGAK
TIVNGNAGIGIGLNTLVLADAINGIAIGSNARANHADSIAMGNGS
QTTRGAQTNYTAYNMDAPQNSVGEFSVGSEDGQRQITNVAAGSAD
TDAVNVGQLKVTDAQVSQNTQSITNLNTQVTNLDTRVTNIENGIG
DIVTTGSTKYFKTNTDGADANAQGKDSVAIGSGSIAAADNSVALG
TGSVADEENTISVGSSTNQRRITNVAAGVNATDAVNVSQLKSSEA
GGVRYDTKADGSIDYSNITLGGGNSGTTRISNVSAGVNNNDAVNY
AQLKQSVQETKQYTDQRMVEMDNKLSKTESKLSGGIASAMAMTGL
PQAYTPGGSGQSLANATAAQQEVLEAQYAMVQHIAKGIRILEARV
ARGGSGGNHTWQQWEEEIEQHEGNLSLLLREAALQVHIAQRDARR
I,

or an amino acid sequence corresponding thereto (e.g., an amino acid sequence having at least 75%, 76%, 77%, 78%, 79%, 80%, 81%, 82%, 830, 84%, 85%, 86%, 87%, 88%, 89%, 90%, 91%, 92%, 93%, 94%, 95%, 96%, 97%, 98% or 99% sequence similarity or identity)
wherein:
   Bold text is a flexible linker; and
   Underlined text corresponds to a VISNA-based SSM.

H. influenzae HIA-VISNA-based SSM CD11
[SEQ ID NO: 221]
MNNNTPVTNKLKAYGDANFNFTNNSIADAEKQVQEAYKGLLNLNE
KNASDKLLVEDNTAATVGNLRKLGWVLSSKNGTRNEKSQQVKHAD
EVLFEGKGGVQVTSTSENGKHTITFALAKDLGVKTATVSDTLTIG
GGAAAGATTTPKVNVTSTTDGLKFAKDAAGANGDTTVHLNGIGST
LTDTLVGSPATHIDGGDQSTHYTRAASIKDVLNAGWNIKGVKAGS
TTGQSENVDFVHTYDTVEFLSADTETTTVTVDSKENGKRTEVKIG
AKTSVIKEKDGKLFTGKANKETNKVDGANATEDADEGKGLVTAKD
VIDAVNKTGWRIKTTDANGQNGDFATVASGTNVTFASGNGTTATV
TNGTDGITVKYDAKVGDGLKLDGDKIAADTTALTVNDGKNANNPK
GKVADVASTDEKKLVTAKGLVTALNSLSWTTTAAEADGGTLDGNA
SEQEVKAGDKVTFKAGKNLKVKQEGANFTYSLQDALTGLTSITLG
TGNNGAKTEINKDGLTITPANGAGANNANTISVTKDGISAGGQSV
KNVVSGLKKFGDANFDPLTSSADNLTKQNDDAYKGLTNLDEKGTD
KQTPVVADNTAATVGDLRGLGWVISADKTTGGSTEYHDQVRNANE
VKFKSGNGINVSGKTVNGRREITFELAKGEVVKSNEFTVKETNGK
ETSLVKVGDKYYSKEDIDLTTGQPKLKDGNTVAAKYQDKGGKVVS
VTDNTEATITNKGSGYVTGNQVADAIAKSGFELGLADEADAKAAF
DDKTKALSAGTTEIVNAHDKVRFANGLNTKVSAATVESTDANGDK
VTTTFVKTDVELPLTQIYNTDANGKKITKVVKDGQTKWYELNADG
TADMTKEVTLGNVDSDGKKVVKDNDGKWYHAKADGTADKTKGEVS
NDKVSTDEKHVVSLDPNDQSKGKGVVIDNVANGDISATSTDAING
SQLYAVAKGVTNLAGQVNNLEGKVNKVGKRADAGTASALAASQLP
QATMPGGSGQSLANATAAQQEVLEAQYAMVQHIAKGIRILEARVA
RGGSGGNHTWQQWEEEIEQHEGNLSLLLREAALQVHIAQRDARR
I,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
   Bold text is a flexible linker; and
   Underlined text corresponds to a VISNA-based SSM.

N. meningitidis NadA-VISNA-based SSM CD11
[SEQ ID NO: 222]
MATNDDDVKKAATVAIAAAYNNGQEINGFKAGETIYDIDEDGTIT
KKDATAADVEADDFKGLGLKKVVTNLTKTVNENKQNVDAKVKAAE
SEIEKLTTKLADTDAALADTDAALDATTNALNKLGENITTFAEET
KTNIVKIDEKLEAVADTVDKHAEAFNDIADSLDETNTKADEAVKT
ANEAKQTAEETKQNVDAKVKAAETAAGKAEAAAGTANTAADKAEA
VAAKVTDIKADIATNKDNIAKKANSADVYTREESDSKFVRIDGLN
ATTEKLDTRLASAEKSIADHDTRLNGLDKTVSDLRKETRQGLAEQ
AALSGLFQPYNVGGSGQSLANATAAQQEVLEAQYAMVQHIAKGIR
ILEARVARGGSGGNHTWQQWEEEIEQHEGNLSLLLREAALQVHIA
QRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
   Bold text is a flexible linker; and
   Underlined text corresponds to a VISNA-based SSM.

N. meningitidis NhhA-VISNA-based SSM CD11
[SEQ ID NO: 223]
MNNEEQEEDLYLDPVQRTVAVLIVNSDKEGTGEKEKVEENSDWAV
YFNEKGVLTAREITLKAGDNLKIKQNGTNFTYSLKKDLTDLTSVG
TEKLSFSANGNKVNITSDTKGLNFAKETAGTNGDTTVHLNGIGST
LTDTLLNTGATTNVTNDNVTDDEKKRAASVKDVLNAGWNIKGVKP
GTTASDNVDFVRTYDTVEFLSADTKTTTVNVESKDNGKKTEVKIG
AKTSVIKEKDGKLVTGKDKGENGSSTDEGEGLVTAKEVIDAVNKA
GWRMKTTTANGQTGQADKFETVTSGTNVTFASGKGTTATVSKDDQ
GNITVMYDVNVGDALNVNQLQNSGWNLDSKAVAGSSGKVISGNVS
PSKGKMDETVNINAGNNIEITRNGKNIDIATSMTPQFSSVSLGAG
ADAPTLSVDGDALNVGSKKDNKPVRITNVAPGVKEGDVTNVAQLK
GVAQNLNNRIDNVDGNARAGIAQAIATAGLVQAYLPGGSGQSLAN
ATAAQQEVLEAQYAMVQHIAKGIRILEARVARGGSGGNHTWQQWE
EEIEQHEGNLSLLLREAALQVHIAQRDARRI,

for an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
   Bold text is a flexible linker; and
   Underlined text corresponds to a VISNA-based SSM.

Y. enterocolitica YadA-VISNA-based SSM CD11
[SEQ ID NO: 224]
MDDYDGIPNLTAVQISPNADPALGLEYPVRPPVPGAGGLNASAKG
IHSIAIGATAEAAKGAAVAVGAGSIATGVNSVAIGPLSKALGDSA
VTYGAASTAQKDGVAIGARASTSDTGVAVGFNSKADAKNSVAIGH
SSHVAANHGYSIAIGDRSKTDRENSVSIGHESLNRQLTHLAAGTK
DTDAVNVAQLKKEIEKTQENTNKRSAELLANANAYADNKSSSVLG
IANNYTDSKSAETLENARKEAFAQSKDVLNMAKAHSNSVARTTLE
TAEEHANSVARTTLETAEEHANKKSAEALASANVYADSKSSHTLK
TANSYTDVTVSNSTKKAIRESNQYTDHKFRQLDNRLDKLDTRVDK
GLASSAALNSLFQPYGVGGSGQSLANATAAQQEVLEAQYAMVQHI
AKGIRILEARVARGGSGGNHTWQQWEEEIEQHEGNLSLLLREAAL
QVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
   Bold text is a flexible linker; and
   Underlined text corresponds to a VISNA-based SSM.

hMPV Fusion protein Clamp2s
[SEQ ID NO: 260]
MSWKVVIIFSLLITPQHGLKESYLEESCSTITEGYLSVLRTGWYT
NVFTLEVGDVENLTCADGPSLIKTELDLTKSALRELRTVSADQLA
REEQIENPRQSRFVLGAIALGVATAAAVTAGVAIAKTIRLESEVT
AIKNALKKTNEAVSTLGNGVRVLATAVRELKDFVSKNLTRAINKN
KCDIADLKMAVSFSQFNRRFLNVVRQFSDNAGITPAISLDLMTDA
ELARAVSNMPTSAGQIKLMLENRAMVRRKGFGILIGVYGSSVIYM
VQLPIFGVIDTPCWIVKAAPSCSEKKGNYACLLREDQGWYCQNAG
STVYYPNEKDCETRGDHVFCDTAAGINVAEQSKECNINISTTNYP
CKVSTGRHPISMVALSPLGALVACYKGVSCSIGSNRVGIIKQLNK
GCSYITNQDADTVTIDNTVYQLSKVEGEQHVIKGRPVSSSFDPVK
FPEDQFNVALDQVFESIENSQALVDQSNRIGSGLANATAAQQEVL
EAQYAMVQHIAKGIRILEARVARGGSGGNHTTWQQWEEEIENHTG
NLTLLLREAANQTHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of hMPV Fusion protein;
Bold text is a flexible linker; and
Underlined text corresponds to a VISNA-based SSM.

HTLV-1 glycoprotein Clamp2s
[SEQ ID NO: 261]
MGWSCIILFLVATATGVHSESRCTLTIGVSSYHSKPCNPAQPVCS
WTLDLLALSADQALQPPCPNLVSYSNYHATYSLYLFPHWIKKPNR
NGGGYYSASYSDPCSLKCPYLGCQSWTCPYTGAVSSPYWKFQQDV
NFTQEVSRLNINLHFSKCGFPFSLLVDAPGYDPIWLLNTEPSQLP
PTAPPLLPHSNLDHILEPSIPWKSKLLTLVQLTLQSTNYTCIVCI
DRASLSTWHVLYSPNISIPSSSSTPLLYPSLALPAPHLTLPFNWT
HCFDPQIQAIVSSPCHNSLILPPFSLSPVPTLRSRSRRGGGGVSA
LAMGTGIAGGITGSMSLASGKNLLHEVDKDISQLTQAIVKNHKNL
LKIAQYAAQNRRGLDLLFWEQGGLCKALQEQCCFLNITNSHVSIL
QERPPLEGSGLANATAAQQEVLEAQYAMVQHIAKGIRILEARVAR
GGSGGNHTTWQQWEEEIENHTGNLTLLLREAANQTHIAQRDARR
I,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of HTLV-1 Glycoprotein;
Bold text is a flexible linker; and
Underlined text corresponds to a VISNA-based SSM.

hPIV3 Fusion protein Clamp2s
[SEQ ID NO: 262]
MPTSILLIITTMIMASFCQIDITKLQHVGVLVNSPKGMKISQNFE
TRYLILSLIPKIEDSNSCGDQQIKQYKRLLDRLIIPLYDGLRLQK
DVIVTNQESNENTDPRTERFFGGVIGTIALGVATSAQITAAVALV
EAKQAKSDIEKLKEAIRDTNKAVQSVQSSVGNLIVAIKSVQDYVN
KEIVPSIARLGCEAAGLQLGIALTQHYSELTNIFGDNIGSLQEKG
IKLQGIASLYRTNITEIFTTSTVDKYDIYDLLFTESIKVRVIDVD
LNDYSITLQVRLPLLTRLLNTQIYKVDSISYNIQNREWYIPLPSH
IMTKGAFLGGADVKECIEAFSSYICPSDPGFVLNHEMESCLSGNI
SQCPRTTVTSDIVPRYAFVNGGVVANCITTTCTCNGIGNRINQPP
DQGVKIITHKECNTIGINGMLFNTNKEGTLAFYTPDDITLNNSVA
LDPIDISIELNKAKSDLEESKEWIRRSNQKGSGLANATAAQQEVL
EAQYAMVQHIAKGIRILEARVARGGSGGNHTTWQQWEEEIENHTG
NLTLLLREAANQTHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the signal peptide of hPIV3 Fusion protein;
Bold text is a flexible linker; and
Underlined text corresponds to a VISNA-based SSM.

Chlamydia trachomatis Major Outer Membrane
Protein Clamp2
[SEQ ID NO: 263]
MKKLLKSVLVFAALSSASSLQALPVGNPAEPSLMIDGILWEGFGG
DPCDPCTTWCDAISMRVGYYGDFVFDRVLKTDVNKEFQMGAAPTT
SDVAGLQNDPTINVARPNPAYGKHMQDAEMFTNAAYMALNIWDRF
DVFCTLGATTGYLKGNSASFNLVGLFGTKTQSSSFNTAKLIPNTA
LNEAVVELYINTTFAWSVGARAALWECGCATLGASFQYAQSKPKV
EELNVLCNASEFTINKPKGYVGAEFPLNITAGTEAATGTKDASID
YHEWQASLALSYRLNMFTPYIGVKWSRVSFDADTIRIAQPKLAEA
ILDVTTLNRTTAGKGSVVSAGTDNELADTMQIVSLQLNKMKSRKS
CGIAVGTTIVDADKYAVTVEARLIDERAAHVNAQFRFGSGLANAT
AAQQEVLEAQYAMVQHIAKGIRILEARVARGGSGGNHTWQQWEEE
IEQHEGNLSLLLREAALQVHIAQRDARRI,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Italicized text corresponds to the leader sequence;
Bold text is a flexible linker; and
Underlined text corresponds to a VISNA-based SSM.

Chlamydia trachomatis PGP3 Clamp2
[SEQ ID NO: 264]
MANATAAQQEVLEAQYAMVQHIAKGIRILEARVARGGSGGNHTWQ
QWEEEIEQHEGNLSLLLREAALQVHIAQRDARRIGGSGGNSGFYL
YNTENCVFADNIKVGQMTEPLKDQQIILGTTSTPVAAKMTASDGI
SLTVSNNSSTNASITIGLDAEKAYQLILEKLGNQILDGIADTIVD
STVQDILDKITTDPSLGLLKAFNNFPITNKIQCNGLFTPSNIETL
LGGTEIGKFTVTPKSSGSMFLVSADIIASRMEGSVVLALVREGDS
KPCAISYGYSSGVPNLCSLRTSITNTGLTPTTYSLRVGGLESGVV
WVNALSNGNDILGITNTSNVSFLEVIPQTNA,

or an amino acid sequence corresponding thereto (e.g., an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence similarity or identity)
wherein:
Bold text is a flexible linker; and
Underlined text corresponds to a VISNA-based SSM.

### 2.4 Use of Structure-Stabilizing Moiety as a Universal Oligomerization Domain

In addition to its utility in stabilizing an ectodomain polypeptide of the invention against rearrangement to a post-fusion conformation, the structure-stabilizing moiety is useful as a universal oligomerization domain (UOD) for oligomerizing any heterologous molecules of interest into oligomers, particularly trimers. In specific embodiments, a UOD is fused upstream or downstream of a heterologous proteinaceous molecule (referred to herein as “first (poly)peptide”) to form a chimeric polypeptide. Typically, the UOD is fused downstream of the heterologous proteinaceous molecule. As with the ectodomain embodiments described herein, association of the complementary heptad repeats of the UOD to one another under conditions suitable for their association (e.g., in aqueous solution) results in formation of an anti-parallel, two-helix bundle that trimerizes to form a highly stable six-helix bundle, thus permitting trimerization of the chimeric polypeptide to form a trimeric polypeptide complex.

Thus, the invention provides, in a second aspect, a chimeric polypeptide comprising a first (poly)peptide operably connected downstream to a structure-stabilizing moiety, wherein said structure-stabilizing moiety is as defined in connection with the first aspect of the invention; wherein preferably the first (poly)peptide is a therapeutic (poly)peptide.

The heterologous proteinaceous molecule (i.e., the “first (poly)peptide”) may be a natural or non-natural polypeptide. In certain embodiments, the heterologous (poly)peptide is or comprises a therapeutic (poly)peptide. A vast variety of therapeutic (poly)peptides, including both ligands and receptors, are known in the art to be useful for treating or preventing a variety of diseases.

### 2.5 Methods of Preparing Chimeric Polypeptide and Complexes Thereof

The chimeric polypeptides of the present disclosure may be prepared by chemical synthesis or recombinant means. Usually, the polypeptides are prepared by expression of a recombinant construct that encodes the modified or chimeric polypeptide in suitable host cells, although any suitable methods can be used. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., Escherichia coli, Bacillus subtilis, and Streptococcus spp.), yeast cells (e.g., Saccharomyces cerevisiae, Candida albicans, Candida maltosa, Hansenula polymorphs, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophile) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Suitable mammalian cells include, for example, Chinese hamster ovary (CHO) cells, human embryonic kidney cells (HEK293 cells), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx© cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Appropriate insect cell expression systems, such as Baculovirus systems, are known to those of skill in the art and described in, e.g., Summers and Smith, Texas Agricultural Experiment Station Bulletin No. 1555 (1987). Materials and methods for Baculovirus/insert cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Similarly, bacterial and mammalian cell expression systems are also known in the art and described in, e.g., Yeast Genetic Engineering (Barr et al., eds., 1989) Butterworths, London.

Recombinant constructs encoding the modified or chimeric polypeptides of the present disclosure can be prepared in suitable vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable Baculovirus expression vector, such as pFastBac (Invitrogen), can be used to produce recombinant Baculovirus particles. The Baculovirus particles are amplified and used to infect insect cells to express recombinant protein. For expression in mammalian cells, a vector that will drive expression of the construct in the desired mammalian host cell (e.g., Chinese hamster ovary cells) is used.

The modified or chimeric polypeptides can be purified using any applicable method. Suitable methods for purifying desired proteins including precipitation and various types of chromatography, such as hydrophobic interaction, ion exchange, affinity, chelating and size exclusion are well-known in the art. Appropriate purification schemes can be created using two or more of these or other suitable methods. If desired, the modified or chimeric polypeptides can include a purification moiety or “tag” that facilitates purification, as described for example supra. Such tagged polypeptides can conveniently be purified, for example from conditioned media, by chelating chromatography or affinity chromatography.

The modified or chimeric polypeptides may include additional sequences. For example, for expression purposes, the natural leader peptide of a heterologous polypeptide of interest (e.g., the natural leader peptide of a bacterial or viral surface polypeptide, e.g., the leader peptide of an enveloped virus fusion protein or of a bacterial outer membrane polypeptide) may be substituted for a different one.

The invention provides, in a fifth aspect, a method of producing a chimeric polypeptide complex, wherein the method comprises: combining chimeric polypeptides of the invention under conditions suitable for the formation of a chimeric polypeptide complex, whereby a chimeric polypeptide complex is produced that comprises three chimeric polypeptide subunits and is characterized by a six-helix bundle formed by homo-trimerization of the structure-stabilizing moieties of the three chimeric polypeptides.

### 3. Polynucleotides and Nucleic Acid Constructs for Endogenous or Heterologous Production of Chimeric Polypeptides

The present disclosure also contemplates polynucleotides and nucleic acid constructs for endogenous or heterologous (i.e., recombinant) production of the chimeric polypeptides in a host organism, suitably a vertebrate animal, preferably a mammal such as a human.

More specifically, the invention provides, in a third aspect, a nucleic acid comprising a polynucleotide sequence encoding a chimeric polypeptide as defined in embodiments disclosed herein in connection with the first or second aspect of the invention.

In preferred embodiments, the nucleic acid further comprises a promoter operably linked to the polynucleotide sequence encoding the chimeric polypeptide; wherein the promoter is preferably a mammalian promoter. The skilled person will be able to select a promoter that suitably functions for controlling the expression of the chimeric polypeptide in the specific host organism envisaged.

Generally, polynucleotides contemplated herein comprise a coding sequence for the chimeric polypeptide of the disclosure. These polynucleotides are useful for making nucleic acid constructs from which a chimeric polypeptide coding sequence is expressible for immunizing subjects. In some embodiments, these polynucleotides are themselves useful for immunizing subjects directly. In representative embodiments of this type, the polynucleotides comprise at least one ribonucleic acid (RNA) or deoxyribonucleic acid (DNA) having an open reading frame encoding a polypeptide of the present disclosure. In some embodiments, the RNA is a messenger RNA (mRNA) having an open reading frame that codes for a polypeptide disclosed herein.

Polynucleotides of the present disclosure, in some embodiments, are codon optimized. Codon optimization methods are known in the art and may be used for optimizing expression of the polypeptides disclosed herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art. Non-limiting examples include services from GeneArt (Life Technologies), DNA2.0 (Menlo Park Calif.) and/or proprietary methods. In some embodiments, the open reading frame (ORF) sequence is optimized using optimization algorithms. In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

In some embodiments, the RNA polynucleotides of the present disclosure may further comprise a sequence comprising or encoding an additional sequence, for example, one or more functional domain(s), one or more further regulatory sequence(s), and/or an engineered 5′ cap. Thus, in some embodiments, the RNA vaccines comprise a 5′UTR element, an optionally codon optimized open reading frame incorporating or not incorporating non-natural bases (or non-natural nucleotides) to reduce innate immune response triggering, and a 3′UTR element, a poly(A) sequence and/or a polyadenylation signal wherein the RNA is or is not modified.

The RNA polynucleotide may be transcribed in vitro from template DNA, referred to as an “in vitro transcription template”. In some embodiments, an in vitro transcription template encodes a 5′ untranslated (UTR) region, contains an open reading frame, and encodes a 3′ UTR and a polyA tail. The particular nucleotide sequence composition and length of an in vitro transcription template will depend on the mRNA encoded by the template. A “5′ untranslated region” (UTR) refers to a region of an mRNA that is directly upstream (i.e., 5′) from the start codon (i.e., the first codon of an mRNA transcript translated by a ribosome) that does not encode a polypeptide. A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of codons beginning with a start codon (e.g., methionine (ATG)), and ending with a stop codon (e.g., TAA, TAG or TGA) that encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30.40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In some embodiments, the RNA polynucleotide is formulated within a lipid nanoparticle (LNP). 5′-capping of polynucleotides may be completed concomitantly during the in vitro-transcription reaction using the following chemical RNA cap analogs to generate the 5′-guanosine cap structure according to manufacturer protocols: 3′-0-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The present disclosure also contemplates nucleic acid constructs for endogenous production of the polypeptides disclosed herein. The nucleic acid constructs can be self-replicating extra-chromosomal vectors/replicons (e.g., plasmids) or vectors that integrate into a host genome. In specific embodiments, the nucleic acid constructs are viral vectors. Exemplary viral vectors include retroviral vectors, lentiviral vectors, poxvirus vectors, vaccinia virus vectors, adenovirus vectors, adenovirus-associated virus vectors, herpes virus vectors, flavivirus vectors, and alphavirus vectors. Viral vectors may be live, attenuated, replication conditional or replication deficient, and typically is a non-pathogenic (defective), replication competent viral vector.

By way of example, when the viral vector is a vaccinia virus vector, a polynucleotide encoding a chimeric polypeptide of the disclosure may be inserted into a non-essential site of a vaccinia viral vector genome. Such non-essential sites are described, for example, in Perkus et al. (1986. Virology 152:285); Hruby et al. (1983. Proc. Natl. Acad. Sci. USA 80:3411); Weir et al. (1983. J. Virol. 46:530). Suitable promoters for use with vaccinia viruses include but are not limited to P7.5 (see, e.g., Cochran et al. 1985. J. Virol. 54:30); P11 (see, e.g., Bertholet, et al., 1985. Proc. Natl. Acad. Sci. USA 82:2096); and CAE-1 (see, e.g., Patel et al., 1988. Proc. Natl. Acad. Sci. USA 85:9431). Highly attenuated strains of vaccinia are more acceptable for use in humans and include Lister, NYVAC, which contains specific genome deletions (see, e.g., Guerra et al., 2006. J. Virol. 80:985-998); Tartaglia et al., 1992. AIDS Research and Human Retroviruses 8:1445-1447), or MVA (see, e.g., Gheradi et al., 2005. J. Gen. Virol. 86:2925-2936); Mayr et al., 1975. Infection 3:6-14). See also Hu et al. (2001. J. Virol. 75:10300-10308), describing use of a Yaba-Like disease virus as a vector for cancer therapy); U.S. Pat. Nos. 5,698,530 and 6,998,252. See also, e.g., U.S. Pat. No. 5,443,964. See also U.S. Pat. Nos. 7,247,615 and 7,368,116.

In certain embodiments, an adenovirus vector may be used for expressing a chimeric polypeptide of interest. The adenovirus on which a viral transfer vector may be based may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087. Any adenovirus, even a chimeric adenovirus, can be used as the source of the viral genome for an adenoviral vector. For example, a human adenovirus can be used as the source of the viral genome for a replication-deficient adenoviral vector. Further examples of adenoviral vectors can be found in Molin et al. (1998. J. Virol. 72:8358-8361), Narumi et al. (1998. Am J. Respir. Cell Mol. Biol. 19:936-941) Mercier et al. (2004. Proc. Natl. Acad. Sci. USA 101:6188-6193), U.S. Publication Nos. 20150093831, 20140248305, 20120283318, 20100008889, 20090175897 and 20090088398 and U.S. Pat. Nos. 6,143,290; 6,596,535; 6,855,317; 6,936,257; 7,125,717; 7,378,087; 7,550,296.

The viral vector can also be based on adeno-associated viruses (AAVs). For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757. The AAV vectors may also be self-complementary (sc) AAV vectors, which are described, for example, in U.S. Patent Publications 2007/01110724 and 2004/0029106, and U.S. Pat. Nos. 7,465,583 and 7,186,699.

Herpes simplex virus (HSV)-based viral vectors are also suitable for endogenous production of the chimeric polypeptides of the disclosure. Many replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. For a description of HSV-based vectors, see, for example, U.S. Pat. Nos. 5,837,532, 5,846,782, 5,849,572, and 5,804,413, and International Patent Applications WO 91/02788, WO 96/04394, WO 98/15637, and WO 99/06583.

Retroviral vectors may include those based upon murine leukemia virus (MuLV), gibbon ape leukemia virus (GaLV), ecotropic retroviruses, simian immunodeficiency virus (SW), human immunodeficiency virus (HIV), and combinations (see, e.g., Buchscher et al., 1992. J. Virol. 66:2731-2739; Johann et al., 1992. J. Virol. 66:1635-1640; Sommerfelt et al., 1990. Virology 176:58-59; Wilson et al., 1989. J. Virol. 63:2374-2378; Miller et al., 1991. J. Virol. 65:2220-2224; Miller et al., 1990. Mol. Cell Biol. 10:4239; Kolberg, 1992. NIH Res. 4:43; Cornetta et al., 1991. Hum. Gene Ther. 2:215).

In specific embodiments, the retroviral vector is a lentiviral vector. As would be understood by the skilled person, a viral vector, such as a lentiviral vector, generally refers to a viral vector particle that comprises the viral vector genome. For example, a lentiviral vector particle may comprise a lentiviral vector genome. With respect to lentiviral vectors, the vector genome can be derived from any of a large number of suitable, available lentiviral genome-based vectors, including those identified for human gene therapy applications (see, e.g., Pfeifer et al., 2001. Annu. Rev. Genomics Hum. Genet. 2:177-211). Suitable lentiviral vector genomes include those based on Human Immunodeficiency Virus (HIV-1), HIV-2, feline immunodeficiency virus (FIV), equine infectious anemia virus, Simian Immunodeficiency Virus (SIV), and Maedi-Visna virus. A desirable characteristic of lentiviruses is that they are able to infect both dividing and non-dividing cells, although target cells need not be dividing cells or be stimulated to divide. Generally, the genome and envelope glycoproteins will be based on different viruses, such that the resulting viral vector particle is pseudotyped. Safety features of the viral vector are desirably incorporated. Safety features include self-inactivating LTR and integration deficiency as described in more detail herein. In certain embodiments integration deficiency may be conferred by elements of the vector genome but may also derive from elements of the packaging system (e.g., a non-functional integrase protein that may not be part of the vector genome but supplied in trans). Exemplary vectors contain a packaging signal (psi), a Rev-responsive element (RRE), splice donor, splice acceptor, optionally a central poly-purine tract (cPPT), and WPRE element. In certain exemplary embodiments, the viral vector genome comprises sequences from a lentivirus genome, such as the HIV-1 genome or the SIV genome. The viral genome construct may comprise sequences from the 5′ and 3′ LTRs of a lentivirus, and in particular may comprise the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. Typically, the LTR sequences are HIV LTR sequences.

The vector genome may comprise an inactivated or self-inactivating 3′ LTR (see, e.g., Zufferey et al., 1998. J. Virol. 72: 9873; Miyoshi et al., 1998. J. Virol. 72:8150). A self-inactivating vector generally has a deletion of the enhancer and promoter sequences from the 3′ long terminal repeat (LTR), which is copied over into the 5′ LTR during vector integration. In one instance, the U3 element of the 3′ LTR contains a deletion of its enhancer sequence, the TATA box, Spl and NF-kappa B sites. As a result of the self-inactivating 3′ LTR, the provirus that is generated following entry and reverse transcription will comprise an inactivated 5′ LTR. The rationale is to improve safety by reducing the risk of mobilization of the vector genome and the influence of the LTR on nearby cellular promoters. The self-inactivating 3′ LTR may be constructed by any method known in the art.

Optionally, the U3 sequence from the lentiviral 5′ LTR may be replaced with a promoter sequence in the viral construct, such as a heterologous promoter sequence. This can increase the titer of virus recovered from the packaging cell line. An enhancer sequence may also be included. Any enhancer/promoter combination that increases expression of the viral RNA genome in the packaging cell line may be used. In one example, the CMV enhancer/promoter sequence is used (see, e.g., U.S. Pat. Nos. 5,385,839 and 5,168,062).

In certain embodiments, the risk of insertional mutagenesis is minimized by constructing the lentiviral vector to be integration defective. A variety of approaches can be pursued to produce a non-integrating vector genome. These approaches entail engineering a mutation(s) into the integrase enzyme component of the pol gene, such that it encodes a protein with an inactive integrase. The vector genome itself can be modified to prevent integration by, for example, mutating or deleting one or both attachment sites, or making the 3′ LTR-proximal polypurine tract (PPT) non-functional through deletion or modification. In addition, non-genetic approaches are available; these include pharmacological agents that inhibit one or more functions of integrase. The approaches are not mutually exclusive, that is, more than one of them can be used at a time. For example, both the integrase and attachment sites can be non-functional, or the integrase and PPT site can be non-functional, or the attachment sites and PPT site can be non-functional, or all of them can be non-functional. Exemplary lentivirus vectors are described for example in U.S. Publication Nos. 20150224209, 20150203870, 20140335607, 20140248306, 20090148936, and 20080254008.

The viral vectors may also be based on an alphavirus. Alphaviruses include Sindbis virus (and Venezuelan equine encephalitis virus (VEEV)), Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus (SEV), Southern elephantseal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, SFV, and VEEV have all been used to develop viral transfer vectors for transgene delivery. Pseudotyped viruses may be formed by combining alphaviral envelope glycoproteins and retroviral capsids. Examples of alphaviral vectors can be found in U.S. Publication Nos.

Alternatively, the viral vectors can be based on a flavivirus. Flaviviruses include Japanese encephalitis virus, Dengue virus (e.g., Dengue-1, Dengue-2, Dengue-3, Dengue-4), Yellow fever virus, Murray Valley encephalitis virus, St. Louis encephalitis virus, West Nile virus, Kunjin virus, Rocio encephalitis virus, Ilheus virus, Tick-borne encephalitis virus, Central European encephalitis virus, Siberian encephalitis virus, Russian Spring-Summer encephalitis virus, Kyasanur Forest Disease virus, Omsk Hemorrhagic fever virus, Louping ill virus, Powassan virus, Negishi virus, Absettarov virus, Hansalova virus, Apoi virus, and Hypr virus. Examples of flavivirus vectors can be found in U.S. Publication Nos. 20150231226, 20150024003, 20140271708, 20140044684, 20130243812, 20120294889, 20120128713, 20110135686, 20110014229, 20110003884, 20100297167, 20100184832, 20060159704, 20060088937, 20030194801 and 20030044773.

### 4. Host Cells

The invention also contemplates, in a fourth aspect, a host cell comprising the nucleic acid as defined in accordance with the third aspect of the invention.

In some embodiments, the host cell is (i) a prokaryotic host cell; or (ii) a eukaryotic host cell.

Exemplary eukaryotic host cells are, without intended to be limiting, yeast cells (e.g., Saccharomyces cerevisiae, Pichia pastoris), insect cells (e.g., Spodoptera frugiperda) or mammalian cells. Examples of preferred mammalian host cells are the human embryonic kidney cell lines (e.g., the cell line HEK-293), and the cell lines derived from Chinese hamster ovary (CHO) cells such as, for example, the ExpiCHO (Thermo Fisher) cell line as used in the herein disclosed examples. Preferably, the host cell bears a glycosylation machinery (endogenously or genetically engineered) that allows glycosylation (preferably N-glycosylation) of the expressed polypeptide with glycans that are commonly expressed in the particular mammalian subject species (e.g., Homo sapiens) envisaged for being administered with the so expressed polypeptide (i.e., the chimeric polypeptide(s) or complexes thereof as contemplated herein).

In a preferred embodiment, the host cells are those cells (preferably mammalian cells) which have been stably transfected with the nucleic acid(s) as contemplated herein. In the case of stably transfected cells, the expression system is incorporated into the genome of the target cell and remains in the genome in a stable manner. In contrast to transient transfection, the transferred gene is here not only not degraded but doubled with each cell division and passed onto the daughter cells. The latter thus retain the ability to prepare the desired protein over a long period of time. Processes for preparing transfected, in particular stably transfected, cells are known in the art. The host cell may be transformed, for example, by means of electroporation in which permeabilization of the cell membrane, due to briefly applying an electric field, allows nucleic acids to be taken up into the cell, or by way of transfection or infection with a viral vector as also described herein. Besides transient expression of the recombinant protein, the expression system used may also allow clonal selection of the transfected host cells so that it is possible to select clonal cell lines having a suitable expression efficiency.

Exemplary prokaryotic host cells include, without limitation, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species. A particularly preferred prokaryotic host cell is Escherichia coli.

### 5. Chimeric Polypeptide Complexes and Methods of Production Thereof

The invention provides, in a fifth aspect, a method of producing a chimeric polypeptide complex, wherein the method comprises: combining chimeric polypeptides as defined in accordance with the first or the second aspect of the invention under conditions suitable for the formation of a chimeric polypeptide complex, whereby a chimeric polypeptide complex is produced that comprises three chimeric polypeptide subunits and is characterized by a six-helix bundle formed by homo-trimerization of the structure-stabilizing moieties of the three chimeric polypeptides.

Preferably, the six-helix bundle is composed of an inner trimer of three parallel oriented, substantially α-helical FHRRs against which three substantially α-helical SHRRs are packed in an anti-parallel orientation relative to the FHRRs.

The invention provides, in a sixth aspect, a chimeric polypeptide complex that comprises three chimeric polypeptide subunits, wherein each subunit is a chimeric polypeptide as defined in accordance with the first or the second aspect of the invention, and wherein the complex is characterized by a six-helix bundle formed by homo-trimerization of the structure-stabilizing moieties of the three chimeric polypeptides.

In some embodiments, the six-helix bundle is composed of an inner trimer of three parallel oriented, substantially α-helical FHRRs against which three substantially α-helical SHRRs are packed in an anti-parallel orientation relative to the FHRRs.

Whereas it is generally preferred that the so produced chimeric polypeptide complex comprises three identical chimeric polypeptides, also such complexes are conceivable and expressly contemplated herein wherein the individual chimeric polypeptides are distinct from each other, e.g., with respect to any one or more of the components (e.g., the structure-stabilizing moiety (SSM) and/or the microbial polypeptide (e.g., the enveloped virus fusion ectodomain polypeptide or the bacterial outer membrane polypeptide)) comprised in the chimeric polypeptide as defined herein.

In some embodiments, the chimeric polypeptide subunits each comprise an enveloped virus fusion ectodomain polypeptide, and the complex comprises at least one pre-fusion epitope of an enveloped virus fusion protein.

### 6. Screening Methods

The present invention also encompasses methods of screening for agents that bind, preferably specifically bind, with a microbial polypeptide (preferably a fusion protein of an enveloped virus or a bacterial outer membrane polypeptide (e.g., a TAA polypeptide of a bacterium), and/or a respective complex (e.g., a complex of the fusion protein or the TAA polypeptide). In specific embodiments, a compound library is screened for binding to a microbial polypeptide-containing chimeric polypeptide (preferably an enveloped virus fusion ectodomain polypeptide-containing chimeric polypeptide or a bacterial outer membrane polypeptide-containing chimeric polypeptide), or complex thereof.

Thus, the invention provides, in an eighth aspect, a method of identifying an agent that binds with: a microbial polypeptide or a complex thereof, wherein the method comprises:
(i) contacting a candidate agent with a microbial polypeptide-containing chimeric polypeptide as defined in accordance with the first aspect of the invention or a microbial polypeptide-containing chimeric polypeptide complex as defined in accordance with the sixth aspect of the invention; and
(ii) detecting binding of the candidate agent to the chimeric polypeptide or chimeric polypeptide complex; wherein preferably the candidate agent is part of a compound library (e.g., small molecule or macromolecule library).

In preferred embodiments, the method further comprises:
(i) contacting the candidate agent with the microbial polypeptide or the complex thereof and detecting binding of the candidate agent to the microbial polypeptide or the complex thereof;
and/or
(ii) isolating the candidate agent.

In preferred embodiment of the latter aspect, the microbial polypeptide or the complex thereof is:
(a) a fusion protein of an enveloped virus, or a complex of the fusion protein, respectively, wherein the method comprises:
   (i) contacting the candidate agent with an enveloped virus fusion ectodomain polypeptide-containing chimeric polypeptide as defined in accordance with the first aspect of the invention or an enveloped virus fusion ectodomain polypeptide-containing chimeric polypeptide complex as defined in accordance with the sixth aspect of the invention, wherein the enveloped virus fusion ectodomain polypeptide corresponds to the fusion protein of the enveloped virus; and
   (ii) detecting binding of the candidate agent to the chimeric polypeptide or chimeric polypeptide complex;
   or
(b) an outer membrane polypeptide of a bacterium or a complex of the outer membrane polypeptide, respectively, wherein the method comprises:
   (i) contacting the candidate agent with a bacterial outer membrane polypeptide-containing chimeric polypeptide as defined in accordance with the first aspect of the invention or a bacterial outer membrane polypeptide-containing chimeric polypeptide complex as defined in accordance with the sixth aspect of the invention, wherein the bacterial outer membrane polypeptide corresponds to the outer membrane polypeptide of the bacterium; and
   (ii) detecting binding of the candidate agent to the chimeric polypeptide or chimeric polypeptide complex.

In preferred embodiments of the latter embodiment, the method further comprises:
(i) (i-a) contacting the candidate agent of item (a) with the fusion protein or the complex of the fusion protein and detecting binding of the candidate agent to the fusion protein or the complex of the fusion protein; or
   (i-b) contacting the candidate agent of item (b) with the outer membrane polypeptide or the complex of the outer membrane polypeptide and detecting binding of the candidate agent to the outer membrane polypeptide or the complex of the outer membrane polypeptide;
and/or
(ii) isolating the candidate agent.

In preferred embodiments, the outer membrane polypeptide of a bacterium or the complex thereof is:
(a) a trimeric autotransporter adhesin (TAA) polypeptide of a bacterium, or a complex of the TAA polypeptide, wherein the method comprises:
   (i) contacting the candidate agent with a TAA polypeptide-containing chimeric polypeptide as defined in accordance with the first aspect of the invention or a TAA polypeptide-containing chimeric polypeptide complex as defined in accordance with the sixth aspect of the invention, wherein the TAA polypeptide corresponds to the TAA polypeptide of the bacterium; and
   (ii) detecting binding of the candidate agent to the chimeric polypeptide or chimeric polypeptide complex;
or
(b) a major outer membrane protein (MOMP) polypeptide of a Chlamydia bacterium or a complex thereof, wherein the method comprises:
   (i) contacting the candidate agent with a Chlamydia MOMP polypeptide-containing chimeric polypeptide as defined in accordance with the first aspect of the invention or a Chlamydia MOMP polypeptide-containing chimeric polypeptide complex as defined in accordance with the sixth aspect of the invention, wherein the Chlamydia MOMP polypeptide corresponds to the MOMP polypeptide of the Chlamydia bacterium; and
   (ii) detecting binding of the candidate agent to the chimeric polypeptide or chimeric polypeptide complex.

In preferred embodiments of item (a) of the latter embodiment, the method further comprises:
(i) (i-a) contacting the candidate agent with the TAA polypeptide or the complex of the TAA polypeptide and detecting binding of the candidate agent to the TAA polypeptide or the complex of the TAA polypeptide; or
   (i-b) contacting the candidate agent with the MOMP polypeptide or the complex of the MOMP polypeptide;
and/or
(ii) isolating the candidate agent.

In preferred embodiments of the latter embodiments, the candidate agent binds specifically to the chimeric polypeptide or chimeric polypeptide complex.

In other or even more preferred embodiments, the candidate agent binds specifically to the microbial polypeptide or the complex thereof.

In even more preferred embodiments, the candidate agent binds specifically to:
(i) the enveloped virus fusion protein or the complex of the fusion protein; or
(ii) the outer membrane polypeptide or the complex of the outer membrane polypeptide.

In even more preferred embodiments of item (ii) of the latter embodiment, the candidate agent binds specifically to:
(i) the TAA polypeptide or the complex of the TAA polypeptide; or
(ii) the MOMP polypeptide or the complex of the MOMP polypeptide.

Candidate agents encompass numerous chemical classes including small molecules, such as small organic compounds and macromolecules such as peptides, polypeptides and polysaccharides. Candidate agents comprise functional groups necessary for structural interaction with proteins, particularly hydrogen bonding, and typically include at least an amine, carbonyl, hydroxyl or carboxyl group, desirably at least two of the functional chemical groups. The candidate compounds may comprise cyclical carbon or heterocyclic structures or aromatic or polyaromatic structures substituted with one or more of the above functional groups. Candidate agents are also found among biomolecules including, but not limited to: peptides, polypeptides, saccharides, fatty acids, steroids, purines, pyrimidines, derivatives, structural analogues or combinations thereof. The compound library may comprise natural compounds in the form of bacterial, fungal, plant and animal extracts. Alternatively, or in addition, the compound library may include natural or synthetically produced compounds.

In particularly preferred embodiments also envisaged herein, the candidate agent is a peptide, an antibody, an antibody-fragment (e.g., a single-chain fragment variable (scFv)) or any alternative binding protein scaffold and expressed as part of a combinatorial library, such as used in high-throughput (HT) combinatorial library-based display and selection methods, for example, without intending to being limiting, phage display, ribosome display, mRNA display, and cell surface display (e.g. yeast display). Candidate agents that specifically bind with a microbial polypeptide (e.g., a fusion protein of an enveloped virus or complex thereof or a bacterial surface polypeptide, such as a bacterial outer membrane polypeptide (e.g., a TAA polypeptide)) or a complex thereof can be routinely selected by using such known display and selection approaches in connection with a microbial polypeptide-containing chimeric polypeptide or complex thereof (e.g., an enveloped virus fusion ectodomain polypeptide-containing chimeric polypeptide or complex thereof or a bacterial surface polypeptide-containing chimeric polypeptide or complex thereof, such as a bacterial outer membrane polypeptide-containing chimeric polypeptide or complex thereof (e.g., a TAA polypeptide-containing chimeric polypeptide or complex thereof)) as respective antigen against which the selection is performed.

Methods for determining whether an agent binds to a target protein and/or for determining the affinity of an agent for a target protein are known in the art. For example, the binding of an agent to a target protein can be detected and/or quantified using a variety of techniques such as, but not limited to, BioLayer Interferometry (BLI), Western blot, dot blot, surface plasmon resonance method (SPR), enzyme-linked immunosorbent assay (ELISA), AlphaScreen® or AlphaLISA® assays, or mass spectrometry-based methods.

In some embodiments, agents can be assayed using any surface plasmon resonance (SPR)-based assays known in the art for characterizing the kinetic parameters of the interaction of the agent with a microbial polypeptide-containing chimeric polypeptide or complex thereof (e.g., an enveloped virus fusion ectodomain polypeptide-containing chimeric polypeptide or complex thereof or a bacterial surface polypeptide-containing chimeric polypeptide or complex thereof, such as a bacterial outer membrane polypeptide-containing chimeric polypeptide or complex thereof (e.g., a TAA polypeptide-containing chimeric polypeptide or complex thereof)). Any SPR instrument commercially available including, but not limited to, BIAcore Instruments (Biacore AB; Uppsala, Sweden); IAsys instruments (Affinity Sensors; Franklin, Mass.); IBIS system (Windsor Scientific Limited; Berks, UK), SPR-CELLIA systems (Nippon Laser and Electronics Lab; Hokkaido, Japan), and SPR Detector Spreeta (Texas Instruments; Dallas, Tex.) can be used in the methods described herein. See, e.g., Mullett et al. (2000) Methods 22: 77-91; Dong et al. (2002) Reviews in Mol Biotech 82: 303-323; Fivash et al. (1998) Curr Opin Biotechnol 9: 97-101; and Rich et al. (2000) Curr Opin Biotechnol 11: 54-61.

In some embodiments, the biomolecular interactions between the agents and a microbial polypeptide-containing chimeric polypeptide or complex thereof (e.g., an enveloped virus fusion ectodomain polypeptide-containing chimeric polypeptide or complex thereof or a bacterial surface polypeptide-containing chimeric polypeptide or complex thereof, such as a bacterial outer membrane polypeptide-containing chimeric polypeptide or complex thereof (e.g., a TAA polypeptide-containing chimeric polypeptide or complex thereof)) can be assayed using BLI on an Octet (ForteBio Inc.). BLI is a label-free optical analytical technique that senses binding between a ligand (such as an ectodomain polypeptide-containing chimeric polypeptide or complex of the invention) that is immobilized on a biosensor tip and an analyte (such as a test compound) in solution by measuring the change in the thickness of the protein layer on the biosensor tip in real-time.

In some embodiments, AlphaScreen (PerkinElmer) assays can be used to characterize binding of test agents to a microbial polypeptide-containing chimeric polypeptide or complex thereof (e.g., an enveloped virus fusion ectodomain polypeptide-containing chimeric polypeptide or complex thereof or a bacterial surface polypeptide-containing chimeric polypeptide or complex thereof, such as a bacterial outer membrane polypeptide-containing chimeric polypeptide or complex thereof (e.g., a TAA polypeptide-containing chimeric polypeptide or complex thereof). The acronym ALPHA stands for Amplified Luminescent Proximity Homogeneous Assay. AlphaScreen is a bead-based proximity assay that senses binding between molecules (such as a subject chimeric polypeptide, or complex and a test compound) attached to donor and acceptor beads by measuring the signal produced by energy transfer between the donor and acceptor beads. (See e.g., Eglen et al. (2008) Curr Chem Genomics 1:2-10).

In some embodiments, AlphaLISA® (PerkinElmer) assays can be used to characterize binding of test agents to the chimeric polypeptide or complex of the invention. AlphaLISA is modified from the AlphaScreen assay described above to include europium-containing acceptor beads and functions as an alternative to traditional ELISA assays. (See, e.g., Eglen et al. (2008) Curr Chem Genomics 1:2-10.) A variety of immunoassay techniques, including competitive and non-competitive immunoassays, can be used.

The term “immunoassay” encompasses techniques including, without limitation, flow cytometry, FACS, enzyme immunoassays (EIA), such as enzyme multiplied immunoassay technique (EMIT), enzyme-linked immunosorbent assay (ELISA), IgM antibody capture ELISA (MAC ELISA) and microparticle enzyme immunoassay (MEIA), furthermore capillary electrophoresis immunoassays (CEIA), radio-immunoassays (RIA), immunoradiometric assays (IRMA), fluorescence polarization immunoassays (FPIA) and chemiluminescence assays (CL). If desired, such immunoassays can be automated. Immunoassays can also be used in conjunction with laser induced fluorescence. Liposome immunoassays, such as flow-injection liposome immunoassays and liposome immunosensors, are also suitable for use in the present invention. In addition, nephelometry assays, in which, for example, the formation of protein/antibody complexes results in increased light scatter that is converted to a peak rate signal as a function of the marker concentration, are suitable for use in the methods of the present invention.

In some embodiments, binding of test agents to the subject chimeric polypeptide, or complex can be assayed using thermal denaturation methods involving differential scanning fluorimetry (DSF) and differential static light scattering (DSLS).

In some embodiments, binding of test agents to the chimeric polypeptide or complex of the invention can be assayed using a mass spectrometry-based method such as, but not limited to, an affinity selection coupled to mass spectrometry (AS-MS) platform. This is a label-free method where the protein and test compound are incubated, unbound molecules are washed away and protein-ligand complexes are analyzed by MS for ligand identification following a decomplexation step.

In some embodiments, binding of test agents to the subject chimeric polypeptide or complex can be quantitated using, for example, detectably labeled proteins such as radiolabeled (e.g., 32P, 35S, 14C or 3H), fluorescently labeled (e.g., FITC), or enzymatically labeled chimeric polypeptide or complex or test compound, by immunoassay, or by chromatographic detection.

In some embodiments, the present invention contemplates the use of fluorescence polarization assays and fluorescence resonance energy transfer (FRET) assays in measuring, either directly or indirectly, the degree of interaction between a chimeric polypeptide or complex and a test compound.

All of the above embodiments are suitable for development into high-throughput platforms.

Compounds may be further tested in the animal models to identify those compounds having the most potent in vivo effects, e.g., those that bind specifically to a microbial polypeptide or a complex thereof (e.g., a fusion protein of an enveloped virus, or a complex of the fusion protein, or a bacterial surface polypeptide or complex thereof, such as a bacterial outer membrane polypeptide or complex thereof (e.g., a TAA polypeptide-containing chimeric polypeptide or complex thereof) and preferably stimulate or enhance a therapeutically useful effect, e.g., reduced microbial (e.g., viral or bacterial) load, reduced infection or symptoms associated therewith. These molecules may serve as “lead compounds” for the further development of pharmaceuticals by, for example, subjecting the compounds to sequential modifications, molecular modeling, and other routine procedures employed in rational drug design.

### 7. Antigen-Binding Molecules and Methods of Production Thereof

The invention provides, in a ninth aspect, a method of producing an antigen-binding molecule that specifically binds to:
a microbial polypeptide, or a complex thereof, wherein the method comprises:
(1) immunizing a subject with a microbial polypeptide-containing chimeric polypeptide as defined in accordance with the first aspect of the invention, or a microbial polypeptide-containing chimeric polypeptide complex as defined in accordance with the sixth aspect of the invention, or a composition thereof as defined in accordance with the seventh aspect of the invention;
(2) identifying and/or isolating a B cell from the immunised subject, which specifically binds to the microbial polypeptide or complex thereof; and
(3) producing the antigen-binding molecule expressed by that B cell.

In preferred embodiments of the latter aspect, the microbial polypeptide or the complex thereof is:
(a) an ectodomain of a fusion protein of an enveloped virus, or complex of the fusion protein, respectively, wherein the method comprises: (1) immunizing a subject with an ectodomain polypeptide-containing chimeric polypeptide as defined in accordance with the first aspect of the invention, or an ectodomain polypeptide-containing chimeric polypeptide complex as defined in accordance with the sixth aspect of the invention, or a composition thereof as defined in accordance with the seventh aspect of the invention, wherein the ectodomain polypeptide corresponds to the fusion protein of the enveloped virus; (2) identifying and/or isolating a B cell from the immunised subject, which specifically binds to the ectodomain of the fusion protein or complex thereof; and (3) producing the antigen-binding molecule expressed by that B cell;
or
(b) an outer membrane polypeptide of a bacterium, or a complex of the outer membrane polypeptide, wherein the method comprises: (1) immunizing a subject with a bacterial outer membrane polypeptide-containing chimeric polypeptide as defined in accordance with the first aspect of the invention, or a bacterial outer membrane polypeptide-containing chimeric polypeptide complex as defined in accordance with the sixth aspect of the invention, or a composition thereof as defined in accordance with the seventh aspect of the invention, wherein the bacterial outer membrane polypeptide corresponds to the outer membrane polypeptide of the bacterium; (2) identifying and/or isolating a B cell from the immunised subject, which specifically binds to the bacterial outer membrane polypeptide or complex thereof; and (3) producing the antigen-binding molecule expressed by that B cell.

In preferred embodiments of the latter embodiment, the outer membrane polypeptide or the complex thereof is:
(i) a TAA polypeptide of a bacterium or a complex thereof, respectively, wherein the method comprises: (1) immunizing a subject with a TAA polypeptide-containing chimeric polypeptide as defined in accordance with the first aspect of the invention, or a TAA polypeptide-containing chimeric polypeptide complex as defined in accordance with the sixth aspect of the invention, or a composition thereof as defined in accordance with the seventh aspect of the invention, wherein the TAA polypeptide corresponds to the TAA polypeptide of the bacterium; (2) identifying and/or isolating a B cell from the immunised subject, which specifically binds to the TAA polypeptide or complex thereof; and (3) producing the antigen-binding molecule expressed by that B cell;
or
(ii) a major outer membrane protein (MOMP) polypeptide of a Chlamydia bacterium or a complex thereof, respectively, wherein the method comprises: (1) immunizing a subject with a Chlamydia MOMP polypeptide-containing chimeric polypeptide as defined in accordance with the first aspect of the invention, or a Chlamydia MOMP polypeptide-containing chimeric polypeptide complex as defined in accordance with the sixth aspect of the invention, or a composition thereof as defined in accordance with the seventh aspect of the invention, wherein the Chlamydia MOMP polypeptide corresponds to the MOMP polypeptide of the Chlamydia bacterium; (2) identifying and/or isolating a B cell from the immunised subject, which specifically binds to the Chlamydia MOMP polypeptide or complex thereof; and (3) producing the antigen-binding molecule expressed by that B cell.

The invention provides, in a tenth aspect, an antigen-binding molecule that specifically binds to:
the microbial polypeptide-containing chimeric polypeptide as defined in accordance with the first aspect of the invention and/or the microbial polypeptide of one or more subunits of a microbial polypeptide-containing chimeric polypeptide complex as defined in accordance with the sixth aspect of the invention.

In preferred embodiments of the latter aspect, the antigen-binding molecule specifically binds to:
(i) the ectodomain of an enveloped virus fusion ectodomain polypeptide-containing chimeric polypeptide as defined in accordance with the first aspect of the invention; and/or the ectodomain of one or more subunits of an enveloped virus fusion ectodomain polypeptide-containing chimeric polypeptide complex as defined in accordance with the sixth aspect of the invention;
or
(ii) the bacterial outer membrane polypeptide of a bacterial outer membrane polypeptide-containing chimeric polypeptide as defined in accordance with the first aspect of the invention; and/or the bacterial outer membrane polypeptide of a bacterial outer membrane polypeptide-containing chimeric polypeptide complex as defined in accordance with the sixth aspect of the invention.

In preferred embodiments of the latter embodiment, the antigen-binding molecule specifically binds to:
(i) the TAA polypeptide of a TAA polypeptide-containing chimeric polypeptide as defined in accordance with the first aspect of the invention; and/or the TAA polypeptide of a TAA polypeptide-containing chimeric polypeptide complex as defined in accordance with the sixth aspect of the invention; or
(ii) the Chlamydia major outer membrane protein (MOMP) polypeptide of a Chlamydia MOMP polypeptide-containing chimeric polypeptide as defined in accordance with the first aspect of the invention; and/or the Chlamydia MOMP polypeptide of a Chlamydia MOMP polypeptide-containing chimeric polypeptide complex as defined in accordance with the sixth aspect of the invention.

The invention provides, in an eleventh aspect, an antigen-binding molecule that is obtainable by the method according to items (a) or (b) of the ninth aspect of the invention.

### 8. Compositions

The invention provides, in a seventh aspect, a composition comprising a chimeric polypeptide as defined in accordance with the first or second aspect of the invention, or a chimeric polypeptide complex as defined in accordance with the sixth aspect of the invention, and a pharmaceutically acceptable carrier, diluent or adjuvant.

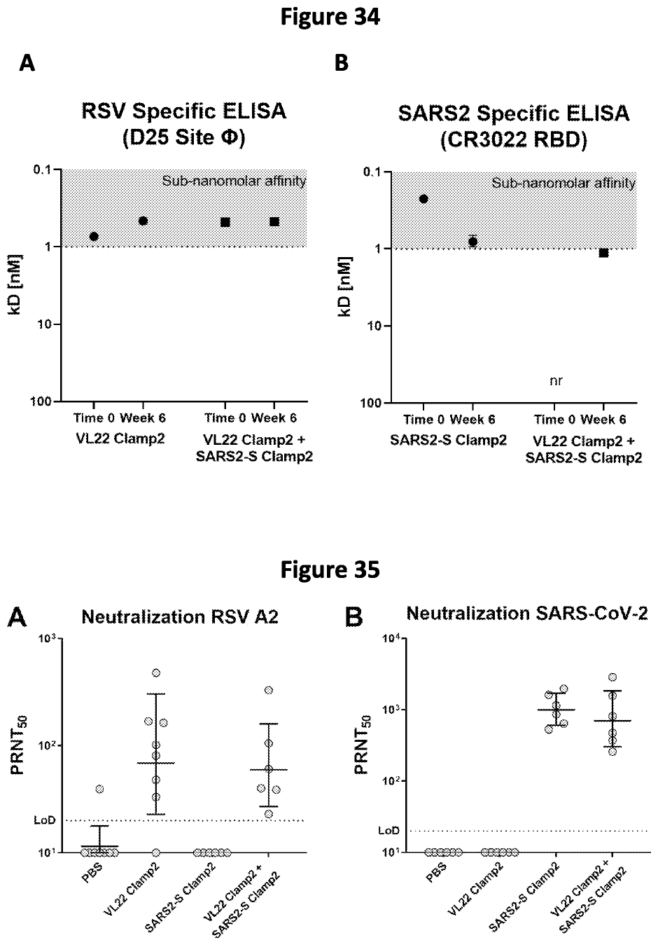

As demonstrated in Example 18 and corresponding FIGS. 34 and 35, the present technology also provides compositions which comprise at least two different chimeric polypeptide complexes, i.e., having antigen portions derived from the fusion ectodomain polypeptides from two different viruses (e.g., SARS-CoV-2 and RSV). The results of these experiments show that at least two different clamp stabilized antigens can be suitably combined into a single (bivalent) formulation (and stored over more than 6 weeks at 4° C.) without affecting the stability of either antigen or interfering with the neutralising immune response elicited to either antigen.

Thus, in particularly preferred embodiments of the composition according to the seventh aspect of the invention, the composition comprises at least two different variants of a chimeric polypeptide as defined in accordance with the first or second aspect of the invention; or at least two different variants of a chimeric polypeptide complex as defined in accordance with the sixth aspect of the invention. It will be understood that the expression “at least two different variants” of a chimeric polypeptide (or a chimeric polypeptide complex) is used herein synonymously with “at least two different types” of a chimeric polypeptide (or a chimeric polypeptide complex) or “at least two different” chimeric polypeptides (or chimeric polypeptide complexes).

In preferred embodiments of the latter embodiments, said variants differ with respect to their microbial polypeptides, e.g., their enveloped virus fusion ectodomain polypeptides or bacterial outer membrane polypeptides.

In even more preferred embodiments, said different enveloped virus fusion ectodomain polypeptides are derived from different viruses, more preferably, at least one from an orthopneumovirus (e.g., RSV) and at least one from a coronavirus (e.g., SARS-CoV-2). In alternative preferred embodiments, said different enveloped virus fusion ectodomain polypeptides are derived from an orthopneumovirus (e.g., RSV) and from a metapneumovirus; or from an orthopneumovirus (e.g., RSV) and from a parainfluenza virus (PIV); or from an orthopneumovirus (e.g., RSV) and from a parainfluenza virus (PIV) and from a metapneumovirus.

In other embodiments, said variants differ with respect to their bacterial outer membrane polypeptides, e.g., their TAA polypeptides; in particular, the TAA polypeptides may be derived from different bacteria (e.g., from different bacteria which belong to different genera, or from different bacteria that belong to the same genus, or from different bacteria that belong to the same species but different subtypes of said species.

The invention provides, in a twelfth aspect, a composition comprising an antigen-binding molecule as defined in accordance with the tenth or eleventh aspect of the invention, and a pharmaceutically acceptable carrier, diluent or adjuvant. The composition may be, e.g., an immune-modulating composition.

The invention provides, in a thirteenth aspect, a composition comprising the nucleic acid as defined in accordance with the third aspect of the invention.

In some embodiments, the chimeric polypeptide, which is encoded by the polynucleotide sequence and comprised in the nucleic acid, is:
(i) a microbial polypeptide-containing chimeric polypeptide as defined in accordance with the first aspect of the invention; or
(ii) a first (poly)peptide-containing chimeric polypeptide as defined in accordance with the second aspect of the invention.

In preferred embodiments of item (i) of the latter embodiment, the microbial polypeptide-containing chimeric polypeptide is:
(i) an enveloped virus fusion ectodomain polypeptide-containing chimeric polypeptide as defined in accordance with the first aspect of the invention; or
(ii) a bacterial outer membrane polypeptide-containing chimeric polypeptide as defined in accordance with the first aspect of the invention.

In preferred embodiments of item (ii) of the latter embodiment, the bacterial outer membrane polypeptide-containing chimeric polypeptide is:
(i) a TAA polypeptide-containing chimeric polypeptide as defined in accordance with the first aspect of the invention; or
(ii) a Chlamydia major outer membrane protein (MOMP) polypeptide-containing chimeric polypeptide as defined in accordance with the first aspect of the invention.

In some embodiments, the nucleic acid comprises or consists of RNA.

In some embodiments, the RNA comprises at least one modified nucleotide; wherein preferably the at least modified nucleotide is a modified uridine, more preferably, a methylated derivative of uridine, most preferably a N1-methyl-pseudouridine. In even further preferred embodiments, every uridine within the RNA is replaced by a methylated derivative of uridine, preferably by a N1-methyl-pseudouridine.

In even more preferred embodiments, every uridine within the RNA is replaced by N1-methyl-pseudouridine, and the RNA comprises a 5′-cap and a poly-A tail.

In some embodiments, the RNA is formulated in a delivery vehicle which is a liposome, lipoplex or lipid nanoparticle; wherein preferably the lipid nanoparticle comprises a cationic lipid, a neutral lipid, a steroid, and/or a PEGylated lipid.

In some embodiments, the nucleic acid comprises or consists of DNA; wherein preferably the DNA is comprised in a plasmid.

### 9. Medical Applications

The invention provides, in a fourteenth aspect, a chimeric polypeptide as defined in accordance with the first or second aspect of the invention, a nucleic acid as defined in accordance with the third aspect of the invention, a host cell as defined in accordance with the fourth aspect of the invention, a chimeric polypeptide complex as defined in accordance with the sixth aspect of the invention, a composition as defined in accordance with the seventh aspect of the invention, an antigen-binding molecule as defined in accordance with the tenth or eleventh aspect of the invention, or a composition as defined in accordance with the twelfth or thirteenth aspect of the invention for use as a medicament.

The invention provides, in a fifteenth aspect, a method of eliciting an immune response to: a microbial polypeptide, or complex thereof, in a subject, wherein the method comprises administering to the subject:
(i) a microbial polypeptide-containing chimeric polypeptide as defined in accordance with the first aspect of the invention, a microbial polypeptide-containing chimeric polypeptide complex as defined in accordance with the sixth aspect of the invention, or a composition thereof as defined in accordance with the seventh aspect of the invention; or
(ii) a composition as defined in accordance with the thirteenth aspect of the invention.

In preferred embodiments of the latter aspect, the microbial polypeptide, or the complex thereof, is:
(a) a fusion protein of an enveloped virus, or complex of the fusion protein, respectively, wherein the method comprises administering to the subject (i) an enveloped virus fusion ectodomain-containing chimeric polypeptide as defined in accordance with the first aspect of the invention, an enveloped virus fusion ectodomain-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; wherein an ectodomain polypeptide subunit of the chimeric polypeptide complex corresponds to, or substantially corresponds to, the fusion protein of the enveloped virus;
or
(b) an outer membrane polypeptide of a bacterium, or a complex of the outer membrane polypeptide, respectively, wherein the method comprises administering to the subject: (i) a bacterial outer membrane polypeptide-containing chimeric polypeptide as defined in accordance with the first aspect of the invention, a bacterial outer membrane polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; wherein a bacterial outer membrane polypeptide subunit of the chimeric polypeptide complex corresponds to, or substantially corresponds to, a bacterial outer membrane polypeptide expressed by the bacterium.

In preferred embodiments of the latter embodiment, the bacterial outer membrane polypeptide, or the complex thereof, is:
(a) TAA polypeptide of a bacterium, or a complex of the TAA polypeptide, respectively, wherein the method comprises administering to the subject: (i) a TAA polypeptide-containing chimeric polypeptide as defined in accordance with the first aspect of the invention, a TAA polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; wherein a TAA polypeptide subunit of the chimeric polypeptide complex corresponds to, or substantially corresponds to, a TAA polypeptide expressed by the bacterium;
or
(b) Chlamydia major outer membrane protein (MOMP) polypeptide, or a complex of the Chlamydia MOMP polypeptide, respectively, wherein the method comprises administering to the subject: (i) a Chlamydia MOMP polypeptide-containing chimeric polypeptide as defined in accordance with the first aspect of the invention, a Chlamydia MOMP polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; wherein a Chlamydia MOMP polypeptide subunit of the chimeric polypeptide complex corresponds to, or substantially corresponds to, a Chlamydia MOMP polypeptide expressed by the bacterium.

As used herein, the expression that a microbial polypeptide comprised in the chimeric polypeptide “substantially corresponds to” a certain referred microbial polypeptide means that the microbial polypeptide as comprised in the chimeric polypeptide has at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity or similarity to a naturally occurring polypeptide expressed by a corresponding microorganism.

The invention provides, in a sixteenth aspect, a method for treating or preventing: a microbial infection in a subject, wherein the method comprises administering to the subject an effective amount of:
(i) a microbial polypeptide-containing chimeric polypeptide as defined in accordance with the first aspect of the invention, a microbial polypeptide-containing chimeric polypeptide complex as defined in accordance with the sixth aspect of the invention, or a composition thereof as defined in accordance with the seventh aspect of the invention;
(ii) an antigen-binding molecule as defined in accordance with the tenth or eleventh aspect of the invention, or a composition thereof as defined in accordance with the twelfth aspect of the invention; or
(iii) a composition as defined in accordance with the thirteenth aspect of the invention.

In preferred embodiments of the latter aspect, the microbial infection is: (a) an enveloped virus infection in a subject, wherein the method comprises administering to the subject an effective amount of: (i) an enveloped virus fusion ectodomain-containing chimeric polypeptide as defined in accordance with the first aspect of the invention, an enveloped virus fusion ectodomain-containing chimeric polypeptide complex as defined in accordance with the sixth aspect of the invention, or a composition thereof as defined in accordance with the seventh aspect of the invention; (ii) an antigen-binding molecule as defined in accordance with the tenth or eleventh aspect of the invention, or a composition thereof as defined in accordance with the twelfth aspect of the invention; or (iii) a composition as defined in accordance with the thirteenth aspect of the invention;
or
(b) a bacterial infection in a subject, wherein the method comprises administering to the subject an effective amount of: (i) a bacterial outer membrane polypeptide-containing chimeric polypeptide as defined in accordance with the first aspect of the invention, a bacterial outer membrane polypeptide-containing chimeric polypeptide complex as defined in accordance with the sixth aspect of the invention, or a composition thereof as defined in accordance with the seventh aspect of the invention; (ii) an antigen-binding molecule as defined in accordance with the tenth or eleventh aspect of the invention, or a composition thereof as defined in accordance with the twelfth aspect of the invention; or (iii) a composition as defined in accordance with the thirteenth aspect of the invention.

In preferred embodiments of the latter embodiment, the bacterial infection is:
(a) an infection by a TAA-expressing bacterium, wherein the method comprises administering to the subject an effective amount of: (i) a TAA polypeptide-containing chimeric polypeptide as defined in accordance with the first aspect of the invention, a TAA polypeptide-containing chimeric polypeptide complex as defined in accordance with the sixth aspect of the invention, or a composition thereof as defined in accordance with the seventh aspect of the invention; (ii) an antigen-binding molecule as defined in accordance with the tenth or eleventh aspect of the invention or a composition thereof as defined in accordance with the twelfth aspect of the invention; or (iii) a composition as defined in accordance with the thirteenth aspect of the invention;
or
(b) an infection by a Chlamydia bacterium, wherein the method comprises administering to the subject an effective amount of: (i) a Chlamydia MOMP polypeptide-containing chimeric polypeptide as defined in accordance with the first aspect of the invention, a Chlamydia MOMP polypeptide-containing chimeric polypeptide complex as defined in accordance with the sixth aspect of the invention, or a composition thereof as defined in accordance with the seventh aspect of the invention; (ii) an antigen-binding molecule as defined in accordance with the tenth or eleventh aspect of the invention, or a composition thereof as defined in accordance with the twelfth aspect of the invention; or (iii) a composition as defined in accordance with the thirteenth aspect of the invention.

The invention provides, in a seventeenth aspect, a vaccine comprising:
(i) a microbial polypeptide-containing chimeric polypeptide as defined in accordance with the first aspect of the invention, a microbial polypeptide-containing chimeric polypeptide complex as defined in accordance with the sixth aspect of the invention, or a composition thereof as defined in accordance with the seventh aspect of the invention; or
(ii) a composition as defined in accordance with the thirteenth aspect of the invention;
for use in a method of eliciting an immune response to a microbial polypeptide, or a complex of the microbial polypeptide, in a subject.

In preferred embodiments of the latter aspect, the microbial polypeptide is:
(a) an enveloped virus fusion ectodomain polypeptide, and the vaccine comprises: (i) an enveloped virus fusion ectodomain polypeptide-containing chimeric polypeptide as defined in accordance with the first aspect of the invention, an enveloped virus fusion ectodomain polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; for use in a method of eliciting an immune response to a fusion protein of an enveloped virus, or a complex of the fusion protein, in a subject, and wherein an ectodomain polypeptide subunit of the chimeric polypeptide complex corresponds to the fusion protein of the enveloped virus;
or
(b) a bacterial outer membrane polypeptide, and the vaccine comprises: (i) a bacterial outer membrane polypeptide-containing chimeric polypeptide as defined in accordance with the first aspect of the invention, a bacterial outer membrane polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; for use in a method of eliciting an immune response to an outer membrane polypeptide of a bacterium, or a complex of the outer membrane polypeptide, in a subject, and wherein a bacterial outer membrane polypeptide subunit of the chimeric polypeptide complex corresponds to an outer membrane polypeptide of the bacterium.

In preferred embodiments of the latter embodiment, the bacterial outer membrane polypeptide is:
(a) a trimeric autotransporter adhesin (TAA) polypeptide, and the vaccine comprises: (i) a TAA polypeptide-containing chimeric polypeptide as defined in accordance with the first aspect of the invention, a TAA polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; for use in a method of eliciting an immune response to a TAA polypeptide of a bacterium, or a complex of the TAA polypeptide, in a subject, and wherein a TAA polypeptide subunit of the chimeric polypeptide complex corresponds to a TAA polypeptide of the bacterium;
or
(b) a Chlamydia MOMP polypeptide, and the vaccine comprises: (i) a Chlamydia MOMP polypeptide-containing chimeric polypeptide as defined in accordance with the first aspect of the invention, a Chlamydia MOMP polypeptide-containing chimeric polypeptide complex as defined in accordance with the sixth aspect of the invention, or a composition thereof as defined in accordance with the seventh aspect of the invention; or (ii) a composition as defined in accordance with the thirteenth aspect of the invention; for use in a method of eliciting an immune response to a MOMP polypeptide of a Chlamydia bacterium, or a complex of the MOMP polypeptide, in a subject, and wherein a MOMP polypeptide subunit of the chimeric polypeptide complex corresponds to a MOMP polypeptide of the Chlamydia bacterium.

The invention provides, in an eighteenth aspect, a microbial polypeptide-containing chimeric polypeptide as defined in accordance with the first aspect of the invention, a microbial polypeptide-containing chimeric polypeptide complex as defined in accordance with the sixth aspect of the invention, or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing a microbial infection in a subject.

It is understood that the microbial infection (which may also be referred to as “microbial infectious disease”) which in accordance with the use of the eighteenth aspect is treated or prevented is an infection caused by a microorganism which expresses a microbial polypeptide corresponding to or having at least 70% (or, with increasing preference, at least 71%, 72%, 73%, 74%, 75%, 76%, 7700, 780, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the microbial polypeptide as comprised in the chimeric polypeptide.

In preferred embodiments of the latter aspect, the microbial polypeptide is:
(a) an enveloped virus fusion ectodomain polypeptide, and provided is an enveloped virus fusion ectodomain polypeptide-containing chimeric polypeptide as defined in accordance with the first aspect of the invention, an enveloped virus fusion ectodomain-containing chimeric polypeptide complex as defined in accordance with the sixth aspect of the invention or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing an enveloped virus infection in a subject;
or
(b) a bacterial outer membrane polypeptide, and provided is a bacterial outer membrane polypeptide-containing chimeric polypeptide as defined in accordance with the first aspect of the invention, a bacterial outer membrane polypeptide-containing chimeric polypeptide complex as defined in accordance with the sixth aspect of the invention or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing a bacterial infection in a subject.

It is understood that the enveloped virus infection which, in accordance with item (a) of the forgoing embodiment, is treated or prevented is an infection caused by an enveloped virus which expresses an enveloped virus fusion ectodomain polypeptide corresponding to, or having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the enveloped virus fusion ectodomain polypeptide as comprised in the chimeric polypeptide.

Similarly, it is understood that the bacterial infection which, in accordance with item (b) of the forgoing embodiment, is treated or prevented is an infection caused by bacteria which express an outer membrane polypeptide corresponding to, or having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the bacterial outer membrane polypeptide as comprised in the chimeric polypeptide.

In preferred embodiments of the latter embodiment, the bacterial outer membrane polypeptide is:
(a) a bacterial trimeric autotransporter adhesin (TAA) polypeptide, and provided is a bacterial trimeric autotransporter adhesin (TAA) polypeptide-containing chimeric polypeptide as defined in accordance with the first aspect of the invention, a TAA polypeptide-containing chimeric polypeptide complex as defined in accordance with the sixth aspect of the invention or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing a bacterial infection by a TAA-expressing bacterium in a subject; or
(b) a Chlamydia MOMP polypeptide, and provided is a Chlamydia MOMP polypeptide-containing chimeric polypeptide as defined in accordance with the first aspect of the invention, a Chlamydia MOMP polypeptide-containing chimeric polypeptide complex as defined in accordance with the sixth aspect of the invention or a composition thereof as defined in accordance with the seventh aspect of the invention, or an antigen-binding molecule as defined in accordance with the tenth or eleventh aspect of the invention or a composition thereof as defined in accordance with the twelfth aspect of the invention, or a composition as defined in accordance with the thirteenth aspect of the invention, for use in a method for treating or preventing a Chlamydia infection in a subject.

It is understood that the bacterial infection which in accordance with item (a) of the forgoing embodiment is treated or prevented is an infection caused by bacteria which express a TAA polypeptide corresponding to or having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the TAA polypeptide as comprised in the chimeric polypeptide.

It is understood that the Chlamydia infection which in accordance with item (b) of the forgoing embodiment is treated or prevented is a Chlamydia infection caused by Chlamydia bacteria which express a Chlamydia MOMP polypeptide corresponding to or having at least 70% (or, with increasing preference, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 100%) sequence identity to the Chlamydia MOMP polypeptide as comprised in the chimeric polypeptide.

It is particularly preferred that the bacterial infection to be treated or prevented is caused by the bacterium from which the bacterial surface polypeptide (e.g., the bacterial outer membrane polypeptide, such as the TAA polypeptide) which is comprised in the chimeric polypeptide is derived. For example, if the chimeric polypeptide comprises a TAA polypeptide which is Haemophilus influenzae adhesin (HiA), then it is preferred that the bacterial infection to be treated or prevented is an infection caused by Haemophilus influenzae.

It will be understood that each of the products and compositions described in the fourteenth to eighteenth aspects of the invention may also be used in the manufacture of a medicament for the therapeutic or prophylactic applications described herein.

### 10. Dosages and Routes of Administration

The composition is administered in an “effective amount” that is, an amount effective to achieve an intended purpose in a subject. The dose of active compound(s) administered to a patient should be sufficient to achieve a beneficial response in the subject over time such as a reduction in at least one symptom associated with an infection. The quantity or dose frequency of the pharmaceutically active compounds(s) to be administered may depend on the subject to be treated inclusive of the age, sex, weight, and general health condition thereof. In this regard, precise amounts of the active compound(s) for administration will depend on the judgment of the practitioner. One skilled in the art would be able, by routine experimentation, to determine an effective, non-toxic amount of a chimeric polypeptide or complex described herein to include in a pharmaceutical composition of the present disclosure for the desired therapeutic outcome.

In general, a (pharmaceutical) composition of the present disclosure can be administered in a manner compatible with the route of administration and physical characteristics of the recipient (including health status) and in such a way that it elicits the desired effect(s) (i.e., therapeutically effective, immunogenic and/or protective). For example, the appropriate dosage of a pharmaceutical composition of the present disclosure may depend on a variety of factors including, but not limited to, a subject's physical characteristics (e.g., age, weight, sex), whether the compound is being used as single agent or adjuvant therapy, the type of MHC restriction of the patient, the progression (i.e., pathological state) of a virus infection, and other factors that may be recognized by one skilled in the art. Various general considerations that may be considered when determining an appropriate dosage of a pharmaceutical composition of the present disclosure are described, for example, in Gennaro (2000) “Remington: The Science and Practice of Pharmacy”, 20th edition, Lippincott, Williams, & Wilkins; and Gilman et al., (Eds), (1990), “Goodman And Gilman's: The Pharmacological Bases of Therapeutics”, Pergamon Press.

In some embodiments, an “effective amount” of a subject chimeric polypeptide or complex, or a chimeric polypeptide-encoding polynucleotide, or a nucleic acid construct from which the chimeric polypeptide or complex is expressible, is an amount sufficient to achieve a desired prophylactic or therapeutic effect, e.g., to reduce a symptom associated with infection, and/or to reduce the number of infectious agents in the individual. In these embodiments, an effective amount reduces a symptom associated with infection and/or reduces the number of infectious agents in an individual by at least about 10%, at least about 20%, at least about 30%, at least about 40%, at least about 50%, at least about 60%, at least about 70%, at least about 80%, or at least about 90%, or more, when compared to the symptom or number of infectious agents in an individual not treated with the chimeric polypeptide or complex. Symptoms of infection by a pathogenic organism, as well as methods for measuring such symptoms, are known in the art. Methods for measuring the number of pathogenic organisms in an individual are standard in the art.

In some embodiments, an “effective amount” of a subject chimeric polypeptide or complex, or a chimeric polypeptide-encoding polynucleotide, or a nucleic acid construct from which the chimeric polypeptide or complex is expressible, is an amount that is effective in a selected route of administration to elicit an immune response to an enveloped virus fusion ectodomain polypeptide comprised in the chimeric polypeptide of the invention.

In some embodiments, e.g., where the chimeric polypeptide comprises a heterologous antigen, an “effective amount” is an amount that is effective to facilitate elicitation of an immune response against that antigen. For example, where the heterologous antigen is an antigen from a different pathogenic organism than the one from which the ectodomain polypeptide is derived), an “effective amount” of a subject chimeric polypeptide or complex, or a chimeric polypeptide-encoding polynucleotide, or a nucleic acid construct from which the chimeric polypeptide or complex is expressible, is an amount that is effective for elicitation of an immune response against that antigen and preferably protection of the host against infection, or symptoms associated with infection, by that pathogenic organism. In these embodiments, an effective amount reduces a symptom associated with infection by the pathogenic organism and/or reduces the number of infectious agents corresponding to the pathogenic organism in an individual by at least about 10%, at least about 20%, at least about 30%, at least about 40%, at least about 50%, at least about 60%, at least about 70%, at least about 80%, or at least about 90%, or more, when compared to the symptom or number of infectious agents in an individual not treated with the chimeric polypeptide or complex, or a chimeric polypeptide-encoding polynucleotide, or a nucleic acid construct from which the chimeric polypeptide or complex is expressible. Symptoms of infection by a pathogenic organism, as well as methods for measuring such symptoms, are known in the art.

Alternatively, where a heterologous antigen is a cancer- or tumor-associated antigen, an “effective amount” of a chimeric polypeptide or complex, or a chimeric polypeptide-encoding polynucleotide, or a nucleic acid construct from which the chimeric polypeptide or complex is expressible, is an amount that is effective in a route of administration to elicit an immune response effective to reduce or inhibit cancer or tumor cell growth, to reduce cancer or tumor cell mass or cancer or tumor cell numbers, or to reduce the likelihood that a cancer or tumor will form. In these embodiments, an effective amount reduces tumor growth and/or the number of tumor cells in an individual by at least about 10%, at least about 20%, at least about 30%, at least about 40%, at least about 50%, at least about 60%, at least about 70%, at least about 80%, or at least about 90%, or more, when compared to the tumor growth and/or number of tumor cells in an individual not treated with the chimeric polypeptide or complex, or a chimeric polypeptide-encoding polynucleotide, or a nucleic acid construct from which the chimeric polypeptide or complex is expressible. Methods of measuring tumor growth and numbers of tumor cells are known in the art.

In various embodiments, the amount of chimeric polypeptide or complex, or a chimeric polypeptide-encoding polynucleotide, or a nucleic acid construct from which the chimeric polypeptide or complex is expressible in each dose is selected as an amount that induces an immune response to the encoded ectodomain polypeptide, and/or that induces an immune-protective or other immunotherapeutic response without significant, adverse side effects generally associated with typical vaccines. Such amount may vary depending upon which specific chimeric polypeptide or complex, or chimeric polypeptide-encoding polynucleotide, or a nucleic acid construct from which the chimeric polypeptide or complex is expressible is employed, whether or not the vaccine formulation comprises an adjuvant, and a variety of host-dependent factors.

A pharmaceutical composition of the present disclosure can be administered to a recipient by standard routes, including, but not limited to, parenteral (e.g., intravenous), intramuscular, subcutaneous or intradermal.

A pharmaceutical composition of the present disclosure may be administered to a recipient in isolation or in conjunction with additional therapeutic agent(s). In embodiments where a pharmaceutical composition is concurrently administered with therapeutic agent(s), the administration may be simultaneous or sequential (i.e., pharmaceutical composition administration followed by administration of the agent(s) or vice versa).

Typically, in treatment applications, the treatment may be for the duration of the disease state or condition. Further, it will be apparent to one of ordinary skill in the art that the optimal quantity and spacing of individual dosages will be determined by the nature and extent of the disease state or condition being treated, the form, route and site of administration, and the nature of the particular individual being treated. Optimum conditions can be determined using conventional techniques.

In many instances (e.g., preventative applications), it may be desirable to have several or multiple administrations of a pharmaceutical composition of the present disclosure. For example, a pharmaceutical composition may be administered 1, 2, 3, 4, 5, 6, 7, 8, 9, 10, or more times. The administrations may be from about one- to about twelve-week intervals, and in certain embodiments from about one to about four-week intervals. Periodic re-administration may be desirable in the case of recurrent exposure to a particular pathogen or other disease-associated component targeted by a pharmaceutical composition of the present disclosure. It will also be apparent to one of ordinary skill in the art that the optimal course of administration can be ascertained using conventional course of treatment determination tests.

Where two or more entities are administered to a subject “in conjunction” or “concurrently” they may be administered in a single composition at the same time, or in separate compositions at the same time, or in separate compositions separated in time.

Certain embodiments of the present disclosure involve the administration of pharmaceutical compositions in multiple separate doses. Accordingly, the methods for the prevention (i.e., vaccination) and treatment of infection described herein encompass the administration of multiple separated doses to a subject, for example, over a defined period of time. Accordingly, the methods for the prevention (i.e., vaccination) and treatment of infection disclosed herein include administering a priming dose of a pharmaceutical composition of the present disclosure. The priming dose may be followed by a booster dose. The booster may be for the purpose of re-vaccination. In various embodiments, the pharmaceutical composition or vaccine is administered at least once, twice, three times or more.

Methods for measuring the immune response are known to persons of ordinary skill in the art. Exemplary methods include solid-phase heterogeneous assays (e.g., enzyme-linked immunosorbent assay), solution phase assays (e.g., electrochemiluminescence assay), amplified luminescent proximity homogeneous assays, flow cytometry, intracellular cytokine staining, functional T-cell assays, functional B-cell assays, functional monocyte-macrophage assays, dendritic and reticular endothelial cell assays, measurement of NK cell responses, IFN-γ production by immune cells, quantification of virus RNA/DNA in tissues or biological fluids (e.g., quantification of viral RNA or DNA in serum or other fluid or tissue/organ), oxidative burst assays, cytotoxic-specific cell lysis assays, pentamer binding assays, and phagocytosis and apoptosis evaluation.

It will be appreciated by persons skilled in the art that numerous variations and/or modifications may be made to the disclosure as shown in the specific embodiments without departing from the spirit or scope of the present disclosure as broadly described. The present embodiments are, therefore, to be considered in all respects as illustrative and not restrictive.

Sequences referred to herein are also presented in the form of a sequence listing which is filed together with the present disclosure. In case of any deviation(s) between the respective sequences, the present invention specifically and individually relates to each one of the corresponding sequences, and it preferably relates to the sequences disclosed in the present specification.

Regarding the embodiments characterized in this specification, in particular in the claims, it is intended that each embodiment mentioned in a dependent claim is combined with each embodiment of each claim (independent or dependent) said dependent claim depends on. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously, individually and specifically discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly and individually disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

In order that the disclosure may be readily understood and put into practical effect, particular preferred embodiments will now be described by way of the following non-limiting examples.

### EXAMPLES

### Example 1: Preparation of Constructs

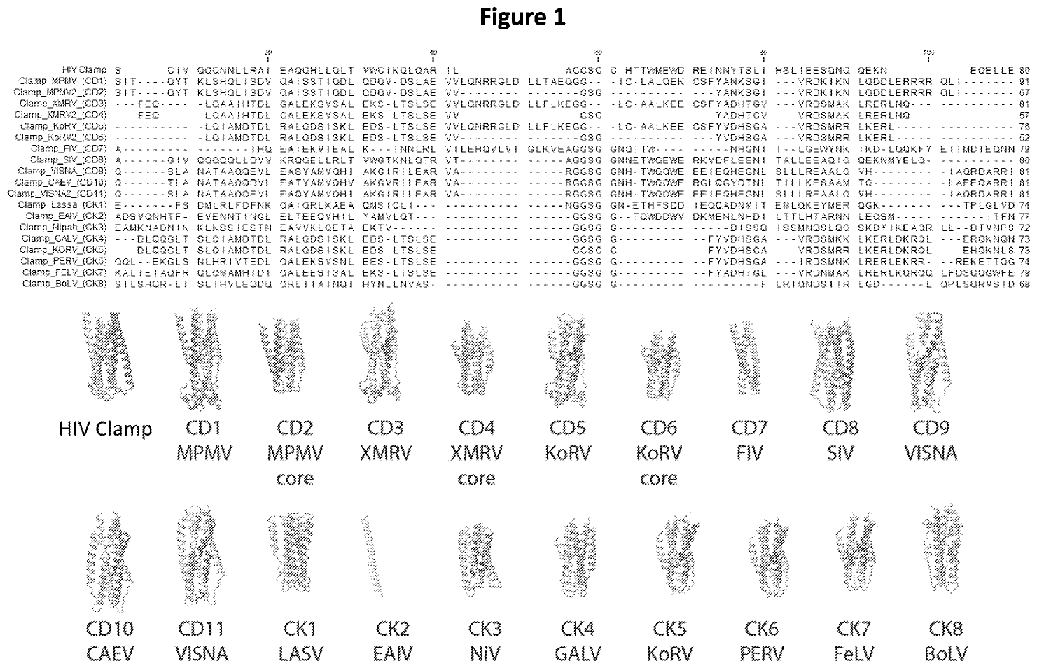

In order to overcome the HIV diagnostic interference observed with the original HIV-based clamp (HIV-clamp SEQ ID NO: 1), nineteen putative clamp sequences derived from the trimerization domains from proteins from animal viruses known not to commonly infect humans were designed (Table 3; FIG. 1; SEQ ID NOs: 2-20). Predicted first and second heptad repeat regions (FHRR and SHRR, respectively) were joined by flexible linker sequences (GG, GSG or GGSGG) to facilitate engagement between the FHRR and SHRR.

The resulting panel of putative clamp sequences were appended to the C-terminal ends of the respiratory syncytial virus fusion protein ectodomain amino acids 1-511 (Genbank Accession: AHL84194; SEQ ID NO: 21 (66K)) with a flexible GS linker included between T¹⁰³ and G¹⁴⁵. An additional GSG linker sequence was included between the C-terminus of the ectodomain and the N-terminus of each of the 19 putative clamp sequences SEQ ID NOS: 2-20. The resulting panel were referred to as VL22_66K_CD1-11/CK1-8 (representative sequence shown for VL22_66K_CD11 in SEQ ID NO: 22).

The same panel of clamp sequences were similarly appended to the C-terminal ends of the respiratory syncytial virus fusion protein ectodomain amino acids 1-511 (Genbank Accession: AHL84194; SEQ ID NO: 23 (66E)) with a flexible GS linker included between T¹⁰³ and G¹⁴⁵. An additional GSG linker sequence was included as a hinge region between the C-terminus of the ectodomain and the N-terminus of each of the 19 putative clamp sequences SEQ ID NOs: 2-20. The resulting panel were referred to as VL22_66E_CD1-11/CK1-8 (representative sequence shown for VL22_66E_CD11 in SEQ ID NO: 24.

Previously a prefusion stabilized RSV F protein was produced that consisted of fusion protein ectodomain amino acids 1-511 (Genbank Accession: AI008046; SEQ ID NO: 25) with a flexible GS linker included between T¹⁰³ and G¹⁴⁵ and a HIV gp41 based clamp (SEQ ID NO: 1) appended to the C-terminus of the ectodomain. This original antigen is referred to herein as F HIV-clamp (SEQ ID NO: 26).

Primers and gene fragment blocks (gBlocks™) were synthesised by Integrated DNA Technologies and cloned into digested expression vector using an In-Fusion cloning reaction (Takara Bio Kusatsu, Shiga, Japan). The resulting plasmids were transformed into Escherichia coli HST08 Stellar™ Competent Cells (Takara Bio Kusatsu, Shiga, Japan). Plasmids from selected colonies were screened for correct insertions by colony PCR and were subjected to subsequent DNA sequencing to confirm sequence fidelity.

TABLE 3
Putative clamp sequences

| | | | | Linker | |
|---|---|---|---|---|---|
| Name | Virus | SEQ ID NO: | FHRR Sequence | Sequence | SHRR Sequence |
| HIV- | Source | SEQ ID NO: 1 | SGIVQQQNNLLRAIEAQQ | GGSGG | HTTWMEWDREINNYTSLIH |
| clamp | | | HLLQLTVWGIKQLQARILA | | SLIEESQNQQEKNEQELLE |
| CD1 | MMPV | SEQ ID NO: 2 | SITQYTKLSHQLISDVQAIS | GG | ICLALQEKCSFYANKSGIVRD |
| | | | STIQDLQDQVDSLAEVVLQ | | KIKNLQDDLERRRRQLI |
| | | | NRRGLDLLTAEQ | | |
| CD2 | MMPV | SEQ ID NO: 3 | SITQYTKLSHQLISDVQAIS | GSG | ANKSGIVRDKIKNLQDDLER |
| | | | STIQDLQDQVDSLAEVV | | RRROLI |
| CD3 | XMRV | SEQ ID NO: 4 | FEQLQAAIHTDLGALEKSV | GG | LCAALKEECSFYADHTGVVR |
| | | | SALEKSLTSLSEVVLQNRRG | | DSMAKLRERLNQ |
| | | | LDLLFLKE | | |
| CD4 | XMRV | SEQ ID NO: 5 | FEQLQAAIHTDLGALEKSV | GSG | YADHTGVVRDSMAKLRERL |
| | | | SALEKSLTSLSEVV | | NQ |
| CD5 | KoRV | SEQ ID NO: 6 | LQIAMDTDLRALQDSISKL | GG | LCAALKEECSFYVDHSGAVR |
| | | | EDSLTSLSEVVLQNRRGLD | | DSMRRLKERL |
| | | | LLFLKE | | |
| CD6 | KoRV | SEQ ID NO: 7 | AMDTDLRALQDSISKLEDS | GSG | YVDHSGAVRDSMRRLKERL |
| | | | LTSLSEVV | | |
| CD7 | FIV | SEQ ID NO: 8 | ATHQEAIEKVTEALKINNLR | GGSGG | NQTIWNHGNITLGEWYNK |
| | | | LVTLEHQVLVIGLKVEA | | TKDLQQKFYEIIMDIEQNN |
| CD8 | SIV | SEQ ID NO: 9 | AGIVQQQQQLLDVVKRQ | GGSGG | NNETWQEWERKVDFLEENI |
| | | | QELLRLTVWGTKNLQTRV | | TALLEEAQIQQEKNMYELQ |
| | | | TA | | |
| CD9 | VISNA | SEQ ID NO: 10 | QSLANATAAQQEVLEASY | GGSGG | NHTWQQWEEEIEQHEGNL |
| | | | AMVQHIAKGIRILEARVAR | | SLLLREAALQVHIAQRDARR |
| | | | | | I |
| CD10 | CAEV | SEQ ID NO: 11 | QTLANATAAQQDVLEATY | GGSGG | NHTWQQWERGLQGYDTN |
| | | | AMVQHVAKGVRILEARVA | | LTILLKESAAMTQLAEEQAR |
| | | | R (SEQ ID NO: 81) | | RI (SEQ ID NO: 83) |
| CD11 | VISNA | SEQ ID NO: 12 | QSLANATAAQQEVLEAQY | GGSGG | NHTWQQWEEEIEQHEGNL |
| | | | AMVQHIAKGIRILEARVAR | | SLLLREAALQVHIAQRDARR |
| | | | (SEQ ID NO: 80) | | I (SEQ ID NO: 82) |
| CK1 | Lassa | SEQ ID NO: 13 | EFSDMLRLFDFNKQAIQRL | GGSGG | NETHFSDDIEQQADNMITE |
| | | | KAEAQMSIQLIN | | MLQKEYMERQGKTPLGLV |
| | | | | | D |
| CK2 | EAIV | SEQ ID NO: 14 | ADSVQNHTFEVENNTING | GGSGG | TQWDDWVDKMENLNHDI |
| | | | LELTEEQVHILYAMVLQT | | LTTLHTARNNLEQSMITFN |
| CK3 | NIV | SEQ ID NO: 15 | EAMKNADNINKLKSSIEST | GGSGG | DISSQISSMNQSLQQSKDYI |
| | | | NEAVVKLQETAEKTV | | KEAQRLLDTVNPS |
| CK4 | GALV | SEQ ID NO: 16 | DLQQGLTSLQIAMDTDLR | GGSGG | FYVDHSGAVRDSMKKLKER |
| | | | ALQDSISKLEDSLTSLSE | | LDKRQLERQKNQN |
| CK5 | KoRV | SEQ ID NO: 17 | DLQQGLTSLQIAMDTDLR | GGSGG | FYVDHSGAVRDSMRRLKER |
| | | | ALQDSISKLEDSLTSLSE | | LDKRQLEHQKNLS |
| CK6 | PERV | SEQ ID NO: 18 | QQLEKGLSNLHRIVTEDLQ | GGSGG | FYVDHSGAIRDSMNKLRER |
| | | | ALEKSVSNLEESLTSLSE | | LEKRRREKETTQG |
| CK7 | FeLV | SEQ ID NO: 19 | KALIETAQFRQLQMAMHT | GGSGG | FYADHTGLVRDNMAKLRER |
| | | | DIQALEESISALEKSLTSLSE | | LKQRQQLFDSQQGWFE |
| CK8 | BoLV | SEQ ID NO: 20 | STLSHQRLTSLIHVLEQDQ | GGSGG | FLRIQNDSIIRLGDLQPLSQR |
| | | | QRLITAINQTHYNLLNVAS | | VSTD |

### Example 2: Protein Expression

ExpiCHO™ Chinese hamster ovary (CHO) mammalian cells were transfected with 18 putative clamp-stabilised VL22_66K antigens generated in Example 1 (Table 3, excluding CD5), F HIV-clamp (SEQ ID NO: 26), and a non-stabilised control antigen (Fsol; SEQ ID NO: 27) using the ExpiCHO-S™ expression system (Thermo Fisher Scientific) for transient protein expression. After 6 days of culture the supernatants containing the soluble clamp proteins were harvested and immediately filtered through a 0.22 μm filter.

### Example 3: Screening of Clamp Supernatants

Supernatants obtained from CHO cells transfected with 18 putative clamp-stabilised VL22_66K antigens were tested for binding to RSV-specific antibodies; Motavizumab 101F (McLellan et al., J Virol, 2010, DOI: 10.1128/JVI.01579-10) and MPE8 (Wen et al., Nat Microbiol, 2017, DOI: 10.1038/nmicrobiol.2016.272) in a capture ELISA. Motavizumab 101F is a non-conformationally dependent RSV ectodomain binding antibody (McLellan et al., Nat Struct Mol Biol, 2010, DOI: 10.1038/nsmb.1723) whereas MPE8 is a conformationally dependent antibody specific for the pre-fusion form of the RSV F protein. Antibody 101F was coated on ELISA plates at 50 or 2 μg/mL in carbonate-bicarbonate buffer, and stored overnight at 4° C. Coating buffer was removed and additional protein binding sites on the plate blocked by incubation for 60 min at room temperature with 150 μL of KPL Milk diluent/blocking solution (Seracare, MA USA) in PBST, after which the blocking buffer was removed. 50 μL each of serial dilution of supernatants or purified protein controls in 1×KPL in PBST were added to the wells and incubated at 37° C. for 1 h. Plates were washed three times by immersing in water, and 50 μL of either 101F or MPE8 in 1×KPL Milk diluent/blocking solution (Seracare, MA USA) at a concentration of 2 μg/ml added. Plates were incubated for a further hour at 37° C., washed three times by immersing in water, and incubated with anti-human horse radish peroxidase conjugated antibody (Life Technologies A18829, diluted 1:2000 in KPL blocking buffer). Plates were incubated and washed as described above, then TMB (3,3′,5,5′-tetramethylbenzidine; 50 μL/well) added for 3 min, and the reaction stopped with 1 M sulphuric acid (25 μL/well). Antibody binding was detected using a colorimeter at 450 nm.

ELISA-based supernatant screening of VL22_66K antigen series found that the majority were expressed. Yields of the VL22-K66 clamp antigens were assessed by ELISA (Table 4) and compared to control F HIV-clamp and Fsol. All clamp antigens bound 101F but not all exhibited binding to the pre-fusion conformationally-specific MPE8 monoclonal antibody. VL22_66K_CD1 & CD2 (MPMV); CD3 (XMRV); CD7 (FIV); CD9 (VISNA); CD10 (CAEV); CD11 (VISNA2); and CK8 (BoLV) all bound MPE8 (see Table 4).

### Example 4: Antigen Purification

VL22_66K clamp antigens were purified using an in house made immunoaffinity chromatography column using the RSV-specific antibody 101F and an AKTA pure protein purification system (Cytiva, Marlborough, MA, USA) and pH11.5 elution. Following purification, eluates were immediately neutralised, and buffer exchanged into PBS using Merck Amicon Ultra-4 or Ultra-15 centrifugal filter units. Protein concentration was determined using the NanoDrop One (Thermofisher Scientific) (Table 4), and clamp-antigens ranked for further studies based on yields obtained.

TABLE 4
Yield of protein from supernatants

| | Yield of purified protein | | | |
|---|---|---|---|---|
| | 101F | MPE8 | Nanodrop | Lead |
| | binding | binding | post- | ranking |
| VL22_66K | ELISA | ELISA | purification | based |
| Construct | (mg/L) | (mg/L) | (mg/L) | on yield |
| CD1 | 13.1 | 6.7 | 24.6 | ** |
| CD2 | 27.0 | 8.4 | 50.0 | ** |
| CD3 | 8.9 | 3.9 | 16.3 | * |
| CD4 | 1.6 | 0 | 4.4 | 0 |
| CD5 | Not | Not | Not | N/A |
| | performed | performed | performed | |
| CD6 | 1.7 | 0 | 3.1 | 0 |
| CD7 | 12.9 | 9.5 | 24.7 | ** |
| CD8 | 1.6 | 0 | Not | 0 |
| | | | performed | |
| CD9 | 51.7 | 10.1 | 101.5 | *** |
| CD10 | 40.2 | 28.1 | 72.2 | **** |
| CD11 | 59.0 | 26.1 | 101.9 | **** |
| CK1 | 5.1 | 0 | Not | 0 |
| | | | performed | |
| CK2 | 0.7 | 0 | Not | 0 |
| | | | performed | |
| CK3 | 19.1 | 0 | Not | 0 |
| | | | performed | |
| CK4 | 0.0 | 0 | Not | 0 |
| | | | performed | |
| CK5 | 0.1 | 0 | 0.2 | 0 |
| CK6 | 0.7 | 0 | 0.8 | 0 |
| CK7 | 0.0 | 0 | 0.1 | 0 |
| CK8 | 7.5 | 1.8 | 15.4 | * |
| F HIV-clamp | 10.1 | 10 | 35.5 | ** |
| non-stabilised | 17.2 | 0 | 22.1 | 0 |
| control (Fsol) | | | | |

**** to * highest to lowest rank clamps. Antigens which failed one of the tests were assigned a clamp rank of 0.

### Example 5: Characterization of Purified Antigens

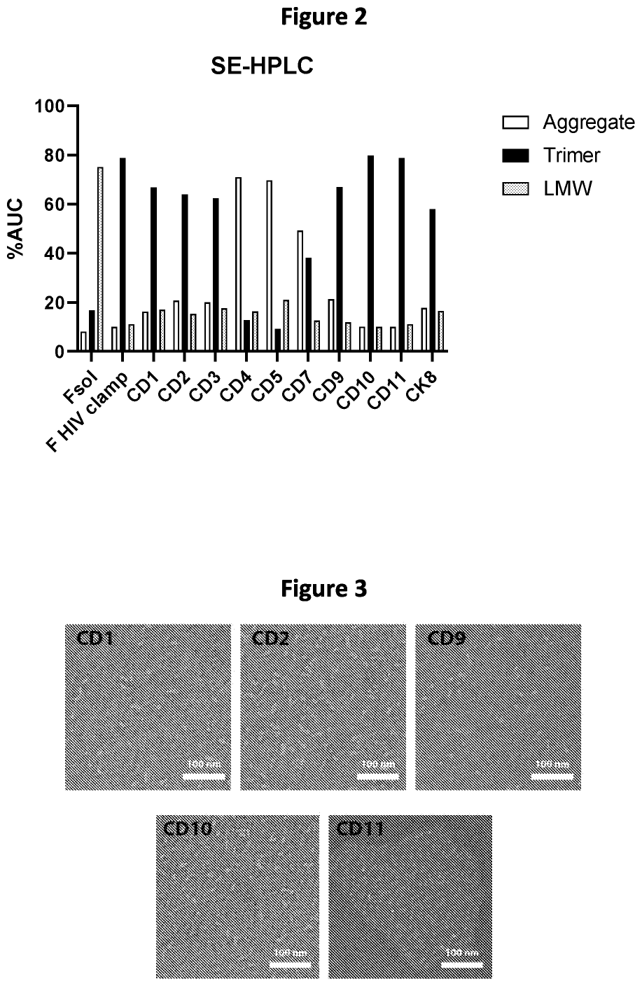

The transient protein expression was repeated for the lead clamp candidates (CD1, CD2, CD3, CD7, CD9, CD10, CD11 and CK8). Control F HIV-clamp and non-stabilised Fsol were again included, as was CD5, which was absent from the previous comparison and CD4 was included as a low promise candidate that produced at low yield and did not bind MPE8. Size exclusion high pressure liquid chromatography (SE-HPLC) analysis was used to evaluate the monodispersity and oligomeric state of the purified clamp antigens. SE-HPLC used a Waters column (XBridge BEH450 7.8×300 mm) and Waters guard column (Xbridge Guard BDH450 7.8×30 mm) using a DPBS mobile phase. The relative proportions of soluble trimer, high molecular weight aggregate and low molecular weight product were estimated by calculation of Area Under Curve (AUC) (FIG. 2). The analysis revealed that multiple candidate clamps facilitated trimer formation and stabilisation compared to the non-stabilised control. CD1, CD2, CD3, CD9, CD10 and CD11 stabilised similar levels of trimer compared to the F HIV-clamp control. CD4, CD5 and CD7 were all substantially aggregated, indicative of poor pre-fusion stabilisation.

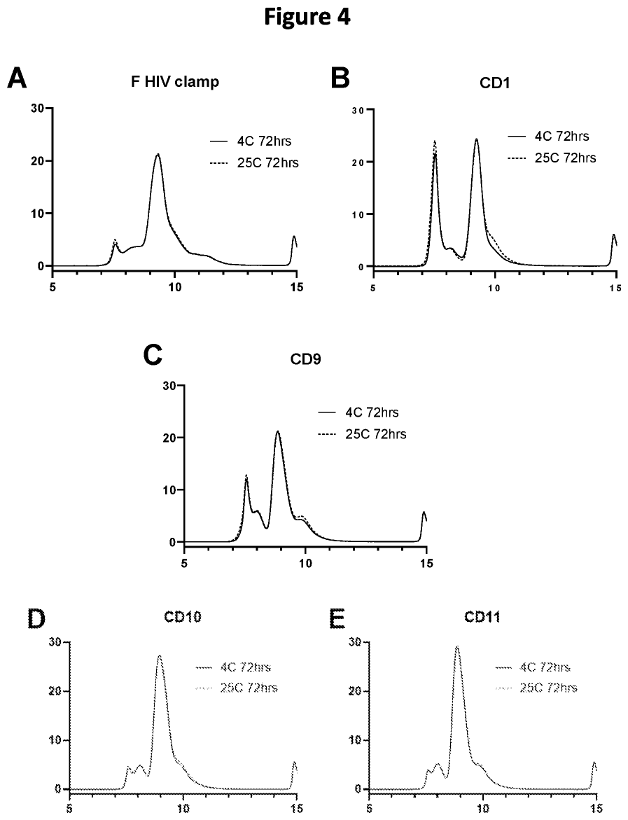

Five of the selected clamp antigens (CD1, CD2, CD9, CD10, and CD11) were then analysed by transition electron microscopy (TEM). CD3 was not analysed due to low yield. Negative stain TEM images revealed homogenous 10-20 nm particles similar to those previously observed of the F HIV-clamp and consistent with the know pre-fusion structure of RSV F (FIG. 3).

Selected VL22_66K clamps were incubated at 4, 25 and 40° C. for 72 hr to assess thermal stability. Clamp antigens were incubated at a concentration of 100 μg/ml at the respective temperatures and then analysed by SE-HPLC using a Waters column (XBridge BEH450 7.8×300 mm) and Waters guard column (XBridge Guard BDH450 7.8×30 mm) using a DPBS mobile phase (FIG. 4). Analysis of the 5 clamp antigens showed that VL22_66K_CD10 and VL22_66K_CD11 formed the most stable trimers and were the least likely to aggregate following incubation in the range of temperatures assessed (FIG. 4). Thermal stability of VL22_66K_CD10 & VL22_66K_CD11 incubated at 4 and 25° C. for 72 hours was similar to the F HIV-clamp.

### Example 6: Expression, Purification and Analysis of Clamp Variants when Appended to VL22-66E

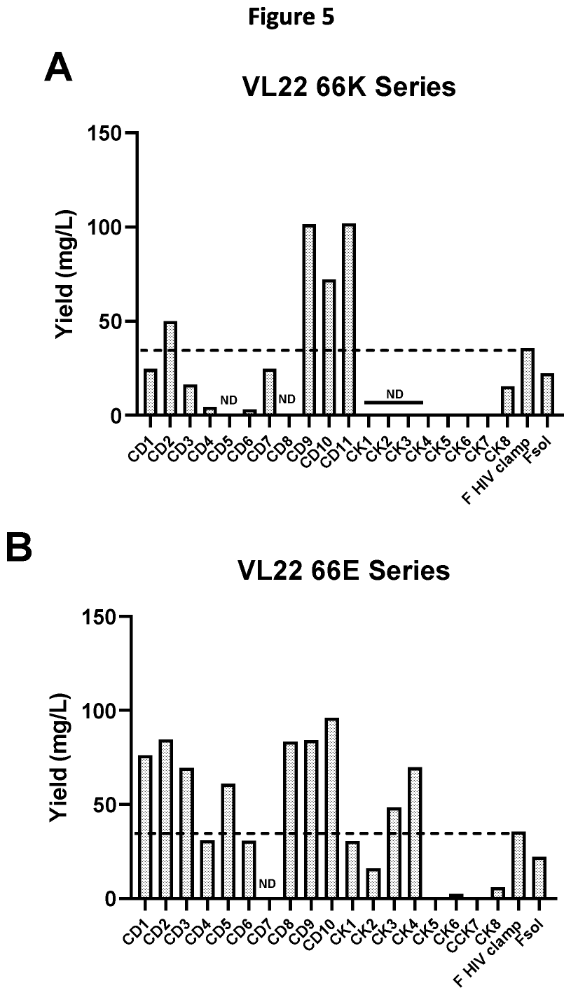

Constructs encoding the clamp series appended to VL22_66E were then expressed in ExpiCHO cells (as per Example 2) and purified by FPLC (as per Example 4). Yields of the VL22_66E clamp antigens were calculated by NanoDrop™ One microvolume UV-Vis spectrophotometry (Thermofisher Scientific) and are compared with the results for the VL22_66K clamp antigens in FIG. 5. Yields of VL22_CD9, 10 and 11 variants were high for both the VL22_66K and VL22_66E antigen series. CD9 was excluded at this point due to it being similar in sequence to CD11 and resulting in slightly more aggregation (as shown in FIG. 4). Hence CD10 and CD11 were selected as preferred leads.

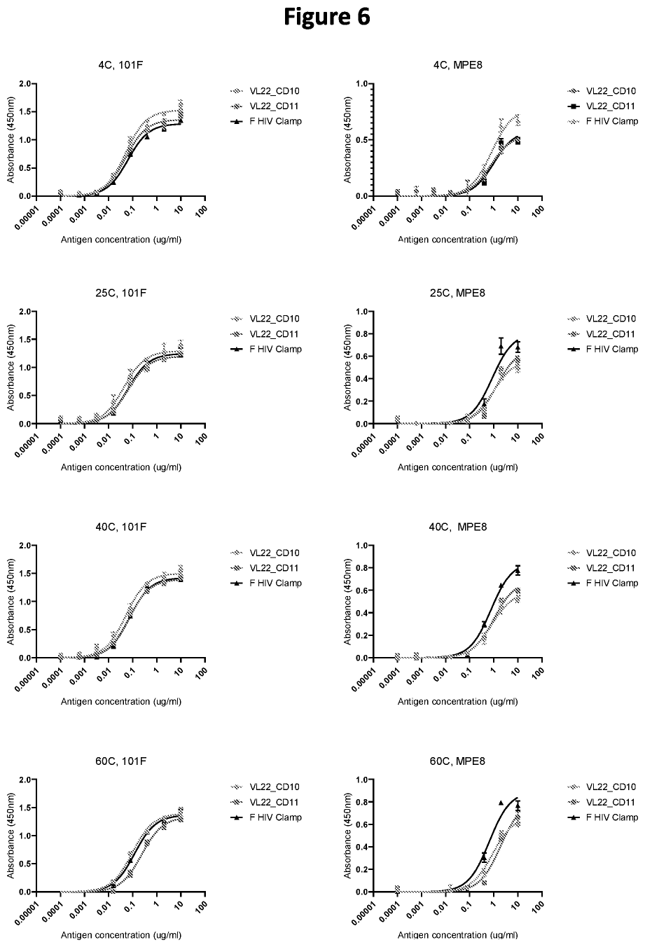

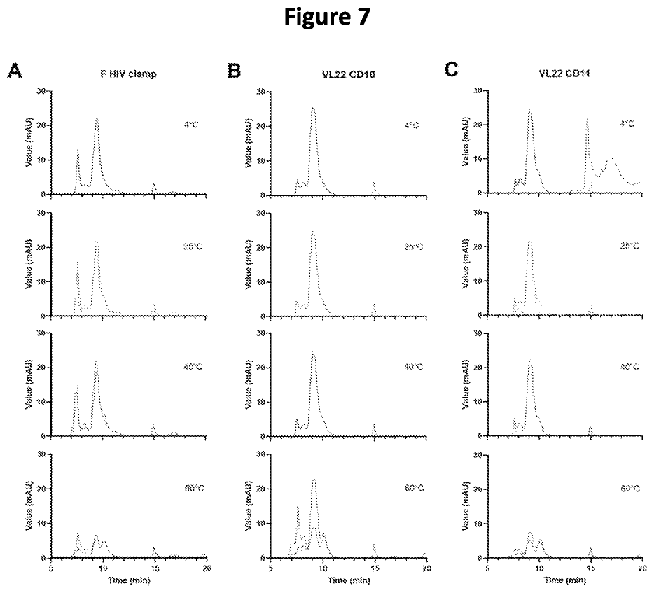

The stability of VL22_66E_CD10 and VL22_66E_CD11 antigens was examined over a wide temperature range. Stability was assessed using ELISA to characterise binding of RSV-specific antibodies 101F and MPE8 as per Example 3 (FIG. 6) and SE-HPLC to determine monodispersity and oligomeric state as per Example 5 (FIG. 7). Prior to analysis by ELISA and SE-HPLC, clamp-antigens were incubated for 72 h at 4, 25, 40 or 60° C. at 100 μg/ml in PBS. Both the VL22_66E_CD10 and VL22_66E_CD11 antigens were found to be highly stable, retain 101F- and MPE8-binding activity after 72 hours at temperatures up to 60° C., and maintaining a stable trimeric state over a range of temperatures. These two lead candidates are referred to as VL22_CD10 and VL22_CD11 in the following examples and figures for simplicity. Both VL22_CD10 and VL22_CD11 were less prone to aggregation than the F HIV-clamp control (FIG. 7) following incubation in temperatures up to 40° C. However, stability of all three clamp antigens began to exhibit altered trimerization stability following incubation at 60° C. for 72 h.

### Example 7: Characterisation of CD11

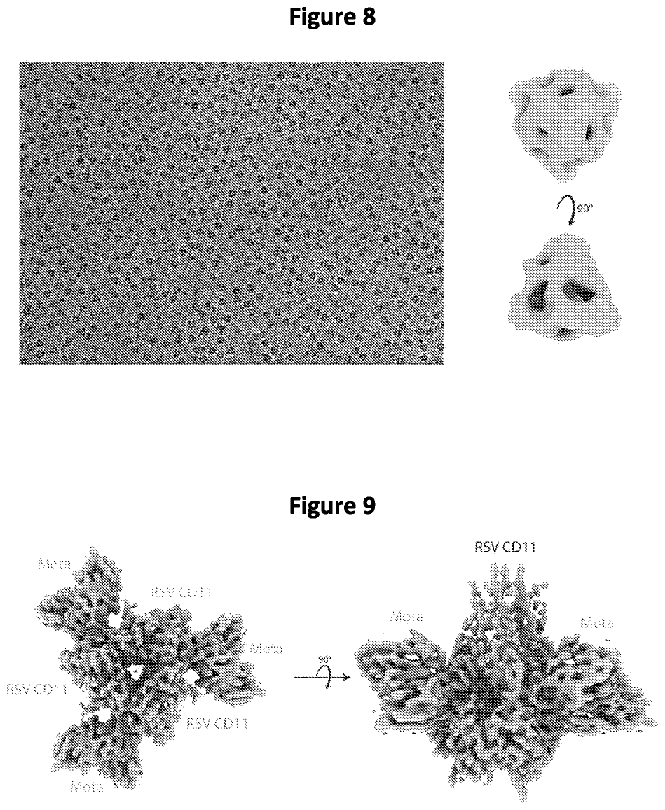

A near-atomic three-dimensional model of VL22_CD11 was generated by single particle analysis (SPA) cryogenic electron microscopy. Purified VL22_CD11 or VL22_CD11 complexed with Fab were adsorbed onto glow-discharged quantifoil grids and plunge frozen using an EMGP2 system (Leica, Mount Waverley, VIC, Australia). Grids were imaged on a CRYO ARM 300 (JEOL, Tokyo, Japan) equipped with a K3 detector (Gatan, Pleasanton, CA, USA). Image processing and SPA was performed in cryoSPARCAs seen in FIG. 8, the data shows the VL22_CD11 trimer adopts a structure resembling a pyramid as opposed to the lollipop-like structure that is characteristic of post-fusion F, but a high-resolution structure was not obtainable. To overcome any orientation bias, which might account for the difficulty obtaining a high-resolution structure, a second structure of VL22_CD11 was determined in complex with Motavizumab Fab (McLellan et al., Nat Struct Mol Biol, 2010, DOI: 10.1038/nsmb.1723).

The motavizumab Fab complexed with VL22_CD11 improved orientation bias and provided a structural landmark for model interpretation. A 3.8 Å map of the complex was determined and is shown in FIG. 9. While, some key pre-fusion specific antibody binding sites, Ø and V, are not well resolved in the structure, it is also clear that hallmarks of the post-fusion structure, such as the characteristic 6-helix bundle are absent. Poor resolution for sites, 0 and V is indicative of a high level of flexibility within poorly resolved regions, that is beyond that described for DS-Cav1 Foldon (McLellan et al., Science 2013 doi: 10.1126/science.1243283). It is hypothesized by the inventors that the antigen adopts a more native and flexible conformation at these sites due to the absence of non-native structure stabilising mutations present in DS-Cav1 Foldon. In addition, the clamp density was not resolved so its structure was not clearly defined. It is hypothesised that the lack of a single, stable conformation of the clamp within the structure is due to the flexible linker (more specifically referred to herein as “hinge”) between the C-terminus of the F ectodomain and the N-terminus of clamp CD11 resulting in inconsistent spatial orientation of the clamp relative to the ectodomain antigen, as opposed to flexibility within the clamp itself.

### Example 8: Assessment of Interference with HIV Diagnostic Assay

Development of a SARS-CoV-2 vaccine utilising an HIV-derived trimerization domain was discontinued as the vaccine induced HIV specific antibodies that resulted in diagnostic interference in HIV testing (Chappell, et al., Lancet Infect Dis, 2021, DOI: 10.1016/51473-3099(21)00200-0). To dispel any concerns that CD10 and CD11 could also interfere with common HIV diagnostic tests, the CD10 and CD11 clamps were tested in a series of assays.

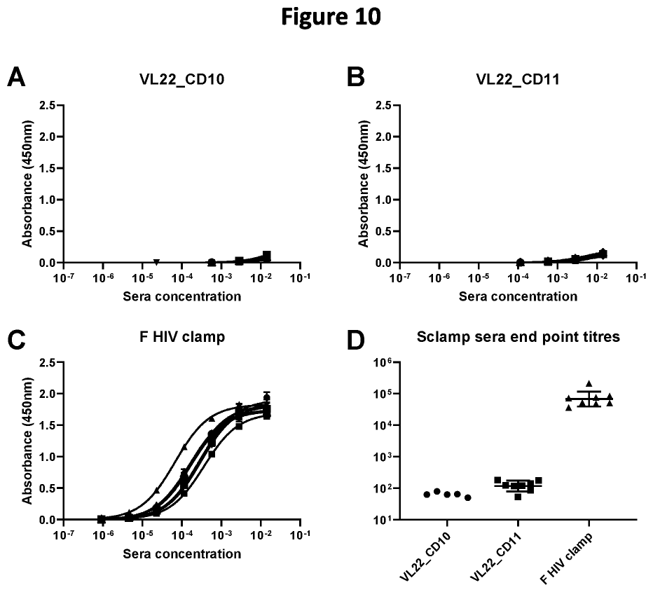

As an initial assessment, mice were immunized twice 3 weeks apart with 5 μg a SARS-Cov-2 Spike (S) ectodomain stabilised with the HIV-clamp (SARS-Cov-2 S HIV-clamp, SEQ ID NO: 28; Watterson, et al., Clin Transl Immunology, 2021, DOI: 10.1002/cti2.1269), and serum collected 3 weeks after the second immunisation. ELISA plates were coated with the F HIV-clamp, VL22_CD10 or VL22_CD11. The results, shown in FIG. 10, demonstrate that the response elicited in mice by the F HIV-clamp exhibits ˜1000 fold less reactivity against the non-HIV derived CD10 and CD11 clamps.

A SARS-Cov-2 Spike protein stabilised with the CD11, SARS-CoV-2 S CD11 (SEQ ID NO: 29), was produced as per methods described in examples 1, 2 and 4, except that Spike specific antibody CR3022 (Tian et al., 2020, DOI: 10.1080/22221751.2020.1729069) was used for immunoaffinity chromatography together with elution at pH 3.0.

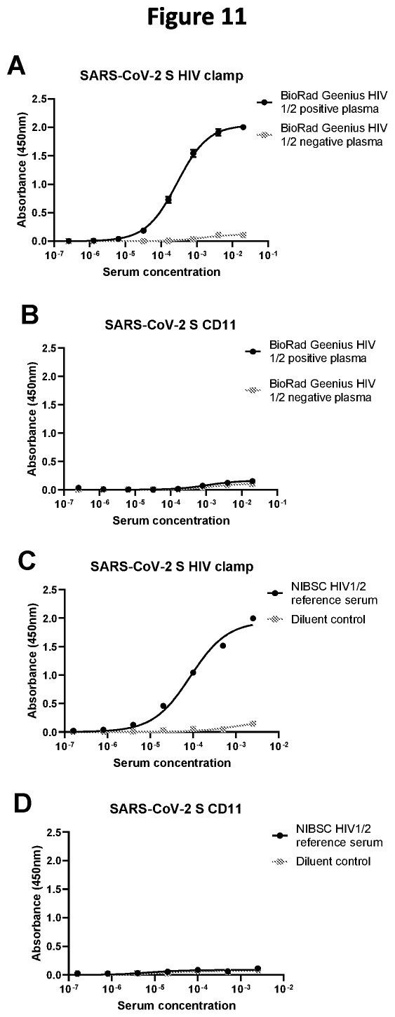

Binding of HIV standard human plasma or serum to CD11 was assessed using BioRad Geenius HIV 1/2 control plasma and NIBSC International HIV reference serum standard. SARS-CoV-2 S HIV-clamp and SARS-CoV-2 Spike-CD11 antigens were used in place of RSV antigens as RSV antibodies are ubiquitous in human plasma/serum. The NIBSC international standard panel included vials of human sera raised against HIV-1 subtypes A, B, C and E; HIV-1 group 0; and HIV-2. Equal volumes of the individual standards were pooled for testing in ELISA. SARS-CoV-2 S HIV-clamp and SARS-CoV-2 S CD11 were coated on ELISA plates, and serial dilutions of the BioRad Geenius HIV 1/2 control plasma and NIBSC International HIV reference serum analysed by ELISA (FIG. 11). Both HIV positive controls reacted strongly with SARS-CoV-2 S HIV-clamp but not with SARS-CoV-2 S CD11, indicating the absence of cross-reactivity between CD11 and HIV gp41.

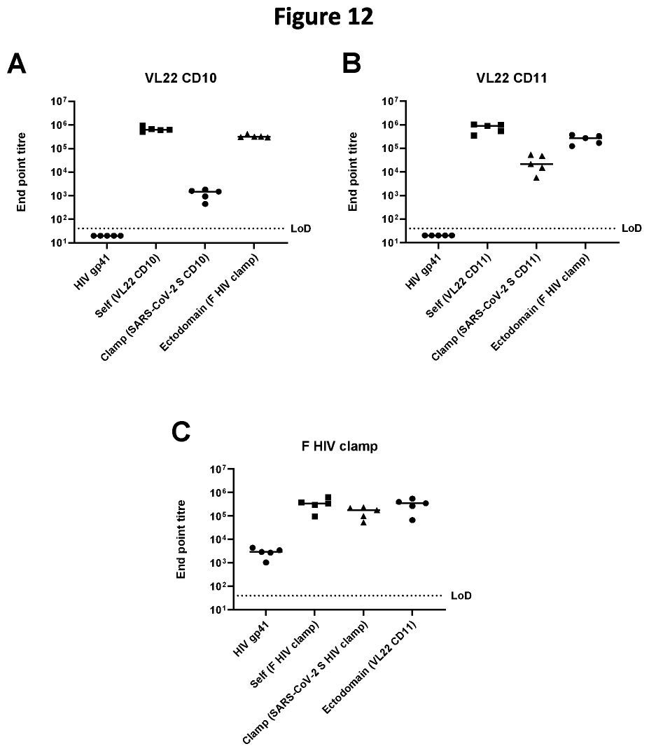

To further assess potential for HIV diagnostic cross reactivity, groups of five mice were immunised twice, three weeks apart, with each dose consisting of 25 μl AddaVax (Invivogen) squalene-based oil-in-water nano-emulsion adjuvant and 5 μg of VL22_CD10, VL22_CD11 or F HIV-clamp in 25 μl of PBS pH 7.4. A group of three mice was included as a negative control. Serum collected from mice 3 weeks after the second immunisation was tested by ELISA against the antigen HIV-1 gp41 (#ab49070, Abcam, Cambridge, United Kingdom), which is commonly used in HIV testing and the antigen used for immunisation. In addition, sera were tested for binding to the clamp (using a SARS-CoV-2 S HIV-clamp, SARS-CoV-2 S CD10 and SARS-CoV-2 S CD11) and for binding to the ectodomain (using an RSV with a mismatched clamp domain). The results, shown in FIG. 12, indicate that sera generated to the CD10 and CD11 clamps does not bind HIV-1 gp41, while sera generated to the HIV-clamp does. Additionally, the immune response elicited to CD10 and CD11 appear to be lower than HIV-clamp.

### Example 9: Analysis of the Ability of VL22_CD10 and VL22_CD11 to Induce Neutralising Antibodies in Mice

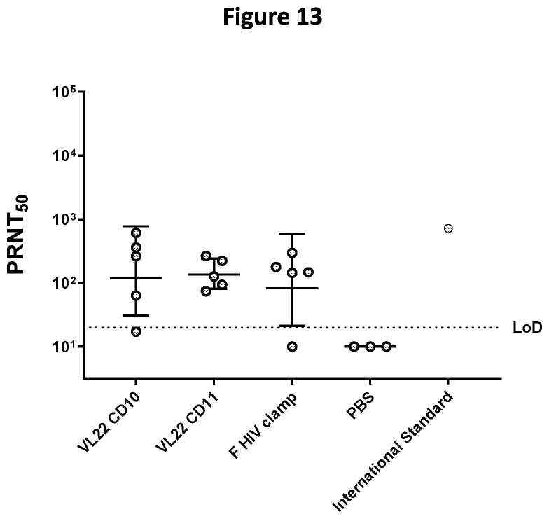

Terminal sera from mice immunised in Example 8 were tested for the present of neutralising antibodies against RSV A2 by Plaque Reduction Neutralization Test (PRNT) as per methods outlined in Isaacs et al., Viruses 2021 DOI: 10.3390/v13101942. Due to the small group sizes, it is not possible to make statistically relevant comparisons between the new clamps and the control F HIV-clamp. However, the results of the PRNT, shown in FIG. 13, indicate that the RSV F antigens stabilised with the CD10 and CD11 clamps were able to induce neutralising antibodies in mice.

### Example 10: Assessment of Candidate Clamps with Other Viral Proteins

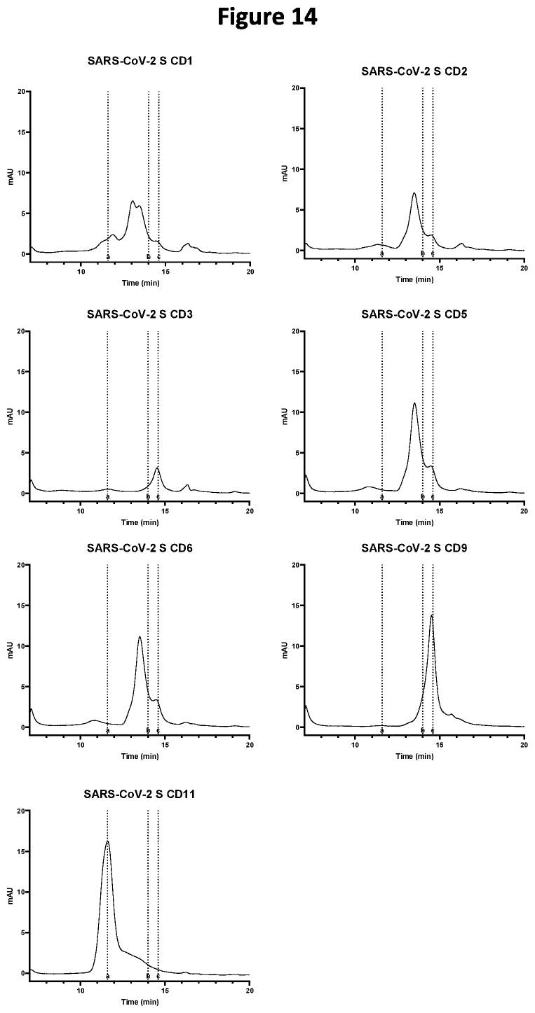

Eight of the candidate clamp moieties were assessed for their ability to stabilise the SARS-CoV-2 Spike protein (S). CD1, CD2, CD3, CD5, CD6, CD9, CD10, and CD11 clamp sequences were appended to the C-terminal end of SARS-CoV-2 S in place of the HIV-clamp sequence (SEQ ID NO: 28) using the molecular cloning methods as described in Example 1, and clamp-antigens were expressed and purified as per Examples 2 and 4. Following purification, monodispersity and oligomeric state of antigens were analysed via SE-HPLC (FIG. 14). Only SARS-CoV-2 S CD11 was found to form a species consistent with the trimeric protein (molecular weight ˜600 kDa) The calculated molecular weight of all other clamp-antigens (CD1, CD2, CD3, CD5, CD6, CD9, CD10) was consistent with monomeric protein (˜200 kDa) or degraded protein.

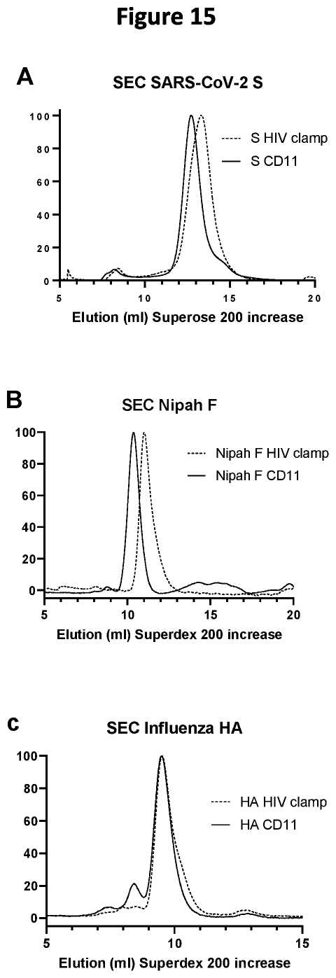

The molecular species of SARS-CoV-2 S CD11 (SEQ ID NO: 29) was assessed by size exclusion chromatography (SEC) column Superose 6 Increase 10/300GL (Cytiva) for comparison to the same construct with the HIV-clamp (FIG. 15A). The two constructs are shown to be highly comparable with the calculated molecular weight of the primary species of both clamp-antigens consistent with a trimer (˜600 kDa).

The HIV-clamp and the CD11 clamp were appended to the C-terminus of Nipah virus Fusion protein (F) (SEQ ID NO: 30 and 31) as per Examples 1, 2, and 4. Specific antibody mAb66 (Avanzato et al., 2019, DOI: 10.1073/pnas.1912503116) was used for immunoaffinity chromatography. The two constructs were analysed by SEC on a Superdex 200 Increase 10/300GL (Cytiva). The two constructs are shown to be highly comparable with the calculated molecular weight of the primary species of both clamp-antigens consistent with a trimer (˜200-250 kDa) (FIG. 15B).

The HIV-clamp and the CD11 clamp were appended to the C-terminal of Influenza A (strain H1N1pdm California09) hemagglutinin protein (HA) (SEQ ID NO: 32 and 33) as per Examples 1, 2, and 4, and specific antibody 5J8 (Krause et al., 2019, DOI: 10.1128/JVI.00700-11) was used for immunoaffinity chromatography. The two constructs were analysed by SEC on a Superdex 200 Increase 10/300GL (Cytiva). The two constructs are shown to be highly comparable with as the calculated molecular weight of the primary species of both clamp-antigens consistent a trimer (˜250 kDa) (FIG. 15C).

### Example 11: Comparison of CD11 and HIV-Clamp Stabilised Viral Antigens to Elicit a Neutralising Immune Response Upon Vaccination in Mice

Groups of Balb/c mice (n=8) were immunised with either PBS placebo or the purified antigens; SARS-CoV-2 Spike, Nipah F and Influenza HA stabilised with either the HIV-clamp or CD11. Two intramuscular injections were given 3 weeks apart with each dose consisting of 25 μl AddaVax (Invivogen) squalene-based oil-in-water nano-emulsion adjuvant and 5 μg purified protein antigen in 25 μl of PBS pH 7.4. Total volume delivered was 50 μl. Three weeks following administration of the second dose mice were culled and blood collected by cardiac puncture. Virus neutralisation activity of blood serum was quantified against SARS-CoV-2 or Influenza A H1N1pdm California09 by plaque reduction neutralisation testing as per published methodology (Amarilla et al., Front Microbiol. 2021, DOI: 10.3389/fmicb.2021.625136 and McMillan et al., NPJ Vaccines. 2021, DOI: 10.1038/s41541-021-00395-4) or against Nipah virus pseudovirus as per published methodology (Isaacs et al., Viruses 2021 DOI: 10.3390/v13101942).

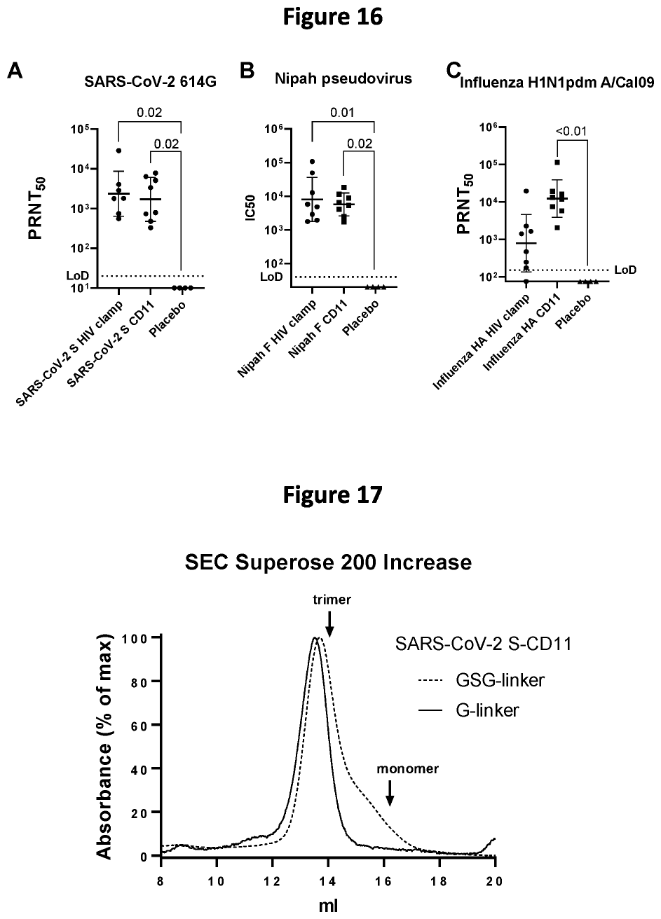

Virus neutralisation against SARS-COV-2 and Nipah virus was found to be equivalent for the respective antigens containing HIV-clamp and CD11 stabilisation domains (FIG. 16A/B). Virus neutralisation against Influenza virus was enhanced by the inclusion of CD11 relative to the HIV-clamp (FIG. 16C).

### Example 12: Modification of Hinge Region Improved Trimer Stabilisation

Analysis of the CD11-appended SARS-CoV-2 Spike (S) antigen showed that a small fraction of the protein formed a species with calculated molecular weight indicative of monomer. The SARS-CoV-2 S CD11 antigen was modified by shortening the “hinge” region between the C-terminus of SARS-CoV-2 S and the N-terminus of CD11 from ‘GSG’ to ‘G’ to generate antigen SARS-CoV-2 Spike_CD11_SG (SEQ ID NO: 34). Analysis of this shortened antigen showed that the clamp-antigen forms a single species characteristic of trimer (FIG. 17).

### Example 13: Generation and Analysis of Immune-Silenced CD11 Clamp Variants

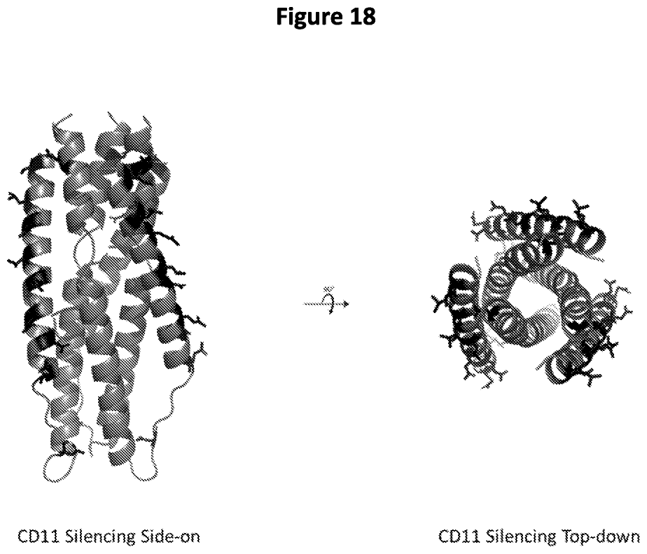

Immune responses to VL22_CD11 are directed both at the RSV F ectodomain (VL22) and to the CD11 clamp sequence. To reduce the immune response to the clamp sequence, additional glycosylation sites were introduced into the CD11 sequence at sites identified in FIG. 18 to create ‘silenced’ variants. Plasmids encoding 15 CD11 ‘silenced’ variants (SEQ ID NOs: 35-49; FIG. 19) appended to VL22 were generated as per Example 1 and proteins expressed in ExpiCHO cells and purified as per Example 2 and 4. Purified ‘silenced’ variants were analysed by ELISA to measure binding of the antibodies 101F and MPE8.

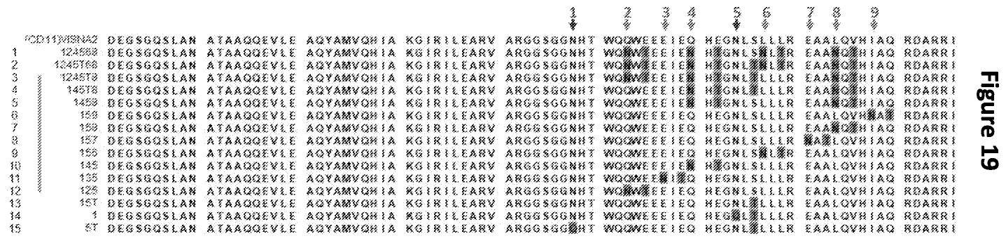

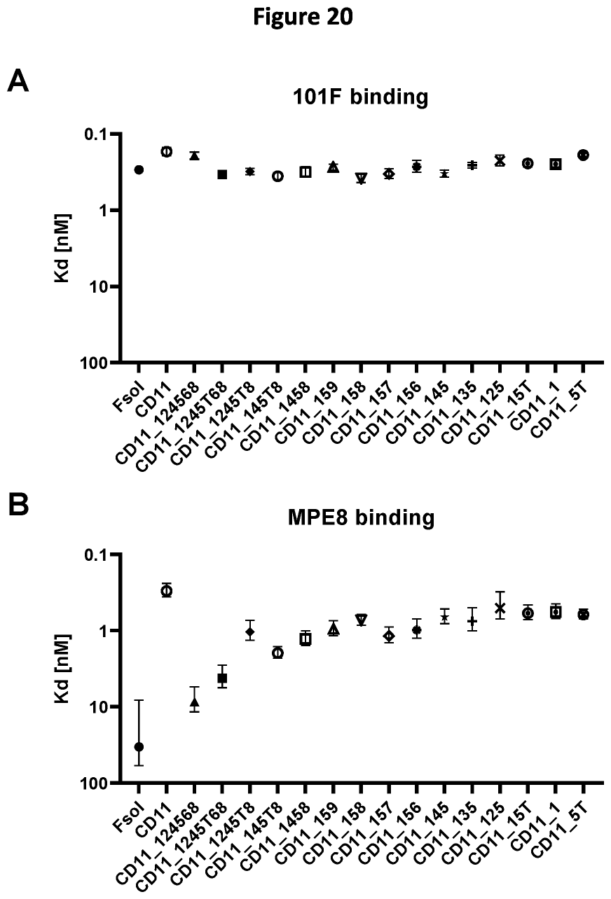

All of the silenced VL22_CD11 variants bound 101F. The first two constructs, VL22_CD11_124568 and 1245T68, showed a greater than 10-fold reduction in binding to MPE8 relative to CD11, however all other silenced variants retained low nM binding of MPE8 (FIG. 20).

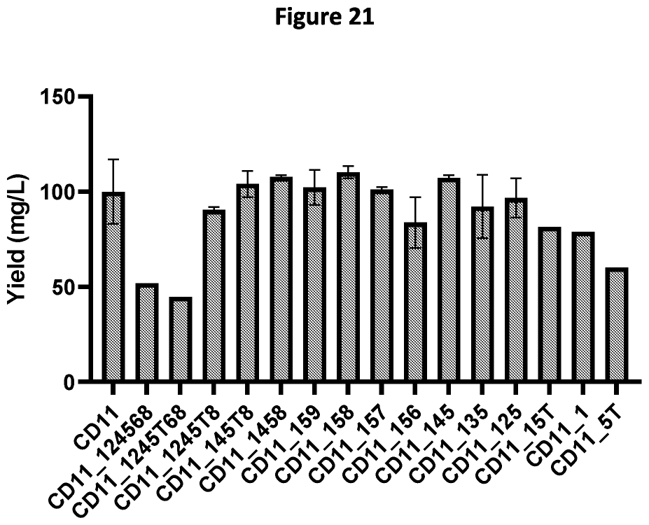

Most of the silenced VL22_CD11 series gave similar yields to VL22_CD11 (FIG. 21), with the yields of only VL22_CD11_5T, 124568 and 1245T68 reduced by roughly 50%.

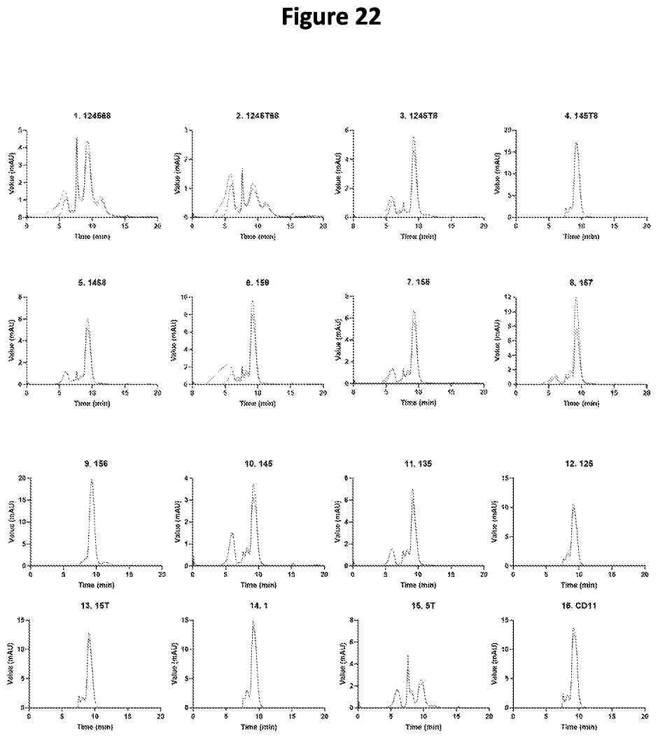

All 15 of the silenced CD11 antigens were analysed by SE-HPLC. The traces are shown in FIG. 22. Silenced clamp antigens VL22_CD11_1245T8, 145T8, 1458, 156, 158, 157, 156, 145, 135, 125, 15T, and 1 all produced favourable traces with minimal aggregate formation and a well-defined peak representative of trimer.

Silenced clamp antigens that displayed significantly reduced MPE8 binding and/or less favourable SE-HPLC traces (CD11_124568, 1245T68 and 5T) were not pursued further. The two constructs which had the fewest glycosylation sites (CD11_15T, and 1) were also not pursued further.

To further distinguish between the variants, a thermostability analysis of constructs VL22_CD11_1245T8, 145T8, 1458, 156, 158, 157, 156, 145, 135, and 125 (indicated by the grey bar in FIG. 19) was carried out. The antigens were incubated in PBS at 4 or 40° C. for 1 week and then analysed by SE-HPLC. The resulting traces are in FIG. 23 and indicate that all of the silenced CD11 variants analysed were stable under the tested conditions.

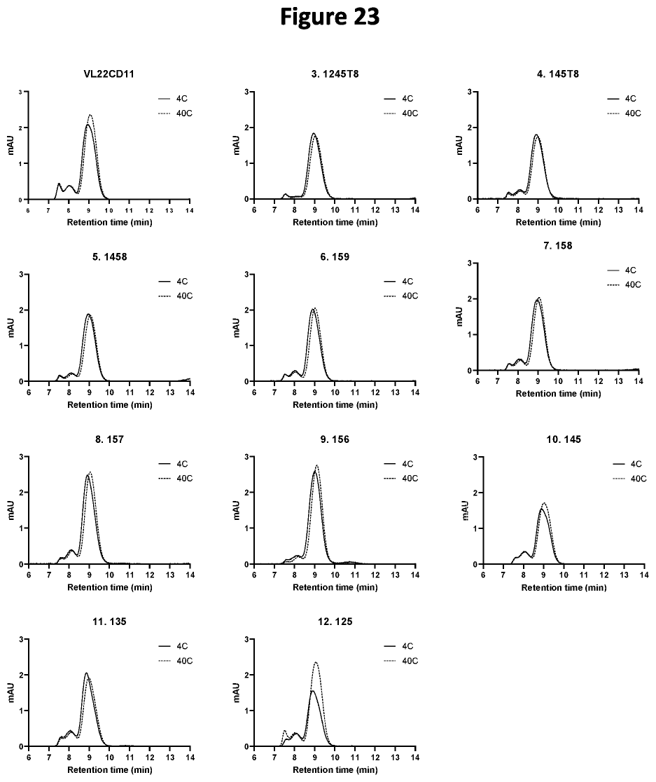

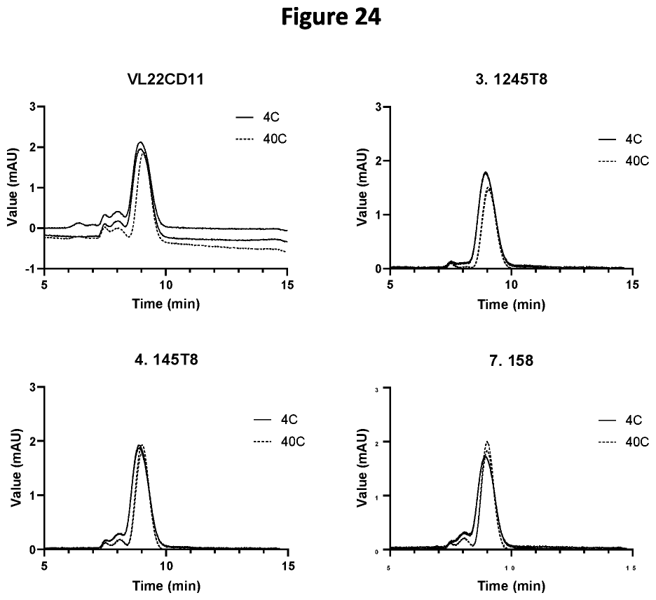

As all of the tested antigens (VL22_CD11_1245T8, 145T8, 1458, 156, 158, 157, 156, 145, 135, and 125) proved to be stable after one week at 40° C., the two with the most glycosylation sites (VL22_CD11_1245T8 and CD11_145T8) were selected for further analysis. VL22_CD11_158 and the original VL22_CD11 were also analysed further for thermal stability. All four antigens were found to be stable after incubation at 40° C. for 38 days (FIG. 24).

### Example 14: Glycoform Analysis Of ‘Silenced’ CD11 Clamped Proteins

The two theoretically most glycosylated variants that had good SE-FPLC traces, MPE8 binding and yield data, VL22_CD11_145T8 and VL22_CD11_1245T8, were chosen as leads. The occupancy of the glycosylation sites was examined for the two leads and unsilenced VL22_CD11.

Two separate digests (trypsin and Glu-C) were performed on each protein and one aliquot of each of the digests were subjected to treatment with PNGase F in ¹⁸O water. Peptides, glycopeptides, and de-glycosylated peptides were measured with LC-MS/MS. For the PNGase F treated samples, deamidation and incorporation of ¹⁸O at Asn in N-linked sequons was identified using Proteome Discoverer and site-specific N-glycosylation occupancy was quantified using an in-house script.

The use of ¹⁸O water for N-glycosylation site mapping can potentially introduce analytical artefacts, including incorporation of ¹⁸O into the C-terminus of peptides if residual protease activity occurs and contamination from unlabelled species due to improperly dried samples, exposure to air or reincorporation of ¹⁶O after the samples have been re-suspended in water for analyses. The samples that were prepared without ¹⁸O water and PNGase F treatment were used to validate ¹⁸O occupancy data. Glycoforms were identified with Byonic™ and occupancy was calculated using published software and scripts3. Sites that could be reliably measured (containing only one N-linked site) were used to confirm ¹⁸O occupancy data.

Another potential caveat is deglycosylation inefficiency due to reduced enzyme activity of PNGase F after drying and reconstitution in ¹⁸O water. To investigate this, the data obtained from the PNGase F treated samples was additionally processed by the above glycoform data analysis pipeline to confirm complete or near-complete deglycosylation. Finally, in measuring site-specific N-glycosylation occupancy, it is critical that the sites of ¹⁸O incorporation within a peptide are confidently assigned. This is especially important for peptides that contain multiple, closely spaced NxS/T N-glycosylation sequons. The inventors therefore manually validated non-redundant spectral matches on peptides with multiple N-glycosylation sequons, paying careful attention to those within a few amino acid residues of each other.

The location and sequence of N-glycosylation sequons in the clamp of each protein is shown in FIG. 25A. High occupancy was observed at the sites of RSV F (N27, N70 and N461, FIG. 25B). High occupancy was also observed at CD11 sites N480 and N518 in unmodified CD11 as well as VL22_CD11_145T8 and VL22_CD11_1245T8. Low occupancy was observed at site N534 in unmodified CD11, High occupancy was observed at N534 in VL22_CD11_145T8 and 1245T8, where Serine 537 was substituted with Threonine. Site N523, which is present in protein 1245T8 only, had low occupancy and site N530, which is present in proteins 145T8 and 1245T8 only, had low/no occupancy.

In summary, each of the lead silenced clamps (CD11_1245T8 and 145T8) had high occupancy at four sites within CD11 compared to the two occupied in unmodified CD11. Each of the two lead silenced clamps are likely to have very similar properties. Variants with more glycosylation sites than these had a higher proportion of aggregate to trimer in the SE-HPLC trace and binding of MPE8 was impacted.

### Example 15: Immunogenicity of New ‘Silenced’ Clamp Leads and Controls

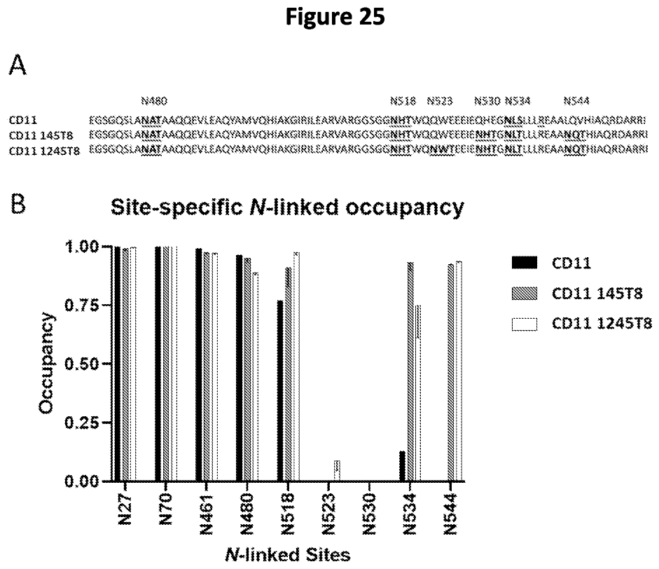

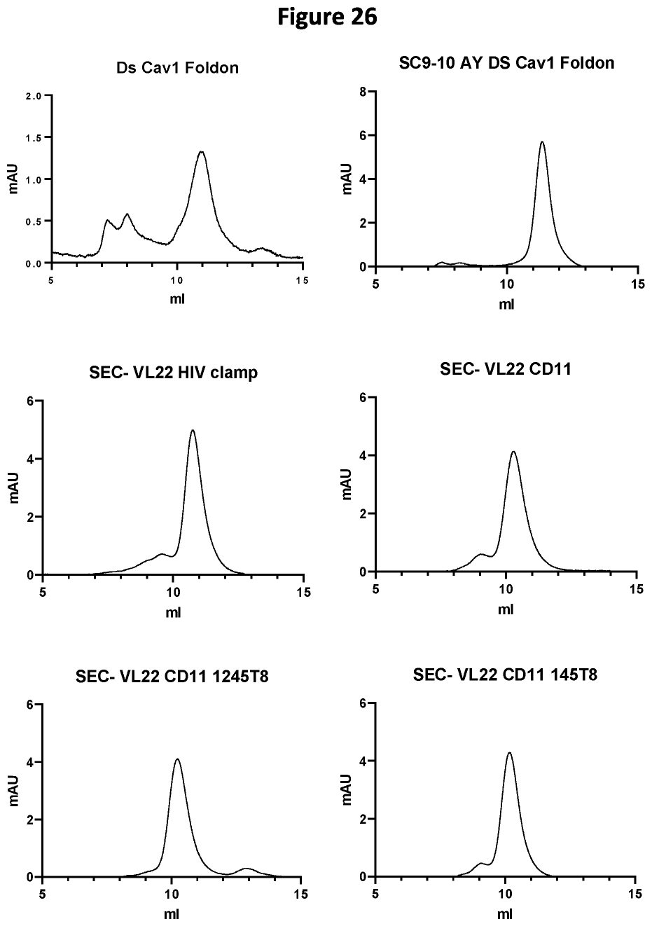

To enable direct comparison with the VL22_CD11 (SEQ ID NO: 24), VL22_CD11_1245T8 (SEQ ID NO: 50) and VL22_CD11_145T8 (SEQ. ID NO: 51), the RSV F ectodomain sequence (VL22) was appended to the HIV-clamp via the cloning methods described in Example 1 to produce antigen F HIV-clamp (SEQ ID NO: 52). Best in class control antigens described in the literature DS-Cav1 Foldon (McLellan et al., Science 2013 doi: 10.1126/science.1243283; SEQ ID NO: 53) and SC9-10 DS-Cav AY Foldon (Joyce et al., Nat Struct Mol Biol 2016 DOI: 10.1038/nsmb.3267; SEQ ID NO: 54) were also cloned as per the methods described in Example 1. DS-Cav1 Foldon, SC9-10 DS-Cav1 AY Foldon, F HIV-clamp, VL22_CD11, VL22_CD11_1245T8 and VL22_CD11_145T8 were expressed and purified as per Examples 2 and 4. SEC analysis showed that there was minimal aggregate formation in all of the antigen preparations, with the exception of DS-Cav1 Foldon (FIG. 26).

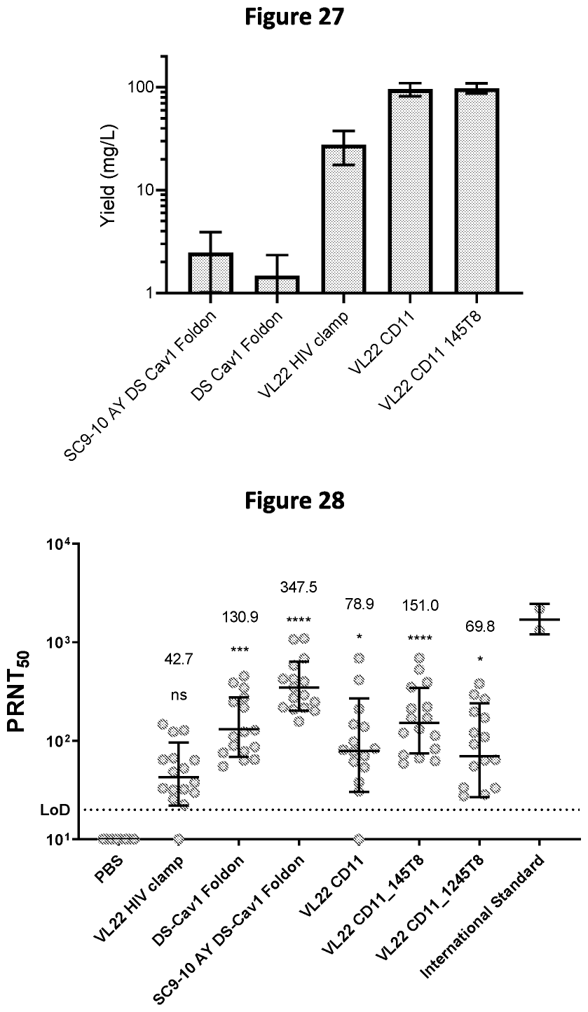

The yield of each respective antigen obtained from 3-5 separate purification runs was assessed. The yield obtained for DS-Cav1 Foldon and SC9-10 AY DS-Cav1 Foldon was consistently 10- to 50-fold lower in comparison to VL22 stabilised with either the HIV-clamp, CD11 or CD11_145T8 (FIG. 27). In addition, the VL22 incorporating either CD11 or CD11_145T8 produced at consistently higher yields than the F HIV-clamp.

Groups of 16 Balb/c mice were immunised twice, three weeks apart, with 1 μg of antigen mixed with Addavax adjuvant. A negative control PBS group of 8 mice was also included. Serum was collected from the mice three weeks after first immunisation by terminal bleed. PRNT analysis of terminal serum samples indicated that VL22_CD11 and the two silenced CD11 antigens all gave rise to strong neutralising responses in naïve mice (FIG. 28). The amplitudes of the responses were similar to those for DS-Cav1 Foldon. SC9-10 DS-Cav1 AY Foldon, the optimised DS-Cav Foldon comparator, performed significantly better than all of the antigens except for VL22_CD11_145T8. Therefore, the lead antigen VL22_CD11_145T8 is potentially comparable to SC9-10 DS-Cav1 AY Foldon in its ability to induce a neutralising response.

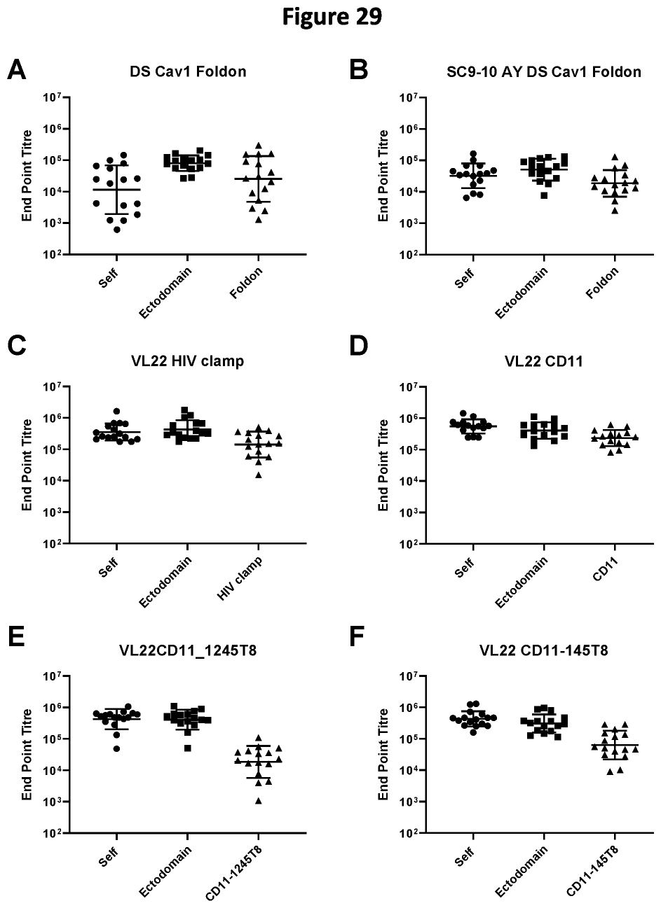

In order to determine the relative antibody responses directed towards the antigen ectodomain and stabilisation domains included in different antigens, Nipah F ectodomain containing Foldon, CD11_1245T8 and CD11_145T8 (SEQ ID NOs: 55-57), were produced as per methods in Example 1, 2 and 4 for use in ELISA. Serum collected from the terminal bleeds in a mouse immunisation study was then assessed for binding to the antigen used for immunisation (self), antigen contain the RSV F ectodomain with an alternate (stabilisation domain (CD11 or HIV-clamp) or the Nipah F antigen containing the identical stabilisation domain (Foldon, HIV-clamp, CD11, CD11_145T8 or CD11_1245T8). Endpoint titres were calculated for each serum sample and the geometric mean for each group calculated (FIG. 29). Response to Foldon stabilisation domain was 3.2- and 2.7-fold lower than the ectodomain response for DS Cav1 Foldon and SC9-10 AY DS-Cav1 Foldon, respectively. The response to HIV-clamp was 3.0-fold lower than the ectodomain response and the response to CD11 was 1.7-fold lower. Responses to the ‘silenced’ CD11_1245T8 and CD11_145T8 were 22.1-fold and 4.9-fold lower than the ectodomain confirming that the relative antibody response to the stabilisation domain had been reduced through the incorporation of additional N-linked glycosylation sites.

### Example 16: Generation of Improved CD11 Constructs

An overlay of the crystal structures available for the HIV-1 gp41 six-helical bundle (from which the HIV clamp is derived) and the Visna six-helical bundle (from which the CD11 clamp is derived) (Protein Data Bank (PDB) entries 6KTS and 1JEK, respectively) was used to guide selection of potential mutations to increase stability.

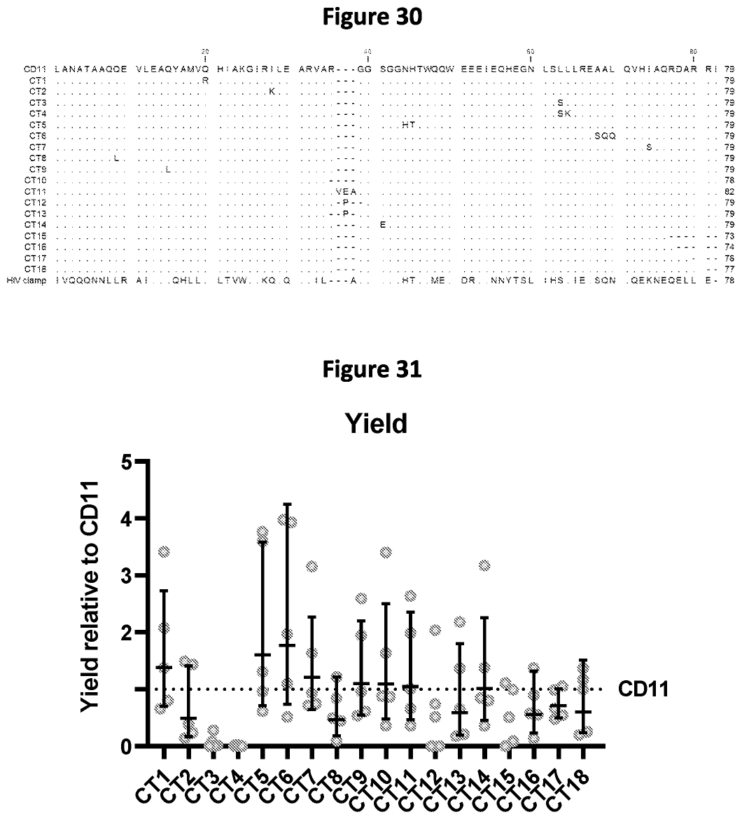

A panel of site-directed changes were designed with the objective to maximise stability by either (i) increasing charged interactions, (ii) increasing hydrophobic interactions, (iii) incorporation of changes to linker region (between FHRR and SHRR) to enhance engagement between FHRR and SHIRR, or (iv) change to C-terminus of SHIRR (see CT1 to CT18 (SEQ ID NOs: 55-72) in Table 5; FIG. 30).

TABLE 5
Structure-based changes to CD11

| | SEQ | | | |
|---|---|---|---|---|
| CD11 | ID | | | |
| Change | NO: | Mutation | Sequence | Notes |
| CT1 | 55 | Q22R | QSLANATAAQQEVLEAQYAMVRHIAKGIRILEARVARGGSGGN | Increasing |
| | | | HTWQQWEEEIEQHEGNLSLLLREAALQVHIAQRDARRI | charged |
| CT2 | 56 | 130K | QSLANATAAQQEVLEAQYAMVQHIAKGIRKLEARVARGGSGG | interactions |
| | | | NHTWQQWEEEIEQHEGNLSLLLREAALQVHIAQRDARRI | |
| CT3 | 57 | L62S | QSLANATAAQQEVLEAQYAMVQHIAKGIRILEARVARGGSGGN | |
| | | | HTWQQWEEEIEQHEGNLSSLLREAALQVHIAQRDARRI | |
| CT4 | 58 | L62S | QSLANATAAQQEVLEAQYAMVQHIAKGIRILEARVARGGSGGN | |
| | | L63K | HTWQQWEEEIEQHEGNLSSKLREAALQVHIAQRDARRI | |
| CT5 | 59 | N43H | QSLANATAAQQEVLEAQYAMVQHIAKGIRILEARVARGGSGGH | |
| | | H44T | TTWQQWEEEIEQHEGNLSLLLREAALQVHIAQRDARRI | |
| CT6 | 60 | A67S | QSLANATAAQQEVLEAQYAMVQHIAKGIRILEARVARGGSGGN | |
| | | A68Q | HTWQQWEEEIEQHEGNLSLLLRESQQQVHIAQRDARRI | |
| | | L69Q | | |
| CT7 | 61 | 173S | QSLANATAAQQEVLEAQYAMVQHIAKGIRILEARVARGGSGGN | |
| | | | HTWQQWEEEIEQHEGNLSLLLREAALQVHSAQRDARRI | |
| CT8 | 62 | Q11L | QSLANATAAQLEVLEAQYAMVQHIAKGIRILEARVARGGSGGN | Increasing |
| | | | HTWQQWEEEIEQHEGNLSLLLREAALQVHIAQRDARRI | hydrophobic |
| CT9 | 63 | Q17L | QSLANATAAQQEVLEALYAMVQHIAKGIRILEARVARGGSGGN | interactions |
| | | | HTWQQWEEEIEQHEGNLSLLLREAALQVHIAQRDARRI | |
| CT10 | 64 | R37del | QSLANATAAQQEVLEAQYAMVQHIAKGIRILEARVAGGSGGN | Change to |
| | | | HTWQQWEEEIEQHEGNLSLLLREAALQVHIAQRDARRI | linker region |
| CT11 | 65 | 38insVEA | QSLANATAAQQEVLEAQYAMVQHIAKGIRILEARVARVEAGGS | to enhance |
| | | | GGNHTWQQWEEEIEQHEGNLSLLLREAALQVHIAQRDARRI | engagement |
| CT12 | 66 | G38P | QSLANATAAQQEVLEAQYAMVQHIAKGIRILEARVARPGSGGN | between |
| | | | HTWQQWEEEIEQHEGNLSLLLREAALQVHIAQRDARRI | FHRR and |
| CT13 | 67 | R37P | QSLANATAAQQEVLEAQYAMVQHIAKGIRILEARVAPGGSGGN | SHRR |
| | | | HTWQQWEEEIEQHEGNLSLLLREAALQVHIAQRDARRI | |
| CT14 | 68 | S40E | QSLANATAAQQEVLEAQYAMVQHIAKGIRILEARVARGGEGG | |
| | | | NHTWQQWEEEIEQHEGNLSLLLREAALQVHIAQRDARRI | |
| CT15 | 69 | Q75stop | QSLANATAAQQEVLEAQYAMVQHIAKGIRILEARVARGGSGGN | Change to |
| | | | HTWQQWEEEIEQHEGNLSLLLREAALQVHIAQ | C-terminus |
| CT16 | 70 | R76stop | QSLANATAAQQEVLEAQYAMVQHIAKGIRILEARVARGGSGGN | of SHRR |
| | | | HTWQQWEEEIEQHEGNLSLLLREAALQVHIAQR | |
| CT17 | 71 | A78stop | QSLANATAAQQEVLEAQYAMVQHIAKGIRILEARVARGGSGGN | |
| | | | HTWQQWEEEIEQHEGNLSLLLREAALQVHIAQRDA | |
| CT18 | 72 | R79stop | QSLANATAAQQEVLEAQYAMVQHIAKGIRILEARVARGGSGGN | |
| | | | HTWQQWEEEIEQHEGNLSLLLREAALQVHIAQRDAR | |

Each of the 18 clamp variants (CT1-CT18) was cloned C-terminal to two different SARS-CoV-2 Spike proteins from different virus strains: SARS-CoV-2 Sand SARS-Cov-2 (Delta) S to yield SARS-CoV-2 S CD11 (SEQ ID NO: 29), SARS-CoV-2 (Delta) S CD11 (SEQ ID NO: 73), and SARS-CoV-2 (Delta) CD11-QS (SEQ ID NO: 74). The latter construct (“-QS”) included a modification at the region linking the C-terminus of spike and the N-terminus of CD11 as demonstrated in Example 12, however amino acids ‘QS’ were deleted from the N-terminus of the CD11 FHRR as opposed to amino acids ‘SG’, which correspond to the flexible linker.

Expression level was assessed via ELISA for all 3 antigens and via Nanodrop post purification for the two delta panels. All 5 measures of expression level are presented in Table 6 as relative to unmodified CD11 in FIG. 31.

TABLE 6
Protein yield estimates for SARS-CoV-2 antigens with changes to CD11

| | ELISA-based estimate of | | | Nanodrop-based estimate of antigen | |
|---|---|---|---|---|---|
| | antigen expression (mg/L) | | | expression (mg/L) post-purification | |
| | | SARS-CoV-2 | SARS-CoV-2 | SARS-CoV-2 | SARS-CoV-2 |
| | SARS-CoV-2 | (Delta) S | (Delta) S | (Delta) S | (Delta) S |
| | S CD11 | CD11 | CD11-QS | CD11 | CD11-QS |
| Control | 2.72 | 5.682 | 5.41 | 1.45 | 1.66 |
| CT1 | 1.784 | 19.4 | 11.22 | 1.992 | 1.328 |
| CT2 | 0.3953 | 8.466 | 7.76 | 0.34 | 0.65 |
| CT3 | 0 | 0.03475 | 0.47 | 0.02 | 0.46 |
| CT4 | 0 | 0.03791 | 0.07 | 0.04 | 0 |
| CT5 | 1.673 | 21.41 | 19.45 | 1.9 | 1.6 |
| CT6 | 1.395 | 22.33 | 21.52 | 1.6 | 3.27 |
| CT7 | 1.945 | 9.292 | 17.08 | 1.08 | 1.56 |
| CT8 | 1.214 | 2.797 | 6.56 | 0.144 | 1.41 |
| CT9 | 1.459 | 14.73 | 10.53 | 1.4 | 1.01 |
| CT10 | 0.9958 | 9.28 | 18.41 | 1.26 | 1.46 |
| CT11 | 2.747 | 11.29 | 14.29 | 0.52 | 1.104 |
| CT12 | 1.397 | 11.57 | 0 | 1.08 | 0 |
| CT13 | 0.5675 | 7.778 | 11.8 | 0.255 | 1.088 |
| CT14 | 0.9803 | 7.848 | 17.16 | 1.23 | 1.332 |
| CT15 | 0.2432 | 5.64 | 6.007 | 0.74 | 0 |
| CT16 | 0.3807 | 5.08 | 7.424 | 0.8 | 0.96 |
| CT17 | 1.297 | 5.61 | 5.718 | 0.79 | 1.104 |
| CT18 | 0.6896 | 5.66 | 1.071 | 1.7 | 2.276 |

Based on the expression level eight candidates (CT1, CT5, CT6, CT7, CT9, CT10, CT14, and CT18) were progressed into analysis via SEC for both the SARS-Cov-2 (Delta) S CD11 and CD11-QS panels. Two additional candidates (CT12 and CT16) were progressed for only the SARS-Cov-2 (Delta) S CD11 panel and three additional candidates (CT3, CT8, and CT17) for only the SARS-Cov-2 (Delta) S CD11-QS panel. Five candidates were excluded at this stage (CT2, CT4, CT11, CT13, and CT15).

Purified proteins were analysed by SEC using a Superose 6 Increase 10/300GL column (Cytiva). The relative amounts of trimer, high molecular weight (HMW) aggregated product and low molecular weight (LMW) product were assessed by calculating area under the curve (AUC) for the SEC trace for each panel (FIG. 32). Control SARS-Cov-2 (Delta) S CD11 displayed a propensity to dissociate into monomers. For this panel there was substantial improvement in trimer presence for CT1, CT5, CT6, CT9, CT10 and CT12. For control SARS-Cov-2 (Delta) S CD11-QS less monomer was present, however substantial aggregate (40%) was present. Improvements in terms of decreased aggregation were not observed. It was evident however that some CT candidates resulted in substantial transition to monomer. CT3, CT8, CT18 were excluded based on high presence of monomer.

From SEC analysis of the two CT panels, candidates were narrowed to five; CT1, CT5, CT6, CT9 and CT10. While other CT candidates (CT7, CT12, CT14, CT16 and CT17) did not appear substantially worse than the control CD11, there was little evidence to suggest any relative improvement. These five candidates (CT1, CT5, CT6, CT9 and CT10) were progressed into an accelerated thermal stability analysis (40° C. incubation for 48 hr, FIG. 33). For the SARS-Cov-2 (Delta) S CD11 panel all 5 modifications showed improvement relative to the control, with CT6 showing the most promise. For the SARS-Cov-2 (Delta) S CD1-QS panel, CT1, CT6 and CT10 showed moderate improvement, however CT9 showed some dissociation into monomer.

### Example 17: Analysis of RSV VL22_CD11 Modifications

Based on results obtained in Example 16, three CD11 modifications (CT1, CT5 and CT9; Table 6) were selected for incorporation into the lead RSV candidates VL22_CD11 (SEQ ID NO: 24). These CD11 modifications (as well as an additional modification, CT6) were also incorporated into RSV candidate VL22_CD11-QS (SEQ ID NO: 75), in which the two amino acids ‘QS’ were removed from the N-terminus of CD11, as per Example 16. An additional silenced CD11 antigen VL22_CD11_145T8-QS (SEQ ID NO: 76) in which the two amino acids ‘QS’ were removed from the N-terminus of CD11, and the three modifications (CT1, CT5 and CT9) were also incorporated into VL22_CD11 145T8-QS (SEQ ID NOs: 77-79). All constructs were cloned as per the methods described in Example 1 and proteins expressed and purified as per Examples 3 and 4.

Purified proteins were analysed by SEC using a Superdex 200 Increase 10/300GL (Cytiva). SEC analysis was conducted either immediately following purification (T=0) or following storage for 19 days at 25° C. or 40° C. The relative amounts of trimer, high-molecular-weight (HMW)-aggregated product, and low-molecular weight (LMW)-product were assessed by calculating area under the curve (AUC) for the SEC trace for each panel (Table 7).

Control VL22_CD11 was predominantly soluble trimer with approximately 15.5% HMW aggregate. There was little change in the soluble trimer following incubation at elevated temperature with only a modest increase in aggregated product from 15.5% to 17%. Some modified constructs displayed reduced aggregate presence at time 0, in particular VL22_CD11CT1 (13.6% aggregate), VL22CD11-QSCT1 (12.8% aggregate) and VL22_CD11-QS_CT6 (8.1% aggregate).

Of the antigens that included additional glycosylation sites introduced into the CD11, VL22_CD11_145T8-QS_CT5 and VL22_CD11_145T8-QSCT9 had the highest percentage of soluble trimer at time 0; 91.5% and 92.5%, respectively. At elevated temperature incubation trimer percentage dropped significantly for VL22_CD11_145T8-QSCT9 (92.5% to 84.2%), while VL22_CD11145T8-QS_CT5 percentage only showed a modest drop in trimer percentage (91.5% to 88.6%).

TABLE 7
AUC analysis of SEC for RSV VL22_CD11 modifications

| Antigen Name | SEQ ID NO: | Storage | HMW | Trimer | LMW |
|---|---|---|---|---|---|
| VL22_CD11 | 24 | T = 0 | 15.5 | 84.5 | 0 |
| | | 19 days @ 25° C. | 15.4 | 84.6 | 0 |
| | | 19 days @ 40° C. | 17 | 83 | 0 |
| VL22_CD11_CT1 | 24 + 55 | T = 0 | 13.6 | 86.4 | 0 |
| | | 19 days @ 25 C. | 15.4 | 84.6 | 0 |
| | | 19 days @ 40 C. | 15.8 | 84.2 | 0 |
| VL22_CD11_CT5 | 24 + 59 | T = 0 | 21.5 | 78.5 | 0 |
| | | 19 days @ 25 C. | 21.2 | 78.8 | 0 |
| | | 19 days @ 40 C. | 19.8 | 80.2 | 0 |
| VL22_CD11_CT9 | 24 + 63 | T = 0 | 22 | 78 | 0 |
| | | 19 days @ 25 C. | 21.8 | 78.2 | 0 |
| | | 19 days @ 40 C. | 23.8 | 76.2 | 0 |
| VL22_CD11-QS_CT1 | 75 + 55 | T = 0 | 12.8 | 87.2 | 0 |
| | | 19 days @ 25 C. | 11.7 | 81.4 | 4 |
| | | 19 days @ 40 C. | 13.2 | 86.8 | 0 |
| VL22_CD11-QS_CT5 | 75 + 59 | T = 0 | 21.5 | 78.5 | 0 |
| | | 19 days @ 25 C. | 18.6 | 81.4 | 0 |
| | | 19 days @ 40 C. | 18.7 | 81.3 | 0 |
| VL22_CD11-QS_CT6 | 75 + 60 | T = 0 | 8.1 | 91.9 | 0 |
| | | 19 days @ 25 C. | 9.7 | 90.3 | 0 |
| | | 19 days @ 40 C. | 10.9 | 89.1 | 0 |
| VL22_CD11-QS_CT9 | 75 + 63 | T = 0 | 18.2 | 81.8 | 0 |
| | | 19 days @ 25 C. | 18.6 | 81.4 | 0 |
| | | 19 days @ 40 C. | 19.3 | 80.7 | 0 |
| VL22_CD11_145T8-QS_CT1 | 77 | T = 0 | 3.6 | 87.7 | 8.7 |
| | | 19 days @ 25 C. | 3 | 88.7 | 8.3 |
| | | 19 days @ 40 C. | 5.6 | 86.4 | 8 |
| VL22_CD11_145T8-QS_CT5 | 78 | T = 0 | 5.1 | 91.5 | 3.4 |
| | | 19 days @ 25 C. | 3.6 | 93.5 | 2.9 |
| | | 19 days @ 40 C. | 7.6 | 88.6 | 3.8 |
| VL22_CD11_145T8-QS_CT9 | 79 | T = 0 | 7.5 | 92.5 | 0 |
| | | 19 days @ 25 C. | 11 | 89 | 0 |
| | | 19 days @ 40 C. | 12.4 | 84.2 | 3.4 |

### Example 18: Combining Multiple Clamp Vaccines into a Single Administration

The goal of this stream was to evaluate whether clamp vaccines developed for different respiratory pathogens could be combined and administered to provide an immune response to multiple pathogens. RSV F antigen containing lead unsilenced Clamp2 (VL22 Clamp2; SEQ ID NO: 75); and SARS-CoV-2 Spike antigen also containing lead unsilenced Clamp2 (SARS2-S Clamp2; SEQ ID NO: 34) were either stored as separate monovalent preparations or as a mixture of the two antigens into a single bivalent formulation. All formulations were prepared in PBS pH 7.4 and stored 4° C.

Antigen integrity was then assessed via antigen specific ELISA at the time of mixing or following storage at 4° C. for 6 weeks. Relative avidity (kD) of pre-fusion RSV F specific mAb D25 was sub-nanomolar for VL22 Clamp2 alone and for VL22 Clamp2+SARS2-S Clamp2 both at time 0 and after incubation at 4° C. for 6 weeks (FIG. 34A). Similarly, the relative avidity (kD) of SARS-CoV-2 Spike Receptor Binding Domain (RBD) specific mAb CR3022 was sub-nanomolar for SARS2-S Clamp2 at time 0 and only showed a relatively small loss in avidity after incubation at 4° C. for 6 weeks, which was comparable for SARS2-S Clamp2 alone and for VL22 Clamp2+SARS2-S Clamp2 (FIG. 34B).

Groups of Balb/c mice (n=6 or n=8) were immunised twice, three weeks apart, via IM injection with either 50 μl of PBS (placebo), or 50 μl total volume comprised of 25 μl AddaVax (Invivogen) squalene-based oil-in-water nano-emulsion adjuvant and 25 μl of PBS containing either 1 μg of VL22 Clamp2, 1 μg of SARS2-S Clamp2, or 1 μg of VL22 Clamp2+1 μg of SARS2-S Clamp2. Monovalent (VL22 Clamp2 and SARS2-S Clamp2) and bivalent (VL22 Clamp2+SARS2-S Clamp2) formulations were incubated at 4° C. for 3 weeks prior to administration of the first dose and for 6 weeks prior to administration of the second dose. A further 3 weeks following administration of the second dose, mice were culled, and blood collected by cardiac puncture.

Virus neutralisation activity of blood serum was quantified against RSV (A2 strain) and SARS-CoV-2 (Prototypic strain) by plaque reduction neutralisation testing (PRNT) as per published methodology (Amarilla et al., Front Microbiol. 2021 DOI: 10.3389/fmicb.2021.625136 and Isaacs et al., Viruses 2021 DOI: 10.3390/v13101942). Virus neutralisation elicited against RSV was found to be equivalent whether mice received 1 μg of VL22 Clamp2 or 1 μg of VL22 Clamp2 and 1 μg of SARS2-S Clamp2 (FIG. 35A). Similarly, virus neutralisation elicited against SARS-CoV-2 was found to be equivalent whether mice received 1 μg of SARS2-S Clamp2 or 1 μg of VL22 Clamp2 and 1 μg of SARS2-S Clamp2 (FIG. 35B).

The results obtained in this example therefore demonstrate that at least two clamp stabilized antigens can be combined into a single formulation without affecting the stability of either antigen or interfering with the neutralising immune response elicited to either antigen.

### Example 19: Applicability to Trimeric Bacterial Autotransporters

Bacterial autotransporters, also commonly known as trimeric autotransporter adhesins (TAAs), are a class of surface localised trimeric proteins found on many Gram-negative bacteria. Examples include Neisseria meningitidis adhesin A (NadA), Neisseria meningitidis hia/hsf homologue (NhhA), E. coli autotransporter G (EhaG), E. coli IgG-binding protein D (EibD), Uropathogenic E. coli autotransporter G (UpaG), Haemophilus influenzae adhesin (HiA) and Yersinia enterocolitica adhesin (YadA). Such proteins are important virulence factors and can mediate adherence to host cells and extracellular matrix proteins as well as biofilm formation.

Bacterial autotransporters are trimeric and therefore well suited to expression and purification using the molecular clamp platform and use as vaccines given their importance as virulence factors for medically important bacteria. The genes encoding the bacterial autotransporters, NadA, NhhA, EhaG, EibD, UpaG, HiA and YadA, were PCR amplified and cloned in-frame with the VISNA-based SSM CD11 sequence (SEQ ID NOs: 218-224) as a 3′-extension into the E. coli expression vector pSU2718. Competent E. coli cells were transformed with the generated expression vectors as well as the empty pSU2718 vector as a negative control. For expression, cells were grown at 37° C. in LB supplemented with chloramphenicol (30 μg/ml) to an optical density at 600 nm (OD₆₀₀ₙₘ) equal to 0.5, and expression was induced by the addition of 0.1 mM IPTG prior to harvesting and pelleting of cells by centrifugation.

Recombinant E. coli cell pellets were resuspended in PBS pH 7.4, 10% sucrose, 1 mM EDTA, 10 μg/ml lysozyme and 10 μl DNase/100 ml at a volume of 5 ml/g wet pellet. Cells were then lysed by sonication and cell debris was removed by centrifugation. Cell lysates were examined by SDS-PAGE (FIG. 36A). Of the seven CD11-stabilised bacterial autotransporters, three (EhaG, HiA, and UpaG) were clearly visible relative to the background E. coli proteins present in the negative control. For more sensitive analysis of protein expression, Western Blotting was performed with a VISNA-based SSM CD11 specific monoclonal antibody previously identified by Phage panning (FIG. 36B). In the western blot analysis, all seven CD11-stabilised bacterial autotransporters were detected at the expected molecular weight.

The three best-expressed CD11-stabilized bacterial autotransporters (EhaG, HiA, and UpaG) were purified via gravity flow chromatography using a VISNA-based SSM CD11 specific affinity resin (AVI-8740) produced by Avitide Pty Ltd. The resin was initially equilibrated with PBS pH7.4. A volume of 5 ml of each recombinant E. coli cell lysate was then added to approximately 1 ml of resin and incubated for 1 hr at room temperature. Unbound protein, flow through, was then collected and column was washed 3 times with 10 ml of PBS pH 7.4. Bound protein was then recovered by elution with sodium acetate pH3.0. Cell lysate, flow through, wash 1, wash 3 and recovered protein were then assessed by SDS-PAGE, which confirmed successful purification of each bacterial autotransporter (FIG. 37).

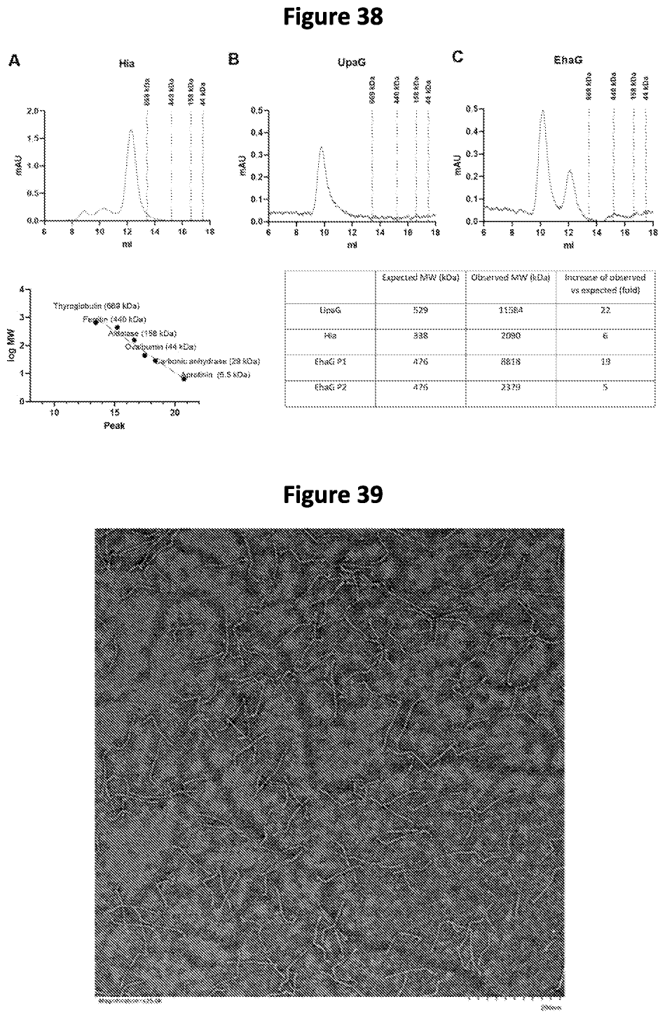

Purified bacterial autotransporters were then analysed by size exclusion chromatography on Superose 6 Increase 10/300GL (Cytiva). The absorbance trace for HiA-CD11 and UpaG-CD11 showed a single peak indicative of a homogeneous protein (FIG. 38A/B), whereas EhaG-CD11 showed two distinct peaks indicative of two distinct species (FIG. 38C). The MW estimates based on size exclusion chromatography were substantially higher than expected, which may be due to the long filamentous proteins running aberrantly on size exclusion chromatography. The higher-than-expected MW is not thought to be due to protein aggregate formation as no protein was detected in the void volume, which is around 7 ml.

To further analyse the purified bacterial autotransporter UpaG-CD11, negative stain Transmission Electron Microscopy was utilized. Long filamentous proteins of between 100-150 nm in length were observed, consistent with the expected length of UpaG (Bassler et al., IJMM 2015 DOI: 10.1016/j.ijmm.2014.12.010) (FIG. 39).

The results obtained in this example demonstrate that by appending the VISNA-based SSM CD11 sequence to the C-terminus of bacterial autotransporters (or certain portions thereof), such proteins can be expressed in E. coli and purified from recombinant E. coli cell lysates. The results further demonstrate that these proteins adopt the expected quaternary structure consisting of a long filamentous trimeric protein and therefore would be expected to elicit an immune response following vaccination that would recognise such filaments on the surface of Gram-negative bacteria.

### Example 20: Delivery Via LNP/mRNA Vaccine

The aim of the mRNA POC study was to demonstrate that the clamp technology can be applied to mRNA-based vaccines. To do this, mRNA encoding a RSV F Clamp2 lead was to be designed and synthesised; a transfection study carried out to confirm that correctly folded protein is produced from the mRNA; and finally the mRNA formulated into lipid nanoparticles and tested for immunogenicity in mice.

Custom mRNA was prepared by Trilink Pty Ltd using their own proprietary methods. The sequence of the open reading frame (ORF) of VL22 Clamp2 (SEQ ID NO: 75) was provided to Trilink, cloned into their standard vector and mRNA synthesis carried out by in vitro transcription (IVT). The Trilink Cleancap, N1-methylpseudouridine, and a poly A tail were incorporated into the mRNA. Generated mRNA was then encapsulated into Lipid Nanoparticles (LNPs) by a standard methodology. LNPs were stored at 4° C. for 4 days post-formulation and then frozen and stored at −20° C. until use.

Groups of Balb/c mice (n=8) were immunised twice, three weeks apart, via IM injection with either 50 μl of Tris buffered saline (TBS) placebo, 50 μl TBS containing either 0.4, 2 or 10 μg of LNP encapsulated mRNA, or protein vaccine control (50 μl total volume comprised of 25 μl AddaVax (Invivogen)). Three weeks following administration of the second dose mice were culled and blood collected by cardiac puncture.

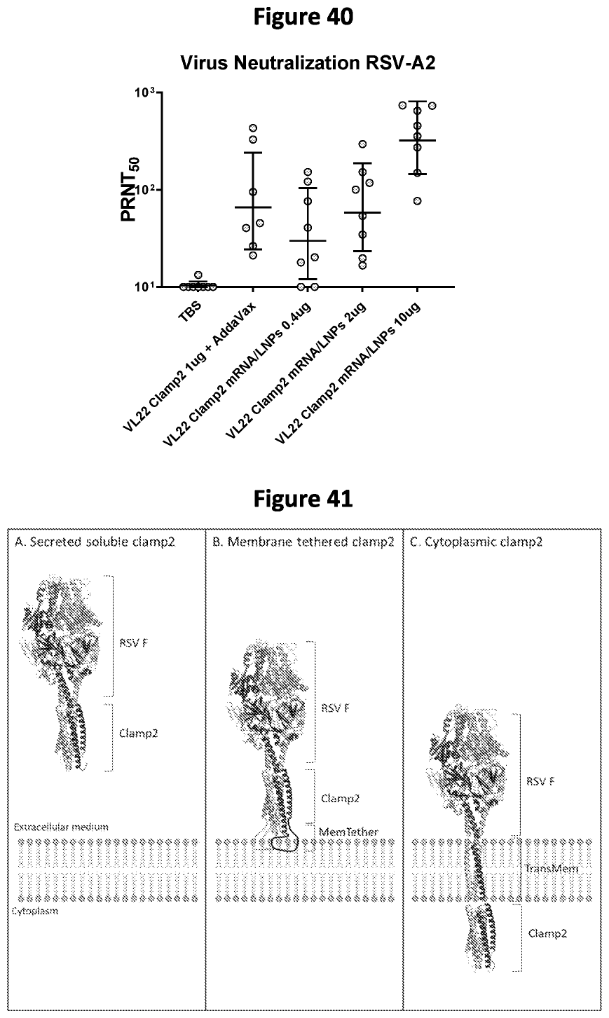

Virus neutralisation activity of blood serum was quantified against RSV (A2 strain) by PRNT as per published methodology (Isaacs et al., Viruses 2021 DOI: 10.3390/v13101942). All doses of mRNA/LNP also gave measurable neutralising immune responses to RSV A2 (FIG. 40). The 2 μg mRNA/LNP dose gave a response of similar amplitude to the 1 μg VL22 Clamp2 protein/adjuvant control while the amplitude of the response to the 10 μg mRNA/LNP dose was increased relative to the protein control.

The results obtained in this example therefore demonstrate that molecular clamp stabilized antigens are compatible with delivery as LNP encapsulated mRNA vaccines.

### Example 21: Development of a Membrane Tethered Clamp Sequence for Use in mRNA Vaccines

The antigen coding sequence investigated in Example 20 as an LNP/mRNA vaccine encoded the soluble Clamp2 stabilised antigen. As this antigen does not include any membrane spanning regions, following administration, the translated protein sequence would be expected to transit through the secretory system and be exported into the extracellular medium (FIG. 41A). This may be beneficial for vaccination in some circumstances as the soluble antigen may have a longer half-life within the body and/or could be more easily transported to draining lymph nodes to stimulate a potent adaptive immune response. Alternatively, for vaccination in other circumstances, an antigen which remains tethered to the cellular surface may be advantageous as the cells and/or cell-derived organelles expressing and consequently displaying high concentration and/or high density of chimeric polypeptide may help to recruit and activate naïve and memory B cells and thereby enhance the stimulation of a potent B-cell mediated neutralizing antibody response.

To enable a vaccine design that can combine the benefits of molecular clamp prefusion stabilisation with presentation at the target cell surface, the inventors sought to design a membrane tethered molecular clamp. Two potential designs were envisaged: inclusion of a membrane tethering (poly)peptide sequence within the flexible linker between the FHRR and SHRR of the Clamp2 or Clamp2s sequence (FIG. 41B) or inclusion of the Clamp2 sequence following the native transmembrane region of a viral class 1 fusion protein so that the clamp2 will be positioned on the cytoplasmic side of the plasma membrane (FIG. 41C).

Based on the structural features of the herein described molecular clamp, a membrane tethering (poly)peptide (as envisaged in FIG. 41B) should presumably enter and leave from the same surface of the lipid bilayer. Additionally, it may be also advantageous if the membrane tethering (poly)peptide itself had the potential to trimerize and if the N- and C-termini were spatially separated in a way that matches with the corresponding ends of the Clamp2/Clamp2s FHRR and SHRR sequences.

Upon searching of the structural depository, the inventors were able to identify a panel of candidate molecules with regions matching properties that were desired. The panel of identified candidates included, Campylobacter concisus phosphoglycosyl transferase C (PgIC) (PDB: 5W7L), chicken acid-sensing ion channels 1a (ASIC1a) (PDB: SWKV), human N-arachidonoylethanolamine (AEA) (PDB: 3QJ9), human ionotropic P2X receptor (PDB: 2A9H), Streptomyces lividans, K⁺-channel KcsA (PDB: 2A9H), Thermotoga maritima CorA (PDB: 2|UB), Bacilus subtilis Membrane-integrating sequence for translation of IM protein constructs (Mistic) (PDB: 1YGM). An additional candidate was designed in silica to adopt the desired properties. The candidate sequences have been ascribed the following nomenclature: alpha, beta, gamma, delta, epsilon, eta, neta, and theta. These sequences and their respective structures are shown in FIG. 42.

Each candidate membrane tethering sequence was cloned within the RSV F antigen containing lead silenced Clamp2s (VL22 Clamp2s; SEQ ID NO: 78) within the flexible linker between the Clamp2s FHRR and SHRR. The amino acid sequences from each construct of the generated panel are listed below (SEQ ID NOs: 225-232).

An additional candidate was produced in which the Clamp2 sequence is included following the native transmembrane region so that the VL22 antigen is located on the extracellular side of the plasma membrane and clamp2 will be positioned on the cytoplasmic side, as per FIG. 41C; native transmembrane sequence of RSV F was inserted into VL22 Clamp2s, as per FIG. 41C (SEQ ID NO: 233). Full-length native RSV F sequence (SEQ ID NO: 234) was included as a positive control for membrane anchoring. Lastly, another control construct was produced based on the previously described pre-fusion stabilized antigen (Joyce et al., 2016; Nat Struct Mol Biol DOI: 10.1038/nsmb.3267). This construct (SEQ ID NO: 235) included optimal structure based stabilising mutations (A149C, S155C, S190F, V207L, L373R, S290C and Y458C) none of which were included in any of clamp stabilised candidates.

Membrane tethered clamp constructs were initially expressed in expiCHO transient expression system (Thermo Fisher). Soluble antigen secreted into the cell culture medium was quantified by capture ELISA with paired RSV specific monoclonal antibodies 101F (Mclellan et al., J Virol, 2010, DOI: 10.1128/JVI.01579-10) and Motavizumab (Mclellan et al., Nat Struct Mol Biol, 2010, DOI: 10.1038/nsmb.1723) (FIG. 43A). Antigen present on the cell surface was quantified by flow cytometry with monoclonal antibody Motavizumab (FIG. 43B). Three membrane tethered clamp constructs (VL22_CT5S_alpha, VL22_CT5S_gamma and VL22_CT5S_epsilon) showed substantially decreased level of antigen secreted into the cell supernatant relative to VL22_CT5S and increased cell number positive for surface bound antigen.

To further characterize RSV antigens expiCHO cells were lysed in SDS-PAGE sample buffer and assessed by SDS-PAGE and western blot with monoclonal antibody motavizumab with and without prior treatment with PNGase to remove N-linked glycans (FIG. 44). In this analysis soluble VL22-CT5s and membrane tethered mVL22-TM-CD11, VL22_CT5S_alpha and VL22_CT5S_gamma showed high expression of each antigen at the expected molecular weight. The expression level appeared to be significantly higher compared to controls mFwt and mDSCavAY. Two other membrane tethered clamp candidates, VL22_CT5S_eta and VL22_CT5S_epsilon, showed lower than expected molecular weight indicative of proteolytic cleavage.

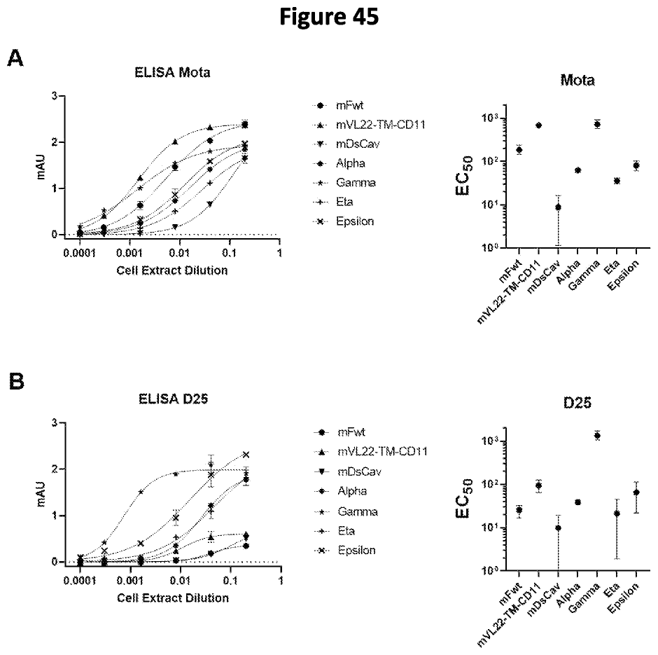

To provide a more quantitative analysis on expression level expiCHO cell extracts were prepared by resuspending cell pellets in lysis buffer containing 50 mM Tris-HCl, 150 mM NaCl, 1 mM EDTA, 10 mM EDTA and 1% polysorbate 80. Cells were lysed by sonication and cell debris removed by centrifugation. Relative amount of RSV F antigen within cell lysate was quantified by capture ELISA using monoclonal antibody 101F for capture and either Motavizumab for detection of total RSV F (FIG. 45A) or D25 (McLellan et al., 2013, Science, DOI: 10.1126/science.1234914) for detection of pre-fusion stabilised RSV F (FIG. 45B). Quantitative analysis of total RSV F revealed that mVL22-TM-CD11 and VL22_CT5S_gamma both produced higher levels of RSV F compared to mFwt. However, quantitative analysis of RSV F in the pre-fusion conformation using D25 revealed that VL22_CT5S_gamma had by far the highest amount of RSV F in the pre-fusion conformation.

The results obtained in this example therefore demonstrate that molecular Clamp2s design that included the membrane tethering (poly)peptide sequence from chicken ASIC1a (gamma) inserted between the FHRR and the SHRR was able to effectively stabilize the pre-fusion conformation of RSV F and to facilitate the presentation of a high level of antigen at the surface of transfected cells. The identified membrane tethered clamp (CT5S_gamma (SEQ ID NO: 227)) is therefore expected to be ideal for inclusion into mRNAvaccines for class I viral fusion proteins to enable the dual benefits of pre-fusion stabilisation with presentation at the target cell surface.

### FURTHER REFERENCES

Amarilla et al. An Optimized High-Throughput Immuno-Plaque Assay for SARS-CoV-2. Front Microbiol. 2021 Feb. 12; 12:625136. (DOI: 10.3389/fmicb.2021.625136).
Avanzato et al. A structural basis for antibody-mediated neutralization of Nipah virus reveals a site of vulnerability at the fusion glycoprotein apex. Proc Natl Acad Sci USA. 2019 Dec. 10; 116(50):25057-25067. (DOI: 10.1073/pnas.1912503116).
Chappell et al. Safety and immunogenicity of an MF59-adjuvanted spike glycoprotein-clamp vaccine for SARS-CoV-2: a randomised, double-blind, placebo-controlled, phase 1 trial. Lancet Infect Dis. 2021 October; 21(10):1383-1394. (DOI: 10.1016/S1473-3099(21)00200-0).
Isaacs et al. Combinatorial F-G Immunogens as Nipah and Respiratory Syncytial Virus Vaccine Candidates. Viruses. 2021 Sep. 28; 13(10):1942. (DOI: 10.3390/v13101942).
Joyce et al. Iterative structure-based improvement of a fusion-glycoprotein vaccine against RSV. Nat Struct Mol Biol. 2016 September; 23(9):811-820. (DOI: 10.1038/nsmb.3267).
McLellan et al. Structure of a major antigenic site on the respiratory syncytial virus fusion glycoprotein in complex with neutralizing antibody 101F. J Virol. 2010 December; 84(23):12236-44. (DOI: 10.1128/JVI.01579-10).
McLellan et al., Structural basis of respiratory syncytial virus neutralization by motavizumab. Nat Struct Mol Biol 17, 248-250 (2010). (DOI: 10.1038/nsmb.1723).
McLellan et al. Structure-based design of a fusion glycoprotein vaccine for respiratory syncytial virus. Science. 2013 Nov. 1; 342(6158):592-8. (DOI: 10.1126/science.1243283).
Wen et al. Structural basis for antibody cross-neutralization of respiratory syncytial virus and human metapneumovirus. Nat Microbiol 2:16272(2017). (DOI: 10.1038/nmicrobiol.2016.272).
McMillan et al. Development of molecular clamp stabilized hemagglutinin vaccines for Influenza A viruses. NPJ Vaccines. 2021 Nov. 8; 6(1):135. (DOI: 10.1038/s41541-021-00395-4).
Tian et al. Potent binding of 2019 novel coronavirus spike protein by a SARS coronavirus-specific human monoclonal antibody, Emerging Microbes & Infections. 2020; 9:1, 382-385, (DOI: 10.1080/22221751.2020.1729069).
Watterson, et al. Preclinical development of a molecular clamp-stabilised subunit vaccine for severe acute respiratory syndrome coronavirus 2. Clin Transl Immunology. 2021 Apr. 5; 10(4):e1269. (DOI: 10.1002/cti2.1269).

LIST OF SEQUENCES
Original HIV-clamp
SEQ ID NO: 1
IVQQQNNLLRAIEAQQHLLQLTVWGIKQLQARILAGGSGGHTTWMEWDREINNYTSLIHSLIEESQNQQEKNEQELLE
CD1: MMPV-based SSM
SEQ ID NO: 2
SITQYTKLSHQLISDVQAISSTIQDLQDQVDSLAEVVLQNRRGLDLLTAEQGGICLALQEKCSFYANKSGIVRDKIKN
LQDDLERRRRQLI
CD2: MMPV-based SSM
SEQ ID NO: 3
SITQYTKLSHQLISDVQAISSTIQDLQDQVDSLAEVVGSGYANKSGIVRDKIKNLQDDLERRRRQLI
CD3: XMRV-based SSM
SEQ ID NO: 4
FEQLQAAIHTDLGALEKSVSALEKSLTSLSEVVLQNRRGLDLLFLKEGGLCAALKEECSFYADHTGVVRDSMAKLRERLNQ
CD4: XMRV-based SSM
SEQ ID NO: 5
FEQLQAAIHTDLGALEKSVSALEKSLTSLSEVVGSGYADHTGVVRDSMAKLRERLNQ
CD5: KoRV-based SSM
SEQ ID NO: 6
LQIAMDTDLRALQDSISKLEDSLTSLSEVVLQNRRGLDLLFLKEGGLCAALKEECSFYVDHSGAVRDSMRRLKERL
CD6: KoRV-based SSM
SEQ ID NO: 7
AMDTDLRALQDSISKLEDSLTSLSEVVGSGYVDHSGAVRDSMRRLKERL
CD7: FIV-based SSM
SEQ ID NO: 8
ATHQEAIEKVTEALKINNLRLVTLEHQVLVIGLKVEAGGSGGNQTIWNHGNITLGEWYNKTKDLQQKFYEIIMDIEQNN
CD8: SIV-based SSM
SEQ ID NO: 9
AGIVQQQQQLLDVVKRQQELLRLTVWGTKNLQTRVTAGGSGGNNETWQEWERKVDFLEENITALLEEAQIQQEKNMYELQ
CD9: VISNA-based SSM
SEQ ID NO: 10
QSLANATAAQQEVLEASYAMVQHIAKGIRILEARVARGGSGGNHTWQQWEEEIEQHEGNLSLLLREAALQVHIAQRDARRI
CD10: CAEV-based SSM
SEQ ID NO: 11
QTLANATAAQQDVLEATYAMVQHVAKGVRILEARVARGGSGGNHTWQQWERGLQGYDTNLTILLKESAAMTQLAEEQARRI
CD11: VISNA-based SSM
SEQ ID NO: 12
QSLANATAAQQEVLEAQYAMVQHIAKGIRILEARVARGGSGGNHTWQQWEEEIEQHEGNLSLLLREAALQVHIAQRDARRI
CK1: Lassa-based SSM
SEQ ID NO: 13
EFSDMLRLFDFNKQAIQRLKAEAQMSIQLINGGSGGNETHFSDDIEQQADNMITEMLQKEYMERQGKTPLGLVD
CK2: EAIV-based SSM
SEQ ID NO: 14
ADSVQNHTFEVENNTINGLELTEEQVHILYAMVLQTGGSGGTQWDDWVDKMENLNHDILTTLHTARNNLEQSMITFN
CK3: NiV-based SSM
SEQ ID NO: 15
EAMKNADNINKLKSSIESTNEAVVKLQETAEKTVGGSGGDISSQISSMNQSLQQSKDYIKEAQRLLDTVNPS
CK4: GALV-based SSM
SEQ ID NO: 16
DLQQGLTSLQIAMDTDLRALQDSISKLEDSLTSLSEGGSGGFYVDHSGAVRDSMKKLKERLDKRQLERQKNQN
CK5: KoRV-based SSM
SEQ ID NO: 17
DLQQGLTSLQIAMDTDLRALQDSISKLEDSLTSLSEGGSGGFYVDHSGAVRDSMRRLKERLDKRQLEHQKNLS
CK6: PERV-based SSM
SEQ ID NO: 18
QQLEKGLSNLHRIVTEDLQALEKSVSNLEESLTSLSEGGSGGFYVDHSGAIRDSMNKLRERLEKRRREKETTQG
CK7: FELV-based SSM
SEQ ID NO: 19
KALIETAQFRQLQMAMHTDIQALEESISALEKSLTSLSEGGSGGFYADHTGLVRDNMAKLRERLKQRQQLFDSQQGWFE
CK8: BoLV-based SSM
SEQ ID NO: 20
STLSHQRLTSLIHVLEQDQQRLITAINQTHYNLLNVASGGSGGFLRIQNDSIIRLGDLQPLSQRVSTD
RSV Fusion protein 66K (aa 1-574)-unmodified sequence Genbank Accession: AHL84194
SEQ ID NO: 21
MELLILKANAITTILTAVTFCFASGQNITEEFYQSTCSAVSKGYLSALRTGWYTSVITIELSNIKKNKCNGTDAKVKLIKQELDKYK
NAVTELQLLMQSTPATNNRARRELPRFMNYTLNNAKKTNVTLSKKRKRRFLGFLLGVGSAIASGVAVSKVLHLEGEVNKIKSALLST
NKAVVSLSNGVSVLTSKVLDLKNYIDKQLLPIVNKQSCSISNIETVIEFQQKNNRLLEITREFSVNAGVTTPVSTYMLTNSELLSLI
NDMPITNDQKKLMSNNVQIVRQQSYSIMSIIKEEVLAYVVQLPLYGVIDTPCWKLHTSPLCTTNTKEGSNICLTRTDRGWYCDNAG
SVSFFPQAETCKVQSNRVFCDTMNSLTLPSEVNLCNVDIFNPKYDCKIMTSKTDVSSSVITSLGAIVSCYGKTKCTASNKNRGIIKT
FSNGCDYVSNKGVDTVSVGNTLYYVNKQEGKSLYVKGEPIINFYDPLVFPSDEFDASISQVNEKINQSLAFIRKSDELLHNVNAGK
STTNIMITTIIIVIIVILLSLIAVGLLLYCKARSTPVTLSKDQLSGINNIAFSN
wherein:
Italicized text corresponds to the signal peptide of RSV Fusion protein
RSV Fusion protein 66K-T¹⁰³-GS-^{G145}-E⁵¹¹-GSG-VISNA-based SSM
Referred to as ‘VL22_66K_CD11’
SEQ ID NO: 22
MELLILKANAITTILTAVTFCFASGQNITEEFYQSTCSAVSKGYLSALRTGWYTSVITIELSNIKKNKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGQSLANATAAQQEVLEAQYAMVQHIAKGIRILEARVARGGSGGNHTW
QQWEEEIEQHEGNLSLLLREAALQVHIAQRDARRI
wherein:
Italicized text corresponds to the signal peptide of RSV Fusion protein;
Bold text is a flexible linker; and
Underlined text corresponds to a VISNA-based SSM (CD11)
RSV Fusion protein 66E (aa 1-574)-unmodified sequence Genbank Accession: QGW56794
SEQ ID NO: 23
MELLILKANAITTILTAVTFCFASGQNITEEFYQSTCSAVSKGYLSALRTGWYTSVITIELSNIKENKCNGTDAKVKLIKQELDKYK
NAVTELQLLMQSTPATNNRARRELPRFMNYTLNNAKKTNVTLSKKRKRRFLGFLLGVGSAIASGVAVSKVLHLEGEVNKIKSALLST
NKAVVSLSNGVSVLTSKVLDLKNYIDKQLLPIVNKQSCSISNIETVIEFQQKNNRLLEITREFSVNAGVTTPVSTYMLTNSELLSLI
NDMPITNDQKKLMSNNVQIVRQQSYSIMSIIKEEVLAYVVQLPLYGVIDTPCWKLHTSPLCTTNTKEGSNICLTRTDRGWYCDNAG
SVSFFPQAETCKVQSNRVFCDTMNSLTLPSEVNLCNVDIFNPKYDCKIMTSKTDVSSSVITSLGAIVSCYGKTKCTASNKNRGIIKT
FSNGCDYVSNKGVDTVSVGNTLYYVNKQEGKSLYVKGEPIINFYDPLVFPSDEFDASISQVNEKINQSLAFIRKSDELLHNVNAGK
STTNIMITTIIIVIIVILLSLIAVGLLLYCKARSTPVTLSKDQLSGINNIAFSN
wherein:
Italicized text corresponds to the signal peptide of RSV Fusion protein
RSV Fusion protein 66E-T¹⁰³-GS-^{G145}-E⁵¹¹-GSG-VISNA-based SSM
Referred to as ‘VL22_66E_CD11’ in Examples 1-6 or for simplicity ‘VL22_CD11’ from Example 7
SEQ ID NO: 24
MELLILKANAITTILTAVTFCFASGQNITEEFYQSTCSAVSKGYLSALRTGWYTSVITIELSNIKENKCNGTDAKVKLIKQELDKYKN
AVTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQL
PLYGVIDTPCWKLHTSPLCTTNTKEGSNICLTRTDRGWYCDNAGSVSFFPQAETCKVQSNRVFCDTMNSLTLPSEVNLCNVDIFNPKY
DCKIMTSKTDVSSSVITSLGAIVSCYGKTKCTASNKNRGIIKTFSNGCDYVSNKGVDTVSVGNTLYYVNKQEGKSLYVKGEPIINFYD
PLVFPSDEFDASISQVNEKINQSLAFIRKSDEGSGQSLANATAAQQEVLEAQYAMVQHIAKGIRILEARVARGGSGGNHTWQQWEEEI
EQHEGNLSLLLREAALQVHIAQRDARRI
wherein:
Italicized text corresponds to the signal peptide of RSV Fusion protein;
Bold text is a flexible linker; and
Underlined text corresponds to a VISNA-based SSM
RSV Fusion protein 66K/101Q (aa 1-574)-unmodified sequence Genbank AIO08046
SEQ ID NO: 25
MELLILKANAITTILTAVTFCFASGQNITEEFYQSTCSAVSKGYLSALRTGWYTSVITIELSNIKKNKCNGTDAKVKLIKQELDKYK
NAVTELQLLMQSTQATNNRARRELPRFMNYTLNNAKKTNVTLSKKRKRRFLGFLLGVGSAIASGVAVSKVLHLEGEVNKIKSALLST
NKAVVSLSNGVSVLTSKVLDLKNYIDKQLLPIVNKQSCSISNIETVIEFQQKNNRLLEITREFSVNAGVTTPVSTYMLTNSELLSLI
NDMPITNDQKKLMSNNVQIVRQQSYSIMSIIKEEVLAYVVQLPLYGVIDTPCWKLHTSPLCTTNTKEGSNICLTRTDRGWYCDNAG
SVSFFPQAETCKVQSNRVFCDTMNSLTLPSEVNLCNVDIFNPKYDCKIMTSKTDVSSSVITSLGAIVSCYGKTKCTASNKNRGIIKT
FSNGCDYVSNKGVDTVSVGNTLYYVNKQEGKSLYVKGEPIINFYDPLVFPSDEFDASISQVNEKINQSLAFIRKSDELLHNVNAGK
STTNIMITTIIIVIIVILLSLIAVGLLLYCKARSTPVTLSKDQLSGINNIAFSN
wherein:
Italicized text corresponds to the signal peptide of RSV Fusion protein
RSV Fusion protein 66K/101Q-N¹⁰⁵-GS-^{G145}-E⁵¹¹-GSG-HIV-1 GP41-based SSM
Referred to as ‘F HIV-clamp’
SEQ ID NO: 26
MELLILKANAITTILTAVTFCFASGQNITEEFYQSTCSAVSKGYLSALRTGWYTSVITIELSNIKKNKCNGTDAKVKLIKQELDKYKN
AVTELQLLMQSTQATNNGSGSAIASGVAVSKVLHLEGEVNKIKSALLSTNKAVVSLSNGVSVLTSKVLDLKNYIDKQLLPIVNKQSCS
ISNIETVIEFQQKNNRLLEITREFSVNAGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCKVQSNRVFCDTMNSLTLPSEVNLCNV
DIFNPKYDCKIMTSKTDVSSSVITSLGAIVSCYGKTKCTASNKNRGIIKTFSNGCDYVSNKGVDTVSVGNTLYYVNKQEGKSLYVK
GEPIINFYDPLVFPSDEFDASISQVNEKINQSLAFIRKSDEGSGIVQQQNNLLRAIEAQQHLLQLTVWGIKQLQARILAGGSGGHT
TWMEWDREINNYTSLIHSLIEESQNQQEKNEQELLE
wherein:
Italicized text corresponds to the signal peptide of RSV Fusion protein;
Bold text is a flexible linker; and
Underlined text corresponds to a HIV gp41-based SSM
RSV Fusion protein 66K/101Q-V⁵¹⁶-His-Tag
Referred to as ‘Fsol’
SEQ ID NO: 27
MELLILKANAITTILTAVTFCFASGQNITEEFYQSTCSAVSKGYLSALRTGWYTSVITIELSNIKKNKCNGTDAKVKLIKQELDKYK
NAVTELQLLMQSTQATNNRARRELPRFMNYTLNNAKKTNVTLSKKRKRRFLGFLLGVGSAIASGVAVSKVLHLEGEVNKIKSALLST
NKAVVSLSNGVSVLTSKVLDLKNYIDKQLLPIVNKQSCSISNIETVIEFQQKNNRLLEITREFSVNAGVTTPVSTYMLTNSELLSLI
NDMPITNDQKKLMSNNVQIVRQQSYSIMSIIKEEVLAYVVQLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKIMTSKTDVSSSVITSLGAIVSCYGKTKCTASNKNRGIIKTF
SNGCDYVSNKGVDTVSVGNTLYYVNKQEGKSLYVKGEPIINFYDPLVFPSDEFDASISQVNEKINQSLAFIRKSDELLHNVGGGG
HHHHHH
wherein:
Italicized text corresponds to the signal peptide of RSV Fusion protein;
Bold text is a flexible linker; and Underlined text corresponds to a His Tag
SARS-CoV-2 Spike protein-N⁶⁷⁹-GSG-S⁶⁹¹-G¹²⁰⁴-SG-HIV GP41-based SSM
Referred to as ‘SARS-CoV-2 S HIV-clamp’
SEQ ID NO: 28
MFVFLVLLPLVSSQCVNLTTRTQLPPAYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPIGINITRFQTLLALHRSYLTPGDSSSGW
TAGAAAYYVGYLQPRTFLLKYNENGTITDAVDCALDPLSETKCTLKSFTVEKGIYQTSNFRVQPTESIVRFPNITNLCPFGEVENA
TRFASVYAWNRKRISNCVADYSVLYNSASFSTFKCYGVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSYECDIPIGAGICASYQTQTNGSGSII
AYTMSLGAENSVAYSNNSIAIPTNFTISVTTEILPVSMTKTSVDCTMYICGDSTECSNLLLQYGSFCTQLNRALTGIAVEQDKNT
QEVFAQVKQIYKTPPIKDFGGFNFSQILPDPSKPSKRSFIEDLLFNKVTLADAGFIKQYGDCLGDIAARDLICAQKFNGLTVLPP
LLTDEMIAQYTSALLAGTITSGWTFGAGAALQIPFAMQMAYRFNGIGVTQNVLYENQKLIANQFNSAIGKIQDSLSSTASALGKL
QDVVNQNAQALNTLVKQLSSNFGAISSVLNDILSRLDKVEAEVQIDRLITGRLQSLQTYVTQQLIRAAEIRASANLAATKMSECV
LGQSKRVDFCGKGYHLMSFPQSAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDRLNEVAKNLNESLIDLQEL
GSGIVQQQNNLLRAIEAQQHLLQLTVWGIKQLQARILAGGSGGHTTWMEWDREINNYTSLIHSLIEESQNQQEKNEQELLE
wherein:
Italicized text corresponds to the signal peptide of SARS-CoV-2 spike protein;
Bold text is a flexible linker; and
Underlined text corresponds to a HIV GP41-based SSM
SARS-CoV-2 Spike protein-N⁶⁷⁹-GSG-S⁶⁹¹-G¹²⁰⁴-VISNA-based SSM CD11
Referred to as ‘SARS-CoV-2 S CD11’
SEQ ID NO: 29
MFVFLVLLPLVSSQCVNLTTRTQLPPAYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPIGINITRFQTLLALHRSYLTPGDSSSGW
TAGAAAYYVGYLQPRTFLLKYNENGTITDAVDCALDPLSETKCTLKSFTVEKGIYQTSNFRVQPTESIVRFPNITNLCPFGEVENA
TRFASVYAWNRKRISNCVADYSVLYNSASFSTFKCYGVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSYECDIPIGAGICASYQTQTNGSGSII
AYTMSLGAENSVAYSNNSIAIPTNFTISVTTEILPVSMTKTSVDCTMYICGDSTECSNLLLQYGSFCTQLNRALTGIAVEQDKNT
QEVFAQVKQIYKTPPIKDFGGFNFSQILPDPSKPSKRSFIEDLLFNKVTLADAGFIKQYGDCLGDIAARDLICAQKFNGLTVLPP
LLTDEMIAQYTSALLAGTITSGWTFGAGAALQIPFAMQMAYRFNGIGVTQNVLYENQKLIANQFNSAIGKIQDSLSSTASALGKL
QDVVNQNAQALNTLVKQLSSNFGAISSVLNDILSRLDKVEAEVQIDRLITGRLQSLQTYVTQQLIRAAEIRASANLAATKMSECV
LGQSKRVDFCGKGYHLMSFPQSAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDRLNEVAKNLNESLIDLQEL
GSGQSLANATAAQQEVLEAQYAMVQHIAKGIRILEARVARGGSGGNHTWQQWEEEIEQHEGNLSLLLREAALQVHIAQRDARRI
wherein:
Italicized text corresponds to the signal peptide of SARS-CoV-2 spike protein;
Bold text is a flexible linker; and
Underlined text corresponds to a VISNA-based SSM CD11
Nipah virus Fusion-HIV GP41-based SSM
Referred to as ‘Nipah F HIV-clamp’
SEQ ID NO: 30
MVVILDKRCYCNLLILILMISECSVGILHYEKLSKIGLVKGVTRKYKIKSNPLTKDIVIKMIPNVSNMSQCTGSVMENYKTRLNGILTP
IKGALEIYKNNTHDLVGDVRLAGVIMAGVAIGIATAAQITAGVALYEAMKNADNINKLKSSIESTNEAVVKLQETAEKTVYVLTAL
QDYINTNLVPTIDKISCKQTELSLDLALSKYLSDLLFVFGPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIVPNFILVRNTLISNIEIGFCLITKRSVICNQDYATPMTNNMR
ECLTGSTEKCPRELVVSSHVPRFALSNGVLFANCISVTCQCQTTGRAISQSGEQTLLMIDNTTCPTAVLGNVIISLGKYLGSVNYNS
EGIAIGPPVFTDKVDISSQISSMNQSLQQSKDYIKEAQRLLDTGSGIVQQQNNLLRAIEAQQHLLQLTVWGIKQLQARILAGGSG
GHTTWMEWDREINNYTSLIHSLIEESQNQQEKNEQELLE
wherein:
Italicized text corresponds to the signal peptide of Nipah
virus Fusion protein; Bold text is a flexible linker; and
Underlined text corresponds to a HIV GP41-based SSM
Nipah virus Fusion-VISNA-based SSM
Referred to as ‘Nipah F CD11’
SEQ ID NO: 31
MVVILDKRCYCNLLILILMISECSVGILHYEKLSKIGLVKGVTRKYKIKSNPLTKDIVIKMIPNVSNMSQCTGSVMENYKTRLNGILTP
IKGALEIYKNNTHDLVGDVRLAGVIMAGVAIGIATAAQITAGVALYEAMKNADNINKLKSSIESTNEAVVKLQETAEKTVYVLTAL
QDYINTNLVPTIDKISCKQTELSLDLALSKYLSDLLFVFGPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIVPNFILVRNTLISNIEIGFCLITKRSVICNQDYATPMTNNMR
ECLTGSTEKCPRELVVSSHVPRFALSNGVLFANCISVTCQCQTTGRAISQSGEQTLLMIDNTTCPTAVLGNVIISLGKYLGSVNYNS
EGIAIGPPVFTDKVDISSQISSMNQSLQQSKDYIKEAQRLLDTGSGQSLANATAAQQEVLEAQYAMVQHIAKGIRILEARVARGG
SGGNHTWQQWEEEIEQHEGNLSLLLREAALQVHIAQRDARRI
wherein:
Italicized text corresponds to the signal peptide of Nipah virus
Fusion protein;
Bold text is a flexible linker; and
Underlined text corresponds to a VISNA-based SSM CD11
Influenza Hemagglutinin protein R⁵²⁶-HIV GP41-based SSM
Referred to as ‘Influenza HA HIV-clamp’
SEQ ID NO: 32
MKAILVVLLYTFATANADTLCIGYHANNSTDTVDTVLEKNVTVTHSVNLLEDKHNGKLCKLRGVAPLHLGKCNIAGWILGNPECE
SLSTASSWSYIVETPSSDNGTCYPGDFIDYEELREQLSSVSSFERFEIFPKTSSWPNHDSNKGVTAACPHAGAKSFYKNLIWLVKK
GNSYPKLSKSYINDKGKEVLVLWGIHHPSTSADQQSLYQNADTYVFVGSSRYSKKFKPEIAIRPKVRDQEGRMNYYWTLVEPGDK
ITFEATGNLVVPRYAFAMERNAGSGIIISDTPVHDCNTTCQTPKGAINTSLPFQNIHPITIGKCPKYVKSTKLRLATGLRNIPSIQ
SRGLFGAIAGFIEGGWTGMVDGWYGYHHQNEQGSGYAADLKSTQNAIDEITNKVNSVIEKMNTQFTAVGKEFNHLEKRIENLN
KKVDDGFLDIWTYNAELLVLLENERTLDYHDSNVKNLYEKVRSQLKNNAKEIGNGCFEFYHKCDNTCMESVKNGTYDYPKYSEE
AKLNREEIDGVKLESTRGSGIVQQQNNLLRAIEAQQHLLQLTVWGIKQLQARILAGGSGGHTTWMEWDREINNYTSLIHSLIEE
SQNQQEKNEQELLE
wherein:
Italicized text corresponds to the signal peptide of Influenza
Hemagglutinin protein;
Bold text is a flexible linker; and
Underlined text corresponds to a HIV-1 GP41-based SSM
Influenza Hemagglutinin protein R⁵²⁶-VISNA-based SSM
‘Influenza HA CD11’
SEQ ID NO: 33
MKAILVVLLYTFATANADTLCIGYHANNSTDTVDTVLEKNVTVTHSVNLLEDKHNGKLCKLRGVAPLHLGKCNIAGWILGNPECE
SLSTASSWSYIVETPSSDNGTCYPGDFIDYEELREQLSSVSSFERFEIFPKTSSWPNHDSNKGVTAACPHAGAKSFYKNLIWLVKK
GNSYPKLSKSYINDKGKEVLVLWGIHHPSTSADQQSLYQNADTYVFVGSSRYSKKFKPEIAIRPKVRDQEGRMNYYWTLVEPGD
KITFEATGNLVVPRYAFAMERNAGSGIIISDTPVHDCNTTCQTPKGAINTSLPFQNIHPITIGKCPKYVKSTKLRLATGLRNIPSIQS
RGLFGAIAGFIEGGWTGMVDGWYGYHHQNEQGSGYAADLKSTONAIDEITNKVNSVIEKMNTQFTAVGKEFNHLEKRIENLN
KKVDDGFLDIWTYNAELLVLLENERTLDYHDSNVKNLYEKVRSQLKNNAKEIGNGCFEFYHKCDNTCMESVKNGTYDYPKYSEE
AKLNREEIDGVKLESTRGSGQSLANATAAQQEVLEAQYAMVQHIAKGIRILEARVARGGSGGNHTWQQWEEEIEQHEGNLSL
LLREAALQVHIAQRDARRI
wherein:
Italicized text corresponds to the signal peptide of Influenza
Hemagglutinin protein;
Bold text is a flexible linker; and
Underlined text corresponds to a VISNA-based SSM CD11
SARS-CoV-2 Spike protein-N⁶⁷⁹-GSG-S⁶⁹¹-G¹²⁰⁴-VISNA-based SSM CD11 (-SG)
Referred to as ‘SARS-CoV-2 S CD11_SG’
SEQ ID NO: 34
MFVFLVLLPLVSSQCVNLTTRTQLPPAYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPIGINITRFQTLLALHRSYLTPGDSSSGW
TAGAAAYYVGYLQPRTFLLKYNENGTITDAVDCALDPLSETKCTLKSFTVEKGIYQTSNFRVQPTESIVRFPNITNLCPFGEVENA
TRFASVYAWNRKRISNCVADYSVLYNSASFSTFKCYGVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSYECDIPIGAGICASYQTQTNGSGSII
AYTMSLGAENSVAYSNNSIAIPTNFTISVTTEILPVSMTKTSVDCTMYICGDSTECSNLLLQYGSFCTQLNRALTGIAVEQDKNT
QEVFAQVKQIYKTPPIKDFGGFNFSQILPDPSKPSKRSFIEDLLFNKVTLADAGFIKQYGDCLGDIAARDLICAQKFNGLTVLPP
LLTDEMIAQYTSALLAGTITSGWTFGAGAALQIPFAMQMAYRFNGIGVTQNVLYENQKLIANQFNSAIGKIQDSLSSTASALGKL
QDVVNQNAQALNTLVKQLSSNFGAISSVLNDILSRLDKVEAEVQIDRLITGRLQSLQTYVTQQLIRAAEIRASANLAATKMSECV
LGQSKRVDFCGKGYHLMSFPQSAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDRLNEVAKNLNESLIDLQEL
GQSLANATAAQQEVLEAQYAMVQHIAKGIRILEARVARGGSGGNHTWQQWEEEIEQHEGNLSLLLREAALQVHIAQRDARRI
wherein:
Italicized text corresponds to the signal peptide of SARS-CoV-2
spike protein;
Bold text is a flexible linker; and
Underlined text corresponds to a VISNA-based SSM CD11
CD11_124568
SEQ ID NO: 35
QSLANATAAQQEVLEAQYAMVQHIAKGIRILEARVARGGSGGNHTWQNWTEEIENHTGNLSNLTREAANQTHIAQRDARRI
CD11_1245T68
SEQ ID NO: 36
QSLANATAAQQEVLEAQYAMVQHIAKGIRILEARVARGGSGGNHTWQNWTEEIENHTGNLTNLTREAANQTHIAQRDARRI
CD11_1245T8
SEQ ID NO: 37
QSLANATAAQQEVLEAQYAMVQHIAKGIRILEARVARGGSGGNHTWQNWTEEIENHTGNLTLLLREAANQTHIAQRDARRI
CD11_145T8
SEQ ID NO: 38
QSLANATAAQQEVLEAQYAMVQHIAKGIRILEARVARGGSGGNHTWQQWEEEIENHTGNLTLLLREAANQTHIAQRDARRI
CD11_1458
SEQ ID NO: 39
QSLANATAAQQEVLEAQYAMVQHIAKGIRILEARVARGGSGGNHTWQQWEEEIENHTGNLSLLLREAANQTHIAQRDARRI
CD11_159
SEQ ID NO: 40
QSLANATAAQQEVLEAQYAMVQHIAKGIRILEARVARGGSGGNHTWQQWEEEIEQHEGNLSLLLREAALQVHNATRDARRI
CD11_158
SEQ ID NO: 41
QSLANATAAQQEVLEAQYAMVQHIAKGIRILEARVARGGSGGNHTWQQWEEEIEQHEGNLSLLLREAANQTHIAQRDARRI
CD11_157
SEQ ID NO: 42
QSLANATAAQQEVLEAQYAMVQHIAKGIRILEARVARGGSGGNHTWQQWEEEIEQHEGNLSLLLRNATLQVHIAQRDARRI
CD11_156
SEQ ID NO: 43
QSLANATAAQQEVLEAQYAMVQHIAKGIRILEARVARGGSGGNHTWQQWEEEIEQHEGNLSNLTREAALQVHIAQRDARRI
CD11_145
SEQ ID NO: 44
QSLANATAAQQEVLEAQYAMVQHIAKGIRILEARVARGGSGGNHTWQQWEEEIENHTGNLSLLLREAALQVHIAQRDARRI
CD11_135
SEQ ID NO: 45
QSLANATAAQQEVLEAQYAMVQHIAKGIRILEARVARGGSGGNHTWQQWEENITQHEGNLSLLLREAALQVHIAQRDARRI
CD11_125
SEQ ID NO: 46
QSLANATAAQQEVLEAQYAMVQHIAKGIRILEARVARGGSGGNHTWQNWTEEIEQHEGNLSLLLREAALQVHIAQRDARRI
CD11_15T
SEQ ID NO: 47
QSLANATAAQQEVLEAQYAMVQHIAKGIRILEARVARGGSGGNHTWQQWEEEIEQHEGNLTLLLREAALQVHIAQRDARRI
CD11_1
SEQ ID NO: 48
QSLANATAAQQEVLEAQYAMVQHIAKGIRILEARVARGGSGGNHTWQQWEEEIEQHEGQLTLLLREAALQVHIAQRDARRI
CD11_5T
SEQ ID NO: 49
QSLANATAAQQEVLEAQYAMVQHIAKGIRILEARVARGGSGGQHTWQQWEEEIEQHEGNLTLLLREAALQVHIAQRDARRI
RSV Fusion protein 66E-T¹⁰³-GS-^{G145}-E⁵¹¹-GSG-HIV-GP41-based SSM
Referred to as ‘F HIV-clamp’
SEQ ID NO: 50
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGIVQQQNNLLRAIEAQQHLLQLTVWGIKQLQARILAGGSGGHTTW
MEWDREINNYTSLIHSLIEESQNQQEKNEQELLE
wherein:
Italicized text corresponds to the signal peptide of RSV Fusion protein;
Bold text is a flexible linker; and
Underlined text corresponds to a HIV-1 GP41-based SSM
RSV Fusion protein 66E-T¹⁰³-GS-^{G145}-E⁵¹¹-GSG-VISNA-based SSM with additional N-linked
glycosylation sequences (CD11_1245T8)
Referred to as ‘VL22_CD11_1245T8’
SEQ ID NO: 51
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGQSLANATAAQQEVLEAQYAMVQHIAKGIRILEARVARGGSGGNHT
WQNWTEEIENHTGNLTLLLREAANQTHIAQRDARRI
wherein:
Italicized text corresponds to the signal peptide of RSV Fusion protein;
Bold text is a flexible linker; and
Underlined text corresponds to a VISNA-based SSM
RSV Fusion protein 66E-T¹⁰³-GS-G¹⁴⁵-E⁵¹¹-GSG-VISNA-based SSM with
additional N-linked glycosylation sequences (CD11_145T8)
Referred to as ‘VL22_CD11_145T8’
SEQ ID NO: 52
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGQSLANATAAQQEVLEAQYAMVQHIAKGIRILEARVARGGSGGNHT
WQQWEEEIENHTGNLTLLLREAANQTHIAQRDARRI
wherein:
Italicized text corresponds to the signal peptide of RSV Fusion protein;
Bold text is a flexible linker; and
Underlined text corresponds to a VISNA-based SSM
RSV Fusion protein DS Cav1 Foldon(Mclellan et al., Science 2013
doi: 10.1126/science.1243283)
Referred to as ‘DS Cav1 Foldon’
SEQ ID NO: 53
MELLILKANAITTILTAVTFCFASGQNITEEFYQSTCSAVSKGYLSALRTGWYTSVITIELSNIKKNKCNGTDAKVKLIKQELDKYK
NAVTELQLLMQSTQATNNRARRELPRFMNYTLNNAKKTNVTLSKKRKRRFLGFLLGVGSAIASGVAVCKVLHLEGEVNKIKSALLST
NKAVVSLSNGVSVLTFKVLDLKNYIDKQLLPILNKQSCSISNIETVIEFQQKNNRLLEITREFSVNAGVTTPVSTYMLTNSELLSLI
NDMPITNDQKKLMSNNVQIVRQQSYSIMCIIKEEVLAYVVQLPLYGVIDTPCWKLHTSPLCTTNTKEGSNICLTRTDRGWYCDNAG
SVSFFPQAETCKVQSNRVFCDTMNSLTLPSEVNLCNVDIFNPKYDCKIMTSKTDVSSSVITSLGAIVSCYGKTKCTASNKNRGIIKT
FSNGCDYVSNKGVDTVSVGNTLYYVNKQEGKSLYVKGEPIINFYDPLVFPSDEFDASISQVNEKINQSLAFIRKSDELLSAIGGYIPE
APRDGQAYVRKDGEWVLLSTFLGGLVPRGSGSAWSHPQFEK
wherein:
Italicized text corresponds to the signal peptide of RSV Fusion protein;
Bold text is a flexible linker; and
Underlined text corresponds to Foldon SSM
RSV Fusion protein SC9-10 AY DS Cav1 Foldon
(Joyce et al., Nat Struct Mol Biol 2016 DOI: 10.1038/nsmb.3267)
Referred to as ‘SC9-10 AY DS Cav1 Foldon’
SEQ ID NO: 54
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAICSGVAVCKVLHLEGEVNKIKSALLSTNKAVVSLSNGVSVLTFKVLDLKNYIDKQLLPILNKQSCSISN
IETVIEFQQKNNRLLEITREFSVNAGVTTPVSTYMLTNSELLSLINDMPITNDQKKLMSNNVQIVRQQSYSIMCIIKEEVLAYVVQL
PLYGVIDTPCWKLHTSPLCTTNTKEGSNICLTRTDRGWYCDNAGSVSFFPQAETCKVQSNRVFCDTMNSRTLPSEVNLCNVDIF
NPKYDCKIMTSKTDVSSSVITSLGAIVSCYGKTKCTASNKNRGIIKTFSNGCDYVSNKGVDTVSVGNTLYCVNKQEGKSLYVKGEP
IINFYDPLVFPSDEFDASISQVNEKINQSLAFIRKSDELLSAIGGYIPEAPRDGQAYVRKDGEWVLLSTFLGGLVPR
wherein:
Italicized text corresponds to the signal peptide of RSV Fusion protein;
Bold text is a flexible linker; and
Underlined text corresponds to Foldon SSM
CT1
SEQ ID NO: 55
QSLANATAAQQEVLEAQYAMVRHIAKGIRILEARVARGGSGGNHTWQQWEEEIEQHEGNLSLLLREAALQVHIAQRDARRI
CT2
SEQ ID NO: 56
QSLANATAAQQEVLEAQYAMVQHIAKGIRKLEARVARGGSGGNHTWQQWEEEIEQHEGNLSLLLREAALQVHIAQRDARRI
CT3
SEQ ID NO: 57
QSLANATAAQQEVLEAQYAMVQHIAKGIRILEARVARGGSGGNHTWQQWEEEIEQHEGNLSSLLREAALQVHIAQRDARRI
CT4
SEQ ID NO: 58
QSLANATAAQQEVLEAQYAMVQHIAKGIRILEARVARGGSGGNHTWQQWEEEIEQHEGNLSSKLREAALQVHIAQRDARRI
CT5
SEQ ID NO: 59
QSLANATAAQQEVLEAQYAMVQHIAKGIRILEARVARGGSGGHTTWQQWEEEIEQHEGNLSLLLREAALQVHIAQRDARRI
CT6
SEQ ID NO: 60
QSLANATAAQQEVLEAQYAMVQHIAKGIRILEARVARGGSGGNHTWQQWEEEIEQHEGNLSLLLRESQQQVHIAQRDARRI
CT7
SEQ ID NO: 61
QSLANATAAQQEVLEAQYAMVQHIAKGIRILEARVARGGSGGNHTWQQWEEEIEQHEGNLSLLLREAALQVHSAQRDARRI
CT8
SEQ ID NO: 62
QSLANATAAQLEVLEAQYAMVQHIAKGIRILEARVARGGSGGNHTWQQWEEEIEQHEGNLSLLLREAALQVHIAQRDARRI
CT9
SEQ ID NO: 63
QSLANATAAQQEVLEALYAMVQHIAKGIRILEARVARGGSGGNHTWQQWEEEIEQHEGNLSLLLREAALQVHIAQRDARRI
CT10
SEQ ID NO: 64
QSLANATAAQQEVLEAQYAMVQHIAKGIRILEARVAGGSGGNHTWQQWEEEIEQHEGNLSLLLREAALQVHIAQRDARRI
CT11
SEQ ID NO: 65
QSLANATAAQQEVLEAQYAMVQHIAKGIRILEARVARVEAGGSGGNHTWQQWEEEIEQHEGNLSLLLREAALQVHIAQRDAR
RI
CT12
SEQ ID NO: 66
QSLANATAAQQEVLEAQYAMVQHIAKGIRILEARVARPGSGGNHTWQQWEEEIEQHEGNLSLLLREAALQVHIAQRDARRI
CT13
SEQ ID NO: 67
QSLANATAAQQEVLEAQYAMVQHIAKGIRILEARVAPGGSGGNHTWQQWEEEIEQHEGNLSLLLREAALQVHIAQRDARRI
CT14
SEQ ID NO: 68
QSLANATAAQQEVLEAQYAMVQHIAKGIRILEARVARGGEGGNHTWQQWEEEIEQHEGNLSLLLREAALQVHIAQRDARRI
CT15
SEQ ID NO: 69
QSLANATAAQQEVLEAQYAMVQHIAKGIRILEARVARGGSGGNHTWQQWEEEIEQHEGNLSLLLREAALQVHIAQ
CT16
SEQ ID NO: 70
QSLANATAAQQEVLEAQYAMVQHIAKGIRILEARVARGGSGGNHTWQQWEEEIEQHEGNLSLLLREAALQVHIAQR
CT17
SEQ ID NO: 71
QSLANATAAQQEVLEAQYAMVQHIAKGIRILEARVARGGSGGNHTWQQWEEEIEQHEGNLSLLLREAALQVHIAQRDA
CT18
SEQ ID NO: 72
QSLANATAAQQEVLEAQYAMVQHIAKGIRILEARVARGGSGGNHTWQQWEEEIEQHEGNLSLLLREAALQVHIAQRDAR
SARS-CoV-2 (Delta) Spike protein-N⁶⁷⁹-GSG-S⁶⁹¹-G¹²⁰⁴-VISNA-based SSM CD11
Referred to as ‘SARS-CoV-2 (Delta) S CD11’
SEQ ID NO: 73
MFVFLVLLPLVSSQCVNLRTRTQLPPAYTNSFTRGVYYPDKVFRSSVLHSTQDLFLPFFSNVTWFHAIHVSGTNGTKRFDNPVLP
FNDGVYFASTEKSNIIRGWIFGTTLDSKTQSLLIVNNATNVVIKVCEFQFCNDPFLGVYYHKNNKSWMESGVYSSANNCTFEYVS
QPFLMDLEGKQGNFKNLREFVFKNIDGYFKIYSKHTPINLVRDLPQGFSALEPLVDLPIGINITRFQTLLALHRSYLTPGDSSSG
WTAGAAAYYVGYLQPRTFLLKYNENGTITDAVDCALDPLSETKCTLKSFTVEKGIYQTSNFRVQPTESIVRFPNITNLCPFGEVF
NATRFASVYAWNRKRISNCVADYSVLYNSASFSTFKCYGVSPTKLNDLCFTNVYADSFVIRGDEVRQIAPGQTGKIADYNYKLPD
CDFTGVIAWNSNNLDSKVGGNYNYRYRLFRKSNLKPFERDISTEIYQAGSKPCNGVEGFNCYFPLQSYGFQPTNGVGYQPYRVVV
LSFELLHAPATVCGPKKSTNLVKNKCVNFNFNGLTGTGVLTESNKKFLPFQQFGRDIADTTDAVRDPQTLEILDITPCSFGGVSV
ITPGTNTSNQVAVLYQGVNCTEVPVAIHADQLTPTWRVYSTGSNVFQTRAGCLIGAEHVNNSYECDIPIGAGICASYQTQTNGSG
SIIAYTMSLGAENSVAYSNNSIAIPTNFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQYGDCLGDIAARDLICAQKFNGLTVLP
PLLTDEMIAQYTSALLAGTITSGWTFGAGAALQIPFAMQMAYRFNGIGVTQNVLYENQKLIANQFNSAIGKIQDSLSSTASALGKL
QNVVNQNAQALNTLVKQLSSNFGAISSVLNDILSRLDKVEAEVQIDRLITGRLQSLQTYVTQQLIRAAEIRASANLAATKMSECVL
GQSKRVDFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDLQELGSG
QSLANATAAQQEVLEAQYAMVQHIAKGIRILEARVARGGSGGNHTWQQWEEEIEQHEGNLSLLLREAALQVHIAQRDARRI
wherein:
Italicized text corresponds to the signal peptide of SARS-CoV-2 spike protein;
Bold text is a flexible linker; and
Underlined text corresponds to a VISNA-based SSM CD11
SARS-CoV-2 (Delta) Spike protein-N⁶⁷⁹-GSG-S⁶⁹¹-G¹²⁰⁴-VISNA-based SSM (CD11)
and QS removed from CD11 FHRR
Referred to as ‘SARS-CoV-2 (Delta) S CD11-QS’
SEQ ID NO: 74
MFVFLVLLPLVSSQCVNLRTRTQLPPAYTNSFTRGVYYPDKVFRSSVLHSTQDLFLPFFSNVTWFHAIHVSGTNGTKRFDNPVLP
FNDGVYFASTEKSNIIRGWIFGTTLDSKTQSLLIVNNATNVVIKVCEFQFCNDPFLGVYYHKNNKSWMESGVYSSANNCTFEYVS
QPFLMDLEGKQGNFKNLREFVFKNIDGYFKIYSKHTPINLVRDLPQGFSALEPLVDLPIGINITRFQTLLALHRSYLTPGDSSSG
WTAGAAAYYVGYLQPRTFLLKYNENGTITDAVDCALDPLSETKCTLKSFTVEKGIYQTSNFRVQPTESIVRFPNITNLCPFGEVE
NATRFASVYAWNRKRISNCVADYSVLYNSASFSTFKCYGVSPTKLNDLCFTNVYADSFVIRGDEVRQIAPGQTGKIADYNYKLPD
DFTGCVIAWNSNNLDSKVGGNYNYRYRLFRKSNLKPFERDISTEIYQAGSKPCNGVEGFNCYFPLQSYGFQPTNGVGYQPYRVVV
LSFELLHAPATVCGPKKSTNLVKNKCVNFNFNGLTGTGVLTESNKKFLPFQQFGRDIADTTDAVRDPQTLEILDITPCSFGGVSV
ITPGTNTSNQVAVLYQGVNCTEVPVAIHADQLTPTWRVYSTGSNVFQTRAGCLIGAEHVNNSYECDIPIGAGICASYQTQTNGSG
SIIAYTMSLGAENSVAYSNNSIAIPTNFTISVTTEILPVSMTKTSVDCTMYICGDSTECSNLLLQYGSFCTQLNRALTGIAVEQD
KNTQEVFAQVKQIYKTPPIKDFGGFNFSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLYENQKLIANQFNSAIGKIQDSLSSTASAL
GKLQNVVNQNAQALNTLVKQLSSNFGAISSVLNDILSRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGKAHFPREGVFVSNGTHWFVTQRNFYEPQII
TTDNTFVSGNCDVVIGIVNNTVYDPLQPELDSFKEELDKYFKNHTSPDVDLGDISGINASVVNIQKEIDRLNEVAKNLNESLIDL
QELGSGLANATAAQQEVLEAQYAMVQHIAKGIRILEARVARGGSGGNHTWQQWEEEIEQHEGNLSLLLREAALQVHIAQRDARRI
wherein:
Italicized text corresponds to the signal peptide of Influenza Hemagglutinin protein;
Bold text is a flexible linker; and
Underlined text corresponds to a VISNA-based SSM CD11
RSV Fusion protein 66E-T¹⁰³-GS-^{G145}-E⁵¹¹-GSG-VISNA-based SSM (CD11)
and QS removed from CD11 FHRR Referred to as ‘VL22_CD11-QS’
SEQ ID NO: 75
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGSGGNHTW
QQWEEEIEQHEGNLSLLLREAALQVHIAQRDARRI
wherein:
Italicized text corresponds to the signal peptide of RSV Fusion protein;
Bold text is a flexible linker; and
Underlined text corresponds to a VISNA-based SSM
RSV Fusion protein 66E-T193-GS-^{G145}-E⁵¹¹-GSG-VISNA-based SSM with additional
N-linked glycosylation
sequences and QS removed from CD11 FHRR (CD11_145T8-QS)
Referred to as ‘VL22_CD11_145T8-QS’
SEQ ID NO: 76
MELLILKANAITTILTAVTFCFASGQNITEEFYQSTCSAVSKGYLSALRTGWYTSVITIELSNIKENKCNGTDAKVKLIKQELDKYKN
AVTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGSGGNHTW
QQWEEEIENHTGNLTLLLREAANQTHIAQRDARRI
wherein:
Italicized text corresponds to the signal peptide of RSV Fusion protein;
Bold text is a flexible linker; and
Underlined text corresponds to a VISNA-based SSM
RSV Fusion protein 66E T¹⁰³-GS-^{G145}-E⁵¹¹-GSG-VISNA-based SSM with additional N-linked
glycosylation sequences and QS removed from CD11 FHRR and CT1 mutation included
(CD11_145T8-QS) Referred to as ‘VL22_CD11_145T8-QS_CT1'
SEQ ID NO: 77
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVRHIAKGIRILEARVARGGSGGNHTW
QQWEEEIENHTGNLTLLLREAANQTHIAQRDARRI
wherein:
Italicized text corresponds to the signal peptide of RSV Fusion protein;
Bold text is a flexible linker; and
Underlined text corresponds to a VISNA-based SSM
RSV Fusion protein 66E-T¹⁰³-GS-^{G145}-E⁵¹¹-GSG-VISNA-based SSM with additional N-linked
glycosylation sequences and QS removed from CD11 FHRR and CTS mutation included
(CD11_145T8-QS) Referred to as ‘VL22_CD11_145T8-QS_CT5’
SEQ ID NO: 78
MELLILKANAITTILTAVTFCFASGQNITEEFYQSTCSAVSKGYLSALRTGWYTSVITIELSNIKENKCNGTDAKVKLIKQELDKYKN
AVTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGSGGNHTT
WQQWEEEIENHTGNLTLLLREAANQTHIAQRDARRI
wherein:
Italicized text corresponds to the signal peptide of RSV Fusion protein;
Bold text is a flexible linker; and
Underlined text corresponds to a VISNA-based SSM
RSV Fusion protein 66E T¹⁰³-GS-^{G145}-E⁵¹¹-GSG-VISNA-based SSM with additional N-linked
glycosylation sequences and QS removed from CD11 FHRR and CT9 mutation included
(CD11_145T8-QS) Referred to as ‘VL22_CD11_145T8-QS_CT9'
SEQ ID NO: 79
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGSGGNHTW
QQWEEEIENHTGNLTLLLREAANQTHIAQRDARRI
wherein:
Italicized text corresponds to the signal peptide of RSV Fusion protein;
Bold text is a flexible linker; and
Underlined text corresponds to a VISNA-based SSM
FHRR of CD11
SEQ ID NO: 80
QSLANATAAQQEVLEAQYAMVQHIAKGIRILEARVAR
FHRR of CD10
SEQ ID NO: 81
QTLANATAAQQDVLEATYAMVQHVAKGVRILEARVAR
SHRR of CD11
SEQ ID NO: 82
NHTWQQWEEEIEQHEGNLSLLLREAALQVHIAQRDARRI
SHRR of CD10
SEQ ID NO: 83
NHTWQQWERGLQGYDTNLTILLKESAAMTQLAEEQARRI
Linker in CD11
SEQ ID NO: 84
GGSGG
Linker in CT12 (see Example 16, Table 5)
SEQ ID NO: 85
PGSGG
E. coli EhaG-CD11
SEQ ID NO: 218
MNAGNDNGQGVDYGSGSAGDGWVAIGKGAKANTFMNTSGSSTAVGYDAIAEGQYSSAIGSKTHAIGGASMAFGVSAISEGDR
SIALGASSYSLGQYSMALGRYSKALGKLSIAMGDSSKAEGANAIALGNATKATEIMSIALGDTANASKAYSMALGASSVASEE
NAIAIGAETEAAENATAIGNNAKAKGTNSMAMGFGSLADKVNTIALGNGSQALADNAIAIGQGNKADGVDAIALGNGSQSRGL
NTIALGTASNATGDKSLALGSNSSANGINSVALGADSIADLDNTVSVGNSSLKRKIVNVKNGAIKSDSYDAINGSQLYAISDS
VAKRLGGGAAVDVDDGTVTAPTYNLKNGSKNNVGAALAVLDENTLQWDQTKGKYSAAHGTSSPTASVITDVADGTISASSKDAV
NGSQLKATNDDVEANTANIATNTSNIATNTANIATNTTNITNLTDSVGDLQADALLWNETKKAFSAAHGQDTTSKITNVKDAD
LTADSTDAVNGSQLKTTNDAVATNTTNIANNTSNIATNTTNISNLTETVTNLGEDALKWDKDNGVFTAAHGTETTSKITNVKDG
DLTTGSTDAVNGSQLKTTNDAVATNTTNIATNTTNISNLTETVTNLGEDALKWDKDNGVFTAAHGNNTASKITNILDGTVTATS
SDAINGSQLYDLSSNIATYFGGNASVNTDGVFTGPTYKIGETNYYNVGDALAAINSSFSTSLGDALLWDATAGKFSAKHGTNGD
ASVITDVADGEISDSSSDAVNGSQLHGVSSYVVDALGGGAEVNADGTITAPTYTIANADYDNVGDALNAIDTTLDDALLWDAD
AGENGAFSAAHGKDKTASVITNVANGAISAASSDAINGSQLYTTNKYIADALGGDAEVNADGTITAPTYTIANAEYNNVGDALD
ALDDNALLWDETANGGAGAYNASHDGKASIITNVANGSISEDSTDAVNGSQLNATNMMIEQNTQIINQLAGNTDATYIQENG
AGINYVRTNDDGLAFNDASAQGVGATAIGYNSVAKGDSSVAIGQGSYSDVDTGIALGSSSVSSRVIAKGSRDTSITENGVVIGYD
TTDGELLGALSIGDDGKYRQIINVADGSEAHDAVTVRQLQNAIGAVATTPTKYFHANSTEEDSLAVGTDSLAMGAKTIVNGDKG
IGIGYGAYVDANALNGIAIGSNAQVIHVNSIAIGNGSTTTRGAQTNYTAYNMDAPQNSVGEFSVGSADGQRQITNVAAGSADT
DAVNVGQLKVTDAQVSQNTQSITNLDNRVTNLDSRVTNIENGIGDIVTTGSTKYFKTNTDGVDASAQGKDSVAIGSGSIAAAD
NSVALGTGSVATEENTISVGSSTNQRRITNVAAGKNATDAVNVAQLKSSEAGGVRYDTKADGSIDYSNITLGGGNGGTTRISNV
SAGVNNNDVVNYAQLKQSVQETKQYTDQRMVEMDNKLSKTESKLSGGIASAMAMTGLPQAYTPGGSGQSLANATAAQQE
VLEAQYAMVQHIAKGIRILEARVARGGSGGNHTWQQWEEEIEQHEGNLSLLLREAALQVHIAQRDARRI
wherein:
Bold text is a flexible linker; and
Underlined text corresponds to a VISNA-based SSM
E. coli EibD-CD11
SEQ ID NO: 219
MQNGTYSVLQDDSQKSGPVKYGSTYEVVKTVDNGNFRYEVKEKKNDKRTLFKFDSEGNVTVKGKGITHTLHDPALKDFARTAE
GKKNEQNGNTPPHKLTDSAVRGVYNKVYGLEKTEITGFSVEDGENGKVSLGSDAKASGEFSVAVGNGARATEKASTAVGSWA
AADGKQSTALGVGTYAYANASTALGSVAFVDNTATYGTAAGNRAKVDKDATEGTALGAKATVTNKNSVALGANSVTTRDNEV
YIGYKTGTESDKTYGTRVLGGLSDGTRNSDAATVGQLNRKVGGVYDDVKARITVESEKQKKYTDQKTSEVNEKVEARTTVGVDS
DGKLTRAEGATKTIAVNDGLVALSGRTDRIDYAVGAIDGRVTRNTQSIEKNSKAIAANTRTLQQHSARLDSQQRQINENHKEMK
RAAAQSAALTGLFQPYSVGGSGQSLANATAAQQEVLEAQYAMVQHIAKGIRILEARVARGGSGGNHTWQQWEEEIEQHEGN
LSLLLREAALQVHIAQRDARRI
wherein:
Bold text is a flexible linker; and
Underlined text corresponds to a VISNA-based SSM
E. coli UpaG-CD11
SEQ ID NO: 220
MDNYTGQPTDYGDGSAGDGWVAIGKGAKANTFMNTSGASTALGYDAIAEGEYSSAIGSKTLATGGASMAFGVSAKAMGDR
SVALGASSVANGDRSMAFGRYAKTNGFTSLAIGDSSLADGEKTIALGNTAKAYEIMSIALGDNANASKEYAMALGASSKAGGA
DSLAFGRKSTANSTGSLAIGADSSSSNDNAIAIGNKTQALGVNSMALGNASQASGESSIALGNTSEASEQNAIALGQGSIASKVN
SIALGSNSLSSGENAIALGEGSAAGGSNSLAFGSQSRANGNDSVAIGVGAAAATDNSVAIGAGSTTDASNTVSVGNSATKRKIV
NMAAGAISNTSTDAINGSQLYTISDSVAKRLGGGATVGSDGTVTAVSYALRSGTYNNVGDALSGIDNNTLQWNKTAGAFSAN
HGANATNKITNVAKGTVSATSTDVVNGSQLYDLQQDALLWNGTAFSAAHGTEATSKITNVTAGNLTAGSTDAVNGSQLKTTN
DNVTTNTTNIATNTTNITNLTDAVNGLGDDSLLWNKAAGAFSAAHGTEATSKITNVTAGNLTAGSTDAVNGSQLKTTNDNVTT
NTTNIATNTTNITNLTDAVNGLGDDSLLWNKTAGAFSAAHGTDATSKITNVTAGNLTAGSTDAVNGSQLKTTNDNVTTNTTNI
ATNTTNITNLTDAVNGLGDDSLLWNKTAGAFSAAHGTDATSKITNVKAGDLTAGSTDAVNGSQLKTTNDNVSTNTTNITNLTD
AVNGLGDDSLLWNKTAGAFSAAHGTDATSKITNVKAGDLTAGSTDAVNGSQLKTTNDNVSTNTTNITNLTDSVGDLKDDSLLW
NKAAGAFSAAHGTEATSKITNLLAGKISSNSTDAINGSQLYGVADSFTSYLGGGADISDTGVLSGPTYTIGGTDYTNVGDALA
AINTSFSTSLGDALLWDATAGKFSAKHGINNAPSVITDVANGAVSSTSSDAINGSQLYGVSDYIADALGGNAVVNTDGSITTP
TYAIAGGSYNNVGDALEAIDTTLDDALLWDTTANGGNGAFSAAHGKDKTASVITNVANGAVSATSNDAINGSQLYSTNKYIAD
ALGGDAEVNADGTITAPTYTIANTDYNNVGEALDALDNNALLWDEDAGAYNASHDGNASKITNVAAGDLSTTSTDAVNGSQLN
ATNILVTQNSQMINQLAGNTSETYIEENGAGINYVRTNDSGLAFNDASASGIGATAVGYNAVASHASSVAIGQDSISEVDTGI
ALGSSSVSSRVIVKGTRNTSVSEEGVVIGYDTTDGELLGALSIGDDGKYRQIINVADGSEAHDAVTVRQLQNAIGAVATTPTK
YYHANSTAEDSLAVGEDSLAMGAKTIVNGNAGIGIGLNTLVLADAINGIAIGSNARANHADSIAMGNGSQTTRGAQTNYTAYN
MDAPQNSVGEFSVGSEDGQRQITNVAAGSADTDAVNVGQLKVTDAQVSQNTQSITNLNTQVTNLDTRVTNIENGIGDIVTTGS
TKYFKTNTDGADANAQGKDSVAIGSGSIAAADNSVALGTGSVADEENTISVGSSTNQRRITNVAAGVNATDAVNVSQLKSSEA
GGVRYDTKADGSIDYSNITLGGGNSGTTRISNVSAGVNNNDAVNYAQLKQSVQETKQYTDQRMVEMDNKLSKTESKLSGGIAS
AMAMTGLPQAYTPGGSGQSLANATAAQQEVLEAQYAMVQHIAKGIRILEARVARGGSGGNHTWQQWEEEIEQHEGNLSLLLRE
AALQVHIAQRDARRI
wherein:
Bold text is a flexible linker; and
Underlined text corresponds to a VISNA-based SSM
H. influenzae HIA-CD11
SEQ ID NO: 221
MNNNTPVTNKLKAYGDANFNFTNNSIADAEKQVQEAYKGLLNLNEKNASDKLLVEDNTAATVGNLRKLGWVLSSKNGTRNEK
SQQVKHADEVLFEGKGGVQVTSTSENGKHTITFALAKDLGVKTATVSDTLTIGGGAAAGATTTPKVNVTSTTDGLKFAKDAAGA
NGDTTVHLNGIGSTLTDTLVGSPATHIDGGDQSTHYTRAASIKDVLNAGWNIKGVKAGSTTGQSENVDFVHTYDTVEFLSADTE
TTTVTVDSKENGKRTEVKIGAKTSVIKEKDGKLFTGKANKETNKVDGANATEDADEGKGLVTAKDVIDAVNKTGWRIKTTDAN
GQNGDFATVASGTNVTFASGNGTTATVTNGTDGITVKYDAKVGDGLKLDGDKIAADTTALTVNDGKNANNPKGKVADVASTD
EKKLVTAKGLVTALNSLSWTTTAAEADGGTLDGNASEQEVKAGDKVTFKAGKNLKVKQEGANFTYSLQDALTGLTSITLGTGN
NGAKTEINKDGLTITPANGAGANNANTISVTKDGISAGGQSVKNVVSGLKKFGDANFDPLTSSADNLTKQNDDAYKGLTNLDE
KGTDKQTPVVADNTAATVGDLRGLGWVISADKTTGGSTEYHDQVRNANEVKFKSGNGINVSGKTVNGRREITFELAKGEVVKS
NEFTVKETNGKETSLVKVGDKYYSKEDIDLTTGQPKLKDGNTVAAKYQDKGGKVVSVTDNTEATITNKGSGYVTGNQVADAIA
KSGFELGLADEADAKAAFDDKTKALSAGTTEIVNAHDKVRFANGLNTKVSAATVESTDANGDKVTTTFVKTDVELPLTQIYNT
DANGKKITKVVKDGQTKWYELNADGTADMTKEVTLGNVDSDGKKVVKDNDGKWYHAKADGTADKTKGEVSNDKVSTDEKHVV
SLDPNDQSKGKGVVIDNVANGDISATSTDAINGSQLYAVAKGVTNLAGQVNNLEGKVNKVGKRADAGTASALAASQLPQAT
MPGGSGQSLANATAAQQEVLEAQYAMVQHIAKGIRILEARVARGGSGGNHTWQQWEEEIEQHEGNLSLLLREAALQVHIAQ
RDARRI
wherein:
Bold text is a flexible linker; and
Underlined text corresponds to a VISNA-based SSM
N. meningitidis NadA-CD11
SEQ ID NO: 222
MATNDDDVKKAATVAIAAAYNNGQEINGFKAGETIYDIDEDGTITKKDATAADVEADDFKGLGLKKVVTNLTKTVNENKQNVD
AKVKAAESEIEKLTTKLADTDAALADTDAALDATTNALNKLGENITTFAEETKTNIVKIDEKLEAVADTVDKHAEAFNDIADS
LDETNTKADEAVKTANEAKQTAEETKQNVDAKVKAAETAAGKAEAAAGTANTAADKAEAVAAKVTDIKADIATNKDNIAKKAN
SADVYTREESDSKFVRIDGLNATTEKLDTRLASAEKSIADHDTRLNGLDKTVSDLRKETRQGLAEQAALSGLFQPYNVGGSGQ
SLANATAAQQEVLEAQYAMVQHIAKGIRILEARVARGGSGGNHTWQQWEEEIEQHEGNLSLLLREAALQVHIAQRDARRI
wherein:
Bold text is a flexible linker; and
Underlined text corresponds to a VISNA-based SSM
N. meningitidis NhhA-CD11
SEQ ID NO: 223
MNNEEQEEDLYLDPVQRTVAVLIVNSDKEGTGEKEKVEENSDWAVYFNEKGVLTAREITLKAGDNLKIKQNGTNFTYSLKKDLT
DLTSVGTEKLSFSANGNKVNITSDTKGLNFAKETAGTNGDTTVHLNGIGSTLTDTLLNTGATTNVTNDNVTDDEKKRAASVKDV
LNAGWNIKGVKPGTTASDNVDFVRTYDTVEFLSADTKTTTVNVESKDNGKKTEVKIGAKTSVIKEKDGKLVTGKDKGENGSSTD
EGEGLVTAKEVIDAVNKAGWRMKTTTANGQTGQADKFETVTSGTNVTFASGKGTTATVSKDDQGNITVMYDVNVGDALNV
NQLQNSGWNLDSKAVAGSSGKVISGNVSPSKGKMDETVNINAGNNIEITRNGKNIDIATSMTPQFSSVSLGAGADAPTLSVDG
DALNVGSKKDNKPVRITNVAPGVKEGDVTNVAQLKGVAQNLNNRIDNVDGNARAGIAQAIATAGLVQAYLPGGSGQSLANA
TAAQQEVLEAQYAMVQHIAKGIRILEARVARGGSGGNHTWQQWEEEIEQHEGNLSLLLREAALQVHIAQRDARRI
wherein:
Bold text is a flexible linker; and
Underlined text corresponds to a VISNA-based SSM
Y. enterocolitica YadA-CD11
SEQ ID NO: 224
MDDYDGIPNLTAVQISPNADPALGLEYPVRPPVPGAGGLNASAKGIHSIAIGATAEAAKGAAVAVGAGSIATGVNSVAIGPLSK
ALGDSAVTYGAASTAQKDGVAIGARASTSDTGVAVGFNSKADAKNSVAIGHSSHVAANHGYSIAIGDRSKTDRENSVSIGHESL
NRQLTHLAAGTKDTDAVNVAQLKKEIEKTQENTNKRSAELLANANAYADNKSSSVLGIANNYTDSKSAETLENARKEAFAQSKD
VLNMAKAHSNSVARTTLETAEEHANSVARTTLETAEEHANKKSAEALASANVYADSKSSHTLKTANSYTDVTVSNSTKKAIRESN
QYTDHKFRQLDNRLDKLDTRVDKGLASSAALNSLFQPYGVGGSGQSLANATAAQQEVLEAQYAMVQHIAKGIRILEARVARG
GSGGNHTWQQWEEEIEQHEGNLSLLLREAALQVHIAQRDARRI
wherein:
Bold text is a flexible linker; and
Underlined text corresponds to a VISNA-based SSM
VL22 with membrane tethered silenced Clamp2 alpha (VL22_CTSs_alpha)
SEQ ID NO: 225
MELLILKANAITTILTAVTFCFASGQNITEEFYQSTCSAVSKGYLSALRTGWYTSVITIELSNIKENKCNGTDAKVKLIKQELDKYKN
AVTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGKRVIDGFLLI
LTSPIIIATAILSGGNHTTWQQWEEEIENHTGNLTLLLREAANQTHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the membrane tethering
(poly)peptide alpha
VL22 with membrane tethered silenced Clamp2 beta (VL22_CTSs_beta)
SEQ ID NO: 226
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGYRLLSPDLL
RYSGGNHTTWQQWEEEIENHTGNLTLLLREAANQTHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the membrane tethering
(poly)peptide beta
VL22 with membrane tethered silenced Clamp2 gamma (VL22_CTSs_gamma)
SEQ ID NO: 227
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGKAYEVAGLL
GDIGGTNRIQYYFLSGGNHTTWQQWEEEIENHTGNLTLLLREAANQTHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the membrane tethering
(poly)peptide gamma
VL22 with membrane tethered silenced Clamp2 delta (VL22_CTSs_delta)
SEQ ID NO: 228
MELLILKANAITTILTAVTFCFASGQNITEEFYQSTCSAVSKGYLSALRTGWYTSVITIELSNIKENKCNGTDAKVKLIKQELDKYKN
AVTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGLGDLILILR
LPSWFKRLLSLLLKPLFPRLAAFLNSMRPRSAESGGNHTTWQQWEEEIENHTGNLTLLLREAANQTHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the membrane tethering
(poly)peptide delta
VL22 with membrane tethered silenced Clamp2 epsilon (VL22_CTSs_epsilon)
SEQ ID NO: 229
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGGTLNRFTQ
ALVIAYVIGGGLALLGLVNVICDWSGGNHTTWQQWEEEIENHTGNLTLLLREAANQTHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the membrane tethering
(poly)peptide epsilon
VL22 with membrane tethered silenced Clamp2 neta (VL22_CTSs_neta)
SEQ ID NO: 230
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGKVLTIIATIF
MPLGGGVIAVIMVVYFKKKKSGGNHTTWQQWEEEIENHTGNLTLLLREAANQTHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the membrane tethering
(poly)peptide neta
VL22 with membrane tethered silenced Clamp2 theta (VL22_CTSs_theta)
SEQ ID NO: 231
MELLILKANAITTILTAVTFCFASGQNITEEFYQSTCSAVSKGYLSALRTGWYTSVITIELSNIKENKCNGTDAKVKLIKQELDKYKNA
VTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIF
NPKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGKEQLSTAID
RMNEGLDAFIQLYSGGNHTTWQQWEEEIENHTGNLTLLLREAANQTHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the membrane tethering
(poly)peptide theta
VL22 with membrane tethered silenced Clamp2 eta (VL22_CT5s_eta)
SEQ ID NO: 232
MELLILKANAITTILTAVTFCFASGQNITEEFYQSTCSAVSKGYLSALRTGWYTSVITIELSNIKENKCNGTDAKVKLIKQELDKYKN
AVTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVSNKGVDTVSVGNTLYYVNKQEGKSLYVKGEPI
INFYDPLVFPSDEFDASISQVNEKINQSLAFIRKSDEGSGLANATAAQQEVLEAQYAMVQHIAKGIRILEARVARGGHWRAAGA
ATVLLGGVVVMVAGITSYSGGNHTTWQQWEEEIENHTGNLTLLLREAANQTHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the membrane tethering
(poly)peptide eta
VL22 with native transmembrane and cytoplasmic Clamp2 (mVL22-TM-CD11)
SEQ ID NO: 233
MELLILKANAITTILTAVTFCFASGQNITEEFYQSTCSAVSKGYLSALRTGWYTSVITIELSNIKENKCNGTDAKVKLIKQELDKYKN
AVTELQLLMQSTPATGSGSAIASGVAVSKVLHLEGEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQKKLMSNNVQIVRQQSYSIMSIIKEEVLAYVVQ
LPLYGVIDTPCWKLHTSPLCTTNTKEGSNICLTRTDRGWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGCDYVSNKGVDTVSVGNTLYYVNKQEGKSLYVKGEPIIN
FYDPLVFPSDEFDASISQVNEKINQSLAFIRKSDELLHNVNAGKSTTNIMITTIIIVIIVILLSLIAVGLLLYCKGSGLANATAAQQ
EVLEAQYAMVQHIAKGIRILEARVARGGSGGNHTWQQWEEEIEQHEGNLSLLLREAALQVHIAQRDARRI
wherein:
Italicized text corresponds to the signal peptide of RSV Fusion protein;
Bold text is a flexible linker;
Underlined text corresponds to a VISNA-based SSM; and
Underlined and Italicized text corresponds to the native transmembrane
domain of RSV F
Native full length RSV F (mFwt)
SEQ ID NO: 234
MELLILKANAITTILTAVTFCFASGQNITEEFYQSTCSAVSKGYLSALRTGWYTSVITIELSNIKENKCNGTDAKVKLIKQELDKYK
NAVTELQLLMQSTPATNNRARRELPRFMNYTLNNAKKTNVTLSKKRKRRFLGFLLGVGSAIASGVAVSKVLHLEGEVNKIKSALLST
NKAVVSLSNGVSVLTSKVLDLKNYIDKQLLPIVNKQSCSISNIETVIEFQQKNNRLLEITREFSVNAGVTTPVSTYMLTNSELLSLI
NDMPITNDQKKLMSNNVQIVRQQSYSIMSIIKEEVLAYVVQLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKIMTSKTDVSSSVITSLGAIVSCYGKTKCTASNKNRGIIKT
FSNGCDYVSNKGVDTVSVGNTLYYVNKQEGKSLYVKGEPIINFYDPLVFPSDEFDASISQVNEKINQSLAFIRKSDELLHNVNAGK
STTNIMITTIIIVIIVILLSLIAVGLLLYCKARSTPVTLSKDQLSGINNIAFSN
wherein:
Italicized text corresponds to the signal peptide of RSV Fusion protein; and
Underlined and Italicized text corresponds to the native transmembrane
domain of RSV
DSCav1AY stabilized RSV F with transmembrane domain (mDSCavAY)
SEQ ID NO: 235
MELLILKANAITTILTAVTFCFASGQNITEEFYQSTCSAVSKGYLSALRTGWYTSVITIELSNIKENKCNGTDAKVKLIKQELDKYK
NAVTELQLLMQSTPATGSGSAICSGVAVCKVLHLEGEVNKIKSALLSTNKAVVSLSNGVSVLTFKVLDLKNYIDKQLLPILNKQSCS
ISNIETVIEFQQKNNRLLEITREFSVNAGVTTPVSTYMLTNSELLSLINDMPITNDQKKLMSNNVQIVRQQSYSIMCIIKEEVLAYV
VQLPLYGVIDTPCWKLHTSPLCTTNTKEGSNICLTRTDRGWYCDNAGSVSFFPQAETCKVQSNRVFCDTMNSRTLPSEVNLCNVDIF
NPKYDCKIMTSKTDVSSSVITSLGAIVSCYGKTKCTASNKNRGIIKTFSNGCDYVSNKGVDTVSVGNTLYCVNKQEGKSLYVKGEP
IINFYDPLVFPSDEFDASISQVNEKINQSLAFIRKSDELLHNVNAGKSTTNIMITTIIIVIIVILLSLIAVGLLLYCKAR*
wherein:
Italicized text corresponds to the signal peptide of RSV Fusion protein;
Bold text is a flexible linker; and
Underlined and Italicized text corresponds to the native transmembrane
domain of RSV
Membrane tethering (poly)peptide “alpha” (as comprised in SEQ ID NO: 225):
SEQ ID NO: 236
KRVIDGFLLILTSPIIIATAIL
Membrane tethering (poly)peptide “beta” (as comprised in SEQ ID NO: 226):
SEQ ID NO: 237
YRLLSPDLLRY
Membrane tethering (poly)peptide “gamma” (as comprised in SEQ ID NO: 227):
SEQ ID NO: 238
KAYEVAGLLGDIGGTNRIQYYFL
Membrane tethering (poly)peptide “delta” (as comprised in SEQ ID NO: 228);
SEQ ID NO: 239
LGDLILILRLPSWFKRLLSLLLKPLFPRLAAFLNSMRPRSAE
Membrane tethering (poly)peptide “epsilon” (as comprised in SEQ ID NO: 229):
SEQ ID NO: 240
GTLNRFTQALVIAYVIGGGLALLGLVNVICDW
Membrane tethering (poly)peptide “neta” (as comprised in SEQ ID NO: 230):
SEQ ID NO: 241
KVLTIIATIFMPLGGGVIAVIMVVYFKKKK
Membrane tethering (poly)peptide “theta” (as comprised in SEQ ID NO: 231):
SEQ ID NO: 242
KEQLSTAIDRMNEGLDAFIQLY
Membrane tethering (poly)peptide “eta” (as comprised in SEQ ID NO: 232):
SEQ ID NO: 243
HWRAAGAATVLLGGVVVMVAGITSYS
Membrane tethering (poly)peptide “alpha” plus flanking linker residues
(as comprised in SEQ ID NO: 225):
SEQ ID NO: 244
GGKRVIDGFLLILTSPIIIATAILSGG
Membrane tethering (poly)peptide “beta” plus flanking linker residues
(as comprised in SEQ ID NO: 226):
SEQ ID NO: 245
GGYRLLSPDLLRYSGG
Membrane tethering (poly)peptide “gamma” plus flanking linker residues
(as comprised in SEQ ID NO: 227):
SEQ ID NO: 246
GGKAYEVAGLLGDIGGTNRIQYYFLSGG
Membrane tethering (poly)peptide “delta” plus flanking linker residues
(as comprised in SEQ ID NO: 228):
SEQ ID NO: 247
GGLGDLILILRLPSWFKRLLSLLLKPLFPRLAAFLNSMRPRSAESGG
Membrane tethering (poly)peptide “epsilon” plus flanking linker residues
(as comprised in SEQ ID NO: 229):
SEQ ID NO: 248
GGGTLNRFTQALVIAYVIGGGLALLGLVNVICDWSGG
Membrane tethering (poly)peptide “neta” plus flanking linker residues
(as comprised in SEQ ID NO: 230):
SEQ ID NO: 249
GGKVLTIIATIFMPLGGGVIAVIMVVYFKKKKSGG
Membrane tethering (poly)peptide “theta” plus flanking linker residues
(as comprised in SEQ ID NO: 231);
SEQ ID NO: 250
GGKEQLSTAIDRMNEGLDAFIQLYSGG
Membrane tethering (poly)peptide “eta” plus flanking linker residues
(as comprised in SEQ ID NO: 232):
SEQ ID NO: 251
GGHWRAAGAATVLLGGVVVMVAGITSYSGG
E. coli EhaG (as comprised in SEQ ID NO: 218)
SEQ ID NO: 252
MNAGNDNGQGVDYGSGSAGDGWVAIGKGAKANTFMNTSGSSTAVGYDAIAEGQYSSAIGSKTHAIGGASMAFGVSAISEG
DRSIALGASSYSLGQYSMALGRYSKALGKLSIAMGDSSKAEGANAIALGNATKATEIMSIALGDTANASKAYSMALGASSVASEE
NAIAIGAETEAAENATAIGNNAKAKGTNSMAMGFGSLADKVNTIALGNGSQALADNAIAIGQGNKADGVDAIALGNGSQSRG
LNTIALGTASNATGDKSLALGSNSSANGINSVALGADSIADLDNTVSVGNSSLKRKIVNVKNGAIKSDSYDAINGSQLYAISDSVA
KRLGGGAAVDVDDGTVTAPTYNLKNGSKNNVGAALAVLDENTLQWDQTKGKYSAAHGTSSPTASVITDVADGTISASSKDAV
NGSQLKATNDDVEANTANIATNTSNIATNTANIATNTTNITNLTDSVGDLQADALLWNETKKAFSAAHGQDTTSKITNVKDAD
LTADSTDAVNGSQLKTTNDAVATNTTNIANNTSNIATNTTNISNLTETVTNLGEDALKWDKDNGVFTAAHGTETTSKITNVKDG
DLTTGSTDAVNGSQLKTTNDAVATNTTNIATNTTNISNLTETVTNLGEDALKWDKDNGVFTAAHGNNTASKITNILDGTVTATS
SDAINGSQLYDLSSNIATYFGGNASVNTDGVFTGPTYKIGETNYYNVGDALAAINSSFSTSLGDALLWDATAGKFSAKHGTNGD
ASVITDVADGEISDSSSDAVNGSQLHGVSSYVVDALGGGAEVNADGTITAPTYTIANADYDNVGDALNAIDTTLDDALLWDAD
AGENGAFSAAHGKDKTASVITNVANGAISAASSDAINGSQLYTTNKYIADALGGDAEVNADGTITAPTYTIANAEYNNVGDALD
ALDDNALLWDETANGGAGAYNASHDGKASIITNVANGSISEDSTDAVNGSQLNATNMMIEQNTQIINQLAGNTDATYIQENG
AGINYVRTNDDGLAFNDASAQGVGATAIGYNSVAKGDSSVAIGQGSYSDVDTGIALGSSSVSSRVIAKGSRDTSITENGVVIGYD
TTDGELLGALSIGDDGKYRQIINVADGSEAHDAVTVRQLQNAIGAVATTPTKYFHANSTEEDSLAVGTDSLAMGAKTIVNGDKG
IGIGYGAYVDANALNGIAIGSNAQVIHVNSIAIGNGSTTTRGAQTNYTAYNMDAPQNSVGEFSVGSADGQRQITNVAAGSADT
DAVNVGQLKVTDAQVSQNTQSITNLDNRVTNLDSRVTNIENGIGDIVTTGSTKYFKTNTDGVDASAQGKDSVAIGSGSIAAAD
NSVALGTGSVATEENTISVGSSTNQRRITNVAAGKNATDAVNVAQLKSSEAGGVRYDTKADGSIDYSNITLGGGNGGTTRISNV
SAGVNNNDVVNYAQLKQSVQETKQYTDQRMVEMDNKLSKTESKLSGGIASAMAMTGLPQAYTP
E. coli EibD (as comprised in SEQ ID NO: 219)
SEQ ID NO: 253
MQNGTYSVLQDDSQKSGPVKYGSTYEVVKTVDNGNFRYEVKEKKNDKRTLFKFDSEGNVTVKGKGITHTLHDPALKDFARTAE
GKKNEQNGNTPPHKLTDSAVRGVYNKVYGLEKTEITGFSVEDGENGKVSLGSDAKASGEFSVAVGNGARATEKASTAVGSWA
AADGKQSTALGVGTYAYANASTALGSVAFVDNTATYGTAAGNRAKVDKDATEGTALGAKATVTNKNSVALGANSVTTRDNEV
YIGYKTGTESDKTYGTRVLGGLSDGTRNSDAATVGQLNRKVGGVYDDVKARITVESEKQKKYTDQKTSEVNEKVEARTTVGVDS
DGKLTRAEGATKTIAVNDGLVALSGRTDRIDYAVGAIDGRVTRNTQSIEKNSKAIAANTRTLQQHSARLDSQQRQINENHKEMK
RAAAQSAALTGLFQPYSV
E. coli UpaG (as comprised in SEQ ID NO: 220)
SEQ ID NO: 254
MDNYTGQPTDYGDGSAGDGWVAIGKGAKANTFMNTSGASTALGYDAIAEGEYSSAIGSKTLATGGASMAFGVSAKAMGDR
SVALGASSVANGDRSMAFGRYAKTNGFTSLAIGDSSLADGEKTIALGNTAKAYEIMSIALGDNANASKEYAMALGASSKAGGA
DSLAFGRKSTANSTGSLAIGADSSSSNDNAIAIGNKTQALGVNSMALGNASQASGESSIALGNTSEASEQNAIALGQGSIASKVN
SIALGSNSLSSGENAIALGEGSAAGGSNSLAFGSQSRANGNDSVAIGVGAAAATDNSVAIGAGSTTDASNTVSVGNSATKRKIV
NMAAGAISNTSTDAINGSQLYTISDSVAKRLGGGATVGSDGTVTAVSYALRSGTYNNVGDALSGIDNNTLQWNKTAGAFSAN
HGANATNKITNVAKGTVSATSTDVVNGSQLYDLQQDALLWNGTAFSAAHGTEATSKITNVTAGNLTAGSTDAVNGSQLKTTN
DNVTTNTTNIATNTTNITNLTDAVNGLGDDSLLWNKAAGAFSAAHGTEATSKITNVTAGNLTAGSTDAVNGSQLKTTNDNVTT
NTTNIATNTTNITNLTDAVNGLGDDSLLWNKTAGAFSAAHGTDATSKITNVTAGNLTAGSTDAVNGSQLKTTNDNVTTNTTNI
ATNTTNITNLTDAVNGLGDDSLLWNKTAGAFSAAHGTDATSKITNVKAGDLTAGSTDAVNGSQLKTTNDNVSTNTTNITNLTD
AVNGLGDDSLLWNKTAGAFSAAHGTDATSKITNVKAGDLTAGSTDAVNGSQLKTTNDNVSTNTTNITNLTDSVGDLKDDSLLW
NKAAGAFSAAHGTEATSKITNLLAGKISSNSTDAINGSQLYGVADSFTSYLGGGADISDTGVLSGPTYTIGGTDYTNVGDALA
AINTSFSTSLGDALLWDATAGKFSAKHGINNAPSVITDVANGAVSSTSSDAINGSQLYGVSDYIADALGGNAVVNTDGSITTP
TYAIAGGSYNNVGDALEAIDTTLDDALLWDTTANGGNGAFSAAHGKDKTASVITNVANGAVSATSNDAINGSQLYSTNKYIAD
ALGGDAEVNADGTITAPTYTIANTDYNNVGEALDALDNNALLWDEDAGAYNASHDGNASKITNVAAGDLSTTSTDAVNGSQLN
ATNILVTQNSQMINQLAGNTSETYIEENGAGINYVRTNDSGLAFNDASASGIGATAVGYNAVASHASSVAIGQDSISEVDTGI
ALGSSSVSSRVIVKGTRNTSVSEEGVVIGYDTTDGELLGALSIGDDGKYRQIINVADGSEAHDAVTVRQLQNAIGAVATTPTK
YYHANSTAEDSLAVGEDSLAMGAKTIVNGNAGIGIGLNTLVLADAINGIAIGSNARANHADSIAMGNGSQTTRGAQTNYTAYN
MDAPQNSVGEFSVGSEDGQRQITNVAAGSADTDAVNVGQLKVTDAQVSQNTQSITNLNTQVTNLDTRVTNIENGIGDIVTTGS
TKYFKTNTDGADANAQGKDSVAIGSGSIAAADNSVALGTGSVADEENTISVGSSTNQRRITNVAAGVNATDAVNVSQLKSSEA
GGVRYDTKADGSIDYSNITLGGGNSGTTRISNVSAGVNNNDAVNYAQLKQSVQETKQYTDQRMVEMDNKLSKTESKLSGGIAS
AMAMTGLPQAYTP
H. influenzae HIA (as comprised in SEQ ID NO: 221)
SEQ ID NO: 255
MNNNTPVTNKLKAYGDANFNFTNNSIADAEKQVQEAYKGLLNLNEKNASDKLLVEDNTAATVGNLRKLGWVLSSKNGTRNEK
SQQVKHADEVLFEGKGGVQVTSTSENGKHTITFALAKDLGVKTATVSDTLTIGGGAAAGATTTPKVNVTSTTDGLKFAKDAAGA
NGDTTVHLNGIGSTLTDTLVGSPATHIDGGDQSTHYTRAASIKDVLNAGWNIKGVKAGSTTGQSENVDFVHTYDTVEFLSADTE
TTTVTVDSKENGKRTEVKIGAKTSVIKEKDGKLFTGKANKETNKVDGANATEDADEGKGLVTAKDVIDAVNKTGWRIKTTDAN
GQNGDFATVASGTNVTFASGNGTTATVTNGTDGITVKYDAKVGDGLKLDGDKIAADTTALTVNDGKNANNPKGKVADVASTD
EKKLVTAKGLVTALNSLSWTTTAAEADGGTLDGNASEQEVKAGDKVTFKAGKNLKVKQEGANFTYSLQDALTGLTSITLGTGN
NGAKTEINKDGLTITPANGAGANNANTISVTKDGISAGGQSVKNVVSGLKKFGDANFDPLTSSADNLTKQNDDAYKGLTNLDE
KGTDKQTPVVADNTAATVGDLRGLGWVISADKTTGGSTEYHDQVRNANEVKFKSGNGINVSGKTVNGRREITFELAKGEVVKS
NEFTVKETNGKETSLVKVGDKYYSKEDIDLTTGQPKLKDGNTVAAKYQDKGGKVVSVTDNTEATITNKGSGYVTGNQVADAIAK
SGFELGLADEADAKAAFDDKTKALSAGTTEIVNAHDKVRFANGLNTKVSAATVESTDANGDKVTTTFVKTDVELPLTQIYNTDA
NGKKITKVVKDGQTKWYELNADGTADMTKEVTLGNVDSDGKKVVKDNDGKWYHAKADGTADKTKGEVSNDKVSTDEKHVV
SLDPNDQSKGKGVVIDNVANGDISATSTDAINGSQLYAVAKGVTNLAGQVNNLEGKVNKVGKRADAGTASALAASQLPQAT
MP
N. meningitidis NadA (as comprised in SEQ ID NO: 222)
SEQ ID NO: 256
MATNDDDVKKAATVAIAAAYNNGQEINGFKAGETIYDIDEDGTITKKDATAADVEADDFKGLGLKKVVTNLTKTVNENKQNVD
AKVKAAESEIEKLTTKLADTDAALADTDAALDATTNALNKLGENITTFAEETKTNIVKIDEKLEAVADTVDKHAEAFNDIADSLDET
NTKADEAVKTANEAKQTAEETKQNVDAKVKAAETAAGKAEAAAGTANTAADKAEAVAAKVTDIKADIATNKDNIAKKANSAD
VYTREESDSKFVRIDGLNATTEKLDTRLASAEKSIADHDTRLNGLDKTVSDLRKETRQGLAEQAALSGLFQPYNV
N. meningitidis NhhA (as comprised in SEQ ID NO: 223)
SEQ ID NO: 257
MNNEEQEEDLYLDPVQRTVAVLIVNSDKEGTGEKEKVEENSDWAVYFNEKGVLTAREITLKAGDNLKIKQNGTNFTYSLKKDLT
DLTSVGTEKLSFSANGNKVNITSDTKGLNFAKETAGTNGDTTVHLNGIGSTLTDTLLNTGATTNVTNDNVTDDEKKRAASVKDV
LNAGWNIKGVKPGTTASDNVDFVRTYDTVEFLSADTKTTTVNVESKDNGKKTEVKIGAKTSVIKEKDGKLVTGKDKGENGSSTD
EGEGLVTAKEVIDAVNKAGWRMKTTTANGQTGQADKFETVTSGTNVTFASGKGTTATVSKDDQGNITVMYDVNVGDALNV
NQLQNSGWNLDSKAVAGSSGKVISGNVSPSKGKMDETVNINAGNNIEITRNGKNIDIATSMTPQFSSVSLGAGADAPTLSVDG
DALNVGSKKDNKPVRITNVAPGVKEGDVTNVAQLKGVAQNLNNRIDNVDGNARAGIAQAIATAGLVQAYLP
Y. enterocolitica YadA (as comprised in SEQ ID NO: 224)
SEQ ID NO: 258
MDDYDGIPNLTAVQISPNADPALGLEYPVRPPVPGAGGLNASAKGIHSIAIGATAEAAKGAAVAVGAGSIATGVNSVAIGPLSK
ALGDSAVTYGAASTAQKDGVAIGARASTSDTGVAVGFNSKADAKNSVAIGHSSHVAANHGYSIAIGDRSKTDRENSVSIGHESL
NRQLTHLAAGTKDTDAVNVAQLKKEIEKTQENTNKRSAELLANANAYADNKSSSVLGIANNYTDSKSAETLENARKEAFAQSKD
VLNMAKAHSNSVARTTLETAEEHANSVARTTLETAEEHANKKSAEALASANVYADSKSSHTLKTANSYTDVTVSNSTKKAIRESN
QYTDHKFRQLDNRLDKLDTRVDKGLASSAALNSLFQPYGV.

1. A chimeric polypeptide comprising a microbial polypeptide operably connected downstream to a heterologous, structure-stabilizing moiety (SSM), wherein the structure-stabilizing moiety is a polypeptide comprising, in an N- to C-terminal order, a first heptad repeat region (FHRR) and a second heptad repeat region (SHRR), wherein
(i) the FHRR comprises or consists of an amino acid sequence having at least 60% sequence identity to the amino acid sequence set forth in SEQ ID NO: 80 or 81, and the SHRR comprises or consists of an amino acid sequence having at least 40% sequence identity to the amino acid sequence set forth in SEQ ID NO: 82 or 83; and/or
(ii) the FHRR comprises or consists of an amino acid sequence having at least 90% sequence similarity to the amino acid sequence set forth in SEQ ID NO: 80 or 81, and the SHRR comprises or consists of an amino acid sequence having at least 70% sequence similarity to the amino acid sequence set forth in SEQ ID NO: 82 or 83.

2. The chimeric polypeptide of claim 1, wherein:
(i) the FHRR comprises or consists of an amino acid sequence having at least 60% sequence identity to the amino acid sequence set forth in SEQ ID NO: 80, and the SHRR comprises or consists of an amino acid sequence having at least 40% sequence identity to the amino acid sequence set forth in SEQ ID NO: 82; and/or
(ii) the FHRR comprises or consists of an amino acid sequence having at least 90% sequence similarity to the amino acid sequence set forth in SEQ ID NO: 80, and the SHRR comprises or consists of an amino acid sequence having at least 70% sequence similarity to the amino acid sequence set forth in SEQ ID NO: 82.

3. The chimeric polypeptide of claim 1 or 2, wherein the structure-stabilizing moiety is capable of homo-trimerization with the structure-stabilizing moieties of two further chimeric polypeptides; wherein preferably, by the homo-trimerization, a six-helix bundle is formed, wherein the six-helix bundle is composed of an inner trimer of three parallel oriented, substantially α-helical FHRRs against which three substantially α-helical SHRRs are packed in an anti-parallel orientation relative to the FHRRs.

4. The chimeric polypeptide of any one of claims 1 to 3, wherein the FHRR and SHRR each comprise an independently selected, n-times repeated 7-residue motif characterized by a pattern of amino acids, represented as (a-b-c-d-e-f-g-)ₙ or (d-e-f-g-a-b-c-)ₙ, wherein the pattern elements ‘a’ to ‘g’ denote positions at which the amino acids are located and n is a number equal to or greater than 2, and at least 50% of the positions ‘a’ and ‘d’ are occupied by hydrophobic amino acids and at least 50% of the positions ‘b’, ‘c’, ‘e’, ‘f’ and ‘g’ are occupied by hydrophilic amino acids.

5. The chimeric polypeptide of any one of claims 1 to 4, wherein the structure-stabilizing moiety has a glutamine at the position corresponding to position 17 of SEQ ID NO: 80.

6. The chimeric polypeptide of any one of claims 1 to 5, wherein the structure-stabilizing moiety comprises at least one immune-silencing moiety that reduces or inhibits elicitation of an immune response to the structure-stabilizing moiety;
wherein preferably the immune silencing moiety is a glycosylation site.

7. The chimeric polypeptide of any one of claims 1 to 6, wherein the structure-stabilizing moiety comprises at least one glycosylation site;
wherein preferably the at least one glycosylation site is an N-linked glycosylation site, selected from the group consisting of:
(1) -Asn-Xaa-Ser-; and
(2) -Asn-Xaa-Thr-;
wherein Xaa is an amino acid other than Pro;
wherein preferably the glycosylation site is glycosylated with an occupancy level of at least 50%.

8. The chimeric polypeptide of any one of claims 1 to 7, wherein the structure-stabilizing moiety comprises one or more N-linked glycosylation sites at the amino acid positions corresponding to:
(i) positions 5-7 of SEQ ID NO: 80;
(ii) positions 1-3 of SEQ ID NO: 82;
(iii) positions 6-8 of SEQ ID NO: 82;
(iv) positions 13-15 of SEQ ID NO: 82;
(v) positions 17-19 of SEQ ID NO: 82; and/or
(vi) positions 27-29 of SEQ ID NO: 82;
wherein preferably each N-linked glycosylation site is independently -Asn-Xaa-Thr-, wherein Xaa is an amino acid other than Pro.

9. The chimeric polypeptide of claim 8, wherein the structure-stabilizing moiety comprises N-linked glycosylation sites at the amino acid positions corresponding to:
(i) (i-a) positions 5-7 of SEQ ID NO: 80;
(i-b) positions 1-3 of SEQ ID NO: 82; and
(i-c) positions 17-19 of SEQ ID NO: 82;
or
(ii) (ii-a) positions 5-7 of SEQ ID NO: 80;
(ii-b) positions 1-3 of SEQ ID NO: 82;
(ii-c) positions 17-19 of SEQ ID NO: 82; and
(ii-d) positions 27-29 of SEQ ID NO: 82;
or
(iii) (iii-a) positions 5-7 of SEQ ID NO: 80;
(iii-b) positions 1-3 of SEQ ID NO: 82;
(iii-c) positions 13-15 of SEQ ID NO: 82;
(iii-d) positions 17-19 of SEQ ID NO: 82; and
(iii-e) positions 27-29 of SEQ ID NO: 82;
or
(iv) (iv-a) positions 5-7 of SEQ ID NO: 80;
(iv-b) positions 1-3 of SEQ ID NO: 82;
(iv-c) positions 6-8 of SEQ ID NO: 82;
(iv-d) positions 13-15 of SEQ ID NO: 82;
(iv-e) positions 17-19 of SEQ ID NO: 82; and
(iv-f) positions 27-29 of SEQ ID NO: 82;
wherein preferably each N-linked glycosylation site is independently -Asn-Xaa-Thr-, wherein Xaa is an amino acid other than Pro.

10. The chimeric polypeptide of any one of claims 1 to 9, wherein the structure-stabilizing moiety has
(i) an arginine at the amino acid position corresponding to glutamine 22 of SEQ ID NO: 80;
(ii) a histidine at the amino acid position corresponding to asparagine 1 of SEQ ID NO: 82;
(iii) a threonine at the amino acid position corresponding to histidine 2 of SEQ ID NO: 82;
(iv) a serine at the amino acid position corresponding to alanine 25 of SEQ ID NO: 82;
(v) a glutamine at the amino acid position corresponding to alanine 26 of SEQ ID NO: 82;
(vi) an asparagine at the amino acid position corresponding to leucine 27 of SEQ ID NO: 82;
(vii) a leucine at the amino acid position corresponding to glutamine 17 of SEQ ID NO: 80;
(viii) a deletion of the amino acid residue at the position corresponding to arginine 37 of SEQ ID NO: 80; and/or
(ix) a deletion of the amino acid residues at the positions corresponding to glutamine 1 and serine 2 of SEQ ID NO: 80.

11. The chimeric polypeptide of any one of claims 1 to 10, wherein the structure-stabilizing moiety comprises one or more unnatural amino acids.

12. The chimeric polypeptide of claim 11, wherein the one or more unnatural amino acids permit coupling of
(i) polyethylene glycol (PEG);
(ii) an immune-stimulating moiety; or
(iii) a lipid.

13. The chimeric polypeptide of any one of claims 1 to 12, wherein the microbial polypeptide is a viral or bacterial surface polypeptide.

14. The chimeric polypeptide of claim 13, wherein (i) the viral surface polypeptide is an enveloped virus fusion ectodomain polypeptide; or (ii) the bacterial surface polypeptide is a bacterial outer membrane polypeptide.

15. The chimeric polypeptide of claim 14, wherein the enveloped virus fusion ectodomain polypeptide corresponds to, or is a variant of:
(i) a Class I enveloped virus fusion protein ectodomain;
wherein preferably said ectodomain is from a virus selected from orthomyxoviruses, paramyxoviruses, orthopneumoviruses, metapneumoviruses, retroviruses, coronaviruses, filoviruses and arenaviruses; or
(ii) a Class III enveloped virus fusion protein ectodomain;
wherein preferably said ectodomain is from a virus selected from rhabdoviruses and herpesviruses.

16. The chimeric polypeptide of claim 15, wherein the enveloped virus fusion ectodomain polypeptide corresponds to, or is a variant of, an ectodomain of a fusion protein from:
(i) a respiratory syncytial virus;
(ii) a metapneumovirus;
(iii) a coronavirus, preferably a betacoronavirus, more preferable a severe acute respiratory syndrome coronavirus 2 (SARS-CoV-2) or a Middle East respiratory syndrome-related coronavirus (MERS-CoV);
(iv) a henipavirus, preferably a Hendra virus (HeV) or Nipah virus (NiV);
(v) an influenza virus, preferably influenza A or influenza B;
(vi) a parainfluenza virus (PIV), preferably a human parainfluenza virus (HPIV);
(vii) an arena virus, preferably Lassa Fever virus; or
(viii) a retrovirus, preferably human T-lymphotropic virus-1 (HTLV-1).

17. The chimeric polypeptide of claim 14, wherein the bacterial outer membrane polypeptide is:
(i) a Chlamydia major outer membrane protein (MOMP) polypeptide; or
(ii) a trimeric autotransporter adhesin (TAA) polypeptide.

18. The chimeric polypeptide of claim 17, wherein the TAA polypeptide corresponds to, or is a variant of:
(i) a TAA polypeptide from a bacterium of a genus selected from Neisseria, Escherichia, Haemophilus, Yersinia, Salmonella, Bartonella, Vibrio, Acinetobacter and Moraxella; and/or
(ii) a TAA polypeptide selected from Neisseria meningitidis adhesin A (NadA), Neisseria meningitidis hia/hsf homologue (NhhA), Escherichia coli autotransporter G (EhaG), Escherichia coli IgG-binding protein D (EibD), Uropathogenic Escherichia coli autotransporter G (UpaG), Haemophilus influenzae adhesin (HiA) and Yersinia enterocolitica adhesin (YadA).

19. The chimeric polypeptide of claim 17 or 18, wherein the TAA polypeptide comprises or consists of:
(i) a passenger domain; and/or
(ii) a translocator domain.

20. The chimeric polypeptide of any one of claims 1 to 19, wherein the FHRR and the SHRR comprised in the SSM are connected by a linker;
wherein preferably the linker comprises or consists of a (poly)peptide with an amino acid sequence identical to SEQ ID NO: 84 or 85.

21. The chimeric polypeptide of claim 20, wherein the linker comprises or consists of a membrane tethering (poly)peptide.

22. The chimeric polypeptide of any one of claims 1 to 21, further comprising a hinge region which operably connects the microbial polypeptide, preferably the enveloped virus fusion ectodomain polypeptide according to any one of claims 14 to 16, 20 and 21 or the bacterial outer membrane polypeptide according to any one of claims 14 and 17 to 21, with the structure-stabilizing moiety;
wherein preferably the hinge region comprises or consists of:
(i) a (poly)peptide consisting of 3 to 5 amino acid residues selected independently from serine and glycine;
(ii) serine and glycine residues;
(iii) GGSG;
(iv) GSG; or
(v) G.

23. The chimeric polypeptide of claim 22, wherein the hinge region comprises or consists of a transmembrane (poly)peptide.

24. A chimeric polypeptide comprising a first (poly)peptide operably connected downstream to a structure-stabilizing moiety, wherein said structure-stabilizing moiety is as defined in any one of claims 1 to 12, 20 and 21;
wherein preferably the first (poly)peptide is a therapeutic (poly)peptide.

25. A nucleic acid comprising a polynucleotide sequence encoding a chimeric polypeptide as defined in any one of claims 1 to 24.

26. The nucleic acid of claim 25, further comprising a promoter operably linked to the polynucleotide sequence encoding the chimeric polypeptide; wherein the promoter is preferably a mammalian promoter.

27. A host cell comprising the nucleic acid of claim 25 or 26.

28. The host cell of claim 27, wherein the host cell is
(i) a prokaryotic host cell; or
(ii) a eukaryotic host cell.

29. A method of producing a chimeric polypeptide complex, wherein the method comprises: combining chimeric polypeptides according to any one of claims 1 to 24 under conditions suitable for the formation of a chimeric polypeptide complex, whereby a chimeric polypeptide complex is produced that comprises three chimeric polypeptide subunits and is characterized by a six-helix bundle formed by homo-trimerization of the structure-stabilizing moieties of the three chimeric polypeptides.

30. The method of claim 29, wherein the six-helix bundle is composed of an inner trimer of three parallel oriented, substantially α-helical FHRRs against which three substantially α-helical SHRRs are packed in an anti-parallel orientation relative to the FHRRs.

31. A chimeric polypeptide complex that comprises three chimeric polypeptide subunits, wherein each subunit is a chimeric polypeptide according to any one of claims 1 to 24, and wherein the complex is characterized by a six-helix bundle formed by homo-trimerization of the structure-stabilizing moieties of the three chimeric polypeptides.

32. The chimeric polypeptide complex of claim 31, wherein the six-helix bundle is composed of an inner trimer of three parallel oriented, substantially α-helical FHRRs against which three substantially α-helical SHRRs are packed in an anti-parallel orientation relative to the FHRRs.

33. The chimeric polypeptide complex of claim 31 or 32, wherein the chimeric polypeptide subunits each comprise an enveloped virus fusion ectodomain polypeptide, and wherein the complex comprises at least one pre-fusion epitope of an enveloped virus fusion protein.

34. A composition comprising a chimeric polypeptide according to any one of claims 1 to 24, or a chimeric polypeptide complex according to any one of claims 31 to 33, and a pharmaceutically acceptable carrier, diluent or adjuvant.

35. A method of identifying an agent that binds with:
a microbial polypeptide or a complex thereof, wherein the method comprises:
(i) contacting a candidate agent with a microbial polypeptide-containing chimeric polypeptide according to any one of claims 1 to 23 or a microbial polypeptide-containing chimeric polypeptide complex according to any one of claims 31 to 33; and
(ii) detecting binding of the candidate agent to the chimeric polypeptide or chimeric polypeptide complex;
wherein preferably the candidate agent is part of a compound library (e.g., small molecule or macromolecule library).

36. The method of claim 35, wherein the microbial polypeptide or the complex thereof is:
(a) a fusion protein of an enveloped virus, or a complex of the fusion protein, respectively, wherein the method comprises:
(i) contacting the candidate agent with an enveloped virus fusion ectodomain polypeptide-containing chimeric polypeptide according to any one of claims 14 to 16 or 20 to 23 or an enveloped virus fusion ectodomain polypeptide-containing chimeric polypeptide complex according to any one of claims 31 to 33, wherein the enveloped virus fusion ectodomain polypeptide corresponds to the fusion protein of the enveloped virus; and
(ii) detecting binding of the candidate agent to the chimeric polypeptide or chimeric polypeptide complex;
or
(b) an outer membrane polypeptide of a bacterium or a complex of the outer membrane polypeptide, respectively, wherein the method comprises:
(i) contacting the candidate agent with a bacterial outer membrane polypeptide-containing chimeric polypeptide according to any one of claims 14 and 17 to 23 or a bacterial outer membrane polypeptide-containing chimeric polypeptide complex according to claim 31 or 32, wherein the bacterial outer membrane polypeptide corresponds to the outer membrane polypeptide of the bacterium; and
(ii) detecting binding of the candidate agent to the chimeric polypeptide or chimeric polypeptide complex.

37. The method of claim 36(b), wherein the outer membrane polypeptide of a bacterium or the complex thereof is:
(a) a trimeric autotransporter adhesin (TAA) polypeptide of a bacterium, or a complex of the TAA polypeptide, wherein the method comprises:
(i) contacting the candidate agent with a TAA polypeptide-containing chimeric polypeptide according to any one of claims 17 to 23 or a TAA polypeptide-containing chimeric polypeptide complex according to claim 31 or 32, wherein the TAA polypeptide corresponds to the TAA polypeptide of the bacterium; and
(ii) detecting binding of the candidate agent to the chimeric polypeptide or chimeric polypeptide complex; or (b) a major outer membrane protein (MOMP) polypeptide of a Chlamydia bacterium or a complex thereof, wherein the method comprises:
(i) contacting the candidate agent with a Chlamydia MOMP polypeptide-containing chimeric polypeptide according to any one of claims 17 to 23 or a Chlamydia MOMP polypeptide-containing chimeric polypeptide complex according to claim 31 or 32, wherein the Chlamydia MOMP polypeptide corresponds to the MOMP polypeptide of the Chlamydia bacterium; and
(ii) detecting binding of the candidate agent to the chimeric polypeptide or chimeric polypeptide complex.

38. The method of claim 35, further comprising:
(i) contacting the candidate agent of claim 35 with the microbial polypeptide or the complex thereof and detecting binding of the candidate agent to the microbial polypeptide or the complex thereof; and/or
(ii) isolating the candidate agent.

39. The method of claim 36, further comprising:
(i) (i-a) contacting the candidate agent of claim 36(a) with the fusion protein or the complex of the fusion protein and detecting binding of the candidate agent to the fusion protein or the complex of the fusion protein; or
(i-b) contacting the candidate agent of claim 36(b) with the outer membrane polypeptide or the complex of the outer membrane polypeptide and detecting binding of the candidate agent to the outer membrane polypeptide or the complex of the outer membrane polypeptide;
and/or
(ii) isolating the candidate agent.

40. The method of claim 37, further comprising:
(i) (i-a) contacting the candidate agent of claim 37(a) with the TAA polypeptide or the complex of the TAA polypeptide and detecting binding of the candidate agent to the TAA polypeptide or the complex of the TAA polypeptide; or
(i-b) contacting the candidate agent of claim 37(b) with the MOMP polypeptide or the complex of the MOMP polypeptide;
and/or
(ii) isolating the candidate agent.

41. The method of any one of claims 35 to 40, wherein the candidate agent binds specifically to the chimeric polypeptide or chimeric polypeptide complex.

42. The method of any one of claims 35 to 41, wherein the candidate agent binds specifically to the microbial polypeptide or the complex thereof.

43. The method of claim 36 or 39, wherein the candidate agent binds specifically to:
(i) the enveloped virus fusion protein or the complex of the fusion protein; or
(ii) the outer membrane polypeptide or the complex of the outer membrane polypeptide.

44. The method of claim 37 or 40, wherein the candidate agent binds specifically to:
(i) the TAA polypeptide or the complex of the TAA polypeptide; or
(ii) the MOMP polypeptide or the complex of the MOMP polypeptide.

45. A method of producing an antigen-binding molecule that specifically binds to:
a microbial polypeptide, or a complex thereof, wherein the method comprises:
(1) immunizing a subject with a microbial polypeptide-containing chimeric polypeptide according to any one of claims 1 to 23, or a microbial polypeptide-containing chimeric polypeptide complex according to any one of claims 31 to 33, or a composition thereof according to claim 34;
(2) identifying and/or isolating a B cell from the immunised subject, which specifically binds to the microbial polypeptide or complex thereof; and
(3) producing the antigen-binding molecule expressed by that B cell.

46. The method of claim 45, wherein the microbial polypeptide or the complex thereof is:
(a) an ectodomain of a fusion protein of an enveloped virus, or complex of the fusion protein, wherein the method comprises:
(1) immunizing a subject with an ectodomain polypeptide-containing chimeric polypeptide according to any one of claims 14 to 16 or 20 to 23, or an ectodomain polypeptide-containing chimeric polypeptide complex according to any one of claims 31 to 33, or a composition thereof according to claim 34, wherein the ectodomain polypeptide corresponds to the fusion protein of the enveloped virus;
(2) identifying and/or isolating a B cell from the immunised subject, which specifically binds to the ectodomain of the fusion protein or complex thereof; and
(3) producing the antigen-binding molecule expressed by that B cell;
or
(b) an outer membrane polypeptide of a bacterium, or a complex of the outer membrane polypeptide, wherein the method comprises:
(1) immunizing a subject with a bacterial outer membrane polypeptide-containing chimeric polypeptide according to any one of claims 14 and 17 to 23, or a bacterial outer membrane polypeptide-containing chimeric polypeptide complex according to claim 31 or 32, or a composition thereof according to claim 34, wherein the bacterial outer membrane polypeptide corresponds to the outer membrane polypeptide of the bacterium;
(2) identifying and/or isolating a B cell from the immunised subject, which specifically binds to the bacterial outer membrane polypeptide or complex thereof; and
(3) producing the antigen-binding molecule expressed by that B cell.

47. The method of claim 46(b), wherein the outer membrane polypeptide or the complex thereof is:
(i) a TAA polypeptide of a bacterium or a complex thereof, respectively, wherein the method comprises:
(1) immunizing a subject with a TAA polypeptide-containing chimeric polypeptide according to any one of claims 17 to 23, or a TAA polypeptide-containing chimeric polypeptide complex according to claim 31 or 32, or a composition thereof according to claim 34, wherein the TAA polypeptide corresponds to the TAA polypeptide of the bacterium;
(2) identifying and/or isolating a B cell from the immunised subject, which specifically binds to the TAA polypeptide or complex thereof; and
(3) producing the antigen-binding molecule expressed by that B cell;
or
(ii) a major outer membrane protein (MOMP) polypeptide of a Chlamydia bacterium or a complex thereof, respectively, wherein the method comprises:
(1) immunizing a subject with a Chlamydia MOMP polypeptide-containing chimeric polypeptide according to any one of claims 17 to 23, or a Chlamydia MOMP polypeptide-containing chimeric polypeptide complex according to claim 31 or 32, or a composition thereof according to claim 34, wherein the Chlamydia MOMP polypeptide corresponds to the MOMP polypeptide of the Chlamydia bacterium;
(2) identifying and/or isolating a B cell from the immunised subject, which specifically binds to the Chlamydia MOMP polypeptide or complex thereof; and
(3) producing the antigen-binding molecule expressed by that B cell.

48. An antigen-binding molecule that specifically binds to:
the microbial polypeptide of a microbial polypeptide-containing chimeric polypeptide according to any one of claims 1 to 23 and/or the microbial polypeptide of one or more subunits of a microbial polypeptide-containing chimeric polypeptide complex according to any one of claims 31 to 33.

49. The antigen-binding molecule of claim 48, wherein the antigen-binding molecule specifically binds to:
(i) the ectodomain of an enveloped virus fusion ectodomain polypeptide-containing chimeric polypeptide according to any one of claims 14 to 16 or 20 to 23; and/or the ectodomain of one or more subunits of an enveloped virus fusion ectodomain polypeptide-containing chimeric polypeptide complex according to any one of claims 31 to 33;
or
(ii) the bacterial outer membrane polypeptide of a bacterial outer membrane polypeptide-containing chimeric polypeptide according to any one of claims 14 and 17 to 23; and/or the bacterial outer membrane polypeptide of a bacterial outer membrane polypeptide-containing chimeric polypeptide complex according to claim 31 or 32.

50. The antigen-binding molecule of claim 49(ii), wherein the antigen-binding molecule specifically binds to:
(i) the TAA polypeptide of a TAA polypeptide-containing chimeric polypeptide according to any one of claims 17 to 23; and/or the TAA polypeptide of a TAA polypeptide-containing chimeric polypeptide complex according to claim 31 or 32; or
(ii) the Chlamydia major outer membrane protein (MOMP) polypeptide of a Chlamydia MOMP polypeptide-containing chimeric polypeptide according to any one of claims 17 to 23; and/or the Chlamydia MOMP polypeptide of a Chlamydia MOMP polypeptide-containing chimeric polypeptide complex according to according to claim 31 or 32.

51. An antigen-binding molecule that is obtainable by:
(i) the method of any of claims 45 to 47;
(ii) the method of claim 46(a);
(iii) the method of claim 46(b);
(iv) the method of claim 47(i); or
(v) the method of claim 47(ii).

52. A composition comprising an antigen-binding molecule according to any one of claims 48 to 51, and a pharmaceutically acceptable carrier, diluent or adjuvant.

53. A composition comprising the nucleic acid according to claim 25 or 26.

54. The composition of claim 53, wherein the chimeric polypeptide, which is encoded by the polynucleotide sequence comprised in the nucleic acid, is:
(i) a microbial polypeptide-containing chimeric polypeptide according to any one of claims 1 to 23; or
(ii) a first (poly)peptide-containing chimeric polypeptide according to claim 24.

55. The composition of claim 54(i), wherein the microbial polypeptide-containing chimeric polypeptide is:
(i) an enveloped virus fusion ectodomain polypeptide-containing chimeric polypeptide according to any one of claims 14 to 16 and 20 to 23; or
(ii) a bacterial outer membrane polypeptide-containing chimeric polypeptide according to any one of claims 14 and 17 to 23.

56. The composition of claim 55(ii), wherein the bacterial outer membrane polypeptide-containing chimeric polypeptide is:
(i) a TAA polypeptide-containing chimeric polypeptide according to any one of claims 17 to 23; or
(ii) a Chlamydia major outer membrane protein (MOMP) polypeptide-containing chimeric polypeptide according to any one of claims 17 to 23.

57. The composition of any one of claims 53 to 56, wherein the nucleic acid comprises or consists of RNA.

58. The composition of claim 57, wherein the RNA comprises at least one modified nucleotide;
wherein preferably the at least modified nucleotide is a modified uridine, more preferable a methylated derivative of uridine, most preferably a N1-methyl-pseudouridine.

59. The composition of claim 57 or 58, wherein the RNA is formulated in a delivery vehicle which is a liposome, lipoplex or lipid nanoparticle (LNP);
wherein preferably the lipid nanoparticle (LNP) comprises a cationic lipid, a neutral lipid, a steroid, and/or a PEGylated lipid.

60. The composition of any one of claims 53 to 56, wherein the nucleic acid comprises or consists of DNA; wherein preferably the DNA is comprised in a plasmid.

61. A chimeric polypeptide according to any one of claims 1 to 24, a nucleic acid according to claim 25 or 26, a host cell according to claim 27 or 28, a chimeric polypeptide complex according to any one of claims 31 to 33, a composition according to claim 34, an antigen-binding molecule according to any one of claims 48 to 51, or a composition according to any one of claims 52 to 60, for use as a medicament.

62. A method of eliciting an immune response to:
a microbial polypeptide, or a complex thereof, in a subject, wherein the method comprises administering to the subject:
(i) a microbial polypeptide-containing chimeric polypeptide according to any one of claims 1 to 23, a microbial polypeptide-containing chimeric polypeptide complex according to any one of claims 31 to 33, or a composition thereof according to claim 34; or
(ii) a composition according to claim 54(i) or any of its dependent claims 55 to 60.

63. The method of claim 62, wherein the microbial polypeptide, or the complex thereof, is:
(a) a fusion protein of an enveloped virus, or a complex of the fusion protein, respectively, wherein the method comprises administering to the subject:
(i) an enveloped virus fusion ectodomain-containing chimeric polypeptide according to any one of claims 14 to 16 or 20 to 23, an enveloped virus fusion ectodomain-containing chimeric polypeptide complex according to any one of claims 31 to 33, or a composition thereof according to claim 34; or
(ii) a composition according to claim 55(i) or any of its dependent claims 57 to 60;
wherein an ectodomain polypeptide subunit of the chimeric polypeptide complex corresponds to the fusion protein of the enveloped virus;
or
(b) an outer membrane polypeptide of a bacterium, or a complex of the outer membrane polypeptide, respectively, wherein the method comprises administering to the subject:
(i) a bacterial outer membrane polypeptide-containing chimeric polypeptide according to any one of claims 14 and 17 to 23, a bacterial outer membrane polypeptide-containing chimeric polypeptide complex according to claim 31 or 32, or a composition thereof according to claim 34; or
(ii) a composition according to claim 55(ii) or any one of its dependent claims 56 to 60;
wherein a bacterial outer membrane polypeptide subunit of the chimeric polypeptide complex corresponds to, or substantially corresponds to, a bacterial outer membrane polypeptide expressed by the bacterium.

64. The method of claim 63(b), wherein the bacterial outer membrane polypeptide, or the complex thereof, is:
(a) TAA polypeptide of a bacterium, or a complex of the TAA polypeptide, respectively, wherein the method comprises administering to the subject:
(i) a TAA polypeptide-containing chimeric polypeptide according to any one of claims 17 to 23, a TAA polypeptide-containing chimeric polypeptide complex according to claim 31 or 32, or a composition thereof according to claim 34; or
(ii) a composition according to claim 56(i) or any one of its dependent claims 57 to 60;
wherein a TAA polypeptide subunit of the chimeric polypeptide complex corresponds to, or substantially corresponds to, a TAA polypeptide expressed by the bacterium;
or
(b) Chlamydia major outer membrane protein (MOMP) polypeptide, or a complex of the Chlamydia MOMP polypeptide, respectively, wherein the method comprises administering to the subject:
(i) a Chlamydia MOMP polypeptide-containing chimeric polypeptide according to any one of claims 17 to 23, a Chlamydia MOMP polypeptide-containing chimeric polypeptide complex according to claim 31 or 32, or a composition thereof according to claim 34; or
(ii) a composition according to claim 56(ii) or any one of its dependent claims 57 to 60;
wherein a Chlamydia MOMP polypeptide subunit of the chimeric polypeptide complex corresponds to, or substantially corresponds to, a Chlamydia MOMP polypeptide expressed by the bacterium.

65. A method for treating or preventing a microbial infection in a subject, wherein the method comprises administering to the subject an effective amount of:
(i) a microbial polypeptide-containing chimeric polypeptide according to any one of claims 1 to 23, a microbial polypeptide-containing chimeric polypeptide complex according to any one of claims 31 to 33, or a composition thereof according to claim 34;
(ii) an antigen binding molecule according to claim 48 to 51, or a composition thereof according to claim 52; or
(iii) a composition according to claim 54(i) or any of its dependent claims 55 to 60.

66. The method of claim 65, wherein the microbial infection is:
(a) an enveloped virus infection in a subject, wherein the method comprises administering to the subject an effective amount of:
(i) an enveloped virus fusion ectodomain-containing chimeric polypeptide according to any one of claims 14 to 16 or 20 to 23, an enveloped virus fusion ectodomain-containing chimeric polypeptide complex according to any one of claims 31 to 33, or a composition thereof according to claim 34;
(ii) an antigen binding molecule according to claim 49(i) or 51(ii), or a composition thereof according to claim 52; or
(iii) a composition according to claim 55(i) or any one of its dependent claims 57 to 60;
or
(b) a bacterial infection in a subject, wherein the method comprises administering to the subject an effective amount of:
(i) a bacterial outer membrane polypeptide-containing chimeric polypeptide according to any one of claims 14 and 17 to 23, a bacterial outer membrane polypeptide-containing chimeric polypeptide complex according to claim 31 or 32, or a composition thereof according to claim 34;
(ii) an antigen-binding molecule according to claim 49(ii), 50 and 51(iii-v), or a composition thereof according to claim 52; or
(iii) a composition according to claim 55(ii) or any one of its dependent claims 56 to 60.

67. The method of claim 66(b), wherein the bacterial infection is:
(a) an infection by a TAA-expressing bacterium, wherein the method comprises administering to the subject an effective amount of:
(i) a TAA polypeptide-containing chimeric polypeptide according to any one of claims 17 to 23, a TAA polypeptide-containing chimeric polypeptide complex according claim 31 or 32, or a composition thereof according to claim 34;
(ii) an antigen-binding molecule according to claim 50(i) or 51(iv), or a composition thereof according to claim 52; or
(iii) a composition according to claim 56(i) or any one of its dependent claims 57 to 60;
or
(b) an infection by a Chlamydia bacterium, wherein the method comprises administering to the subject an effective amount of:
(i) a Chlamydia MOMP polypeptide-containing chimeric polypeptide according to any one of claims 17 to 23, a Chlamydia MOMP polypeptide-containing chimeric polypeptide complex according claim 31 or 32, or a composition thereof according to claim 34;
(ii) an antigen-binding molecule according to claim 50(ii) or 51(v), or a composition thereof according to claim 52; or
(iii) a composition according to claim 56(ii) or any one of its dependent claims 57 to 60.

68. A vaccine comprising:
(i) a microbial polypeptide-containing chimeric polypeptide according to any one of claims 1 to 23, a microbial polypeptide-containing chimeric polypeptide complex according to any one of claims 31 to 33, or a composition thereof according to claim 34; or
(ii) a composition according to claim 54(i) or any of its dependent claims 55 to 60;
for use in a method of eliciting an immune response to a microbial polypeptide, or a complex of the microbial polypeptide, in a subject.

69. The vaccine for use according to claim 68, wherein the microbial polypeptide is:
(a) an enveloped virus fusion ectodomain polypeptide, and the vaccine comprises:
(i) an enveloped virus fusion ectodomain-containing chimeric polypeptide according to any one of claims 14 to 16 or 20 to 23, an enveloped virus fusion ectodomain polypeptide-containing chimeric polypeptide complex according to any one of claims 31 to 33, or a composition thereof according to claim 34; or
(ii) a composition according to claim 55(i) or any one of its dependent claims 57 to 60;
for use in a method of eliciting an immune response to a fusion protein of an enveloped virus, or a complex of the fusion protein, in a subject, and wherein an ectodomain polypeptide subunit of the chimeric polypeptide complex corresponds to the fusion protein of the enveloped virus;
or
(b) a bacterial outer membrane polypeptide, and the vaccine comprises:
(i) a bacterial outer membrane polypeptide-containing chimeric polypeptide according to any one of claims 14 and 17 to 23, a bacterial outer membrane polypeptide-containing chimeric polypeptide complex according to claim 31 or 32, or a composition thereof according to claim 34; or
(ii) a composition according to claim 55(ii) or any one of its dependent claims 56 to 60;
for use in a method of eliciting an immune response to an outer membrane polypeptide of a bacterium, or a complex of the outer membrane polypeptide, in a subject, and wherein an outer membrane polypeptide subunit of the chimeric polypeptide complex corresponds to an outer membrane polypeptide of the bacterium.

70. The vaccine for use according to claim 69, wherein the bacterial outer membrane polypeptide is:
(a) a bacterial trimeric autotransporter adhesin (TAA) polypeptide, and the vaccine comprises:
(i) a TAA polypeptide-containing chimeric polypeptide according to any one of claims 17 to 23, a TAA polypeptide-containing chimeric polypeptide complex according to claim 31 or 32, or a composition thereof according to claim 34; or
(ii) a composition according to claim 56(i) or any one of its dependent claims 57 to 60;
for use in a method of eliciting an immune response to a TAA polypeptide of a bacterium, or complex of the TAA polypeptide, in a subject, and wherein a TAA polypeptide subunit of the chimeric polypeptide complex corresponds to a TAA polypeptide of the bacterium;
or
(b) a Chlamydia MOMP polypeptide, and the vaccine comprises:
(i) a Chlamydia MOMP polypeptide-containing chimeric polypeptide according to any one of claims 17 to 23, a Chlamydia MOMP polypeptide-containing chimeric polypeptide complex according to claim 31 or 32, or a composition thereof according to claim 34; or
(ii) a composition according to claim 56(ii) or any one of its dependent claims 57 to 60;
for use in a method of eliciting an immune response to a MOMP polypeptide of a Chlamydia bacterium, or complex of the MOMP polypeptide, in a subject, and wherein a MOMP polypeptide subunit of the chimeric polypeptide complex corresponds to a MOMP polypeptide of the Chlamydia bacterium.

71. A microbial polypeptide-containing chimeric polypeptide according to any one of claims 1 to 23, a microbial polypeptide-containing chimeric polypeptide complex according to any one of claims 31 to 33 or a composition thereof according to claim 34, or an antigen-binding molecule according to any one of claims 48 to 51 or a composition thereof according to claim 52, or a composition according to claim 54(i) or any of its dependent claims 55 to 60, for use in a method for treating or preventing a microbial infection in a subject.

72. The microbial polypeptide-containing chimeric polypeptide for use according to claim 71, wherein the microbial polypeptide is:
(a) an enveloped virus fusion ectodomain polypeptide, and provided is an enveloped virus fusion ectodomain polypeptide-containing chimeric polypeptide according to any one of claims 14 to 16 and 20 to 23, an enveloped virus fusion ectodomain-containing chimeric polypeptide complex according to any one of claims 31 to 33 or a composition thereof according to claim 34, or an antigen binding molecule according to claim 49(i) or 51(ii) or a composition thereof according to claim 52, or a composition according to claim 55(i) or any of its dependent claims 57 to 60, for use in a method for treating or preventing an enveloped virus infection in a subject; or
(b) a bacterial outer membrane polypeptide, and provided is a bacterial outer membrane polypeptide-containing chimeric polypeptide according to any one of claims 14 and 17 to 23, a bacterial outer membrane polypeptide-containing chimeric polypeptide complex according to claim 31 or 32 or a composition thereof according to claim 34, or an antigen-binding molecule according to any one of claims 49(ii), 50 and 51(iii-v) or a composition thereof according to claim 52, or a composition according to claim 55(ii) or any one of its dependent claims 56 to 60, for use in a method for treating or preventing a bacterial infection in a subject.

73. The microbial polypeptide-containing chimeric polypeptide for use according to claim 72(b), wherein the bacterial outer membrane polypeptide is:
(a) a bacterial trimeric autotransporter adhesin (TAA) polypeptide, and provided is a bacterial TAA polypeptide-containing chimeric polypeptide according to any one of claims 17 to 23, a TAA polypeptide-containing chimeric polypeptide complex according to claim 31 or 32 or a composition thereof according to claim 34, or an antigen-binding molecule according to claim 50(i) or 51(iv) or a composition thereof according to claim 52, or a composition according to claim 56(i) or any one of its dependent claims 57 to 60, for use in a method for treating or preventing a bacterial infection by a TAA-expressing bacterium in a subject;
or
(b) a Chlamydia major outer membrane protein (MOMP) polypeptide, and provided is a Chlamydia MOMP polypeptide-containing chimeric polypeptide according to any one of claims 17 to 23, a Chlamydia MOMP polypeptide-containing chimeric polypeptide complex according to claim 31 or 32 or a composition thereof according to claim 34, or an antigen-binding molecule according to claim 50(ii) or 51(v) or a composition thereof according to claim 52, or a composition according to claim 56(ii) or any one of its dependent claims 57 to 60, for use in a method for treating or preventing a Chlamydia infection in a subject.


SEQUENCE LISTING (302 sequences)

SEQ ID NO: 1 - protein, 78 aa, synthetic construct
  source 1..78: note = Original HIV-clamp
IVQQQNNLLRAIEAQQHLLQLTVWGIKQLQARILAGGSGGHTTWMEWDREINNYTSLIHSLIEESQNQQEKNEQELLE

SEQ ID NO: 2 - protein, 91 aa, synthetic construct
  source 1..91: note = CD1: MMPV-based SSM
SITQYTKLSHQLISDVQAISSTIQDLQDQVDSLAEVVLQNRRGLDLLTAEQGGICLALQEKCSFYANKSGIVRDKIKNLQDDLERRRRQLI

SEQ ID NO: 3 - protein, 67 aa, synthetic construct
  source 1..67: note = CD2: MMPV-based SSM
SITQYTKLSHQLISDVQAISSTIQDLQDQVDSLAEVVGSGYANKSGIVRDKIKNLQDDLERRRRQLI

SEQ ID NO: 4 - protein, 81 aa, synthetic construct
  source 1..81: note = CD3: XMRV-based SSM
FEQLQAAIHTDLGALEKSVSALEKSLTSLSEVVLQNRRGLDLLFLKEGGLCAALKEECSFYADHTGVVRDSMAKLRERLNQ

SEQ ID NO: 5 - protein, 57 aa, synthetic construct
  source 1..57: note = CD4: XMRV-based SSM
FEQLQAAIHTDLGALEKSVSALEKSLTSLSEVVGSGYADHTGVVRDSMAKLRERLNQ

SEQ ID NO: 6 - protein, 76 aa, synthetic construct
  source 1..76: note = CD5: KoRV-based SSM
LQIAMDTDLRALQDSISKLEDSLTSLSEVVLQNRRGLDLLFLKEGGLCAALKEECSFYVDHSGAVRDSMRRLKERL

SEQ ID NO: 7 - protein, 49 aa, synthetic construct
  source 1..49: note = CD6: KoRV-based SSM
AMDTDLRALQDSISKLEDSLTSLSEVVGSGYVDHSGAVRDSMRRLKERL

SEQ ID NO: 8 - protein, 79 aa, synthetic construct
  source 1..79: note = CD7: FIV-based SSM
ATHQEAIEKVTEALKINNLRLVTLEHQVLVIGLKVEAGGSGGNQTIWNHGNITLGEWYNKTKDLQQKFYEIIMDIEQNN

SEQ ID NO: 9 - protein, 80 aa, synthetic construct
  source 1..80: note = CD8: SIV-based SSM
AGIVQQQQQLLDVVKRQQELLRLTVWGTKNLQTRVTAGGSGGNNETWQEWERKVDFLEENITALLEEAQIQQEKNMYELQ

SEQ ID NO: 10 - protein, 81 aa, synthetic construct
  source 1..81: note = CD9: VISNA-based SSM
QSLANATAAQQEVLEASYAMVQHIAKGIRILEARVARGGSGGNHTWQQWEEEIEQHEGNLSLLLREAALQVHIAQRDARRI

SEQ ID NO: 11 - protein, 81 aa, synthetic construct
  source 1..81: note = CD10: CAEV-based SSM
QTLANATAAQQDVLEATYAMVQHVAKGVRILEARVARGGSGGNHTWQQWERGLQGYDTNLTILLKESAAMTQLAEEQARRI

SEQ ID NO: 12 - protein, 81 aa, synthetic construct
  source 1..81: note = CD11: VISNA-based SSM
QSLANATAAQQEVLEAQYAMVQHIAKGIRILEARVARGGSGGNHTWQQWEEEIEQHEGNLSLLLREAALQVHIAQRDARRI

SEQ ID NO: 13 - protein, 74 aa, synthetic construct
  source 1..74: note = CK1: Lassa-based SSM
EFSDMLRLFDFNKQAIQRLKAEAQMSIQLINGGSGGNETHFSDDIEQQADNMITEMLQKEYMERQGKTPLGLVD

SEQ ID NO: 14 - protein, 77 aa, synthetic construct
  source 1..77: note = CK2: EAIV-based SSM
ADSVQNHTFEVENNTINGLELTEEQVHILYAMVLQTGGSGGTQWDDWVDKMENLNHDILTTLHTARNNLEQSMITFN

SEQ ID NO: 15 - protein, 72 aa, synthetic construct
  source 1..72: note = CK3: NiV-based SSM
EAMKNADNINKLKSSIESTNEAVVKLQETAEKTVGGSGGDISSQISSMNQSLQQSKDYIKEAQRLLDTVNPS

SEQ ID NO: 16 - protein, 73 aa, synthetic construct
  source 1..73: note = CK4: GALV-based SSM
DLQQGLTSLQIAMDTDLRALQDSISKLEDSLTSLSEGGSGGFYVDHSGAVRDSMKKLKERLDKRQLERQKNQN

SEQ ID NO: 17 - protein, 73 aa, synthetic construct
  source 1..73: note = CK5: KoRV-based SSM
DLQQGLTSLQIAMDTDLRALQDSISKLEDSLTSLSEGGSGGFYVDHSGAVRDSMRRLKERLDKRQLEHQKNLS

SEQ ID NO: 18 - protein, 74 aa, synthetic construct
  source 1..74: note = CK6: PERV-based SSM
QQLEKGLSNLHRIVTEDLQALEKSVSNLEESLTSLSEGGSGGFYVDHSGAIRDSMNKLRERLEKRRREKETTQG

SEQ ID NO: 19 - protein, 79 aa, synthetic construct
  source 1..79: note = CK7: FELV-based SSM
KALIETAQFRQLQMAMHTDIQALEESISALEKSLTSLSEGGSGGFYADHTGLVRDNMAKLRERLKQRQQLFDSQQGWFE

SEQ ID NO: 20 - protein, 68 aa, synthetic construct
  source 1..68: note = CK8: BoLV-based SSM
STLSHQRLTSLIHVLEQDQQRLITAINQTHYNLLNVASGGSGGFLRIQNDSIIRLGDLQPLSQRVSTD

SEQ ID NO: 21 - protein, 574 aa, synthetic construct
  source 1..574: note = RSV Fusion protein 66K
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 22 - protein, 556 aa, synthetic construct
  source 1..556: note = RSV Fusion protein 66K T103-GS-G145 E511-GSG-VISNA-based SSM referred to as VL22_66K_CD11
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGQSLANATAAQQEVLEAQYAMVQHIAKGIRILEARVARGGSGGNHTWQQWEEEIEQHEGNLSLLLREAALQVHIAQRDARRI

SEQ ID NO: 23 - protein, 574 aa, synthetic construct
  source 1..574: note = RSV Fusion protein 66E
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 24 - protein, 556 aa, synthetic construct
  source 1..556: note = RSV Fusion protein 66E T103-GS-G145 E511-GSG-VISNA-based SSM Referred to as VL22_66E_CD11 or for simplicity VL22_CD11
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGQSLANATAAQQEVLEAQYAMVQHIAKGIRILEARVARGGSGGNHTWQQWEEEIEQHEGNLSLLLREAALQVHIAQRDARRI

SEQ ID NO: 25 - protein, 574 aa, synthetic construct
  source 1..574: note = RSV Fusion protein 66K/101Q
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 26 - protein, 555 aa, synthetic construct
  source 1..555: note = RSV Fusion protein 66K/101Q N105-GS-G145 E511-GSG-HIV-1 GP41-based SSM referred to as F HIV-clamp
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGIVQQQNNLLRAIEAQQHLLQLTVWGIKQLQARILAGGSGGHTTWMEWDREINNYTSLIHSLIEESQNQQEKNEQELLE

SEQ ID NO: 27 - protein, 526 aa, synthetic construct
  source 1..526: note = RSV Fusion protein 66K/101Q V516-His-Tag referred to as 'Fsol'
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVGGGGHHHHHH

SEQ ID NO: 28 - protein, 1276 aa, synthetic construct
  source 1..1276: note = SARS-CoV-2 Spike protein N679-GSG-S691 G1204-SG-HIV GP41-based SSM referred to as SARS-CoV-2 S HIV-clamp
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 29 - protein, 1279 aa, synthetic construct
  source 1..1279: note = SARS-CoV-2 Spike protein N679-GSG-S691 G1204-VISNA-based SSM CD11 referred to as SARS-CoV-2 S CD11
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGQSLANATAAQQEVLEAQYAMVQHIAKGIRILEARVARGGSGGNHTWQQWEEEIEQHEGNLSLLLREAALQVHIAQRDARRI

SEQ ID NO: 30 - protein, 564 aa, synthetic construct
  source 1..564: note = Nipah virus Fusion -HIV GP41-based SSM referred to as Nipah F HIV-clamp
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTGSGIVQQQNNLLRAIEAQQHLLQLTVWGIKQLQARILAGGSGGHTTWMEWDREINNYTSLIHSLIEESQNQQEKNEQELLE

SEQ ID NO: 31 - protein, 567 aa, synthetic construct
  source 1..567: note = Nipah virus Fusion -VISNA-based SSM referred to as Nipah F CD11
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTGSGQSLANATAAQQEVLEAQYAMVQHIAKGIRILEARVARGGSGGNHTWQQWEEEIEQHEGNLSLLLREAALQVHIAQRDARRI

SEQ ID NO: 32 - protein, 607 aa, synthetic construct
  source 1..607: note = Influenza Hemagglutinin protein R526-HIV GP41-based SSM referred to as Influenza HA HIV-clamp
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRGSGIVQQQNNLLRAIEAQQHLLQLTVWGIKQLQARILAGGSGGHTTWMEWDREINNYTSLIHSLIEESQNQQEKNEQELLE

SEQ ID NO: 33 - protein, 610 aa, synthetic construct
  source 1..610: note = Influenza Hemagglutinin protein R526-VISNA-based SSM referred to as Influenza HA CD11
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRGSGQSLANATAAQQEVLEAQYAMVQHIAKGIRILEARVARGGSGGNHTWQQWEEEIEQHEGNLSLLLREAALQVHIAQRDARRI

SEQ ID NO: 34 - protein, 1277 aa, synthetic construct
  source 1..1277: note = SARS-CoV-2 Spike protein N679-GSG-S691 G1204-VISNA-based SSM CD11 (-SG) referred to as SARS-CoV-2 S CD11_SG
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGQSLANATAAQQEVLEAQYAMVQHIAKGIRILEARVARGGSGGNHTWQQWEEEIEQHEGNLSLLLREAALQVHIAQRDARRI

SEQ ID NO: 35 - protein, 81 aa, synthetic construct
  source 1..81: note = CD11_124568
QSLANATAAQQEVLEAQYAMVQHIAKGIRILEARVARGGSGGNHTWQNWTEEIENHTGNLSNLTREAANQTHIAQRDARRI

SEQ ID NO: 36 - protein, 81 aa, synthetic construct
  source 1..81: note = CD11_1245T68
QSLANATAAQQEVLEAQYAMVQHIAKGIRILEARVARGGSGGNHTWQNWTEEIENHTGNLTNLTREAANQTHIAQRDARRI

SEQ ID NO: 37 - protein, 81 aa, synthetic construct
  source 1..81: note = CD11_1245T8
QSLANATAAQQEVLEAQYAMVQHIAKGIRILEARVARGGSGGNHTWQNWTEEIENHTGNLTLLLREAANQTHIAQRDARRI

SEQ ID NO: 38 - protein, 81 aa, synthetic construct
  source 1..81: note = CD11_145T8
QSLANATAAQQEVLEAQYAMVQHIAKGIRILEARVARGGSGGNHTWQQWEEEIENHTGNLTLLLREAANQTHIAQRDARRI

SEQ ID NO: 39 - protein, 81 aa, synthetic construct
  source 1..81: note = CD11_1458
QSLANATAAQQEVLEAQYAMVQHIAKGIRILEARVARGGSGGNHTWQQWEEEIENHTGNLSLLLREAANQTHIAQRDARRI

SEQ ID NO: 40 - protein, 81 aa, synthetic construct
  source 1..81: note = CD11_159
QSLANATAAQQEVLEAQYAMVQHIAKGIRILEARVARGGSGGNHTWQQWEEEIEQHEGNLSLLLREAALQVHNATRDARRI

SEQ ID NO: 41 - protein, 81 aa, synthetic construct
  source 1..81: note = CD11_158
QSLANATAAQQEVLEAQYAMVQHIAKGIRILEARVARGGSGGNHTWQQWEEEIEQHEGNLSLLLREAANQTHIAQRDARRI

SEQ ID NO: 42 - protein, 81 aa, synthetic construct
  source 1..81: note = CD11_157
QSLANATAAQQEVLEAQYAMVQHIAKGIRILEARVARGGSGGNHTWQQWEEEIEQHEGNLSLLLRNATLQVHIAQRDARRI

SEQ ID NO: 43 - protein, 81 aa, synthetic construct
  source 1..81: note = CD11_156
QSLANATAAQQEVLEAQYAMVQHIAKGIRILEARVARGGSGGNHTWQQWEEEIEQHEGNLSNLTREAALQVHIAQRDARRI

SEQ ID NO: 44 - protein, 81 aa, synthetic construct
  source 1..81: note = CD11_145
QSLANATAAQQEVLEAQYAMVQHIAKGIRILEARVARGGSGGNHTWQQWEEEIENHTGNLSLLLREAALQVHIAQRDARRI

SEQ ID NO: 45 - protein, 81 aa, synthetic construct
  source 1..81: note = CD11_135
QSLANATAAQQEVLEAQYAMVQHIAKGIRILEARVARGGSGGNHTWQQWEENITQHEGNLSLLLREAALQVHIAQRDARRI

SEQ ID NO: 46 - protein, 81 aa, synthetic construct
  source 1..81: note = CD11_125
QSLANATAAQQEVLEAQYAMVQHIAKGIRILEARVARGGSGGNHTWQNWTEEIEQHEGNLSLLLREAALQVHIAQRDARRI

SEQ ID NO: 47 - protein, 81 aa, synthetic construct
  source 1..81: note = CD11_15T
QSLANATAAQQEVLEAQYAMVQHIAKGIRILEARVARGGSGGNHTWQQWEEEIEQHEGNLTLLLREAALQVHIAQRDARRI

SEQ ID NO: 48 - protein, 81 aa, synthetic construct
  source 1..81: note = CD11_1
QSLANATAAQQEVLEAQYAMVQHIAKGIRILEARVARGGSGGNHTWQQWEEEIEQHEGQLTLLLREAALQVHIAQRDARRI

SEQ ID NO: 49 - protein, 81 aa, synthetic construct
  source 1..81: note = CD11_5T
QSLANATAAQQEVLEAQYAMVQHIAKGIRILEARVARGGSGGQHTWQQWEEEIEQHEGNLTLLLREAALQVHIAQRDARRI

SEQ ID NO: 50 - protein, 553 aa, synthetic construct
  source 1..553: note = RSV Fusion protein 66E T103-GS-G145 E511-GSG-HIV-GP41-based SSM referred to as F HIV-clamp
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGIVQQQNNLLRAIEAQQHLLQLTVWGIKQLQARILAGGSGGHTTWMEWDREINNYTSLIHSLIEESQNQQEKNEQELLE

SEQ ID NO: 51 - protein, 556 aa, synthetic construct
  source 1..556: note = RSV Fusion protein 66E T103-GS-G145 E511-GSG-VISNA-based SSM with additional N-linked glycosylation sequences (CD11_1245T8) referred to as VL22_CD11_1245T8
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGQSLANATAAQQEVLEAQYAMVQHIAKGIRILEARVARGGSGGNHTWQNWTEEIENHTGNLTLLLREAANQTHIAQRDARRI

SEQ ID NO: 52 - protein, 556 aa, synthetic construct
  source 1..556: note = RSV Fusion protein 66E T103-GS-G145 E511-GSG-VISNA-based SSM with additional N-linked glycosylation sequences (CD11_145T8) referred to as VL22_CD11_145T8
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGQSLANATAAQQEVLEAQYAMVQHIAKGIRILEARVARGGSGGNHTWQQWEEEIENHTGNLTLLLREAANQTHIAQRDARRI

SEQ ID NO: 53 - protein, 563 aa, synthetic construct
  source 1..563: note = RSV Fusion protein DS Cav1 Foldon referred to as DS Cav1 Foldon
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSGSAWSHPQFEK

SEQ ID NO: 54 - protein, 511 aa, synthetic construct
  source 1..511: note = RSV Fusion protein SC9-10 AY DS Cav1 Foldon referred to as SC9-10 AY DS Cav1 Foldon
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFLGGLVPR

SEQ ID NO: 55 - protein, 81 aa, synthetic construct
  source 1..81: note = CT1
QSLANATAAQQEVLEAQYAMVRHIAKGIRILEARVARGGSGGNHTWQQWEEEIEQHEGNLSLLLREAALQVHIAQRDARRI

SEQ ID NO: 56 - protein, 81 aa, synthetic construct
  source 1..81: note = CT2
QSLANATAAQQEVLEAQYAMVQHIAKGIRKLEARVARGGSGGNHTWQQWEEEIEQHEGNLSLLLREAALQVHIAQRDARRI

SEQ ID NO: 57 - protein, 81 aa, synthetic construct
  source 1..81: note = CT3
QSLANATAAQQEVLEAQYAMVQHIAKGIRILEARVARGGSGGNHTWQQWEEEIEQHEGNLSSLLREAALQVHIAQRDARRI

SEQ ID NO: 58 - protein, 81 aa, synthetic construct
  source 1..81: note = CT4
QSLANATAAQQEVLEAQYAMVQHIAKGIRILEARVARGGSGGNHTWQQWEEEIEQHEGNLSSKLREAALQVHIAQRDARRI

SEQ ID NO: 59 - protein, 81 aa, synthetic construct
  source 1..81: note = CT5
QSLANATAAQQEVLEAQYAMVQHIAKGIRILEARVARGGSGGHTTWQQWEEEIEQHEGNLSLLLREAALQVHIAQRDARRI

SEQ ID NO: 60 - protein, 81 aa, synthetic construct
  source 1..81: note = CT6
QSLANATAAQQEVLEAQYAMVQHIAKGIRILEARVARGGSGGNHTWQQWEEEIEQHEGNLSLLLRESQQQVHIAQRDARRI

SEQ ID NO: 61 - protein, 81 aa, synthetic construct
  source 1..81: note = CT7
QSLANATAAQQEVLEAQYAMVQHIAKGIRILEARVARGGSGGNHTWQQWEEEIEQHEGNLSLLLREAALQVHSAQRDARRI

SEQ ID NO: 62 - protein, 81 aa, synthetic construct
  source 1..81: note = CT8
QSLANATAAQLEVLEAQYAMVQHIAKGIRILEARVARGGSGGNHTWQQWEEEIEQHEGNLSLLLREAALQVHIAQRDARRI

SEQ ID NO: 63 - protein, 81 aa, synthetic construct
  source 1..81: note = CT9
QSLANATAAQQEVLEALYAMVQHIAKGIRILEARVARGGSGGNHTWQQWEEEIEQHEGNLSLLLREAALQVHIAQRDARRI

SEQ ID NO: 64 - protein, 80 aa, synthetic construct
  source 1..80: note = CT10
QSLANATAAQQEVLEAQYAMVQHIAKGIRILEARVAGGSGGNHTWQQWEEEIEQHEGNLSLLLREAALQVHIAQRDARRI

SEQ ID NO: 65 - protein, 84 aa, synthetic construct
  source 1..84: note = CT11
QSLANATAAQQEVLEAQYAMVQHIAKGIRILEARVARVEAGGSGGNHTWQQWEEEIEQHEGNLSLLLREAALQVHIAQRDARRI

SEQ ID NO: 66 - protein, 81 aa, synthetic construct
  source 1..81: note = CT12
QSLANATAAQQEVLEAQYAMVQHIAKGIRILEARVARPGSGGNHTWQQWEEEIEQHEGNLSLLLREAALQVHIAQRDARRI

SEQ ID NO: 67 - protein, 81 aa, synthetic construct
  source 1..81: note = CT13
QSLANATAAQQEVLEAQYAMVQHIAKGIRILEARVAPGGSGGNHTWQQWEEEIEQHEGNLSLLLREAALQVHIAQRDARRI

SEQ ID NO: 68 - protein, 81 aa, synthetic construct
  source 1..81: note = CT14
QSLANATAAQQEVLEAQYAMVQHIAKGIRILEARVARGGEGGNHTWQQWEEEIEQHEGNLSLLLREAALQVHIAQRDARRI

SEQ ID NO: 69 - protein, 75 aa, synthetic construct
QSLANATAAQQEVLEAQYAMVQHIAKGIRILEARVARGGSGGNHTWQQWEEEIEQHEGNLSLLLREAALQVHIAQ

SEQ ID NO: 70 - protein, 76 aa, synthetic construct
  source 1..76: note = CT16
QSLANATAAQQEVLEAQYAMVQHIAKGIRILEARVARGGSGGNHTWQQWEEEIEQHEGNLSLLLREAALQVHIAQR

SEQ ID NO: 71 - protein, 78 aa, synthetic construct
  source 1..78: note = CT17
QSLANATAAQQEVLEAQYAMVQHIAKGIRILEARVARGGSGGNHTWQQWEEEIEQHEGNLSLLLREAALQVHIAQRDA

SEQ ID NO: 72 - protein, 79 aa, synthetic construct
  source 1..79: note = CT18
QSLANATAAQQEVLEAQYAMVQHIAKGIRILEARVARGGSGGNHTWQQWEEEIEQHEGNLSLLLREAALQVHIAQRDAR

SEQ ID NO: 73 - protein, 1277 aa, synthetic construct
  source 1..1277: note = SARS-CoV-2 (Delta) Spike protein N679-GSG-S691 G1204-VISNA-based SSM CD11 referred to as SARS-CoV-2 (Delta) S CD11
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGQSLANATAAQQEVLEAQYAMVQHIAKGIRILEARVARGGSGGNHTWQQWEEEIEQHEGNLSLLLREAALQVHIAQRDARRI

SEQ ID NO: 74 - protein, 1275 aa, synthetic construct
  source 1..1275: note = SARS-CoV-2 (Delta) Spike protein N679-GSG-S691 G1204-VISNA-based SSM (CD11) and QS removed from CD11 FHRR referred to as SARS-CoV-2 (Delta) S CD11-QS
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGLANATAAQQEVLEAQYAMVQHIAKGIRILEARVARGGSGGNHTWQQWEEEIEQHEGNLSLLLREAALQVHIAQRDARRI

SEQ ID NO: 75 - protein, 554 aa, synthetic construct
  source 1..554: note = RSV Fusion protein 66E T103-GS-G145 E511-GSG-VISNA-based SSM (CD11) and QS removed from CD11 FHRR referred to as VL22_CD11-QS
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGSGGNHTWQQWEEEIEQHEGNLSLLLREAALQVHIAQRDARRI

SEQ ID NO: 76 - protein, 554 aa, synthetic construct
  source 1..554: note = RSV Fusion protein 66E T103-GS-G145 E511-GSG-VISNA-based SSM with additional N-linked glycosylation sequences and QS removed from CD11 FHRR (CD11_145T8-QS) referred to as VL22_CD11_145T8-QS
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGSGGNHTWQQWEEEIENHTGNLTLLLREAANQTHIAQRDARRI

SEQ ID NO: 77 - protein, 554 aa, synthetic construct
  source 1..554: note = RSV Fusion protein 66E T103-GS-G145 E511-GSG-VISNA-based SSM with additional N-linked glycosylation sequences and QS removed from CD11 FHRR and CT1 mutation included (CD11_145T8-QS) referred to as VL22_CD11_145T8-QS_CT1
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVRHIAKGIRILEARVARGGSGGNHTWQQWEEEIENHTGNLTLLLREAANQTHIAQRDARRI

SEQ ID NO: 78 - protein, 555 aa, synthetic construct
  source 1..555: note = RSV Fusion protein 66E T103-GS-G145 E511-GSG-VISNA-based SSM with additional N-linked glycosylation sequences and QS removed from CD11 FHRR and CT5 mutation included (CD11_145T8-QS) referred to as VL22_CD11_145T8-QS_CT5
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGSGGNHTTWQQWEEEIENHTGNLTLLLREAANQTHIAQRDARRI

SEQ ID NO: 79 - protein, 554 aa, synthetic construct; identical to SEQ ID NO: 76
  source 1..554: note = RSV Fusion protein 66E T103-GS-G145 E511-GSG-VISNA-based SSM with additional N-linked glycosylation sequences and QS removed from CD11 FHRR and CT9 mutation included (CD11_145T8-QS) referred to as VL22_CD11_145T8-QS_CT9

SEQ ID NO: 80 - protein, 37 aa, synthetic construct
  source 1..37: note = FHRR of CD11
QSLANATAAQQEVLEAQYAMVQHIAKGIRILEARVAR

SEQ ID NO: 81 - protein, 37 aa, synthetic construct
  source 1..37: note = FHRR of CD10
QTLANATAAQQDVLEATYAMVQHVAKGVRILEARVAR

SEQ ID NO: 82 - protein, 39 aa, synthetic construct
  source 1..39: note = SHRR of CD11
NHTWQQWEEEIEQHEGNLSLLLREAALQVHIAQRDARRI

SEQ ID NO: 83 - protein, 39 aa, synthetic construct
  source 1..39: note = SHRR of CD10
NHTWQQWERGLQGYDTNLTILLKESAAMTQLAEEQARRI

SEQ ID NO: 84 - protein, 5 aa, synthetic construct
  source 1..5: note = Linker sequence
GGSGG

SEQ ID NO: 85 - protein, 5 aa, synthetic construct
  source 1..5: note = Linker in CT12
PGSGG

SEQ ID NO: 86 - protein, 529 aa, synthetic construct
  source 1..529: note = Inf A HA Ectodomain 1-529
MKTIIAFSCILCLIFAQKLPGSDNSMATLCLGHHAVPNGTLVKTITDDQIEVTNATELVQSSSTGRICNSPHQILDGKNCTLIDALLGDPHCDDFQNKEWDLFVERSTAYSNCYPYYVPDYATLRSLVASSGNLEFTQESFNWTGVAQDGSSYACRRGSVNSFFSRLNWLYNLNYKYPEQNVTMPNNDKFDKLYIWGVHHPGTDKDQTNLYVQASGRVIVSTKRSQQTVIPNIGSRPWVRGVSSIISIYWTIVKPGDILLINSTGNLIAPRGYFKIQSGKSSIMRSDAHIDECNSECITPNGSIPNDKPFQNVNKITYGACPRYVKQNTLKLATGMRNVPEKQTRGIFGAIAGFIENGWEGMVDGWYGFRHQNSEGTGQAADLKSTQAAINQITGKLNRVIKKTNEKFHQIEKEFSEVEGRIQDLEKYVEDTKIDLWSYNAELLVALENQHTIDLTDSEMSKLFERTRRQLRENAEDMGNGCFKIYHKCDNACIGSIRNGTYDHDIYRNEALNNRFQIKGVQLKSGYKD

SEQ ID NO: 87 - protein, 512 aa, synthetic construct
  source 1..512: note = Inf A HA Ectodomain minus SP18-529
KLPGSDNSMATLCLGHHAVPNGTLVKTITDDQIEVTNATELVQSSSTGRICNSPHQILDGKNCTLIDALLGDPHCDDFQNKEWDLFVERSTAYSNCYPYYVPDYATLRSLVASSGNLEFTQESFNWTGVAQDGSSYACRRGSVNSFFSRLNWLYNLNYKYPEQNVTMPNNDKFDKLYIWGVHHPGTDKDQTNLYVQASGRVIVSTKRSQQTVIPNIGSRPWVRGVSSIISIYWTIVKPGDILLINSTGNLIAPRGYFKIQSGKSSIMRSDAHIDECNSECITPNGSIPNDKPFQNVNKITYGACPRYVKQNTLKLATGMRNVPEKQTRGIFGAIAGFIENGWEGMVDGWYGFRHQNSEGTGQAADLKSTQAAINQITGKLNRVIKKTNEKFHQIEKEFSEVEGRIQDLEKYVEDTKIDLWSYNAELLVALENQHTIDLTDSEMSKLFERTRRQLRENAEDMGNGCFKIYHKCDNACIGSIRNGTYDHDIYRNEALNNRFQIKGVQLKSGYKD

SEQ ID NO: 88 - protein, 452 aa, synthetic construct
  source 1..452: note = Inf A HA Ectodomain minus SP, minus MPER18 469
KLPGSDNSMATLCLGHHAVPNGTLVKTITDDQIEVTNATELVQSSSTGRICNSPHQILDGKNCTLIDALLGDPHCDDFQNKEWDLFVERSTAYSNCYPYYVPDYATLRSLVASSGNLEFTQESFNWTGVAQDGSSYACRRGSVNSFFSRLNWLYNLNYKYPEQNVTMPNNDKFDKLYIWGVHHPGTDKDQTNLYVQASGRVIVSTKRSQQTVIPNIGSRPWVRGVSSIISIYWTIVKPGDILLINSTGNLIAPRGYFKIQSGKSSIMRSDAHIDECNSECITPNGSIPNDKPFQNVNKITYGACPRYVKQNTLKLATGMRNVPEKQTRGIFGAIAGFIENGWEGMVDGWYGFRHQNSEGTGQAADLKSTQAAINQITGKLNRVIKKTNEKFHQIEKEFSEVEGRIQDLEKYVEDTKIDLWSYNAELLVALENQHTIDLTDSEMSKLFERTRR

SEQ ID NO: 89 - protein, 514 aa, synthetic construct
  source 1..514: note = Inf A HA Ectodomain 18 341, 346 - 529 plus altered furin cleavage sites
KLPGSDNSMATLCLGHHAVPNGTLVKTITDDQIEVTNATELVQSSSTGRICNSPHQILDGKNCTLIDALLGDPHCDDFQNKEWDLFVERSTAYSNCYPYYVPDYATLRSLVASSGNLEFTQESFNWTGVAQDGSSYACRRGSVNSFFSRLNWLYNLNYKYPEQNVTMPNNDKFDKLYIWGVHHPGTDKDQTNLYVQASGRVIVSTKRSQQTVIPNIGSRPWVRGVSSIISIYWTIVKPGDILLINSTGNLIAPRGYFKIQSGKSSIMRSDAHIDECNSECITPNGSIPNDKPFQNVNKITYGACPRYVKQNTLKLATGMRNVPERRRKKRGIFGAIAGFIENGWEGMVDGWYGFRHQNSEGTGQAADLKSTQAAINQITGKLNRVIKKTNEKFHQIEKEFSEVEGRIQDLEKYVEDTKIDLWSYNAELLVALENQHTIDLTDSEMSKLFERTRRQLRENAEDMGNGCFKIYHKCDNACIGSIRNGTYDHDIYRNEALNNRFQIKGVQLKSGYKD

SEQ ID NO: 90 - protein, 230 aa, synthetic construct
  source 1..230: note = Inf A HA Stem domain 1-58, 327-401, 442-509 plus linker regions
MKTIIALSYILCLVFAQKLPGNDNSTATLCLGHHAVPNGTIVKTITNDQIEVTNATELGFGQNTLKLATGMRNVPEKQTRGIFGAIAGFIENGWEGMLDGWYGFRHQNSEGRGQAADLKSTQAAIDQINGMLNRLIGSGGSGELLVALLNQHTIDLTDSEMNKLFEKTKKQLRENAEDMGNGCFKIYHKCDNACIGSIRNGTYDHDVYRDEALNNRFQIKGVELKSGYKD

SEQ ID NO: 91 - protein, 344 aa, synthetic construct
  source 1..344: note = Inf A HA Head domain 1-18, 51-328, 403-444 plus linker regions
MKTIIALSYILCLVFAQKEVTNATELVQNSSTGGICDSPHQILDGENCTLIDALLGDPQCDGFQNKKWDLFVERSKAYSNCYPYDVPDYASLRSLVASSGTLEFNNESFNWTGVTQNGTSSACKRGSNNSFFSRLNWLTHSKFKYPALNVTMPNNEEFDKLYIWGVHHPGTDNDQIFLYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNGSGGSGKTNEKFHQIEKEFSEVEGRIQDLEKYVEDTKIDLWSYNAELL

SEQ ID NO: 92 - protein, 547 aa, synthetic construct
  source 1..547: note = Inf B HA Ectodomain 1 547
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHT

SEQ ID NO: 93 - protein, 531 aa, synthetic construct
  source 1..531: note = Inf B HA Ectodomain minus SP 17-547
RICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHT

SEQ ID NO: 94 - protein, 471 aa, synthetic construct
  source 1..471: note = Inf B HA Ectodomain minus SP, minus MPER 17-487
RICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKML

SEQ ID NO: 95 - protein, 532 aa, synthetic construct
  source 1..532: note = Inf B HA Ectodomain minus SP plus altered furin cleavage sites 17-355, 362-547
RICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPARRRKKRAGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHT

SEQ ID NO: 96 - protein, 220 aa, synthetic construct
  source 1..220: note = Inf B HA Stem domain 1-47, 345-417, 457-547 plus linker regions
MKAIIVLLMVVTSNADRICTGITSSNSPHVVKTATQGEVNVTGVIPLGSGLANGTKYRPPAKLLKERGFFGAIAGFLEGGWEGMIAGWHGYTSHGAHGVAVAADLKSTQEAINKITKNLNYLSGSGGSGIELAVLLSNEGIINSEDEHLLALERKLKKMLGPSAVEIGNGCFETKHKCNQTCLDRIAAGTFNAGDFSLPTFDSLNITAASLNDDGLDNHT

SEQ ID NO: 97 - protein, 359 aa, synthetic construct
  source 1..359: note = Inf B HA Head domain 1-17, 48-344, 418-456 plus linker regions
MKAIIVLLMVVTSNADRTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKGSGGSGELEVKNLQRLSGAMNELHDEILELDEKVDDLRADTISSQ

SEQ ID NO: 98 - protein, 524 aa, synthetic construct
  source 1..524: note = RSV F Ectodomain 1 524
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCEIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 99 - protein, 520 aa, synthetic construct
  source 1..520: note = Ectodomain of RSV F (1-520)
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCEIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGK

SEQ ID NO: 100 - protein, 501 aa, synthetic construct
  source 1..501: note = RSV F Ectodomain minus SP 24-524
SGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCEIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 101 - protein, 501 aa, synthetic construct
  source 1..501: note = RSV F Ectodomain minus SP plus altered furin cleavage sites 24-524
SGQNITEEFYQSTCSAVSKGYLSALRTGWYTSVITIELSNIKKNKCNGTDAKVKLIKQELDKYKNAVTELQLLMQSTQATNNNANNELPRFMNYTLNNAKKTNVTLSNNNNNNFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCEIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 102 - protein, 87 aa, synthetic construct
  source 1..87: note = RSV F D25 interaction domain 61-97, 193-240 plus linker regions
LSNIKKNKCNGTDAKVKLIKQELDKYKNAVTELQLLMGGLDLKNYIDKQLLPIVNKQSCSISNIETVIEFQQKNNRLLEITREFSVN

SEQ ID NO: 103 - protein, 539 aa, synthetic construct
  source 1..539: note = illustrative hMPV F precursor
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 104 - protein, 490 aa, synthetic construct
  source 1..490: note = hMPV F Ectodomain 1 490
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTG

SEQ ID NO: 105 - protein, 471 aa, synthetic construct
  source 1..471: note = hMPV F Ectodomain minus SP 20-490
KESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTG

SEQ ID NO: 106 - protein, 471 aa, synthetic construct
  source 1..471: note = hMPV F Ectodomain minus SP plus altered furin cleavage sites 20-490
KESYLEESCSTITEGYLSVLRTGWYTNVFTLEVGDVENLTCADGPSLIKTELDLTKSALRELRTVSADQLAREEQIENPNQSNFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTG

SEQ ID NO: 107 - protein, 493 aa, synthetic construct
  source 1..493: note = PIV F Ectodomain 1 493
MPTSILLIITTMIMASFCQIDITKLQHVGVLVNSPKGMKISQNFETRYLILSLIPKIEDSNSCGDQQIKQYKRLLDRLIIPLYDGLRLQKDVIVSNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTVVTSDIVPRYAFVNGGVVANCITTTCTCNGIGNRINQPPDQGVKIITHKECNTIGINGMLFNTNKEGTLAFYTPNDITLNNSVALDPIDISIELNKAKSDLEESKEWIRRSNQKLDSIGNWHQSSTT

SEQ ID NO: 108 - protein, 474 aa, synthetic construct
  source 1..474: note = PIV F Ectodomain minus SP 20-493
IDITKLQHVGVLVNSPKGMKISQNFETRYLILSLIPKIEDSNSCGDQQIKQYKRLLDRLIIPLYDGLRLQKDVIVSNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTVVTSDIVPRYAFVNGGVVANCITTTCTCNGIGNRINQPPDQGVKIITHKECNTIGINGMLFNTNKEGTLAFYTPNDITLNNSVALDPIDISIELNKAKSDLEESKEWIRRSNQKLDSIGNWHQSSTT

SEQ ID NO: 109 - protein, 474 aa, synthetic construct
  source 1..474: note = PIV F Ectodomain minus SP plus altered furin cleavage sites 20-493
IDITKLQHVGVLVNSPKGMKISQNFETRYLILSLIPKIEDSNSCGDQQIKQYKRLLDRLIIPLYDGLRLQKDVIVSNQESNENTDPNTKNFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTVVTSDIVPRYAFVNGGVVANCITTTCTCNGIGNRINQPPDQGVKIITHKECNTIGINGMLFNTNKEGTLAFYTPNDITLNNSVALDPIDISIELNKAKSDLEESKEWIRRSNQKLDSIGNWHQSSTT

SEQ ID NO: 110 - protein, 493 aa, synthetic construct
  source 1..493: note = MeV F Ectodomain 1 493
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYLIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPILLERLDVGTNLGNAIAKLEDAKELLESSDQILRSMKGLSST

SEQ ID NO: 111 - protein, 469 aa, synthetic construct
  source 1..469: note = MeV F Ectodomain minus SP 25-493
IHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYLIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPILLERLDVGTNLGNAIAKLEDAKELLESSDQILRSMKGLSST

SEQ ID NO: 112 - protein, 469 aa, synthetic construct
  source 1..469: note = MeV F Ectodomain minus SP plus altered furin cleavage sites
IHWGNLSKIGVVGIGSASYKVMTRSSHQSLVIKLMPNITLLNNCTRVEIAEYRRLLRTVLEPIRDALNAMTQNIRPVQSVASSNNHKNFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYLIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPILLERLDVGTNLGNAIAKLEDAKELLESSDQILRSMKGLSST

SEQ ID NO: 113 - protein, 487 aa, synthetic construct
  source 1..487: note = HeV F Ectodomain 1 487
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPS

SEQ ID NO: 114 - protein, 467 aa, synthetic construct
  source 1..467: note = HeV F Ectodomain minus SP 21-487
SLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPS

SEQ ID NO: 115 - protein, 467 aa, synthetic construct
  source 1..467: note = HeV F Ectodomain minus SP plus altered furin cleavage sites 21-487
SLEGLGILHYEKLSKIGLVKGITRKYKIKSNPLTKDIVIKMIPNVSNVSKCTGTVMENYKSRLTGILSPIKGAIELYNNNTHDLVGDVNLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPS

SEQ ID NO: 116 - protein, 487 aa, synthetic construct
  source 1..487: note = NiV F Ectodomain 1 487
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPS

SEQ ID NO: 117 - protein, 467 aa, synthetic construct
  source 1..467: note = NiV F Ectodomain minus SP
SECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPS

SEQ ID NO: 118 - protein, 467 aa, synthetic construct
  source 1..467: note = NiV F Ectodomain minus SP plus altered furin cleavage sites
SECSVGILHYEKLSKIGLVKGVTRKYKIKSNPLTKDIVIKMIPNVSNMSQCTGSVMENYKTRLNGILTPIKGALEIYKNNTHDLVGDVNLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPS

SEQ ID NO: 119 - protein, 688 aa, synthetic construct
  source 1..688: note = HIV GP160 Ectodomain 1 688
MRVKGTRKNYWWRWGTMLLGMLMICSAAEQLWVTVYYGVPVWKEATTTLFCASDAKAVNTEVHNVWATHACVPTDPNPQEVVLENVTENFNMWKNDMVEQMQEDIISLWDQSLKPCVKLTPLCVTLNCTNWDGRNGTMNTTSTRNTTTANISRWEMEGEIKNCSFNVTTSIRNKMHKEYALFYKLDVMPIDNGSSYTLINCNTSVITQACPKVSFEPIPIHYCTPAGFALLKCNDKKFNGTGPCKNVSTVQCTHGIRPVVSTQLLLNGSLAEEEIVIRSENLTDNAKTIIVQLNETVVINCTRPGNNTRKSIHIGPGRAFYATGDIIGDIRQAHCNLSEASWNKTLKQIATKLREQFVNKTIIFNQSSGGDPEIVMHSFNCGGEFFYCDTTQLFNSAWFSNNTGLNYNNGSNTGGNITLPCRIKQIVNRWQEVGKAMYAPPIRGNITCSSNITGLLLTRDGGNNVTNESEIFRPGGGNMKDNWRSELYKYKVVKIEPLGVAPTRAKRRVVQREKRAVGTIGAMFLGFLGAAGSTMGAASLTLTVQARQLLSGIVQQQNNLLRAIEAQQHLLQLTVWGIKQLQARVLAVERYLKDQQLLGIWGCSGRLICTTAVPWNASWSNKSLDDIWNNMTWMQWEKEIDNYTGLIYRLIEESQTQQEKNEQDLLQLDTWASLWNWFSISNWLWYIK

SEQ ID NO: 120 - protein, 660 aa, synthetic construct
  source 1..660: note = HIV GP160 Ectodomain minus SP
EQLWVTVYYGVPVWKEATTTLFCASDAKAVNTEVHNVWATHACVPTDPNPQEVVLENVTENFNMWKNDMVEQMQEDIISLWDQSLKPCVKLTPLCVTLNCTNWDGRNGTMNTTSTRNTTTANISRWEMEGEIKNCSFNVTTSIRNKMHKEYALFYKLDVMPIDNGSSYTLINCNTSVITQACPKVSFEPIPIHYCTPAGFALLKCNDKKFNGTGPCKNVSTVQCTHGIRPVVSTQLLLNGSLAEEEIVIRSENLTDNAKTIIVQLNETVVINCTRPGNNTRKSIHIGPGRAFYATGDIIGDIRQAHCNLSEASWNKTLKQIATKLREQFVNKTIIFNQSSGGDPEIVMHSFNCGGEFFYCDTTQLFNSAWFSNNTGLNYNNGSNTGGNITLPCRIKQIVNRWQEVGKAMYAPPIRGNITCSSNITGLLLTRDGGNNVTNESEIFRPGGGNMKDNWRSELYKYKVVKIEPLGVAPTRAKRRVVQREKRAVGTIGAMFLGFLGAAGSTMGAASLTLTVQARQLLSGIVQQQNNLLRAIEAQQHLLQLTVWGIKQLQARVLAVERYLKDQQLLGIWGCSGRLICTTAVPWNASWSNKSLDDIWNNMTWMQWEKEIDNYTGLIYRLIEESQTQQEKNEQDLLQLDTWASLWNWFSISNWLWYIK

SEQ ID NO: 121 - protein, 639 aa, synthetic construct
  source 1..639: note = HIV GP160 Ectodomain minus SP, minus MPER
EQLWVTVYYGVPVWKEATTTLFCASDAKAVNTEVHNVWATHACVPTDPNPQEVVLENVTENFNMWKNDMVEQMQEDIISLWDQSLKPCVKLTPLCVTLNCTNWDGRNGTMNTTSTRNTTTANISRWEMEGEIKNCSFNVTTSIRNKMHKEYALFYKLDVMPIDNGSSYTLINCNTSVITQACPKVSFEPIPIHYCTPAGFALLKCNDKKFNGTGPCKNVSTVQCTHGIRPVVSTQLLLNGSLAEEEIVIRSENLTDNAKTIIVQLNETVVINCTRPGNNTRKSIHIGPGRAFYATGDIIGDIRQAHCNLSEASWNKTLKQIATKLREQFVNKTIIFNQSSGGDPEIVMHSFNCGGEFFYCDTTQLFNSAWFSNNTGLNYNNGSNTGGNITLPCRIKQIVNRWQEVGKAMYAPPIRGNITCSSNITGLLLTRDGGNNVTNESEIFRPGGGNMKDNWRSELYKYKVVKIEPLGVAPTRAKRRVVQREKRAVGTIGAMFLGFLGAAGSTMGAASLTLTVQARQLLSGIVQQQNNLLRAIEAQQHLLQLTVWGIKQLQARVLAVERYLKDQQLLGIWGCSGRLICTTAVPWNASWSNKSLDDIWNNMTWMQWEKEIDNYTGLIYRLIEESQTQQEKNEQDLLQ

SEQ ID NO: 122 - protein, 660 aa, synthetic construct
  source 1..660: note = HIV GP160 Ectodomain minus SP plus altered furin cleavage sites
EQLWVTVYYGVPVWKEATTTLFCASDAKAVNTEVHNVWATHACVPTDPNPQEVVLENVTENFNMWKNDMVEQMQEDIISLWDQSLKPCVKLTPLCVTLNCTNWDGRNGTMNTTSTRNTTTANISRWEMEGEIKNCSFNVTTSIRNKMHKEYALFYKLDVMPIDNGSSYTLINCNTSVITQACPKVSFEPIPIHYCTPAGFALLKCNDKKFNGTGPCKNVSTVQCTHGIRPVVSTQLLLNGSLAEEEIVIRSENLTDNAKTIIVQLNETVVINCTRPGNNTRKSIHIGPGRAFYATGDIIGDIRQAHCNLSEASWNKTLKQIATKLREQFVNKTIIFNQSSGGDPEIVMHSFNCGGEFFYCDTTQLFNSAWFSNNTGLNYNNGSNTGGNITLPCRIKQIVNRWQEVGKAMYAPPIRGNITCSSNITGLLLTRDGGNNVTNESEIFRPGGGNMKDNWRSELYKYKVVKIEPLGVAPTNANNNVVQREKRAVGTIGAMFLGFLGAAGSTMGAASLTLTVQARQLLSGIVQQQNNLLRAIEAQQHLLQLTVWGIKQLQARVLAVERYLKDQQLLGIWGCSGRLICTTAVPWNASWSNKSLDDIWNNMTWMQWEKEIDNYTGLIYRLIEESQTQQEKNEQDLLQLDTWASLWNWFSISNWLWYIK

SEQ ID NO: 123 - protein, 180 aa, synthetic construct
  source 1..180: note = HIV GP160 GP41 ectodomain 509-688
VVQREKRAVGTIGAMFLGFLGAAGSTMGAASLTLTVQARQLLSGIVQQQNNLLRAIEAQQHLLQLTVWGIKQLQARVLAVERYLKDQQLLGIWGCSGRLICTTAVPWNASWSNKSLDDIWNNMTWMQWEKEIDNYTGLIYRLIEESQTQQEKNEQDLLQLDTWASLWNWFSISNWLWYIK

SEQ ID NO: 124 - protein, 650 aa, synthetic construct
  source 1..650: note = EBOV GP Ectodomain 1 650
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQ

SEQ ID NO: 125 - protein, 623 aa, synthetic construct
  source 1..623: note = EBOV GP Ectodomain minus SP
QRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQ

SEQ ID NO: 126 - protein, 608 aa, synthetic construct
  source 1..608: note = EBOV GP Ectodomain minus SP, minus MPER
QRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTL

SEQ ID NO: 127 - protein, 608 aa, synthetic construct
  source 1..608: note = EBOV GP Ectodomain minus SP plus altered furin cleavage sites
QRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGNNTNNEAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTL

SEQ ID NO: 128 - protein, 475 aa, synthetic construct
  source 1..475: note = EBOV SP Ectodomain minus SP, minus mucin-like domain
QRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVGGNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQ

SEQ ID NO: 129 - protein, 468 aa, synthetic construct
  source 1..468: note = EBOV GP Ectodomain minus mucin-like domain
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTL

SEQ ID NO: 130 - protein, 650 aa, synthetic construct
  source 1..650: note = MARV GP Ectodomain 1 650
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWG

SEQ ID NO: 131 - protein, 631 aa, synthetic construct
  source 1..631: note = MARV GP Ectodomain minus SP 20-650
PILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWG

SEQ ID NO: 132 - protein, 608 aa, synthetic construct
  source 1..608: note = MARV GP Ectodomain minus SP, minus MPER 20-627
PILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQI

SEQ ID NO: 133 - protein, 631 aa, synthetic construct
  source 1..631: note = MARV GP Ectodomain minus SP plus altered furin cleavage sites
PILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFNNNNSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWG

SEQ ID NO: 134 - protein, 453 aa, synthetic construct
  source 1..453: note = MARV GP Ectodomain minus SP, minus mucin-like domain
PILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLGGAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWG

SEQ ID NO: 135 - protein, 1199 aa, synthetic construct
  source 1..1199: note = SARS-CoV S Ectodomain 1 1199
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTNVSAAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKGELDKYFKNHTSPDVDLGDISGINASVVNIQKEIDRLNEVAKNLNESLIDLQELGKYEQYIKWPWYVW

SEQ ID NO: 136 - protein, 1186 aa, synthetic construct
  source 1..1186: note = SARS-CoV S Ectodomain minus SP
SDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTNVSAAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKGELDKYFKNHTSPDVDLGDISGINASVVNIQKEIDRLNEVAKNLNESLIDLQELGKYEQYIKWPWYVW

SEQ ID NO: 137 - protein, 1174 aa, synthetic construct
  source 1..1174: note = SARS-CoV S Ectodomain minus SP, minus MPER
SDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTNVSAAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKGELDKYFKNHTSPDVDLGDISGINASVVNIQKEIDRLNEVAKNLNESLIDLQELGK

SEQ ID NO: 138 - protein, 1301 aa, synthetic construct
  source 1..1301: note = MERS-CoV S Ectodomain 1 1301
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLGVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQFNSSYFKLSIPTNFSFGVTQEYIQTTIQKVTVDCKQYICNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYSPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWL

SEQ ID NO: 139 - protein, 1280 aa, synthetic construct
  source 1..1280: note = MERS-CoV S Ectodomain minus SP 22-1301
GPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLGVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQFNSSYFKLSIPTNFSFGVTQEYIQTTIQKVTVDCKQYICNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYSPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWL

SEQ ID NO: 140 - protein, 1270 aa, synthetic construct
  source 1..1270: note = MERS-CoV S Ectodomain minus SP, minus MPER 22-1291
GPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLGVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQFNSSYFKLSIPTNFSFGVTQEYIQTTIQKVTVDCKQYICNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYSPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTY

SEQ ID NO: 141 - protein, 1280 aa, synthetic construct
  source 1..1280: note = MERS-CoV S Ectodomain minus SP plus altered furin cleavage sites
GPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLGVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPNSVNSVPGEMRLASIAFNHPIQVDQFNSSYFKLSIPTNFSFGVTQEYIQTTIQKVTVDCKQYICNGFQKCEQLLREYGQFCSKINQALHGANLRQDDSVRNLFASVKSSQSSPIIPGFGGDFNLTLLEPVSISTGSNSANSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYSPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWL

SEQ ID NO: 142 - protein, 462 aa, synthetic construct
  source 1..462: note = VSV G Ectodomain 1 462
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWK

SEQ ID NO: 143 - protein, 445 aa, synthetic construct
  source 1..445: note = VSV G Ectodomain minus SP
FTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWK

SEQ ID NO: 144 - protein, 404 aa, synthetic construct
  source 1..404: note = VSV G Ectodomain minus SP, minus MPER
FTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVF

SEQ ID NO: 145 - protein, 458 aa, synthetic construct
  source 1..458: note = RABV GP Ectodomain 1 458
MIPQTLLFVPLLVFSLCFGKFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVSDLDPYDKSLHSRVFPSGKCSGITVSSTYCPTNHDYTIWMPENPRLGTSCDIFTNSRGKRASKGSKTCGFVDERGLYKSLKGACKLKLCGVLGLRLMDGTWAAIQTSDEAKWCPPDQLVNIHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPSTVFKDGDEAEDFVEVHLPDVHKQVSGVDLGLPSWGK

SEQ ID NO: 146 - protein, 438 aa, synthetic construct
  source 1..438: note = RABV GP Ectodomain minus SP
FPIYTIPDKLGPWSPIDIHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVSDLDPYDKSLHSRVFPSGKCSGITVSSTYCPTNHDYTIWMPENPRLGTSCDIFTNSRGKRASKGSKTCGFVDERGLYKSLKGACKLKLCGVLGLRLMDGTWAAIQTSDEAKWCPPDQLVNIHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPSTVFKDGDEAEDFVEVHLPDVHKQVSGVDLGLPSWGK

SEQ ID NO: 147 - protein, 774 aa, synthetic construct
  source 1..774: note = HSV1 Gb Ectodomain 1 774
MRQGAPARGCRWFVVWALLGLTLGVLVASAAPSSPGTPGVAAATQAANGGPATPAPPALGAAPTGDPKPKKNKKPKNPTPPRPAGDNATVAAGHATLREHLRDIKAESTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNP

SEQ ID NO: 148 - protein, 750 aa, synthetic construct
  source 1..750: note = HSV1 Gb Ectodomain minus SP 25-774
VLVASAAPSSPGTPGVAAATQAANGGPATPAPPALGAAPTGDPKPKKNKKPKNPTPPRPAGDNATVAAGHATLREHLRDIKAESTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNP

SEQ ID NO: 149 - protein, 4 aa, synthetic construct
  source 1..4: note = linker sequence
GGSG

SEQ ID NO: 150 - protein, 4 aa, synthetic construct
  source 1..4: note = linker sequence
GSGS

SEQ ID NO: 151 - protein, 4 aa, synthetic construct
  source 1..4: note = linker sequence
GGGS

SEQ ID NO: 152 - protein, 5 aa, synthetic construct
  source 1..5: note = linker sequence
GGGGS

SEQ ID NO: 153 - protein, 10 aa, synthetic construct
  source 1..10: note = linker sequence
GGGGSGGGGS

SEQ ID NO: 154 - protein, 15 aa, synthetic construct
  source 1..15: note = linker sequence
GGGGSGGGGSGGGGS

SEQ ID NO: 155 - protein, 20 aa, synthetic construct
  source 1..20: note = linker sequence
GGGGSGGGGSGGGGSGGGGS

SEQ ID NO: 156 - protein, 6 aa, synthetic construct
  source 1..6: note = linker sequence
GGGGSG

SEQ ID NO: 157 - protein, 4 aa, synthetic construct
  source 1..4: note = linker sequence
GSGG

SEQ ID NO: 158 - protein, 5 aa, synthetic construct
  source 1..5: note = linker sequence
GSGGS

SEQ ID NO: 159 - protein, 4 aa, synthetic construct
  source 1..4: note = linker sequences comprises amino acid sequence
GGGG

SEQ ID NO: 160 - protein, 5 aa, synthetic construct
  source 1..5: note = linker sequences comprises amino acid sequence
GGGGG

SEQ ID NO: 161 - protein, 6 aa, synthetic construct
  source 1..6: note = linker sequence
GGSGGG

SEQ ID NO: 162 - protein, 10 aa, synthetic construct
  source 1..10: note = linker sequence
GGSGGGSGGG

SEQ ID NO: 163 - protein, 14 aa, synthetic construct
  source 1..14: note = linker sequence
GGSGGGSGGGSGGG

SEQ ID NO: 164 - protein, 18 aa, synthetic construct
  source 1..18: note = linker sequence
GGSGGGSGGGSGGGSGGG

SEQ ID NO: 165 - protein, 22 aa, synthetic construct
  source 1..22: note = linker sequence
GGSGGGSGGGSGGGSGGGSGGG

SEQ ID NO: 166 - protein, 26 aa, synthetic construct
  source 1..26: note = linker sequence
GGSGGGSGGGSGGGSGGGSGGGSGGG

SEQ ID NO: 167 - protein, 30 aa, synthetic construct
  source 1..30: note = linker sequence
GGSGGGSGGGSGGGSGGGSGGGSGGGSGGG

SEQ ID NO: 168 - protein, 34 aa, synthetic construct
  source 1..34: note = linker sequence
GGSGGGSGGGSGGGSGGGSGGGSGGGSGGGSGGG

SEQ ID NO: 169 - protein, 38 aa, synthetic construct
  source 1..38: note = linker sequence
GGSGGGSGGGSGGGSGGGSGGGSGGGSGGGSGGGSGGG

SEQ ID NO: 170 - protein, 42 aa, synthetic construct
  source 1..42: note = linker sequence
GGSGGGSGGGSGGGSGGGSGGGSGGGSGGGSGGGSGGGSGGG

SEQ ID NO: 171 - protein, 25 aa, synthetic construct
  source 1..25: note = linker sequence
GGGGSGGGGSGGGGSGGGGSGGGGS

SEQ ID NO: 172 - protein, 30 aa, synthetic construct
  source 1..30: note = linker sequence
GGGGSGGGGSGGGGSGGGGSGGGGSGGGGS

SEQ ID NO: 173 - protein, 35 aa, synthetic construct
  source 1..35: note = linker sequence
GGGGSGGGGSGGGGSGGGGSGGGGSGGGGSGGGGS

SEQ ID NO: 174 - protein, 40 aa, synthetic construct
  source 1..40: note = linker sequence
GGGGSGGGGSGGGGSGGGGSGGGGSGGGGSGGGGSGGGGS

SEQ ID NO: 175 - protein, 45 aa, synthetic construct
  source 1..45: note = linker sequence
GGGGSGGGGSGGGGSGGGGSGGGGSGGGGSGGGGSGGGGSGGGGS

SEQ ID NO: 176 - protein, 50 aa, synthetic construct
  source 1..50: note = linker sequence
GGGGSGGGGSGGGGSGGGGSGGGGSGGGGSGGGGSGGGGSGGGGSGGGGS

SEQ ID NO: 177 - protein, 10 aa, synthetic construct
  source 1..10: note = linker sequence
GGGGGGGGGG

SEQ ID NO: 178 - protein, 15 aa, synthetic construct
  source 1..15: note = linker sequence
GGGGGGGGGGGGGGG

SEQ ID NO: 179 - protein, 20 aa, synthetic construct
  source 1..20: note = linker sequence
GGGGGGGGGGGGGGGGGGGG

SEQ ID NO: 180 - protein, 25 aa, synthetic construct
  source 1..25: note = linker sequence
GGGGGGGGGGGGGGGGGGGGGGGGG

SEQ ID NO: 181 - protein, 30 aa, synthetic construct
  source 1..30: note = linker sequence
GGGGGGGGGGGGGGGGGGGGGGGGGGGGGG

SEQ ID NO: 182 - protein, 35 aa, synthetic construct
  source 1..35: note = linker sequence
GGGGGGGGGGGGGGGGGGGGGGGGGGGGGGGGGGG

SEQ ID NO: 183 - protein, 40 aa, synthetic construct
  source 1..40: note = linker sequence
GGGGGGGGGGGGGGGGGGGGGGGGGGGGGGGGGGGGGGGG

SEQ ID NO: 184 - protein, 45 aa, synthetic construct
  source 1..45: note = linker sequence
GGGGGGGGGGGGGGGGGGGGGGGGGGGGGGGGGGGGGGGGGGGGG

SEQ ID NO: 185 - protein, 50 aa, synthetic construct
  source 1..50: note = linker sequence
GGGGGGGGGGGGGGGGGGGGGGGGGGGGGGGGGGGGGGGGGGGGGGGGGG

SEQ ID NO: 186 - protein, 8 aa, synthetic construct
  source 1..8: note = linker sequence
GGGKGGGG

SEQ ID NO: 187 - protein, 16 aa, synthetic construct
  source 1..16: note = linker sequence
GGGKGGGGGGGKGGGG

SEQ ID NO: 188 - protein, 24 aa, synthetic construct
  source 1..24: note = linker sequence
GGGKGGGGGGGKGGGGGGGKGGGG

SEQ ID NO: 189 - protein, 32 aa, synthetic construct
  source 1..32: note = linker sequence
GGGKGGGGGGGKGGGGGGGKGGGGGGGKGGGG

SEQ ID NO: 190 - protein, 40 aa, synthetic construct
  source 1..40: note = linker sequence
GGGKGGGGGGGKGGGGGGGKGGGGGGGKGGGGGGGKGGGG

SEQ ID NO: 191 - protein, 48 aa, synthetic construct
  source 1..48: note = linker sequence
GGGKGGGGGGGKGGGGGGGKGGGGGGGKGGGGGGGKGGGGGGGKGGGG

SEQ ID NO: 192 - protein, 56 aa, synthetic construct
  source 1..56: note = linker sequence
GGGKGGGGGGGKGGGGGGGKGGGGGGGKGGGGGGGKGGGGGGGKGGGGGGGKGGGG

SEQ ID NO: 193 - protein, 64 aa, synthetic construct
  source 1..64: note = linker sequence
GGGKGGGGGGGKGGGGGGGKGGGGGGGKGGGGGGGKGGGGGGGKGGGGGGGKGGGGGGGKGGGG

SEQ ID NO: 194 - protein, 72 aa, synthetic construct
  source 1..72: note = linker sequence
GGGKGGGGGGGKGGGGGGGKGGGGGGGKGGGGGGGKGGGGGGGKGGGGGGGKGGGGGGGKGGGGGGGKGGGG

SEQ ID NO: 195 - protein, 80 aa, synthetic construct
  source 1..80: note = linker sequence
GGGKGGGGGGGKGGGGGGGKGGGGGGGKGGGGGGGKGGGGGGGKGGGGGGGKGGGGGGGKGGGGGGGKGGGGGGGKGGGG

SEQ ID NO: 196 - protein, 8 aa, synthetic construct
  source 1..8: note = linker sequence
GGGNGGGG

SEQ ID NO: 197 - protein, 16 aa, synthetic construct
  source 1..16: note = linker sequence
GGGNGGGGGGGNGGGG

SEQ ID NO: 198 - protein, 24 aa, synthetic construct
  source 1..24: note = linker sequence
GGGNGGGGGGGNGGGGGGGNGGGG

SEQ ID NO: 199 - protein, 32 aa, synthetic construct
  source 1..32: note = linker sequence
GGGNGGGGGGGNGGGGGGGNGGGGGGGNGGGG

SEQ ID NO: 200 - protein, 40 aa, synthetic construct
  source 1..40: note = linker sequence
GGGNGGGGGGGNGGGGGGGNGGGGGGGNGGGGGGGNGGGG

SEQ ID NO: 201 - protein, 48 aa, synthetic construct
  source 1..48: note = linker sequence
GGGNGGGGGGGNGGGGGGGNGGGGGGGNGGGGGGGNGGGGGGGNGGGG

SEQ ID NO: 202 - protein, 56 aa, synthetic construct
  source 1..56: note = linker sequence
GGGNGGGGGGGNGGGGGGGNGGGGGGGNGGGGGGGNGGGGGGGNGGGGGGGNGGGG

SEQ ID NO: 203 - protein, 64 aa, synthetic construct
  source 1..64: note = linker sequence
GGGNGGGGGGGNGGGGGGGNGGGGGGGNGGGGGGGNGGGGGGGNGGGGGGGNGGGGGGGNGGGG

SEQ ID NO: 204 - protein, 72 aa, synthetic construct
  source 1..72: note = linker sequence
GGGNGGGGGGGNGGGGGGGNGGGGGGGNGGGGGGGNGGGGGGGNGGGGGGGNGGGGGGGNGGGGGGGNGGGG

SEQ ID NO: 205 - protein, 80 aa, synthetic construct
  source 1..80: note = linker sequence
GGGNGGGGGGGNGGGGGGGNGGGGGGGNGGGGGGGNGGGGGGGNGGGGGGGNGGGGGGGNGGGGGGGNGGGGGGGNGGGG

SEQ ID NO: 206 - protein, 8 aa, synthetic construct
  source 1..8: note = linker sequence
GGGCGGGG

SEQ ID NO: 207 - protein, 16 aa, synthetic construct
  source 1..16: note = linker sequence
GGGCGGGGGGGCGGGG

SEQ ID NO: 208 - protein, 24 aa, synthetic construct
  source 1..24: note = linker sequence
GGGCGGGGGGGCGGGGGGGCGGGG

SEQ ID NO: 209 - protein, 32 aa, synthetic construct
  source 1..32: note = linker sequence
GGGCGGGGGGGCGGGGGGGCGGGGGGGCGGGG

SEQ ID NO: 210 - protein, 40 aa, synthetic construct
  source 1..40: note = linker sequence
GGGCGGGGGGGCGGGGGGGCGGGGGGGCGGGGGGGCGGGG

SEQ ID NO: 211 - protein, 48 aa, synthetic construct
  source 1..48: note = linker sequence
GGGCGGGGGGGCGGGGGGGCGGGGGGGCGGGGGGGCGGGGGGGCGGGG

SEQ ID NO: 212 - protein, 56 aa, synthetic construct
  source 1..56: note = linker sequence
GGGCGGGGGGGCGGGGGGGCGGGGGGGCGGGGGGGCGGGGGGGCGGGGGGGCGGGG

SEQ ID NO: 213 - protein, 64 aa, synthetic construct
  source 1..64: note = linker sequence
GGGCGGGGGGGCGGGGGGGCGGGGGGGCGGGGGGGCGGGGGGGCGGGGGGGCGGGGGGGCGGGG

SEQ ID NO: 214 - protein, 72 aa, synthetic construct
  source 1..72: note = linker sequence
GGGCGGGGGGGCGGGGGGGCGGGGGGGCGGGGGGGCGGGGGGGCGGGGGGGCGGGGGGGCGGGGGGGCGGGG

SEQ ID NO: 215 - protein, 80 aa, synthetic construct
  source 1..80: note = linker sequence
GGGCGGGGGGGCGGGGGGGCGGGGGGGCGGGGGGGCGGGGGGGCGGGGGGGCGGGGGGGCGGGGGGGCGGGGGGGCGGGG

SEQ ID NO: 216 - protein, 397 aa, synthetic construct
  source 1..397: note = Chlamydia trachomatis major outer membrane protein (MOMP) (as comprised in SEQ ID NO: 263)
MKKLLKSVLVFAALSSASSLQALPVGNPAEPSLMIDGILWEGFGGDPCDPCTTWCDAISMRVGYYGDFVFDRVLKTDVNKEFQMGAAPTTSDVAGLQNDPTINVARPNPAYGKHMQDAEMFTNAAYMALNIWDRFDVFCTLGATTGYLKGNSASFNLVGLFGTKTQSSSFNTAKLIPNTALNEAVVELYINTTFAWSVGARAALWECGCATLGASFQYAQSKPKVEELNVLCNASEFTINKPKGYVGAEFPLNITAGTEAATGTKDASIDYHEWQASLALSYRLNMFTPYIGVKWSRVSFDADTIRIAQPKLAEAILDVTTLNRTTAGKGSVVSAGTDNELADTMQIVSLQLNKMKSRKSCGIAVGTTIVDADKYAVTVEARLIDERAAHVNAQFRF

SEQ ID NO: 217 - protein, 389 aa, synthetic construct
  source 1..389: note = Chlamydia pneumoniae major outer membrane protein (MOMP); Uniprot entry: P27455
MKKLLKSALLSAAFAGSVGSLQALPVGNPSDPSLLIDGTIWEGAAGDPCDPCATWCDAISLRAGFYGDYVFDRILKVDAPKTFSMGAKPTGSAAANYTTAVDRPNPAYNKHLHDAEWFTNAGFIALNIWDRFDVFCTLGASNGYIRGNSTAFNLVGLFGVKGTTVNANELPNVSLSNGVVELYTDTSFSWSVGARGALWECGCATLGAEFQYAQSKPKVEELNVICNVSQFSVNKPKGYKGVAFPLPTDAGVATATGTKSATINYHEWQVGASLSYRLNSLVPYIGVQWSRATFDADNIRIAQPKLPTAVLNLTAWNPSLLGNATALSTTDSFSDFMQIVSCQINKFKSRKACGVTVGATLVDADKWSLTAEARLINERAAHVSGQFRF

SEQ ID NO: 218 - protein, 1566 aa, synthetic construct
  source 1..1566: note = E. coli EhaG-CD11
MNAGNDNGQGVDYGSGSAGDGWVAIGKGAKANTFMNTSGSSTAVGYDAIAEGQYSSAIGSKTHAIGGASMAFGVSAISEGDRSIALGASSYSLGQYSMALGRYSKALGKLSIAMGDSSKAEGANAIALGNATKATEIMSIALGDTANASKAYSMALGASSVASEENAIAIGAETEAAENATAIGNNAKAKGTNSMAMGFGSLADKVNTIALGNGSQALADNAIAIGQGNKADGVDAIALGNGSQSRGLNTIALGTASNATGDKSLALGSNSSANGINSVALGADSIADLDNTVSVGNSSLKRKIVNVKNGAIKSDSYDAINGSQLYAISDSVAKRLGGGAAVDVDDGTVTAPTYNLKNGSKNNVGAALAVLDENTLQWDQTKGKYSAAHGTSSPTASVITDVADGTISASSKDAVNGSQLKATNDDVEANTANIATNTSNIATNTANIATNTTNITNLTDSVGDLQADALLWNETKKAFSAAHGQDTTSKITNVKDADLTADSTDAVNGSQLKTTNDAVATNTTNIANNTSNIATNTTNISNLTETVTNLGEDALKWDKDNGVFTAAHGTETTSKITNVKDGDLTTGSTDAVNGSQLKTTNDAVATNTTNIATNTTNISNLTETVTNLGEDALKWDKDNGVFTAAHGNNTASKITNILDGTVTATSSDAINGSQLYDLSSNIATYFGGNASVNTDGVFTGPTYKIGETNYYNVGDALAAINSSFSTSLGDALLWDATAGKFSAKHGTNGDASVITDVADGEISDSSSDAVNGSQLHGVSSYVVDALGGGAEVNADGTITAPTYTIANADYDNVGDALNAIDTTLDDALLWDADAGENGAFSAAHGKDKTASVITNVANGAISAASSDAINGSQLYTTNKYIADALGGDAEVNADGTITAPTYTIANAEYNNVGDALDALDDNALLWDETANGGAGAYNASHDGKASIITNVANGSISEDSTDAVNGSQLNATNMMIEQNTQIINQLAGNTDATYIQENGAGINYVRTNDDGLAFNDASAQGVGATAIGYNSVAKGDSSVAIGQGSYSDVDTGIALGSSSVSSRVIAKGSRDTSITENGVVIGYDTTDGELLGALSIGDDGKYRQIINVADGSEAHDAVTVRQLQNAIGAVATTPTKYFHANSTEEDSLAVGTDSLAMGAKTIVNGDKGIGIGYGAYVDANALNGIAIGSNAQVIHVNSIAIGNGSTTTRGAQTNYTAYNMDAPQNSVGEFSVGSADGQRQITNVAAGSADTDAVNVGQLKVTDAQVSQNTQSITNLDNRVTNLDSRVTNIENGIGDIVTTGSTKYFKTNTDGVDASAQGKDSVAIGSGSIAAADNSVALGTGSVATEENTISVGSSTNQRRITNVAAGKNATDAVNVAQLKSSEAGGVRYDTKADGSIDYSNITLGGGNGGTTRISNVSAGVNNNDVVNYAQLKQSVQETKQYTDQRMVEMDNKLSKTESKLSGGIASAMAMTGLPQAYTPGGSGQSLANATAAQQEVLEAQYAMVQHIAKGIRILEARVARGGSGGNHTWQQWEEEIEQHEGNLSLLLREAALQVHIAQRDARRI

SEQ ID NO: 219 - protein, 518 aa, synthetic construct
  source 1..518: note = E. coli EibD-CD11
MQNGTYSVLQDDSQKSGPVKYGSTYEVVKTVDNGNFRYEVKEKKNDKRTLFKFDSEGNVTVKGKGITHTLHDPALKDFARTAEGKKNEQNGNTPPHKLTDSAVRGVYNKVYGLEKTEITGFSVEDGENGKVSLGSDAKASGEFSVAVGNGARATEKASTAVGSWAAADGKQSTALGVGTYAYANASTALGSVAFVDNTATYGTAAGNRAKVDKDATEGTALGAKATVTNKNSVALGANSVTTRDNEVYIGYKTGTESDKTYGTRVLGGLSDGTRNSDAATVGQLNRKVGGVYDDVKARITVESEKQKKYTDQKTSEVNEKVEARTTVGVDSDGKLTRAEGATKTIAVNDGLVALSGRTDRIDYAVGAIDGRVTRNTQSIEKNSKAIAANTRTLQQHSARLDSQQRQINENHKEMKRAAAQSAALTGLFQPYSVGGSGQSLANATAAQQEVLEAQYAMVQHIAKGIRILEARVARGGSGGNHTWQQWEEEIEQHEGNLSLLLREAALQVHIAQRDARRI

SEQ ID NO: 220 - protein, 1756 aa, synthetic construct
  source 1..1756: note = E. coli UpaG-CD11
MDNYTGQPTDYGDGSAGDGWVAIGKGAKANTFMNTSGASTALGYDAIAEGEYSSAIGSKTLATGGASMAFGVSAKAMGDRSVALGASSVANGDRSMAFGRYAKTNGFTSLAIGDSSLADGEKTIALGNTAKAYEIMSIALGDNANASKEYAMALGASSKAGGADSLAFGRKSTANSTGSLAIGADSSSSNDNAIAIGNKTQALGVNSMALGNASQASGESSIALGNTSEASEQNAIALGQGSIASKVNSIALGSNSLSSGENAIALGEGSAAGGSNSLAFGSQSRANGNDSVAIGVGAAAATDNSVAIGAGSTTDASNTVSVGNSATKRKIVNMAAGAISNTSTDAINGSQLYTISDSVAKRLGGGATVGSDGTVTAVSYALRSGTYNNVGDALSGIDNNTLQWNKTAGAFSANHGANATNKITNVAKGTVSATSTDVVNGSQLYDLQQDALLWNGTAFSAAHGTEATSKITNVTAGNLTAGSTDAVNGSQLKTTNDNVTTNTTNIATNTTNITNLTDAVNGLGDDSLLWNKAAGAFSAAHGTEATSKITNVTAGNLTAGSTDAVNGSQLKTTNDNVTTNTTNIATNTTNITNLTDAVNGLGDDSLLWNKTAGAFSAAHGTDATSKITNVTAGNLTAGSTDAVNGSQLKTTNDNVTTNTTNIATNTTNITNLTDAVNGLGDDSLLWNKTAGAFSAAHGTDATSKITNVKAGDLTAGSTDAVNGSQLKTTNDNVSTNTTNITNLTDAVNGLGDDSLLWNKTAGAFSAAHGTDATSKITNVKAGDLTAGSTDAVNGSQLKTTNDNVSTNTTNITNLTDSVGDLKDDSLLWNKAAGAFSAAHGTEATSKITNLLAGKISSNSTDAINGSQLYGVADSFTSYLGGGADISDTGVLSGPTYTIGGTDYTNVGDALAAINTSFSTSLGDALLWDATAGKFSAKHGINNAPSVITDVANGAVSSTSSDAINGSQLYGVSDYIADALGGNAVVNTDGSITTPTYAIAGGSYNNVGDALEAIDTTLDDALLWDTTANGGNGAFSAAHGKDKTASVITNVANGAVSATSNDAINGSQLYSTNKYIADALGGDAEVNADGTITAPTYTIANTDYNNVGEALDALDNNALLWDEDAGAYNASHDGNASKITNVAAGDLSTTSTDAVNGSQLNATNILVTQNSQMINQLAGNTSETYIEENGAGINYVRTNDSGLAFNDASASGIGATAVGYNAVASHASSVAIGQDSISEVDTGIALGSSSVSSRVIVKGTRNTSVSEEGVVIGYDTTDGELLGALSIGDDGKYRQIINVADGSEAHDAVTVRQLQNAIGAVATTPTKYYHANSTAEDSLAVGEDSLAMGAKTIVNGNAGIGIGLNTLVLADAINGIAIGSNARANHADSIAMGNGSQTTRGAQTNYTAYNMDAPQNSVGEFSVGSEDGQRQITNVAAGSADTDAVNVGQLKVTDAQVSQNTQSITNLNTQVTNLDTRVTNIENGIGDIVTTGSTKYFKTNTDGADANAQGKDSVAIGSGSIAAADNSVALGTGSVADEENTISVGSSTNQRRITNVAAGVNATDAVNVSQLKSSEAGGVRYDTKADGSIDYSNITLGGGNSGTTRISNVSAGVNNNDAVNYAQLKQSVQETKQYTDQRMVEMDNKLSKTESKLSGGIASAMAMTGLPQAYTPGGSGQSLANATAAQQEVLEAQYAMVQHIAKGIRILEARVARGGSGGNHTWQQWEEEIEQHEGNLSLLLREAALQVHIAQRDARRI

SEQ ID NO: 221 - protein, 1080 aa, synthetic construct
  source 1..1080: note = H. influenzae HiA-CD11
MNNNTPVTNKLKAYGDANFNFTNNSIADAEKQVQEAYKGLLNLNEKNASDKLLVEDNTAATVGNLRKLGWVLSSKNGTRNEKSQQVKHADEVLFEGKGGVQVTSTSENGKHTITFALAKDLGVKTATVSDTLTIGGGAAAGATTTPKVNVTSTTDGLKFAKDAAGANGDTTVHLNGIGSTLTDTLVGSPATHIDGGDQSTHYTRAASIKDVLNAGWNIKGVKAGSTTGQSENVDFVHTYDTVEFLSADTETTTVTVDSKENGKRTEVKIGAKTSVIKEKDGKLFTGKANKETNKVDGANATEDADEGKGLVTAKDVIDAVNKTGWRIKTTDANGQNGDFATVASGTNVTFASGNGTTATVTNGTDGITVKYDAKVGDGLKLDGDKIAADTTALTVNDGKNANNPKGKVADVASTDEKKLVTAKGLVTALNSLSWTTTAAEADGGTLDGNASEQEVKAGDKVTFKAGKNLKVKQEGANFTYSLQDALTGLTSITLGTGNNGAKTEINKDGLTITPANGAGANNANTISVTKDGISAGGQSVKNVVSGLKKFGDANFDPLTSSADNLTKQNDDAYKGLTNLDEKGTDKQTPVVADNTAATVGDLRGLGWVISADKTTGGSTEYHDQVRNANEVKFKSGNGINVSGKTVNGRREITFELAKGEVVKSNEFTVKETNGKETSLVKVGDKYYSKEDIDLTTGQPKLKDGNTVAAKYQDKGGKVVSVTDNTEATITNKGSGYVTGNQVADAIAKSGFELGLADEADAKAAFDDKTKALSAGTTEIVNAHDKVRFANGLNTKVSAATVESTDANGDKVTTTFVKTDVELPLTQIYNTDANGKKITKVVKDGQTKWYELNADGTADMTKEVTLGNVDSDGKKVVKDNDGKWYHAKADGTADKTKGEVSNDKVSTDEKHVVSLDPNDQSKGKGVVIDNVANGDISATSTDAINGSQLYAVAKGVTNLAGQVNNLEGKVNKVGKRADAGTASALAASQLPQATMPGGSGQSLANATAAQQEVLEAQYAMVQHIAKGIRILEARVARGGSGGNHTWQQWEEEIEQHEGNLSLLLREAALQVHIAQRDARRI

SEQ ID NO: 222 - protein, 412 aa, synthetic construct
  source 1..412: note = N. meningitidis NadA-CD11
MATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVGGSGQSLANATAAQQEVLEAQYAMVQHIAKGIRILEARVARGGSGGNHTWQQWEEEIEQHEGNLSLLLREAALQVHIAQRDARRI

SEQ ID NO: 223 - protein, 571 aa, synthetic construct
  source 1..571: note = N. meningitidis NhhA-CD11
MNNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGGSGQSLANATAAQQEVLEAQYAMVQHIAKGIRILEARVARGGSGGNHTWQQWEEEIEQHEGNLSLLLREAALQVHIAQRDARRI

SEQ ID NO: 224 - protein, 462 aa, synthetic construct
  source 1..462: note = Y. enterocolitica YadA-CD11
MDDYDGIPNLTAVQISPNADPALGLEYPVRPPVPGAGGLNASAKGIHSIAIGATAEAAKGAAVAVGAGSIATGVNSVAIGPLSKALGDSAVTYGAASTAQKDGVAIGARASTSDTGVAVGFNSKADAKNSVAIGHSSHVAANHGYSIAIGDRSKTDRENSVSIGHESLNRQLTHLAAGTKDTDAVNVAQLKKEIEKTQENTNKRSAELLANANAYADNKSSSVLGIANNYTDSKSAETLENARKEAFAQSKDVLNMAKAHSNSVARTTLETAEEHANSVARTTLETAEEHANKKSAEALASANVYADSKSSHTLKTANSYTDVTVSNSTKKAIRESNQYTDHKFRQLDNRLDKLDTRVDKGLASSAALNSLFQPYGVGGSGQSLANATAAQQEVLEAQYAMVQHIAKGIRILEARVARGGSGGNHTWQQWEEEIEQHEGNLSLLLREAALQVHIAQRDARRI

SEQ ID NO: 225 - protein, 577 aa, synthetic construct
  source 1..577: note = VL22 with membrane tethered silenced clamp2 alpha (VL22_CT5s_alpha)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGKRVIDGFLLILTSPIIIATAILSGGNHTTWQQWEEEIENHTGNLTLLLREAANQTHIAQRDARRI

SEQ ID NO: 226 - protein, 566 aa, synthetic construct
  source 1..566: note = VL22 with membrane tethered silenced clamp2 beta (VL22_CT5s_beta)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGYRLLSPDLLRYSGGNHTTWQQWEEEIENHTGNLTLLLREAANQTHIAQRDARRI

SEQ ID NO: 227 - protein, 578 aa, synthetic construct
  source 1..578: note = VL22 with membrane tethered silenced clamp2 gamma (VL22_CT5s_gamma)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGKAYEVAGLLGDIGGTNRIQYYFLSGGNHTTWQQWEEEIENHTGNLTLLLREAANQTHIAQRDARRI

SEQ ID NO: 228 - protein, 597 aa, synthetic construct
  source 1..597: note = VL22 with membrane tethered silenced clamp2 delta (VL22_CT5s_delta)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGLGDLILILRLPSWFKRLLSLLLKPLFPRLAAFLNSMRPRSAESGGNHTTWQQWEEEIENHTGNLTLLLREAANQTHIAQRDARRI

SEQ ID NO: 229 - protein, 587 aa, synthetic construct
  source 1..587: note = VL22 with membrane tethered silenced clamp2 epsilon (VL22_CT5s_epsilon)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGGTLNRFTQALVIAYVIGGGLALLGLVNVICDWSGGNHTTWQQWEEEIENHTGNLTLLLREAANQTHIAQRDARRI

SEQ ID NO: 230 - protein, 585 aa, synthetic construct
  source 1..585: note = VL22 with membrane tethered silenced clamp2 neta (VL22_CT5s_neta)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGKVLTIIATIFMPLGGGVIAVIMVVYFKKKKSGGNHTTWQQWEEEIENHTGNLTLLLREAANQTHIAQRDARRI

SEQ ID NO: 231 - protein, 577 aa, synthetic construct
  source 1..577: note = VL22 with membrane tethered silenced clamp2 theta (VL22_CT5s_theta)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGKEQLSTAIDRMNEGLDAFIQLYSGGNHTTWQQWEEEIENHTGNLTLLLREAANQTHIAQRDARRI

SEQ ID NO: 232 - protein, 580 aa, synthetic construct
  source 1..580: note = VL22 with membrane tethered silenced clamp2 eta (VL22_CT5s_eta)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDEGSGLANATAAQQEVLEAQYAMVQHIAKGIRILEARVARGGHWRAAGAATVLLGGVVVMVAGITSYSGGNHTTWQQWEEEIENHTGNLTLLLREAANQTHIAQRDARRI

SEQ ID NO: 233 - protein, 594 aa, synthetic construct
  source 1..594: note = VL22 with native transmembrane and cytoplasmic clamp2 (mVL22-TM-CD11)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKGSGLANATAAQQEVLEAQYAMVQHIAKGIRILEARVARGGSGGNHTWQQWEEEIEQHEGNLSLLLREAALQVHIAQRDARRI

SEQ ID NO: 234 - protein, 574 aa, synthetic construct; identical to SEQ ID NO: 23
  source 1..574: note = Native full length RSV F (mFwt)

SEQ ID NO: 235 - protein, 514 aa, synthetic construct
  source 1..514: note = DSCav1AY stabilized RSV F with transmembrane domain (mDSCavAY)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKAR

SEQ ID NO: 236 - protein, 22 aa, synthetic construct
  source 1..22: note = Membrane tethering (poly)peptide alpha (as comprised in SEQ ID NO: 225)
KRVIDGFLLILTSPIIIATAIL

SEQ ID NO: 237 - protein, 11 aa, synthetic construct
  source 1..11: note = Membrane tethering (poly)peptide beta (as comprised in SEQ ID NO: 226)
YRLLSPDLLRY

SEQ ID NO: 238 - protein, 23 aa, synthetic construct
  source 1..23: note = Membrane tethering (poly)peptide gamma (as comprised in SEQ ID NO: 227)
KAYEVAGLLGDIGGTNRIQYYFL

SEQ ID NO: 239 - protein, 42 aa, synthetic construct
  source 1..42: note = Membrane tethering (poly)peptide delta (as comprised in SEQ ID NO: 228)
LGDLILILRLPSWFKRLLSLLLKPLFPRLAAFLNSMRPRSAE

SEQ ID NO: 240 - protein, 32 aa, synthetic construct
  source 1..32: note = Membrane tethering (poly)peptide epsilon (as comprised in SEQ ID NO: 229)
GTLNRFTQALVIAYVIGGGLALLGLVNVICDW

SEQ ID NO: 241 - protein, 30 aa, synthetic construct
  source 1..30: note = Membrane tethering (poly)peptide neta (as comprised in SEQ ID NO: 230)
KVLTIIATIFMPLGGGVIAVIMVVYFKKKK

SEQ ID NO: 242 - protein, 22 aa, synthetic construct
  source 1..22: note = Membrane tethering (poly)peptide theta (as comprised in SEQ ID NO: 231)
KEQLSTAIDRMNEGLDAFIQLY

SEQ ID NO: 243 - protein, 26 aa, synthetic construct
  source 1..26: note = Membrane tethering (poly)peptide eta (as comprised in SEQ ID NO: 232)
HWRAAGAATVLLGGVVVMVAGITSYS

SEQ ID NO: 244 - protein, 27 aa, synthetic construct
  source 1..27: note = Membrane tethering (poly)peptide alpha plus flanking linker residues (as comprised in SEQ ID NO: 225)
GGKRVIDGFLLILTSPIIIATAILSGG

SEQ ID NO: 245 - protein, 16 aa, synthetic construct
  source 1..16: note = Membrane tethering (poly)peptide beta plus flanking linker residues (as comprised in SEQ ID NO: 226)
GGYRLLSPDLLRYSGG

SEQ ID NO: 246 - protein, 28 aa, synthetic construct
  source 1..28: note = Membrane tethering (poly)peptide gamma plus flanking linker residues (as comprised in SEQ ID NO: 227)
GGKAYEVAGLLGDIGGTNRIQYYFLSGG

SEQ ID NO: 247 - protein, 47 aa, synthetic construct
  source 1..47: note = Membrane tethering (poly)peptide delta plus flanking linker residues (as comprised in SEQ ID NO: 228)
GGLGDLILILRLPSWFKRLLSLLLKPLFPRLAAFLNSMRPRSAESGG

SEQ ID NO: 248 - protein, 37 aa, synthetic construct
  source 1..37: note = Membrane tethering (poly)peptide epsilon plus flanking linker residues (as comprised in SEQ ID NO: 229)
GGGTLNRFTQALVIAYVIGGGLALLGLVNVICDWSGG

SEQ ID NO: 249 - protein, 35 aa, synthetic construct
  source 1..35: note = Membrane tethering (poly)peptide neta plus flanking linker residues (as comprised in SEQ ID NO: 230)
GGKVLTIIATIFMPLGGGVIAVIMVVYFKKKKSGG

SEQ ID NO: 250 - protein, 27 aa, synthetic construct
  source 1..27: note = Membrane tethering (poly)peptide theta plus flanking linker residues (as comprised in SEQ ID NO: 231)
GGKEQLSTAIDRMNEGLDAFIQLYSGG

SEQ ID NO: 251 - protein, 30 aa, synthetic construct
  source 1..30: note = Membrane tethering (poly)peptide eta plus flanking linker residues (as comprised in SEQ ID NO: 232)
GGHWRAAGAATVLLGGVVVMVAGITSYSGG

SEQ ID NO: 252 - protein, 1481 aa, synthetic construct
  source 1..1481: note = E. coli EhaG (as comprised in SEQ ID NO: 218 )
MNAGNDNGQGVDYGSGSAGDGWVAIGKGAKANTFMNTSGSSTAVGYDAIAEGQYSSAIGSKTHAIGGASMAFGVSAISEGDRSIALGASSYSLGQYSMALGRYSKALGKLSIAMGDSSKAEGANAIALGNATKATEIMSIALGDTANASKAYSMALGASSVASEENAIAIGAETEAAENATAIGNNAKAKGTNSMAMGFGSLADKVNTIALGNGSQALADNAIAIGQGNKADGVDAIALGNGSQSRGLNTIALGTASNATGDKSLALGSNSSANGINSVALGADSIADLDNTVSVGNSSLKRKIVNVKNGAIKSDSYDAINGSQLYAISDSVAKRLGGGAAVDVDDGTVTAPTYNLKNGSKNNVGAALAVLDENTLQWDQTKGKYSAAHGTSSPTASVITDVADGTISASSKDAVNGSQLKATNDDVEANTANIATNTSNIATNTANIATNTTNITNLTDSVGDLQADALLWNETKKAFSAAHGQDTTSKITNVKDADLTADSTDAVNGSQLKTTNDAVATNTTNIANNTSNIATNTTNISNLTETVTNLGEDALKWDKDNGVFTAAHGTETTSKITNVKDGDLTTGSTDAVNGSQLKTTNDAVATNTTNIATNTTNISNLTETVTNLGEDALKWDKDNGVFTAAHGNNTASKITNILDGTVTATSSDAINGSQLYDLSSNIATYFGGNASVNTDGVFTGPTYKIGETNYYNVGDALAAINSSFSTSLGDALLWDATAGKFSAKHGTNGDASVITDVADGEISDSSSDAVNGSQLHGVSSYVVDALGGGAEVNADGTITAPTYTIANADYDNVGDALNAIDTTLDDALLWDADAGENGAFSAAHGKDKTASVITNVANGAISAASSDAINGSQLYTTNKYIADALGGDAEVNADGTITAPTYTIANAEYNNVGDALDALDDNALLWDETANGGAGAYNASHDGKASIITNVANGSISEDSTDAVNGSQLNATNMMIEQNTQIINQLAGNTDATYIQENGAGINYVRTNDDGLAFNDASAQGVGATAIGYNSVAKGDSSVAIGQGSYSDVDTGIALGSSSVSSRVIAKGSRDTSITENGVVIGYDTTDGELLGALSIGDDGKYRQIINVADGSEAHDAVTVRQLQNAIGAVATTPTKYFHANSTEEDSLAVGTDSLAMGAKTIVNGDKGIGIGYGAYVDANALNGIAIGSNAQVIHVNSIAIGNGSTTTRGAQTNYTAYNMDAPQNSVGEFSVGSADGQRQITNVAAGSADTDAVNVGQLKVTDAQVSQNTQSITNLDNRVTNLDSRVTNIENGIGDIVTTGSTKYFKTNTDGVDASAQGKDSVAIGSGSIAAADNSVALGTGSVATEENTISVGSSTNQRRITNVAAGKNATDAVNVAQLKSSEAGGVRYDTKADGSIDYSNITLGGGNGGTTRISNVSAGVNNNDVVNYAQLKQSVQETKQYTDQRMVEMDNKLSKTESKLSGGIASAMAMTGLPQAYTP

SEQ ID NO: 253 - protein, 433 aa, synthetic construct
  source 1..433: note = E. coli EibD (as comprised in SEQ ID NO: 219)
MQNGTYSVLQDDSQKSGPVKYGSTYEVVKTVDNGNFRYEVKEKKNDKRTLFKFDSEGNVTVKGKGITHTLHDPALKDFARTAEGKKNEQNGNTPPHKLTDSAVRGVYNKVYGLEKTEITGFSVEDGENGKVSLGSDAKASGEFSVAVGNGARATEKASTAVGSWAAADGKQSTALGVGTYAYANASTALGSVAFVDNTATYGTAAGNRAKVDKDATEGTALGAKATVTNKNSVALGANSVTTRDNEVYIGYKTGTESDKTYGTRVLGGLSDGTRNSDAATVGQLNRKVGGVYDDVKARITVESEKQKKYTDQKTSEVNEKVEARTTVGVDSDGKLTRAEGATKTIAVNDGLVALSGRTDRIDYAVGAIDGRVTRNTQSIEKNSKAIAANTRTLQQHSARLDSQQRQINENHKEMKRAAAQSAALTGLFQPYSV

SEQ ID NO: 254 - protein, 1671 aa, synthetic construct
  source 1..1671: note = E. coli UpaG (as comprised in SEQ ID NO: 220)
MDNYTGQPTDYGDGSAGDGWVAIGKGAKANTFMNTSGASTALGYDAIAEGEYSSAIGSKTLATGGASMAFGVSAKAMGDRSVALGASSVANGDRSMAFGRYAKTNGFTSLAIGDSSLADGEKTIALGNTAKAYEIMSIALGDNANASKEYAMALGASSKAGGADSLAFGRKSTANSTGSLAIGADSSSSNDNAIAIGNKTQALGVNSMALGNASQASGESSIALGNTSEASEQNAIALGQGSIASKVNSIALGSNSLSSGENAIALGEGSAAGGSNSLAFGSQSRANGNDSVAIGVGAAAATDNSVAIGAGSTTDASNTVSVGNSATKRKIVNMAAGAISNTSTDAINGSQLYTISDSVAKRLGGGATVGSDGTVTAVSYALRSGTYNNVGDALSGIDNNTLQWNKTAGAFSANHGANATNKITNVAKGTVSATSTDVVNGSQLYDLQQDALLWNGTAFSAAHGTEATSKITNVTAGNLTAGSTDAVNGSQLKTTNDNVTTNTTNIATNTTNITNLTDAVNGLGDDSLLWNKAAGAFSAAHGTEATSKITNVTAGNLTAGSTDAVNGSQLKTTNDNVTTNTTNIATNTTNITNLTDAVNGLGDDSLLWNKTAGAFSAAHGTDATSKITNVTAGNLTAGSTDAVNGSQLKTTNDNVTTNTTNIATNTTNITNLTDAVNGLGDDSLLWNKTAGAFSAAHGTDATSKITNVKAGDLTAGSTDAVNGSQLKTTNDNVSTNTTNITNLTDAVNGLGDDSLLWNKTAGAFSAAHGTDATSKITNVKAGDLTAGSTDAVNGSQLKTTNDNVSTNTTNITNLTDSVGDLKDDSLLWNKAAGAFSAAHGTEATSKITNLLAGKISSNSTDAINGSQLYGVADSFTSYLGGGADISDTGVLSGPTYTIGGTDYTNVGDALAAINTSFSTSLGDALLWDATAGKFSAKHGINNAPSVITDVANGAVSSTSSDAINGSQLYGVSDYIADALGGNAVVNTDGSITTPTYAIAGGSYNNVGDALEAIDTTLDDALLWDTTANGGNGAFSAAHGKDKTASVITNVANGAVSATSNDAINGSQLYSTNKYIADALGGDAEVNADGTITAPTYTIANTDYNNVGEALDALDNNALLWDEDAGAYNASHDGNASKITNVAAGDLSTTSTDAVNGSQLNATNILVTQNSQMINQLAGNTSETYIEENGAGINYVRTNDSGLAFNDASASGIGATAVGYNAVASHASSVAIGQDSISEVDTGIALGSSSVSSRVIVKGTRNTSVSEEGVVIGYDTTDGELLGALSIGDDGKYRQIINVADGSEAHDAVTVRQLQNAIGAVATTPTKYYHANSTAEDSLAVGEDSLAMGAKTIVNGNAGIGIGLNTLVLADAINGIAIGSNARANHADSIAMGNGSQTTRGAQTNYTAYNMDAPQNSVGEFSVGSEDGQRQITNVAAGSADTDAVNVGQLKVTDAQVSQNTQSITNLNTQVTNLDTRVTNIENGIGDIVTTGSTKYFKTNTDGADANAQGKDSVAIGSGSIAAADNSVALGTGSVADEENTISVGSSTNQRRITNVAAGVNATDAVNVSQLKSSEAGGVRYDTKADGSIDYSNITLGGGNSGTTRISNVSAGVNNNDAVNYAQLKQSVQETKQYTDQRMVEMDNKLSKTESKLSGGIASAMAMTGLPQAYTP

SEQ ID NO: 255 - protein, 995 aa, synthetic construct
  source 1..995: note = H. influenzae HiA (as comprised in SEQ ID NO: 221)
MNNNTPVTNKLKAYGDANFNFTNNSIADAEKQVQEAYKGLLNLNEKNASDKLLVEDNTAATVGNLRKLGWVLSSKNGTRNEKSQQVKHADEVLFEGKGGVQVTSTSENGKHTITFALAKDLGVKTATVSDTLTIGGGAAAGATTTPKVNVTSTTDGLKFAKDAAGANGDTTVHLNGIGSTLTDTLVGSPATHIDGGDQSTHYTRAASIKDVLNAGWNIKGVKAGSTTGQSENVDFVHTYDTVEFLSADTETTTVTVDSKENGKRTEVKIGAKTSVIKEKDGKLFTGKANKETNKVDGANATEDADEGKGLVTAKDVIDAVNKTGWRIKTTDANGQNGDFATVASGTNVTFASGNGTTATVTNGTDGITVKYDAKVGDGLKLDGDKIAADTTALTVNDGKNANNPKGKVADVASTDEKKLVTAKGLVTALNSLSWTTTAAEADGGTLDGNASEQEVKAGDKVTFKAGKNLKVKQEGANFTYSLQDALTGLTSITLGTGNNGAKTEINKDGLTITPANGAGANNANTISVTKDGISAGGQSVKNVVSGLKKFGDANFDPLTSSADNLTKQNDDAYKGLTNLDEKGTDKQTPVVADNTAATVGDLRGLGWVISADKTTGGSTEYHDQVRNANEVKFKSGNGINVSGKTVNGRREITFELAKGEVVKSNEFTVKETNGKETSLVKVGDKYYSKEDIDLTTGQPKLKDGNTVAAKYQDKGGKVVSVTDNTEATITNKGSGYVTGNQVADAIAKSGFELGLADEADAKAAFDDKTKALSAGTTEIVNAHDKVRFANGLNTKVSAATVESTDANGDKVTTTFVKTDVELPLTQIYNTDANGKKITKVVKDGQTKWYELNADGTADMTKEVTLGNVDSDGKKVVKDNDGKWYHAKADGTADKTKGEVSNDKVSTDEKHVVSLDPNDQSKGKGVVIDNVANGDISATSTDAINGSQLYAVAKGVTNLAGQVNNLEGKVNKVGKRADAGTASALAASQLPQATMP

SEQ ID NO: 256 - protein, 327 aa, synthetic construct
  source 1..327: note = N. meningitidis NadA (as comprised in SEQ ID NO: 222)
MATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNV

SEQ ID NO: 257 - protein, 486 aa, synthetic construct
  source 1..486: note = N. meningitidis NhhA (as comprised in SEQ ID NO: 223)
MNNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLP

SEQ ID NO: 258 - protein, 377 aa, synthetic construct
  source 1..377: note = Y. enterocolitica YadA (as comprised in SEQ ID NO: 224)
MDDYDGIPNLTAVQISPNADPALGLEYPVRPPVPGAGGLNASAKGIHSIAIGATAEAAKGAAVAVGAGSIATGVNSVAIGPLSKALGDSAVTYGAASTAQKDGVAIGARASTSDTGVAVGFNSKADAKNSVAIGHSSHVAANHGYSIAIGDRSKTDRENSVSIGHESLNRQLTHLAAGTKDTDAVNVAQLKKEIEKTQENTNKRSAELLANANAYADNKSSSVLGIANNYTDSKSAETLENARKEAFAQSKDVLNMAKAHSNSVARTTLETAEEHANSVARTTLETAEEHANKKSAEALASANVYADSKSSHTLKTANSYTDVTVSNSTKKAIRESNQYTDHKFRQLDNRLDKLDTRVDKGLASSAALNSLFQPYGV

SEQ ID NO: 259 - protein, 262 aa, synthetic construct
  source 1..262: note = Chlamydia trachomatis PGP3 (sequence corresponds to amino acid residues 3-264 of full-length protein)
NSGFYLYNTENCVFADNIKVGQMTEPLKDQQIILGTTSTPVAAKMTASDGISLTVSNNSSTNASITIGLDAEKAYQLILEKLGNQILDGIADTIVDSTVQDILDKITTDPSLGLLKAFNNFPITNKIQCNGLFTPSNIETLLGGTEIGKFTVTPKSSGSMFLVSADIIASRMEGSVVLALVREGDSKPCAISYGYSSGVPNLCSLRTSITNTGLTPTTYSLRVGGLESGVVWVNALSNGNDILGITNTSNVSFLEVIPQTNA

SEQ ID NO: 260 - protein, 563 aa, synthetic construct
  source 1..563: note = hMPV Fusion protein Clamp2s
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRIGSGLANATAAQQEVLEAQYAMVQHIAKGIRILEARVARGGSGGNHTTWQQWEEEIENHTGNLTLLLREAANQTHIAQRDARRI

SEQ ID NO: 261 - protein, 495 aa, synthetic construct
  source 1..495: note = HTLV-1 glycoprotein Clamp2s
MGWSCIILFLVATATGVHSESRCTLTIGVSSYHSKPCNPAQPVCSWTLDLLALSADQALQPPCPNLVSYSNYHATYSLYLFPHWIKKPNRNGGGYYSASYSDPCSLKCPYLGCQSWTCPYTGAVSSPYWKFQQDVNFTQEVSRLNINLHFSKCGFPFSLLVDAPGYDPIWLLNTEPSQLPPTAPPLLPHSNLDHILEPSIPWKSKLLTLVQLTLQSTNYTCIVCIDRASLSTWHVLYSPNISIPSSSSTPLLYPSLALPAPHLTLPFNWTHCFDPQIQAIVSSPCHNSLILPPFSLSPVPTLRSRSRRGGGGVSALAMGTGIAGGITGSMSLASGKNLLHEVDKDISQLTQAIVKNHKNLLKIAQYAAQNRRGLDLLFWEQGGLCKALQEQCCFLNITNSHVSILQERPPLEGSGLANATAAQQEVLEAQYAMVQHIAKGIRILEARVARGGSGGNHTTWQQWEEEIENHTGNLTLLLREAANQTHIAQRDARRI

SEQ ID NO: 262 - protein, 563 aa, synthetic construct
  source 1..563: note = hPIV3 Fusion protein Clamp2s
MPTSILLIITTMIMASFCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKGSGLANATAAQQEVLEAQYAMVQHIAKGIRILEARVARGGSGGNHTTWQQWEEEIENHTGNLTLLLREAANQTHIAQRDARRI

SEQ ID NO: 263 - protein, 479 aa, synthetic construct
  source 1..479: note = Chlamydia trachomatis Major Outer Membrane Protein Clamp2
MKKLLKSVLVFAALSSASSLQALPVGNPAEPSLMIDGILWEGFGGDPCDPCTTWCDAISMRVGYYGDFVFDRVLKTDVNKEFQMGAAPTTSDVAGLQNDPTINVARPNPAYGKHMQDAEMFTNAAYMALNIWDRFDVFCTLGATTGYLKGNSASFNLVGLFGTKTQSSSFNTAKLIPNTALNEAVVELYINTTFAWSVGARAALWECGCATLGASFQYAQSKPKVEELNVLCNASEFTINKPKGYVGAEFPLNITAGTEAATGTKDASIDYHEWQASLALSYRLNMFTPYIGVKWSRVSFDADTIRIAQPKLAEAILDVTTLNRTTAGKGSVVSAGTDNELADTMQIVSLQLNKMKSRKSCGIAVGTTIVDADKYAVTVEARLIDERAAHVNAQFRFGSGLANATAAQQEVLEAQYAMVQHIAKGIRILEARVARGGSGGNHTWQQWEEEIEQHEGNLSLLLREAALQVHIAQRDARRI

SEQ ID NO: 264 - protein, 346 aa, synthetic construct
  source 1..346: note = Chlamydia trachomatis PGP3 Clamp2
MANATAAQQEVLEAQYAMVQHIAKGIRILEARVARGGSGGNHTWQQWEEEIEQHEGNLSLLLREAALQVHIAQRDARRIGGSGGNSGFYLYNTENCVFADNIKVGQMTEPLKDQQIILGTTSTPVAAKMTASDGISLTVSNNSSTNASITIGLDAEKAYQLILEKLGNQILDGIADTIVDSTVQDILDKITTDPSLGLLKAFNNFPITNKIQCNGLFTPSNIETLLGGTEIGKFTVTPKSSGSMFLVSADIIASRMEGSVVLALVREGDSKPCAISYGYSSGVPNLCSLRTSITNTGLTPTTYSLRVGGLESGVVWVNALSNGNDILGITNTSNVSFLEVIPQTNA

SEQ ID NO: 265 - protein, 79 aa, synthetic construct
  source 1..79: note = Clamp2 (alternatively designated herein CD11-QS)
LANATAAQQEVLEAQYAMVQHIAKGIRILEARVARGGSGGNHTWQQWEEEIEQHEGNLSLLLREAALQVHIAQRDARRI

SEQ ID NO: 266 - protein, 80 aa, synthetic construct
  source 1..80: note = Clamp2s (alternatively designated herein as CD11_145T8-QS_CT5, which corresponds to silenced variant of Clamp2)
LANATAAQQEVLEAQYAMVQHIAKGIRILEARVARGGSGGNHTTWQQWEEEIENHTGNLTLLLREAANQTHIAQRDARRI

SEQ ID NO: 267 - protein, 37 aa, synthetic construct
  source 1..37: note = FHRR of HIV-clamp
SGIVQQQNNLLRAIEAQQHLLQLTVWGIKQLQARILA

SEQ ID NO: 268 - protein, 38 aa, synthetic construct
  source 1..38: note = SHRR of HIV-clamp
HTTWMEWDREINNYTSLIHSLIEESQNQQEKNEQELLE

SEQ ID NO: 269 - protein, 51 aa, synthetic construct
  source 1..51: note = FHRR of CD1
SITQYTKLSHQLISDVQAISSTIQDLQDQVDSLAEVVLQNRRGLDLLTAEQ

SEQ ID NO: 270 - protein, 38 aa, synthetic construct
  source 1..38: note = SHRR of CD1
ICLALQEKCSFYANKSGIVRDKIKNLQDDLERRRRQLI

SEQ ID NO: 271 - protein, 37 aa, synthetic construct
  source 1..37: note = FHRR of CD2
SITQYTKLSHQLISDVQAISSTIQDLQDQVDSLAEVV

SEQ ID NO: 272 - protein, 26 aa, synthetic construct
  source 1..26: note = SHRR of CD2
ANKSGIVRDKIKNLQDDLERRRRQLI

SEQ ID NO: 273 - protein, 47 aa, synthetic construct
  source 1..47: note = FHRR of CD3
FEQLQAAIHTDLGALEKSVSALEKSLTSLSEVVLQNRRGLDLLFLKE

SEQ ID NO: 274 - protein, 32 aa, synthetic construct
  source 1..32: note = SHRR of CD3
LCAALKEECSFYADHTGVVRDSMAKLRERLNQ

SEQ ID NO: 275 - protein, 33 aa, synthetic construct
  source 1..33: note = FHRR of CD4
FEQLQAAIHTDLGALEKSVSALEKSLTSLSEVV

SEQ ID NO: 276 - protein, 21 aa, synthetic construct
  source 1..21: note = SHRR of CD4
YADHTGVVRDSMAKLRERLNQ

SEQ ID NO: 277 - protein, 44 aa, synthetic construct
  source 1..44: note = FHRR of CD5
LQIAMDTDLRALQDSISKLEDSLTSLSEVVLQNRRGLDLLFLKE

SEQ ID NO: 278 - protein, 30 aa, synthetic construct
  source 1..30: note = SHRR of CD5
LCAALKEECSFYVDHSGAVRDSMRRLKERL

SEQ ID NO: 279 - protein, 27 aa, synthetic construct
  source 1..27: note = FHRR of CD6
AMDTDLRALQDSISKLEDSLTSLSEVV

SEQ ID NO: 280 - protein, 19 aa, synthetic construct
  source 1..19: note = SHRR of CD6
YVDHSGAVRDSMRRLKERL

SEQ ID NO: 281 - protein, 37 aa, synthetic construct
  source 1..37: note = FHRR of CD7
ATHQEAIEKVTEALKINNLRLVTLEHQVLVIGLKVEA

SEQ ID NO: 282 - protein, 37 aa, synthetic construct
  source 1..37: note = SHRR of CD7
NQTIWNHGNITLGEWYNKTKDLQQKFYEIIMDIEQNN

SEQ ID NO: 283 - protein, 37 aa, synthetic construct
  source 1..37: note = FHRR of CD8
AGIVQQQQQLLDVVKRQQELLRLTVWGTKNLQTRVTA

SEQ ID NO: 284 - protein, 38 aa, synthetic construct
  source 1..38: note = SHRR of CD8
NNETWQEWERKVDFLEENITALLEEAQIQQEKNMYELQ

SEQ ID NO: 285 - protein, 37 aa, synthetic construct
  source 1..37: note = FHRR of CD9
QSLANATAAQQEVLEASYAMVQHIAKGIRILEARVAR

SEQ ID NO: 286 - protein, 39 aa, synthetic construct; identical to SEQ ID NO: 82
  source 1..39: note = SHRR of CD9

SEQ ID NO: 287 - protein, 31 aa, synthetic construct
  source 1..31: note = FHRR of CK1
EFSDMLRLFDFNKQAIQRLKAEAQMSIQLIN

SEQ ID NO: 288 - protein, 38 aa, synthetic construct
  source 1..38: note = SHRR of CK1
NETHFSDDIEQQADNMITEMLQKEYMERQGKTPLGLVD

SEQ ID NO: 289 - protein, 36 aa, synthetic construct
  source 1..36: note = FHRR of CK2
ADSVQNHTFEVENNTINGLELTEEQVHILYAMVLQT

SEQ ID NO: 290 - protein, 36 aa, synthetic construct
  source 1..36: note = SHRR of CK2
TQWDDWVDKMENLNHDILTTLHTARNNLEQSMITFN

SEQ ID NO: 291 - protein, 34 aa, synthetic construct
  source 1..34: note = FHRR of CK3
EAMKNADNINKLKSSIESTNEAVVKLQETAEKTV

SEQ ID NO: 292 - protein, 33 aa, synthetic construct
  source 1..33: note = SHRR of CK3
DISSQISSMNQSLQQSKDYIKEAQRLLDTVNPS

SEQ ID NO: 293 - protein, 36 aa, synthetic construct
  source 1..36: note = FHRR of CK4
DLQQGLTSLQIAMDTDLRALQDSISKLEDSLTSLSE

SEQ ID NO: 294 - protein, 32 aa, synthetic construct
  source 1..32: note = SHRR of CK4
FYVDHSGAVRDSMKKLKERLDKRQLERQKNQN

SEQ ID NO: 295 - protein, 36 aa, synthetic construct; identical to SEQ ID NO: 293
  source 1..36: note = FHRR of CK5

SEQ ID NO: 296 - protein, 32 aa, synthetic construct
  source 1..32: note = SHRR of CK5
FYVDHSGAVRDSMRRLKERLDKRQLEHQKNLS

SEQ ID NO: 297 - protein, 37 aa, synthetic construct
  source 1..37: note = FHRR of CK6
QQLEKGLSNLHRIVTEDLQALEKSVSNLEESLTSLSE

SEQ ID NO: 298 - protein, 32 aa, synthetic construct
  source 1..32: note = SHRR of CK6
FYVDHSGAIRDSMNKLRERLEKRRREKETTQG

SEQ ID NO: 299 - protein, 39 aa, synthetic construct
  source 1..39: note = FHRR of CK7
KALIETAQFRQLQMAMHTDIQALEESISALEKSLTSLSE

SEQ ID NO: 300 - protein, 35 aa, synthetic construct
  source 1..35: note = SHRR of CK7
FYADHTGLVRDNMAKLRERLKQRQQLFDSQQGWFE

SEQ ID NO: 301 - protein, 38 aa, synthetic construct
  source 1..38: note = FHRR of CK8
STLSHQRLTSLIHVLEQDQQRLITAINQTHYNLLNVAS

SEQ ID NO: 302 - protein, 25 aa, synthetic construct
  source 1..25: note = SHRR of CK8
FLRIQNDSIIRLGDLQPLSQRVSTD